(19) United States
(12) Patent Application Publication
(10) Pub. No.: US 2020/0188660 A1
(43) Pub. Date: Jun. 18, 2020

(54) ELECTRODE CURED AND MANUFACTURED IN THE BODY, AND RELATED METHODS AND DEVICES

(71) Applicant: NEURONOFF, INC., Valencia, CA (US)

(72) Inventors: Manfred FRANKE, Valencia, CA (US); Andrew J. SHOFFSTALL, Aurora, OH (US); Elias VEIZI, Akron, OH (US); John w. SHEETS, Virginia Beach, VA (US)

(21) Appl. No.: 16/620,499

(22) PCT Filed: Jun. 8, 2018
(86) PCT No.: PCT/US2018/036773

§ 371 Date: Dec. 7, 2019

Related U.S. Application Data
(63) Continuation-in-part of application No. PCT/US2017/065929, filed on Sep. 28, 2012.
(60) Provisional application No. 62/643,017, filed on Mar. 14, 2018.
(60) Provisional application No. 62/643,543, filed on Mar. 15, 2018.
(60) Provisional application No. 62/599,533, filed on Dec. 15, 2017.
(60) Provisional application No. 62/564,809, filed on Sep. 28, 2017.
(60) Provisional application No. 62/537,294, filed on Jul. 26, 2017.
(60) Provisional application No. 62/517,082, filed on Jun. 8, 2017.

(51) Int. Cl. A61N 1/36; A61N 1/40

(52) U.S. Cl. CPC A61N 1/36002; A61N 1/36071; A61B 2018/1472; A61N 1/36135; A61N 1/406

(57) ABSTRACT

An injectable electrode which is manufactured in the body by curing from a liquid phase to a solid phase, and therefore molding to the contours of the bodily structures where it is injected.

### RELATED APPLICATIONS

This application claims priority to, and the full benefit of, the following US Provisional Patent Applications: 62/517,082 filed on Jun. 8, 2017; 62/537,294 filed on Jul. 26, 2017; 62/564,809 filed on Sep. 28, 2017; 62/599,533 filed on Dec. 15, 2017; 62/643,017 filed on Mar. 14, 2018; and 62/643,543 filed on Mar. 15, 2018 and incorporates each of them fully as if set forth herein. This application also claims priority to, and the full benefit of, PCT application PCT/US17/65929 filed on Dec. 12, 2017.

### BACKGROUND

Bioelectronic medicine is the application of electronic devices to address medical problems. Prior art biocompatible electrodes, however, have many problems and limitations which have limited bioelectronic medicine to date. Electrodes provide the interface from generally a metallic path for electrical current to the ionic environment surrounding a target such as the interstitial fluid inside in a bodily tissue, whether in a body or in a sample severed for research purposes. In prior art electrodes, regardless of the electrode's material that supplies the mechanical structure, the metal at the actual contact with the target in bodily tissue (“the interface”) is comprised of pre-shaped wires or metallic traces having limited flexibility or ability to be shaped to conform to the unique contours of a target in a bodily tissue. The targets in bodily tissue vary greatly in size and shape. For example, in the peripheral nervous system (“PNS”) neural plexi are highly irregularly shaped bundles of nerves, an example of which is a human brachial plexus as shown in the diagram in FIG. 1A. Similarly, PNS ganglia differ from the cylindrical or oval shape of a PNS nerve like the median nerve in the arm, and ganglia have many different shapes. An image of a rat cranial nerve ganglion is shown in FIG. 1B. A single peripheral nerve in a limb can be cylindrical and fall within a wide range of diameters, from 1 to 25 mm. The range of sizes of the same ganglia also varies greatly among individual humans. One study reports human superior cervical sympathetic chain ganglia as having an axial diameter of 7.7 mm+/−1.8 mm with a range of 4.8-13.2 mm., showing very wide variability among only 53 subjects. (Lee, et al., Superior Cervical Sympathetic Ganglion: Normal Imaging Appearance on 3T-MRI, Korean J. Radiol 1016 September-October; 17(5): 657-663) A cranial nerve can be, at the point of interface, between about 1 and 5 mm and cylindrical. Thus, there is great variability among the sizes and particular shapes of a particular target from individual to individual. A one-size-fits-all neural interface electrode has many problems relating to fit.

Implantation of prior art electrodes generally requires a surgical approach far more invasive than the injection of a drug by needle. In fact, most prior art electrodes designed for a good signal to noise ratio (“SNR”) in neural sensing or selective stimulation applications for the PNS require the surgeon to have a line of sight access to the target in bodily tissue which generally requires a reasonably large incision, blunt dissection and release of the nerve from the adjoining tissue. To describe the invention herein, it is first helpful to point to several prior art electrodes, and to set forth figures showing them.

Most prior art devices for use in bodily tissues do not conform to the contours of the target in bodily tissue, and their shapes in fact are sometimes dictated by the production processes by which they are made. For example, a flat electrode is produced by silicon wafer production techniques with needles extending from the metal contacts from a planar surface, as shown in FIG. 2A and FIG. 2A from U.S. Pat. No. 5,215,088. Another prior art planar electrode from US20150367124 is shown in FIG. 3A and FIG. 3B. These prior art electrodes cannot be easily modified or adapted for use on other targets besides the specific location for which they are designed, and they are not sized according to the individual, and they are therefore limited in adaptability to the many different anatomical shapes and sizes and varied targets. For example, PNS ganglia and plexi have a host of irregular shapes whereas the median nerve in the arm is linear. Not only does the general size of prior art devices result in target mismatch, but the preset locations of the individual electrical contacts in prior art devices also present great potential for mismatch in a given implantation.

Another prior art device is a cuff electrode which is generally a strip of non-conductive material with wiring to metal electrode contacts and the device is wrapped around a PNS target, as shown in the diagram in FIG. 4A from US20060030919 A1 and the image in FIG. 4B (http://www.ardiemmedical.com/wordpress1/wp-content/uploads/2011/01/Cuff-Electrode.jpg)

Prior art deep brain stimulation electrodes have a generally rod-like shape, as shown for example in FIG. 5, which is from US20110191275. Another rod shaped electrode is FIG. 6 from U.S. Pat. No. 8,473,062. FIG. 5 and FIG. 6 depict rod-shaped electrode configurations with one or several electrode contacts aligned linearly. Electrical field lines between two contacts on the same electrode and a distal return are not equidistant and not homogeneous. Attempting to stimulate a neural target next to the rod is not an easy task when other neural side targets are close by. One advantage of rod shaped electrodes 40 is that they are, compared to other prior art electrodes, easier to place through a tunneled approach. That is, the rod shape has a narrow width and the surgeon can implant the entire electrode and electrode system through a keyhole incision and advance the electrode deep into the body to the neural stimulation target structure. There are, however, significant disadvantages. The electrical field emanating from these electrodes is that of a point source instead of a homogeneous field like inside a ring electrode that is placed around a nerve. FIG. 7 shows the rod-shaped electrodes 40 in FIG. 5 and electrical contacts which may have a single electrode contact or a multitude of electrode contacts, here labeled 1-4. Electrical field lines 73 in group B between contacts 1 and 4 and field lines 73 in group A between contacts 2 and 3 on the same electrode and a distal return are not equidistant and not homogeneous. Also, field lines 73 in group C are directed in almost 360 degrees, and can have unintended effects. Attempting to stimulate a neural target (shaded area in FIG. 7 to the right of the electrode) next to the rod is not an easy task when other neural side targets are close by.

The process of encapsulation of the electrode by connective tissue can migrate the electrode away from the nerve. This can change the electrical field lines 73 so much that waveform parameters used for successful stimulation of said nerve might not work after a few weeks, or once an encapsulation has formed as a result of the normal bodily encapsulation response to a foreign, introduced object. The point source will generally depolarize the fascicle(s) inside the nerve that are mechanically closest to the electrode. While this may add selectivity, it also can add unwanted effects of stimulating all small and large fibers of a fascicle closer to the electrode while the large fibers in a more distant fascicle might not be activated, even though the goal of the stimulation might be to activate all large fibers in all the fascicles of a given nerve. A uniform electrical field as can be provided by a ring of metal placed around the nerve as done with a cuff electrode can achieve this equal activation of fibers of the same size in a nerve.

There are additional problems as well. The surgical procedure necessary to insert a large pre-configured electrode next to a biological target can cause great trauma to the target 5 or in the immediate area, causing bleeding and a large inflammatory response which can lead to excessive growth of connective tissue between the electrode interface and the target (such as data presented in FIG. 8). The distance between the prior art device and the target contours can be too great, allowing an insufficient transfer of current and providing unneeded space allowing growth of connective tissue which has significantly higher impedance than interstitial fluid. The fall off of an electric field each contact of a bipolar electrode is 1/r\hat{ }2, where r is the distance from each electrode contact. This means for a unipolar electrode that the normalized field strength at 100 μm distance from the edge of the electrode and mostly only partly into the nerve, only 10% of the initial field strength at the electrode edge may be available. This value drops to about 1/100th for a tripolar electrode. Electrodes that fit more tightly all around the nerve or more tightly against a nerve and are able to provide a more uniform field throughout the nerve (such as by fully surrounding a nerve in a ring like structure such as a cuff) are able to achieve a nerve fiber recruitment profile that is primarily based on fiber diameter and less on fiber location with respect to the edge of the electrode, causing only the outside fibers in a nerve to depolarize if at all. Plonsey, R. Quantitative formulations of electrophysiological sources of potential fields in volume mixtures. IEEE Trans Biomed Eng 31, 868-872 (1984), and Barr, R. C. & Plonsey, R. Propagation of excitation in idealized anisotropic two-dimensional tissue. Biophys J 45, 1191-1202 (1984). Post-implantation in chronic usage, prior art devices have great potential to cause irritation of surrounding tissues and further inflammatory action. Also, a prior art device placed next to a target (without enveloping it) will depolarize the target partially but likely not fully (i.e., the areas more distant from the location of the lead remain in their pre-implantation voltage states). Many prior art devices are also not anchored to the target and so they are pulled away from the target by the normal movements of the body in which it is implanted.

Thus a prior art device may have some functionality in the days or weeks following surgery, but the inflammatory process may within weeks manage to wall the interface off from the target, and thereby reduce or even eliminate the ability of a neural interface to control a target tissue.

The surgical procedure for prior art devices itself is an additional deterrent for doctors who are aware of risks from surgery such as general anesthesia and infection, and time in an operating room is expensive. A patient can also be discouraged from undergoing an elective surgical procedure for implantation by his or her less than optimum health and also by large insurance co-payments necessitated in significant surgery.

The electrical properties of prior art electrodes are also in need of improvement, in that their charge transfer often may incorporate a significant resistive current component in addition to the capacitive charge injections that is fully reversible, and resistive current is likely to produce corrosive by-products over stimulation time.

There is therefore a need for a biocompatible electrode which can be injected, cured and molded to surround and conform to the contours of a target in or on bodily tissue in a minimally invasive or external procedure, and produce far better chronic results at the interface with the target in bodily tissue.

There is furthermore a need for an electrode interface that can be used to inject energies other than electrical energy. Such energies may be, but are not limited to, thermal, magnetic, optical, vibration, so the cured electrode includes not only stimulation and temporary nerve block any more, but also thermal permanent nerve block (“frying”) as well as thermal temporary nerve block (cooling), as well as electrical permanent nerve block (“chemical ablation”/or pH change near a nerve/or direct current nerve ablation etc.), as well as optical temporary nerve block (laser onto nerve), as well as vibration/sound temporary nerve block (US can activate or has the potential effect of block), as well as the magnetic activation, and the guiding of electrical fields to provide a large enough signal that may cause a temporary electrical nerve block.

### BRIEF DESCRIPTION OF THE FIGURES

FIG. 1A shows the peripheral nervous system (“PNS”) neural plexi of a human brachial plexus.

FIG. 1B is an image of a rat cranial nerve ganglion adjacent to a scale.

FIG. 2A and FIG. 2B depict a prior art electrode with a planar integrated circuit that is produced by silicon wafer production techniques with needles extending from the metal contacts from a planar surface, as disclosed in U.S. Pat. No. 5,215,088.

FIG. 3A is an image of a prior art planar electrode from US Patent Application Publication No. 20150367124 and FIG. 3B is a perspective drawing of the same.

FIG. 4A is a perspective drawing of a prior art cuff electrode from US Patent Application Publication No. 20060030919 A1 and perpendicular connection to a wire, as the device is wrapped around a PNS target.

FIG. 4B is an image of a prior art cuff electrode, somewhat similar to that in FIG. 4A. The device is being held in a partially open position by an instrument, thus revealing the interior side of the device (facing the PNS target) where metal contacts are connected by wires. The lead wires to the device contact the device in the same plane of the device.

FIGS. 5 and 6 depict prior art rod-shaped electrode configurations with one or several electrode contacts aligned linearly along the rod. In FIG. 5, from US Patent Application Publication No. 20110191275, the electrode contacts are represented by the darker bands, and dimensions of the electrode contacts and spacing between them are depicted. In FIG. 6, from U.S. Pat. No. 8,473,062, the electrode contacts are represented by pairs of lines.

FIG. 7 contains two duplications of the prior art rod-shaped electrode in the center of FIG. 5. Near the left side rod, a shaded circular area to the right represents the neural target area, and electrical field lines between electrode contacts are shown, some of which run through the neural target area. On the right side rod, the electrical field lines near the end of the rod are depicted as scattering in almost 360 degrees from a single electrode contact.

FIG. 8 is a chart depicting normalized field strength as a function of distance in microns from an electrode for unipolar, bipolar and tripolar electrodes.

FIG. 9 is an image of an embodiment of the cured electrode comprising a silicone carrier material injected into chicken meat. The nerve has been pulled partially out of the cured electrode, i.e., from the groove in the upper middle of the image, which is a portion of the area of the cured electrode in closest contact with the nerve upon curing.

FIG. 10 is a conceptual diagram of the distribution of conductive elements (represented as dark bars) in the carrier material (represented as open ovals) in a cured electrode. The empty space represents pores.

FIG. 11 is an image of a portion of a cured electrode including a nonconductive layer (right side of image) after the cured electrode was removed from a nerve target. The white line is drawn to demarcate the cured electrode from the dark space (left side of image) where the nerve target was formerly located before removal of the cured electrode.

FIGS. 12A, 12B and 12C are conceptual diagrams of the liquid conductor/cured electrode. The black shapes are conductive elements and the circles represent resorbable carrier material. In FIG. 12A the liquid conductor is outside the body and the white background represents air filling any pores. FIG. 12B depicts the pores after the liquid conductor has been injected into a body and interstitial fluid (darkened background) immediately fills up at least a portion of the pores. FIG. 12C represents the cured electrode four to eight weeks post-injection after resorption of carrier material.

FIG. 13 is an image of a Transcutaneous Electrical Neural Stimulation (TENS) system including a signal generator, a least one cable and a TENS pad electrode.

FIGS. 14A-14F are cross-section diagrams of a human forearm depicting steps in the injection of the liquid conductor around the medial nerve, and connecting it to a subcutaneous contact pad, which in turn is in electrical communication with a TENS electrode. The bar arrows represent a general direction of movement of the dispenser tip.

FIG. 15 is a diagram of the chemical structure of PEG in DuraSeal.

FIG. 16 is a diagram of the chemical structure of Trilysine in Duraseal.

FIG. 17 includes examples of amine-reactive functional groups which can be substituted for NHS-ester as the active leaving group.

FIG. 18 is a chart of a function depicting the stability of PEG gels based on the concentration of elements, i.e., conductive elements.

FIG. 19 is the chemical structure of a PEG with a Hexaglycerol core (8-arm).

FIG. 20 is the chemical structure of a PEG with a Tripentaerythritol core (8-arm).

FIG. 21 contains diagrams showing steps of amine reactive crosslinker chemistry delivering stable conjugates and NHS.

FIG. 22 depicts the chemical structure of carbonyldiimidazole zero-order cross linker.

FIGS. 23-24 are diagrams showing how the hydroxyl moiety can be activated for coupling ligands.

FIG. 25 illustrates the use of cyanogen bromide to couple an amine ligand.

FIG. 26 is a diagram of the chemical structure showing the interaction between GLYMO and a silicone as the carrier material and, on the other hand, GLYMO and silver as the conductive element.

FIG. 27 is a diagram of the mechanism of a cured electrode with low aspect ratio conductive elements during bending: as the convex top is bent and conductive elements move apart slightly and reduce conductivity in the area of the bend, but conductive elements at the concave bottom are pressed together and increase conductivity.

FIG. 28 is an image of a collection of different shapes for a silicone carrier material.

FIG. 29 is a representation of the function of surfactant to promote conductivity in a cyanoacrylate based cured electrode with silver conductive elements.

FIG. 30 shows the final common pathway of coagulation cascade for fibrin glue.

FIGS. 31A-31D are images of high-aspect silver flakes manufactured with various grain size sand paper wheels using a Dremel tool.

FIG. 32 is another image showing the same high-aspect ratio silver filings as in FIGS. 31A-31D.

FIG. 33 is an image of gold flakes of various aspect ratios produced with a Dremel tool.

FIG. 34 contains images of high-aspect ratio conductive elements such as gold bonding wire bits.

FIGS. 35A and 35B are idealized section views of a cured electrode in an original linear shape and a subsequent bent position showing, after bending, the high aspect conductive elements (35B) maintain connectivity compared to lower aspect ratio (35A).

FIG. 36 is a diagram of a change of shape for NiTi wire conductive elements.

FIG. 37 is a diagram of a mesh of a cured electrode comprising gold bonding wire continuous loops that interconnect with each other, in place around a target.

FIG. 38 is a depiction of two cured electrodes on the same nerve fiber with different activation thresholds as a result of proximity to nodes of Ranvier.

FIG. 39 depicts four cured electrodes which have been injected along a nerve with a Y-junction, enabling the possibility of selective fascicle stimulation. Section views of the cured electrodes at the location of the bar arrows are shown in A-D.

FIG. 40 depicts a selective interface by positioning a cured electrode to specific fascicles A and B of a nerve.

FIG. 41 depicts a method of loading the liquid mixture and liquid nonconductor in a single chamber dispenser, with the liquid mixture in front (1st) portion nearest the tip and the liquid nonconductor in back (2nd) portion.

FIG. 42 is an image of an embodiment of a low viscosity silicone and silver based cured electrode dispensed through the dispenser in FIG. 41.

FIG. 43 depicts a cross section of a nerve fascicle surrounded by the cured electrode herein in turn surrounded by the nonconductive layer.

FIG. 44 is a diagram of two embodiments of the ring-like portion of a cured electrode, and a first side of each being connected with either the anode or cathode end of a signal generator and each of the other ends being connected optionally to a nerve target.

FIG. 45 depicts a ring like portion of a cured electrode connected to one end of the signal generator and also to the nerve (active cathode), or can be placed at another location to provide a better electrical interface to the surrounding tissue at the location of the distal anode.

FIGS. 46A and 46B are the same cross section of a single vertebra, 46A before injection of a cured electrode, and 46B, after injection, depicting a foramen transversium as location of the anchor of a cured electrode, here a ring like portion around a nerve target.

FIG. 47A contains cross-sections depicting embodiments of a mold for placing around a nerve target, comprising an opening through which a wire can be placed and secured by crimp hooks, and the wire being in electrical communication with a cured electrode dispensed into the space between the hook and the nerve target. The two diagrams on the left side depict the mold before insertion, and the two right side diagrams depict the hooks after placement. The two lower diagrams depict a mold comprising a movable slider capable of sliding out to cover all or a portion of the gap in the mold.

FIG. 47B contains perspective views of (I) a straight sock, (II) a curved sock and (III) a sock at almost 90 degrees, all at the tip of a dispenser through which the liquid mixture is dispensed.

FIG. 48 is a diagram showing a section view of a portion of a prior art cuff electrode around a nerve, showing a void between the metal contact of the prior art electrode 40 (e.g., platinum) and the nerve 5.

FIG. 49A is the same view as in FIG. 48, also showing that a cured electrode may function as a bridge between a prior art metallic electrode contact and the nerve if liquid mixture is placed onto the contact prior to implantation of the cuff.

FIG. 49B is similar to the view in FIGS. 48 and 49A, except that the metallic electrode contact is not present, and the space has been filled completely by a cured electrode.

FIGS. 49C and 49D are similar to the view in FIG. 48, except that the void has been filled by fibrous tissue. FIG. 49D also shows dispersion of the energy field lines.

FIG. 49E depicts the energy field lines traveling to the target when a cured electrode is placed as a bridge, on the left, on a prior art cuff electrode (as in 49A) and, on the right, when the platinum contact is not present (as in 49B).

FIG. 50 depicts a cross section of a needled skin patch electrode with test electronics connected to a subcutaneous contact pad. All but one of the needles is in contact with the contact pad.

FIG. 51 is a representation of a cross-section of the needled skin patch electrode connected electrically to an implantable needle matrix embedded in the contact pad, and the needle matrix and the needles from the exterior electrode are configured to make electrical connection with one another.

FIG. 52 is an image of a connecting feature for a lead wire to a cured electrode, here a helix screw (or, cork screw), held for display by an alligator clip.

FIG. 53 is a representation of a wire loop which is embedded in one portion of a cured electrode which also comprises an interface molded and cured around a nerve target.

FIG. 54 depicts an electrocorticography (“ECoG”] electrode matrix of the present invention in position on human neocortex.

FIG. 55A is an image of a human brain, depicting the sulci and gyri of the neocortex and the midline between the two hemispheres.

FIG. 55B is a representation of a section of neocortex and the underlying white matter showing the depth (and relative inaccessibility) of the areas within the sulci.

FIG. 56A is a representation of a portion of the ECoG electrode matrix in FIG. 54 from the top showing the matrix and wires terminating in holes where the wires make electrical contact with the liquid mixture (as shown in FIG. 56B) injected into the sulci.

FIG. 56B is a cross-section of neocortex and the ECoG electrode matrix including the holes allowing injection of the liquid mixture material deep into the sulci, as shown.

FIG. 57 is a representation of two types of connectors of a neural signal generator to enable an excellent mechanical and electrical connection to the cured electrode.

FIG. 58 is a representation of a neural signal generator encased with a ring-like portion of a cured electrode around a target and an anchor in a foramen (shown in FIG. 46A) for securing the neural signal generator in place. An additional cured electrode is connected to the neural signal generator at the end opposite the target.

FIG. 59A, FIG. 59B and FIG. 59C are representations of how a cured electrode can reestablish successful electrical connection between a chronically implanted electronic prior art electrode and a target, where the prior art electrode has been walled off by the body's encapsulation by the body's fibrous tissue. 59A shows encapsulation of, and blocking signal from, the prior art electrode, 59B shows reestablishment of an electrical connection between the prior art electrode and the target by means of a cured electrode, and 59C shows encapsulation of the arrangement in 59B wherein electrical communication between the prior art electrode and target is maintained.

FIG. 60 is another example of a prior art rod-shaped electrode carrier/lead with disk electrodes as shown in U.S. Pat. No. 8,565,894 B2.

FIG. 61 shows a prior art electrode from U.S. Pat. No. 8,494,641 B2.

FIG. 62 is a side view of a two-chamber dispenser comprising a syringe body comprising two coaxial chambers, a first chamber containing liquid conductor and a second chamber containing liquid nonconductor, said second chamber encircling said first chamber, a first plunger fitted for the first chamber, and a second plunger fitted for the second chamber, a coaxial needle with an exit point for both chambers.

FIG. 62A is an enlargement of a coaxial needle tip in cross section, showing the outer wall of the needle enclosing an outer needle lumen containing liquid nonconductor and extruding it beyond the exit point, the wall of the inner needle lumen extruding liquid conductor also beyond the exit point. Additionally, a pattern of extrusion is shown.

FIG. 62B is similar to FIG. 62A, except that a wire is also being extruded from the inner lumen.

FIG. 62C depicts a two chamber dispenser tip, with each chamber loaded with a wire embedded in liquid mixture, and a portion of the same extruded from both chambers.

FIG. 63 is a side view of an embodiment of the dispenser comprising an insulated stimulator wire with an uninsulated electrical stimulator which is near the exit point of the dispenser.

FIG. 64A is a diagram of one embodiment of a dispenser as a catheter for dispensing liquid conductor or nonconductor.

FIG. 64B is a diagram of another embodiment of the dispenser as a catheter which is able to dispense liquid mixture through a vessel wall to the surrounding tissue.

FIG. 65 depicts the dispenser in one embodiment comprising a light such as an LED attached to the needle.

FIG. 66 is a diagram of a conical frustum for graduated diameter decrease for a dispenser.

FIG. 67 are images of an auger embedded in a dispenser to provide a predictable forward motion of liquid conductor through the dispenser.

FIG. 68 depicts a rollable tube embodiment of the dispenser comprising a nozzle on the front end and optional apparatus at the rear to facilitate the rolling of the tube to force the liquid conductor to the needle.

FIG. 69A shows a needle system that utilizes an open tip as well as an open side port.

FIG. 69B shows a needle system that utilizes a closed and rounded needle tip and a side port near the tip.

FIGS. 70A-FIG. 70C is a sequence of diagrams depicting use of a pre-formed mold, here an inflatable balloon, to facilitate placement of a cured electrode.

FIG. 71 depicts a syringe with a wire with a connecting feature at its forward most point embedded in the liquid conductor.

FIG. 72 contains four images of one embodiment of a manual mixer. Images A and B show two syringes without needles joined by a connector. Image C depicts the syringes and the connector prior to being joined. Image D is an image of the manual mixer comprising a baffle in the lumen of the connector.

FIG. 73 is a schematic of dielectric polarization and heating brought about by RF waves.

FIG. 74 contains a larger diagram of staples with prongs inserted into a connective tissue plain and the staple heads embedded in cured electrodes surrounding a nerve target. Two smaller diagrams are of a staple before insertion (top) and post insertion with head embedded in a cured electrode (bottom).

FIG. 75 depicts staples with a connecting head, the prongs of the staples crimped into a wall of an organ (e.g., bladder), and the connecting head embedded in the liquid conductor/cured electrode.

FIG. 76A shows that by placing the liquid conductor all around the connection point of the three side arms forming the Y provides a means to stimulate all nerve fibers entering and exiting the Y-junction.

FIG. 76B depicts lacing ring-like portions of the liquid conductor around each of the smaller side arms as well as additional liquid conductor around the major remaining arm, all surrounded by a single liquid nonconductor/nonconductive layer.

FIG. 77 contains three diagrams showing steps of tying an adjustable hitch knot integrated with the cured electrode to allow breakage of the cured electrode by pulling on the loop to enable easy removal of the cured electrode.

FIGS. 78A-B. A graphic showing shear forces (arrows denoted F) required for cutting and/or removing are greater for insulated solid wire FIG. 78A than for the cured electrode, FIG. 78B.

FIG. 79 is a diagram illustrating the location of the present invention in an above the knee amputation.

FIG. 80A and FIG. 80B are diagrams depicting examples of placement of liquid mixture “blobs” on prior art electrodes to align field lines through the target.

FIG. 81A is a diagram depicting homogenous electrical field lines and FIG. 81B depicts electrical field lines distorted by examples of placement of liquid conductor “blobs” to align field lines through a target.

FIG. 82 is a diagram showing liquid conductor blobs injected into a nerve without leaving a trace through the epineurium, and cured electrodes outside the epineurium.

FIG. 83 depicts a liquid conductor blob injected into the nerve while leaving a wire-like portion of the cured electrode through the nerve's epineurium, here shown only on the left side.

FIG. 84 depicts a nerve target with a chronically-implanted prior art cuff electrode with two solid metal contacts on opposite sides of the nerve, and the nerve encapsulated in fibrous tissue. Electrical field lines scatter through the nerve and also around the perimeter (the epineurium) and in the encapsulation.

FIG. 85, like FIG. 84, contains a chronically-implanted prior art cuff electrode. FIG. 85, though, illustrates that electrical field lines can be redirected in a revision procedure, by placing liquid conductor just underneath the two cuff electrode contacts on opposite sides of the nerve just inside the cuff electrode, and also placing liquid nonconductor in the fibrous tissue to prevent circumferential electrical field lines.

FIG. 86 shows electrical field lines through a nerve target between (A) disc electrodes, and (B) ring electrodes, either of which may be prior art electrodes or electrodes manufactured and cured in situ.

FIG. 87 is a diagram showing a procedure to create a gap in the fibrous tissue between the previously implanted prior art cuff electrode's contact pads and then to inject liquid conductor to fill that gap, thus bridging the encapsulation.

FIG. 88 is a schematic of a nerve with two electrodes being placed along the nerve.

FIG. 89 is a schematic of resistive and capacitive impedance components on the path from one electrode through interstitial fluid to the axon within a nerve and back.

FIG. 90 is a schematic of the voltage curve measured during current controlled stimulation showing the resistive component (solid curve: vertical lines=IR-drop) and the capacitive component (dV/dt indicating the charging of surface boundaries).

FIG. 91A is a schematic of a lab setup for a neurostimulation study with an LCR meter and a first and a second steel probe for measuring impedances in various animal tissues.

FIG. 91B is similar to 91A, with the addition of a cured electrode in direct contact with the first steel probe, but not in direct contact with the second steel probe.

FIG. 91C is similar to 91B, with the second steel probe being in direct contact with the cured electrode to obtain the impedance of the cured electrode(s), and with the addition of a third probe not in direct contact with the cured electrode.

FIG. 92 is a schematic of a lab setup for a neurostimulation study with an oscilloscope to measure the voltage necessary to apply a current controlled biphasic waveform during TENS stimulation on chicken meat, with and without a cured electrode.

FIG. 93A is an image of an oscilloscope readout of 3.8 volts from the setup in FIG. 92 without a cured electrode injected into the chicken meat.

FIG. 93B is an image of an oscilloscope readout of 1.68 volts from the setup in FIG. 92 with a cured electrode injected into the chicken meat.

FIG. 94A is an image of a rat brachial plexus.

FIG. 94B is an image of the rat brachial plexus as in FIG. 94A, but with a cured electrode on the brachial plexus.

FIG. 94C is an image of a lead wire embedded in the cured electrode in FIG. 94B.

FIG. 94D is an image of a lead wire embedded in a cured electrode formed as a ring around a rat bladder neck and some more cured electrode material added for mechanical matching.

FIG. 95A is an image of a pig brachial plexus and a ring like cured electrode formed in open cut down.

FIG. 95B is an image of forming a knot with a suture in a cured electrode and pulling on the knot with two surgical clamps.

FIG. 95C is an image after pulling on the knot in 95B with two surgical clamps and the pieces of the cured electrode after the suture cut through the cured electrode.

FIG. 96 is a diagram showing placement of TENS patch electrodes on the outside of the skin of a pig, each patch electrode on top of a corresponding cured electrode as a subcutaneous contact pad, each contact pad being connected to a ring electrode attached by a wire acutely to the vagus nerve.

FIG. 97 is an image of the contact pads, from the setup in FIG. 96, next to coins for comparison of size.

FIG. 98 is a chart which plots heart rate (bpm) versus time (seconds) observed from stimulation of the vagus nerve in pigs in the set up diagrammed in FIG. 96, under five different conditions: (1) low amplitude stimulation, (2) mid amplitude stimulation, (3) high amplitude stimulation, (4) removal of the subcutaneously placed contact pad 14 that connected to the cathode to test for leakage driving the HR reduction, with no leakage detected, and (5) removal of the subcutaneously placed contact pad 14.

FIGS. 99A and 99B are two charts showing a comparison of electrodes and their capacitive charge injection capabilities: a prior art cuff (Livallova) 99A and the cured electrode 99B.

FIG. 100A is an image of the readout of impedance on an LCR meter as 2.328 ohms, measured across the length of several turns and twists of the extruded very thin cured electrodes and wires (<1 mm) as shown in FIGS. 100B and 100C.

FIGS. 101A and 101B depict differences in impedance spectrometry for a prior art device (101A) and the cured electrode (101B) of the present invention.

FIG. 102 shows in A and B that a coil concentrates magnetic field lines and, additionally, the cured electrodes in B placed near a target induce further concentration of magnetic energy at the target.

FIG. 103 shows, in dotted line portion A, the top target tissue in an air gap between two magnetically cured electrodes with north and south poles. In dotted line portion B, the cured electrode acts to shield the bottom target from the magnetic field. In dotted line portion C, the effect on magnetic field lines distant from the cured electrodes is minimal.

FIG. 104 shows: A, lower magnetic field density at the target with a coil but without a cured electrode; B, greater field density at the target by adding a cured electrode between the coil and the target; and C, even further concentration than in B by adding a second cured electrode and creation of an air gap at the target.

FIG. 105A shows: I, some concentration of magnetic field lines by a coil; and II, greater concentration of field lines by adding a cured electrode inside the coil.

FIG. 105B depicts a headband situated on the circumference of a head, shown from the top, said band containing coils which correspond to subcutaneous magnetically conductive blobs of cured electrode.

FIG. 106 is a graph showing thermal conductivity of materials.

FIG. 107 depicts a Peltier element embedded between two thermally conductive cured electrodes, one surrounding an artery supplying blood to a tissue, with the Peltier element's cold side towards the artery and the hot side transferring the heat away from the artery and the tissue by means of a second cured electrode.

FIG. 108, somewhat similar to FIG. 107, depicts a Peltier element embedded between two thermally conductive cured electrodes, one cured electrode surrounding an artery supplying tissue, with the Peltier element's cold side towards the artery and the hot side transferring the heat away from the artery to a vein leaving the tissue by means of a second cured electrode.

FIG. 109 is a configuration of thermally conductive cured electrodes for measuring and controlling temperature in a blood vessel, here an artery.

FIG. 110 is a conceptual representation of how a thin-film lead wire high and low structures (A) or holes (B) to allow the liquid mixture to adhere to the lead wire.

FIG. 111 is a diagram of two cured electrodes surrounding a target connected to a diode (D) which is either a voltage or current limiter.

FIG. 112 is a diagram of two cured electrodes surrounding a target connected to a diode (D) which is either a voltage or current limiter, also with capacitors (C).

FIG. 113 contrasts the larger ablation lesion of prior art devices compared to that from the cured electrode.

FIG. 114 depicts an embodiment of the cured electrode for use in ablation, in A, fully surrounding the target and, in B, partially surrounding the target.

FIGS. 115A-C show patch electrodes supplying current, here for ablation, to the cured electrode: A, fully surrounding the target; B, partially surrounding the nerve and C, using wire-like portions of a cured electrode drawn from the cured electrodes surrounding the nerve to a subcutaneous contact pad comprising cured electrode material near each of the patch electrodes.

FIG. 115D depicts transcutaneous transmission of energy to a target surrounded by a cured electrode, and the lesion pattern in the tissue surrounding the target.

FIG. 116 contains images taken in sequence for ablation of chicken leg tissue with a cured electrode: A, shows placement of an electrode before ablation (note the return electrode at top) and B shows the tissue after ablation and removal of the cured electrode, revealing the lesion. C is a zoomed view of B.

FIGS. 117 A-D are IR images showing temperature in degrees Centigrade from RF ablation Experiment 1 on chicken tissue with a cured electrode.

FIG. 118 is an image of the setup from RF Ablation Experiment 2 on chicken tissue with a cured electrode.

FIGS. 119 A-E are six IR images from a video showing time course of the temperature changes in RF Ablation Experiment 2.

FIGS. 120 A-D are four time stamped images from the same sequence in FIG. 119, with the time stamps in the lower left corner of each image.

FIG. 121 is an image of the setup of RF Ablation Experiment 3.

FIGS. 122 A-E are images from an IR video of the time course of the pork RF Ablation Experiment 3.

FIGS. 123 A-B are images of pork muscle tissue in RF Ablation Experiment 4 with cured electrode injected in a cavity (upper) and removed from the cavity (lower).

FIG. 124 is an image of a cured electrode in RF Ablation Experiment 4 stuck between two pieces of pork tissue held during ablation with a toothpick, showing whitened tissue ablated on the left in the pattern of the cured electrode on the right.

FIG. 125 is an image from RF Ablation Experiment 4 with the cured electrode removed from the tissue ablated (whitened).

FIG. 126 is an image from RF Ablation Experiment 4 with aluminum foil crumbled and placed between two pieces of pork tissue, where the aluminum foil has been removed from the whitened spot in the center of the image where it was when energy was applied.

FIG. 127 is an image from RF Ablation Experiment 4 with crumbled aluminum foil (on left) having been removed from the tissue at the arrow, and a cured electrode (on right) has been removed from the tissue at the arrow. Note the much greater ablation (whitening) of the tissue from the cured electrode on the right.

FIG. 128 A-B are section views of heat transfer (shown by arrows) from a cured electrode to surrounding tissue, with RF energy in A from a probe and in B from dispersed sources.

FIG. 129 is a section view of heat transfer (shown by arrows) from small blobs of cured electrodes injected into tissue. Note how the heat emanates from the blobs when they receive RF energy from the surrounding.

FIG. 130 is a section view of a cured electrode inserted on one side of a tumor to stop its progress, and a probe attached for applying energy, as well as a counter-electrode.

FIG. 131 A-C are section views of a metal contact on the skin (A) and with a hydrogel layer sandwiched between the contact and the skin (b), and a microneedle patch on the skin.

FIG. 132 is one embodiment of a waveform for DC ablation.

FIG. 133 is a frontal schematic view of the spinal column and the upper portion of the rib cage (front cut-away) and the sympathetic chain running along both sides of the spinal column.

FIG. 134 is also a frontal schematic of a portion of the rib cage and the sympathetic chain ganglia, showing greater detail (as compared to FIG. 133) of the highly irregular shapes of the sympathetic chain ganglia.

FIG. 135 is a drawing showing foramina as exit points for spinal nerves with placement of liquid conductor or nonconductor in a foramen.

FIG. 136A is a drawing of the basic anatomy of tendons and the Golgi tendon organs at the interface to the muscle fibers.

FIG. 136B is a diagram of Golgi tendon organs with four cured electrode locations.

FIG. 137 is a drawing of placement location for a liquid conductor/cured electrode on the brachial plexus in a human (as in FIG. 1A) with a neural signal generator (not depicted) implanted to electrically connect to the cured electrode and thereby fully depolarize all fibers of the brachial plexus on demand.

FIG. 138 shows a knee joint with multiple thermally cured electrodes cooling arteries supplying blood to the knee joint.

FIGS. 139, 140 and 141 are drawings of the outer ear. 141 shows some innervation patterns from cranial nerves.

FIGS. 142 and 143 are images of external cured electrodes placed on a subject's ear in different locations.

FIG. 144 contains two images of external cured electrodes, after removal from the ear. Note the darkened areas with the greatest concentration of conductive elements.

### TERMS

In addition to additional definitions and explanations supplied throughout this written description, the following definitions apply.

(1) “Capacitive charge injection” means electrical charge injected from the interface into an ionic medium that can be extracted fully without any charge components causing irreversible chemical reactions.
(2) “Resistive charge injection” means electrical charge injected from the interface into an ionic medium that cannot be fully extracted with some charge components causing irreversible chemical reactions, thereby likely to change local pH levels near the interface and the surrounding or nearby (target) tissue.
(3) “Carrier material” means any biocompatible material comprising a liquid (or less viscous) phase curing to a solid or a more viscous phase. A carrier material is one selected from a group consisting of a hydrogel, an elastomer such as silicone, bone cement, cyanoacrylate, dental amalgam, dental resin, fibrin glue, polyethylene glycol, hyaluronic acid, or their components and others.
(4) “Collagen” and “gelatin” are synonymous, unless specifically differentiated.
(5) “Conductive elements” are elements of conductive material which, at the time of placement in a body, comprise at least one dimension of at least one micron. Conductive elements may be produced by a process selected from a group comprising cutting, grinding, etching, extruding and conglomeration of smaller elements.
(6) “Conductive” or “conductivity” means the ability to transfer energy including, without limitation, electrical, magnetic, thermal, light and vibration (including sound).
(7) “Cure” includes, without limitation, polymerizing, crosslinking, going through precipitation and/or going through solvent phase inversion, gelation or other phase transition to a solid which retains its shape when subjected to shear forces expected for a living body in non-extreme conditions. The curing can be substantially instantaneous, a few seconds or minutes, or may occur over a longer period of time.
(8) “Elastomer” means any of various elastic substances resembling rubber, e.g., polyvinyl elastomers which comprise a liquid phase and a solid phase, including without limitation siloxane.
(9) “Fractal surface” means a volume current injector as a result of a conglomeration of smaller pieces which may be roughened as in with a laser (e.g., on Pt foil) prior to being shredded, and also through resorption of materials by the body leaving pores.
(10) “Inject” means introducing into bodily tissue through (a) a dispenser by means of a needle or needle-like structure without the need of an incision besides that of the needle, (b) a catheter in a blood vessel or other bodily structure with a lumen, (c) a pump through a laparoscopic device inserted through a small incision, (d) a hole that has been created with a separate incision, or (e) an auger system transporting the injectable material inside a lumen from which it is expressed near, into or around an interface target.
(11) “Liquid mixture,” comprises a carrier material in a liquid phase and solid conductive elements dispersed throughout, and the liquid carrier material is capable of curing to a solid phase. “Liquid mixture” means not only the liquid carrier material but also the solid conductive elements contained within it. “Liquid mixture” may also include the carrier material being in a combination of liquid and solid phases, in different portions of the same mass of material, and means the same as “liquid mixture/cured electrode.”
(12) “Liquid nonconductor,” means a carrier material in a liquid phase without any conductive elements, or an insufficient concentration of conductive elements to enable energy conductivity. The liquid nonconductor may comprise the same material as the carrier material in use in the liquid mixture (or not), and the liquid nonconductor is also capable of curing to a solid phase and bonding to the liquid mixture. A liquid mixture cures to a solid phase termed a “nonconductive layer.”
(13) “Liquid phase,” means a state in which liquid or material may flow by, for example, injection prior to curing to a later and more solid phase. “Liquid phase” includes, without limitation, a paste or other configurations which do not hold their shape and do not possess the ability to reestablish an earlier shape (akin to a pudding) when subjected to shear forces expected for a living body.
(14) “Network,” means an irregular structure comprising numerous conductive elements of either regular or irregular shape, said conductive elements being either touching one another or disposed in very close proximity to one another.
(15) “Nonconductive layer” is liquid nonconductor which has cured to the solid phase.
(16) “Percolation” means the ability to disperse throughout a mixture while retaining direct mechanical contact and thus either an electrical, magnetic, thermal or optical path or a combination of those mentioned throughout the mixture.
(17) “Phase transition” includes, without limitation, curing, cross-linking (chemical, ionic or other), polymerization, gelation, self-assembly, or fusion/solidification
(18) “Resistive charge injection” means current transferred by the electrical interface into an ionic medium which causes irreversible reactions to occur in the vicinity of the electrode/electrolyte interface inside the ionic medium.
(19) “Solid” means a material which has undergone a phase transition away from the liquid phase and has substantially polymerized, cross-linked, precipitated, gelled, gone through solvent phase inversion, or transitioned otherwise, and retains its shape under shear forces expected for a living body in non-extreme conditions at specific locations chosen by physicians.
(20) “Solid phase,” means a state in which a material has cured substantially to a solid and at least partially retains a shape under shear forces expected for a living body in non-extreme conditions, either flexible or hard and either hydrous or anhydrous, or having these qualities partially or in combination.
(21) “Target” includes without limitation nervous tissue including a nerve, plexus, ganglion, brain, spinal cord and the like, and any other tissue for which electrical, magnetic, thermal, optical or vibratory stimulation (energy) may have an effect such as for example, muscle, blood vessels, organs and tumors. The present invention provides a preferential energy path to prevent unwanted side effects to non-target tissues.

### GENERAL

The present invention solves the above problems, and provides additional advantages unknown in the prior art. The cured electrode 1 of the present invention, in one embodiment, first comprises a liquid mixture in a liquid phase which is capable of being injected through a dispenser 2 comprising a needle 3 to the target 5 without a surgical procedure, where it can be pushed from the needle 3 and molded to the contours of the target and is capable of curing to a solid phase which is capable of retaining the shape of the contours of the target. The present invention produces low impedance values (<100Ω or even <10Ω or <1Ω), low mechanical impedance, low optical impedance, low magnetic reluctance, thus providing a simple approach to connect electrically to a target in bodily tissue in various locations, different patients and within a shorter procedure time when compared to the time needed to place prior art electrodes, especially cuff electrodes.

Another advantage of the present invention is that it is injectable without surgical dissection of tissue leading up to the target by means of scalpel, scissors and the like prior to electrode placement, that is, with little or no disruption to the target or surrounding tissues. The present invention has the ability to form a “negative” from the “positive” target contours. The novel property of curing to the contours of the target not only provides a better electrical/magnetic/optical/thermal/mechanical connection to the target, but also a better mechanical adherence to it, thereby anchoring it. Anchors 4 for the cured electrode 1 may additionally be achieved by injecting either liquid mixture, or liquid nonconductor bonded to the liquid mixture, to non-target structures such as bones.

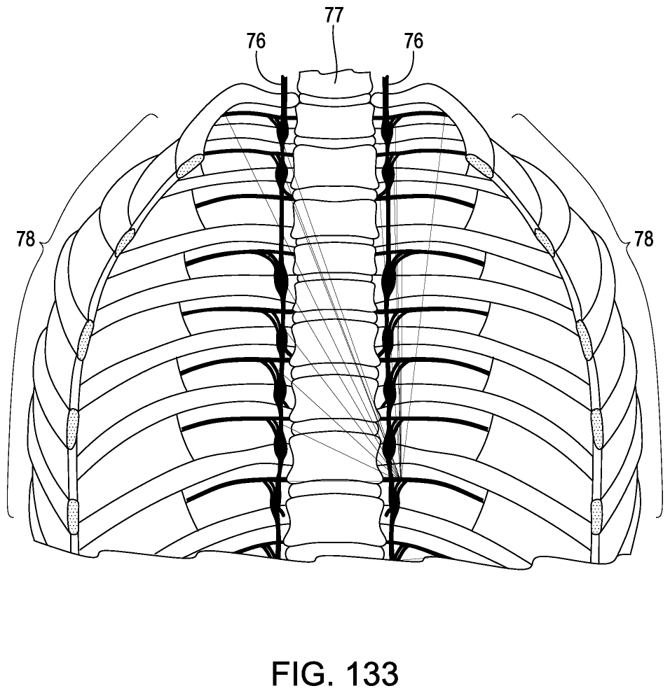

Moreover, through injection the cured electrode of the present invention can be placed in hard to reach locations in the body which a surgeon might be unwilling to place a prior art device with elective general surgery, e.g., ganglia of the sympathetic chain or nerves of the PNS adjacent to major blood vessels and located medially in the body which are difficult to access on a direct line from outside of the body. See e.g., FIG. 133.

The particular mechanical and structural properties of the cured electrode 1 can be varied to match the properties of the tissue targeted, by the choice of the liquid carrier material 7 or by additives thereto, and by the selection of the conductive elements 6. The curing process, i.e., by introducing additional conditions or energies during curing such as ultrasound, cooling or heating or radio-frequency radiation may furthermore be utilized to change the physical properties of what becomes the cured electrode 1.

Additionally, the present invention is generally being put into place without the far greater costs of general surgery, and the attendant risks from general anesthesia and infection. The present invention can be placed by pain physicians accustomed to the placement of pharmacological nerve blocks with or without the aid of palpation, electrical stimulation (as verification) and ultrasound or angiography as means for visualization.

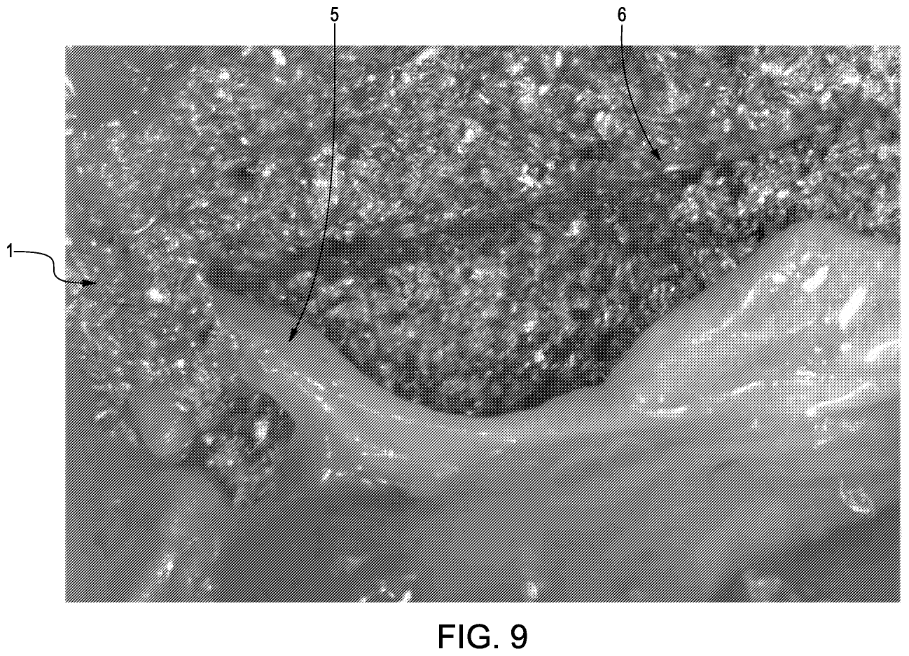

FIG. 9 is an image of an embodiment of the cured electrode 1 with a silicone carrier material injected into chicken meat. The silicone was molded against, and cured to a solid against a target 5, here a nerve partially on the right side of the image and fully on the left side of the image. A few minutes after the injection, the nerve was pulled back from the cured electrode 1. Note the 360 degree covering on the left, the 180+ degree interface on the right, the groove on the left indicating the mechanical match between the nerve and the cured electrode and how well the material matches with the nerve's mechanical structure. This is a fundamental example showing that it is possible to intentionally encase 180-360 degrees around a nerve. The impedance of muscle tissue as measured in rats, chicken and pork is approximately 500 to 700Ω at 1 kHz sinusoidal waveforms. Impedance values of different embodiments of the cured electrode are provided herein. Any material providing a lower impedance than 100Ω is thus at least five times more conductive and any mixture of <10Ω is at least 50 times more conductive than the surrounding bulk, not yet taking into account the additional impedance added by the encapsulation which encases any electrode placed into the body over time in the chronically implanted case, i.e. after three to four weeks post implantation. This difference in conductance results in a preferential current path for electrical current preferentially following the field lines through the low-impedance mixture instead of going around the mixture through the high impedance bodily, such as muscle, tissue. Note how the pliable cured electrode has conformed around the nerve on the left side. The nerve was freed up completely prior to injection in comparison the only partial encasing of the nerve on the middle and right side of the image where the nerve had only been partially exposed. Note further how the electrically conductive cured electrode has reproduced a perfect mechanical imprint from the nerve, indicating how pliable the electrically conductive cured electrode is prior to curing and gentle it may be too sensitive neural structures. Note as well that the mechanical integration of the electrically conductive cured electrode around a nerve may allow for some slight movement of the nerve within the cured electrode (especially when silicone based carriers are used that don't bond to the biological tissue) and how the mechanical integration may be facilitated around a nerve's Y-junction or other anatomical structures that provide means to form the cured electrode around or into, thereby providing a way to mechanically anchor the cured electrode against, into or around the biology.

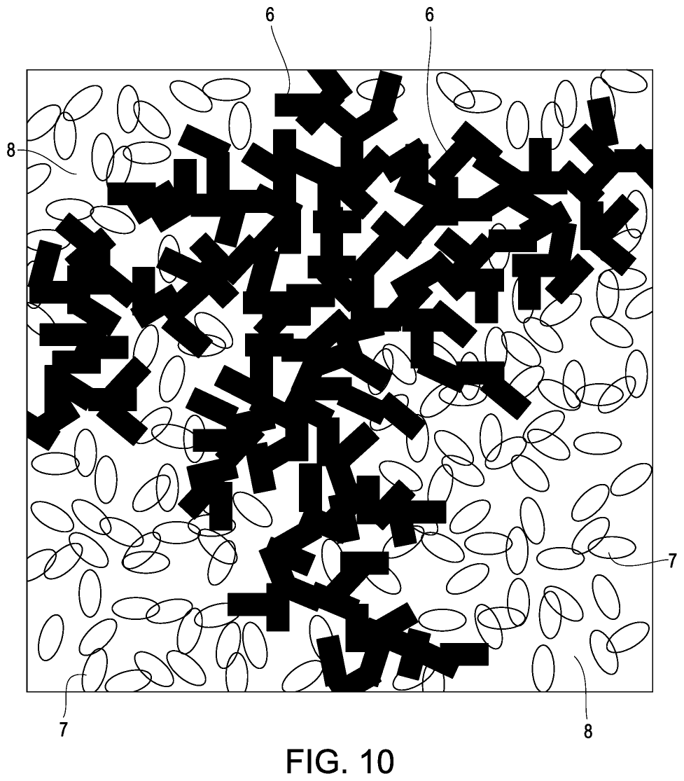

A conceptual diagram of the distribution of conductive elements 6 (represented as rectangles) in the carrier material 7 (represented as ovals) is shown in FIG. 10. The conductive elements form a conductive pathway through the carrier material, either in a liquid phase or a solid phase after curing. The open spaces between the conductive elements 6 and the carrier material 7 represent pores 8.

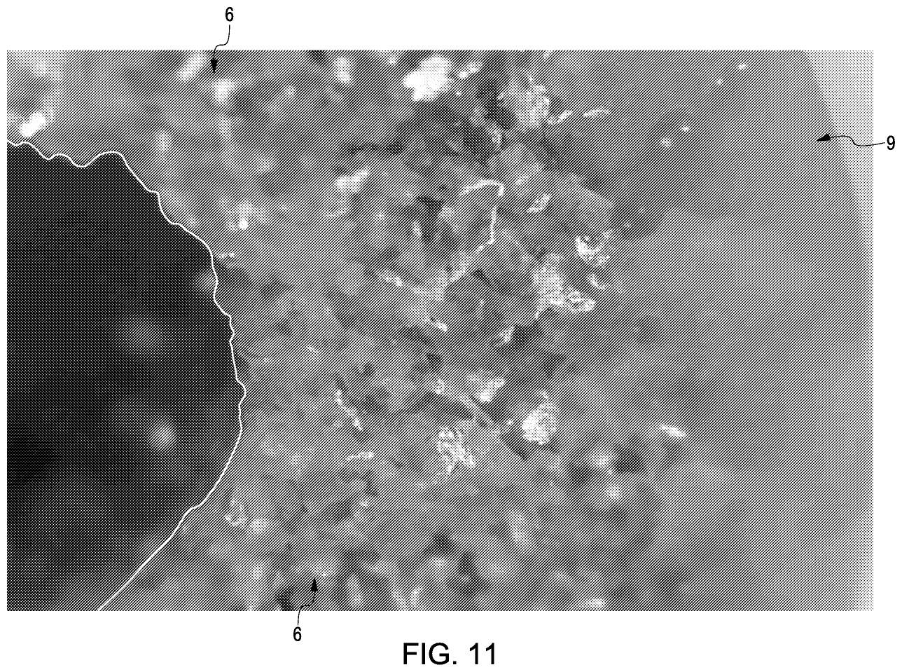

FIG. 11 is an image of a cured electrode 1 including a nonconductive layer 9 removed from a nerve target. The curvature on the left was produced by the molding of liquid mixture/cured electrode against the target (not shown), surrounded by the inner cured electrode with silver conductive elements 6, and the outer portion with few or no conductive elements is the nonconductive layer 9. Note the white line drawn to show how the cured electrode after curing retains the shape of the neural or other bodily target.

The fractal structure as in FIG. 11 is formed by the conductive elements dispersed within the nonconductive material, here silicone, but may likewise be polyethylene-glycol, Hyaluronic acid, or other hydrogels. As water slowly seeps into the cured electrode, either by migrating through the non-hermetic silicone or other nonconductor such as hydrogel, or by following capillary effects at the interface between the metal flakes and the silicone/hydrogel carrier, a large surface area of metal-to-liquid is forming inside the cured electrode. The cured electrode thus becomes a volume interface, where electrical current may transition from the metal conductor to the ionic conductor of the liquid inside the cured electrode, and the interface with the nerve only along the area where the cured electrode directly contacts the nerve. Note the fractal structure of the silver flakes. As the cured electrode gets flooded with interstitial fluid in the body, a large surface area silver-to-ionic liquid forms. This large surface area allows for essentially the entire volume of the electrically conductive cured electrode to conduct electrical energy from the metal to the ionic conductor, meaning a volume effect to inject charge is used on the level of charge transfer at the interface metal-water, while a reasonably small surface area of the cured electrode is exposed to the nerve (hole on the inside circle of the depicted cured electrode), allowing the concentration of the electrical field lines onto that area. In summary, while a volume effect is used to transfer charge from the metal (or i.e. other electrically conductive elements) to the body's ionic conductors (interstitial fluid, etc.), an area interface between the inside of the cured electrode and the nerve transfers all the ionically conducted charge to the nerve. As the conductive elements are in very close proximity to the nerve, only small space remains for connective tissue to be grown around the nerve, further limiting a chronically large impedance between the cured electrode and the nerve, a positive effect. Note the porous structure that forms between the silver (or other metal) flakes which allows for capillary forces to drive aqueous liquid all along the inside of the electrically conductive cured electrode, resulting in a large surface area between the conductive elements of the electrically conductive cured electrode and the ionic current along the conductive elements to travel with low impedance and the entire bulk volume of metal elements to be on pretty conductor the human/animal body provides. Note how the metal elements are all touching in one or more points, allowing for much the same electric potential. This allows the electrically conductive cured electrode to spread a potential from one location applied to the cured electrode to the entire volume and allows for the complete volume to function as a uniform unit, providing a homogeneous electrical field all around the nerve.

The cured electrode relying on e.g. only one type of conductive element, a surfactant and a nonconductive carrier matrix (that may or may not necessarily cure shortly after injection), may thus form a porous interface that fills with interstitial fluid from the body, even without the addition of cells or components (e.g. sugars) to be absorbed via macrophages. Fractal surface reduces resistance and increases capacitance component by, without limitation, (1) reducing thermal noise due to low-impedance conductive materials (e.g., metal flakes in contact with each other); (2) reducing surface impedance through large fractal surface area; and/or (3) increasing capacitance through large fractal surface area—not just the surface touching the nerve but also the surface of conductive elements within the cured electrode.

While the fractal surface reduces the impedance for charge injection from the conductive elements to the surrounding interstitial fluid, the current traveling between the conductive elements does so as electron based conduction as the conductive elements/elements are touching each other. This is different from commonplace hydrogels or conductive mixtures with impedances above the 100 ohm*cm range and especially above the 10 ohm*cm range, as these commonplace hydrogel conductors generally utilize ionic conduction (achieved or improved by doping with ions inside the hydrogel) or semiconductor type conduction.

Stimulation at low current thresholds is furthermore possible with cured (electrically conductive) electrodes 1 by providing a directly touching neural interface that minimizes the gap between the electrode interface and the nerve. As such, the current paths between the cured electrode and the target are much shorter and more direct than the current path between the contacts of traditional electrodes, which are often recessed inside their carrier matrix. Plonsey and Barr (referenced elsewhere herein) had shown that one of the primary factors for nerve activation current thresholds is the distance between an electrode and the nerve cells of interest. Furthermore, by providing a complete surrounding of a neural structure, the cured electrode 1 has the ability to depolarize the neural target structure uniformly. This “cuff effect” of encasing a neural structure all around and as close as possible provides a much more predictable neural activation and block threshold for said neural structure with a chronically placed cured electrode, especially when compared with a traditional electrode that may only be placed “in some proximity” to a neural structure, allowing fibrous tissue growth in the distance between the neural target and the traditional electrode to form much more unpredictable current paths, thereby less reliable and less reproducible nerve activation and block thresholds.

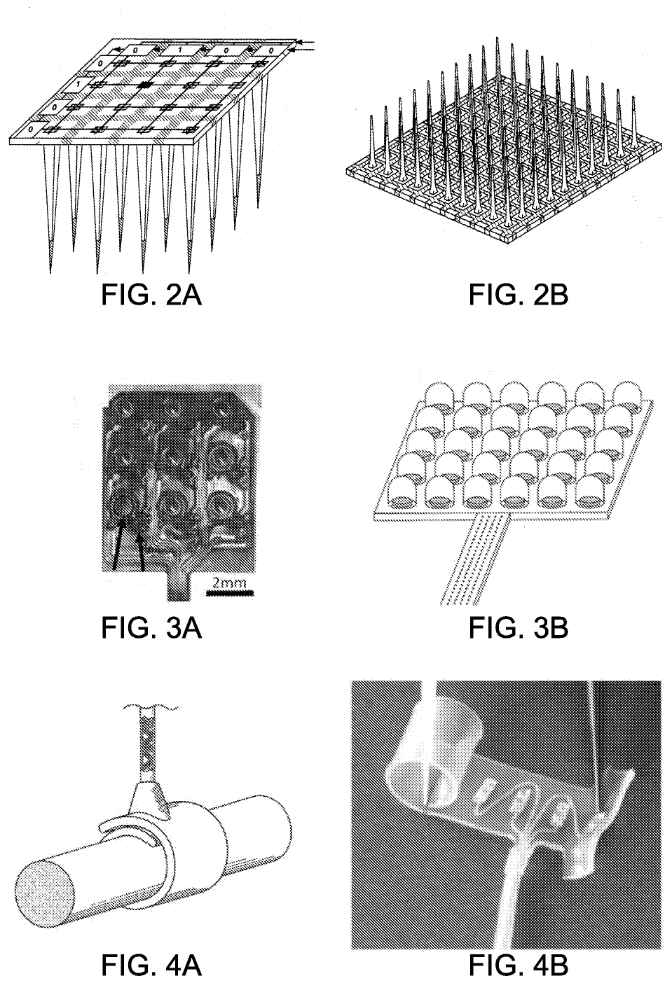

The present invention also has another distinct advantage over the prior art in its superior qualities as an electrical system for bodily tissue. A wire or needle tip (FIG. 2A) or a flat or smooth metal contact (FIG. 4B) has a smaller surface area to inject current capacitively than a rough electrode with greater texture. In one embodiment of the invention, a carrier material 7 such as a hydrogel that becomes porous (partially resorbed between the conductive elements) provides a greatly expanded surface area for the conductive elements interfacing with the surrounding and penetrating interstitial fluid of the body. The charge injection for an implanted electrode may consist of both capacitive and resistive current transfer. In a bodily tissue, when the intent is to temporarily stimulate or block neural tissue, the best way to inject current is via capacitive charge injection which does not lead to irreversible chemical reactions which in turn can lead to the dissolution or corrosion of an electrode or the change in pH levels near the electrode and the nerve, thereby damaging the nerve. Cogan, S., Neural Stimulation and Recording Electrodes, Ann. Rev. Biomed. Eng. 10:275-309 (2008), Shannon, R. V. (April 1992). “A model of safe levels for electrical stimulation”. IEEE Transactions on Biomedical Engineering. 39 (4): 424-426, Cogan S F, Ludwig K A, Welle C G, Takmakov P (2016). “Tissue damage thresholds during therapeutic electrical stimulation,” Journal of Neural Engineering. 13 (2): 021001 (2016). In order for any electrode to provide a large charge injection capacity, two potential pathways are open: (1) increase the electrode's surface area in contact with the electrolyte, either by enlarging the electrode's macro dimensions or by utilizing fractal surface structures (akin to platinum black), and (2) use materials that offer a large charge injection capacity inherently. In one embodiment the present invention uses the ability of the body to dissolve, absorb or resorb the carrier material 7, fully or at least partially, thereby leaving the conductive elements 6 (which are not resorbed) to form pores 8 and a porous shape to which the electrolyte makes intimate contact while both, the conductive elements and the electrolyte in intimate contact are encased by encapsulation of bodily fibrous tissues. This highly increased surface area, forming a “charge injection volume”, of electrical carrier elements in direct electrical contact with each other and in part direct electrical contact with the surrounding ionically conductive interstitial fluid, stands in stark contrast to the generally more or less flat surface of conductive material (such as a prior art platinum disk or foil) that only provides the electrode-electrolyte interface in a more or less planar surface. The current may enter this high surface area porous shape through a wire 10 that is encased in, and in electrical contact with, the conductive elements 6, thereby permitting electron transfer as the primary means for current to travel among the conductive elements. This in turn provides a significant increase in effective electrode-to-electrolyte interface area throughout the whole volume of the conductive elements. The pores 8, filling with interstitial fluid or otherwise watery solutions inside or outside the body during or after the cured electrode placement, embody a larger surface area for the charge injection process than is known in the prior art, much larger than the surface area for a smooth surface of the same volume's outer dimensions.

Calculations have been made of the surface area of the porous cured electrode surface area (S/A) assuming a 1 gram cured electrode which is approximately 0.5 cm³ and these have been compared to prior art macroelectrodes reported in Cogan (2008) cited above. The surface area for the present invention cured electrodes is up to eight orders of magnitude greater than reported in Cogan, as shown in Table One.

TABLE ONE
Comparison of Surface Area to Prior Art Electrodes

| | | | | E | | |
|---|---|---|---|---|---|---|
| A | B | C | D | Con- | | |
| Electrode | Flake | Hydro- | Pow- | ductive | | |
| Formu- | S/A | gel | der | Elements | F | G |
| lation | (m²/g) | (g) | (g) | wt % | m² | μm² |
| Present | 1.0 | 0.2 | 0.8 | 80.0 | 8.00E−o1 | 8.00E+13 |
| Invention, | | | | | | |
| Low S/A | | | | | | |
| Microflake | | | | | | |
| Present | 4.0 | 0.35 | 0.65 | 65.0 | 2.60E+00 | 2.60E+14 |
| Invention, | | | | | | |
| High S/A | | | | | | |
| Microflake | | | | | | |
| Present | 7.0 | 0.5 | 0.5 | 50.0 | 3.50E+00 | 3.50E+14 |
| Invention, | | | | | | |
| High S/A | | | | | | |
| Microflake | | | | | | |
| Cogan | — | — | — | — | 1.00E−09 | 1.00E+05 |
| Macro- | | | | | | |
| electrode | | | | | | |
| Cogan | — | — | — | — | 1.00E−10 | 1.00E+04 |
| Macro- | | | | | | |
| Electrode | | | | | | |

The surface area of the present invention cured electrodes in Columns F and G in Table One might be reduced up to 50% to allow for some surface overlap as these are only calculated values, not measured. The flakes are highly irregular in shape and therefore great compaction is not expected. Even so, the present invention enables a vast increase in surface area of the cured electrode interface over the prior art.

In another embodiment, a carrier material which is not significantly resorbable after curing (e.g., silicone, bone cement, dental resin or amalgam) can be left inside the body chronically which will enable the permeation and in-creeping of water and watery solutions along the interface of non-conductive layer and conductive elements, thereby filling existing pores 8 in the cured electrode or filling pores which may form over time as the mixture is subjected to forces from body movements. That is, pores 8 may form in any cured electrode of the present invention, whether resorbable by the body or not. In another embodiment, a carrier material which is not significantly resorbable after curing (e.g., silicone, bone cement, dental resin or amalgam) can be mixed with other resorbable additives, discussed elsewhere herein, which will enable the creation of pores in the cured electrode after these additives are resorbed.

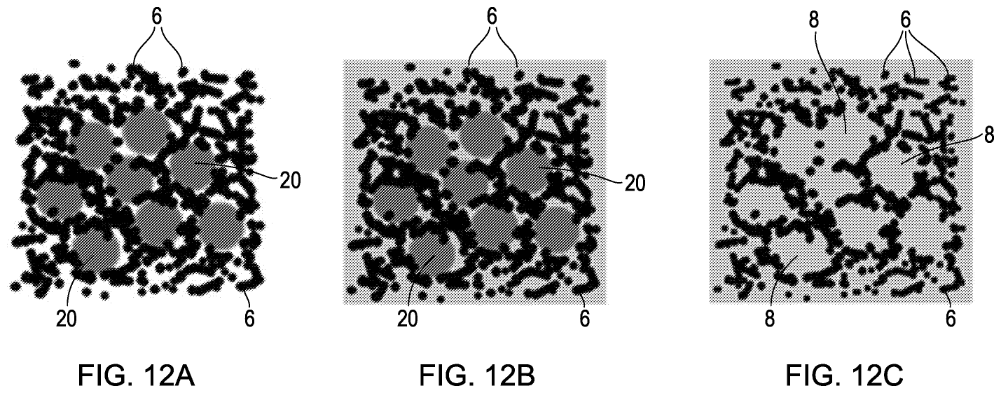

An example of pores which are enabled by a resorbable carrier material is an embodiment of the invention which comprises conductive elements and a carrier material (e.g., hydrogel) in the solid phase which is capable of being resorbed, e.g., within an approximate range of four to eight weeks, in extreme cases several months leading up to a year for full or close to full resorption. After curing and as resorption occurs during the chronic stage, the cured electrode can be somewhat compacted but comprises pores 8 which allow for much larger charge injection capacitance values than possible with an outer-surface-only electrode. The mixture in the liquid phase is injected at the target in bodily tissue and optionally a connector blob is attached to, and then cures to, a solid interface with a wire. The cured electrode thus includes the interface molded to the target 5 and the connector blob 26, the interface being integral to the connector blob 26. The connector blob ensures better connectivity to the wire even as the outside material gets resorbed. This is a system of two or more components, featuring a blob 26 of conductive elements focused to provide a stable interface (and small faradic impedance R) between a wire and the porous material that in turn has a large capacitance thanks to its pseudo-fractal interface surface that is in contact with the electrolyte composed of bodily fluids. FIGS. 12A-C are diagrams representing three stages with pores 8 left after resorption creating large capacitance values. FIG. 12 represents the mixture placed on a dry surface (outside a body, not in a patient, representing composition before injection) and resorbable material (gray spheres) which can be resorbed by the body tissues, e.g., macrophages. In FIG. 12 the liquid mixture has been injected into a body and interstitial fluid immediately fills up some pores between conductive elements 6, as indicated by the gray shading, but substantial resorbable material 20 is still present. Macrophages (not shown) begin digesting the resorbable material. FIG. 12C represents the cured electrode four to eight weeks post-injection. Macrophages have eaten the resorbable material (gray spheres are gone) and left additional pores 8 for interstitial fluid or other in-growing cells (fibrous tissue, etc.) to occupy. The cured electrode's material to electrolyte interface has changed fundamentally from two dimensional (only the outer surface of an electrode volume) to highly three dimensional (outer surface of an electrode volume and all inner surface interface locations between the electron-conducting electrode and the ion-conducting bodily fluid (ionic medium) on both, the outside and the inside of the cured electrode material.

The same is in principal true for magnetic conduction where magnetically permeable material is used instead of electrically conducting material to form the conductive (permeable) element-to-element bridge throughout the mixture which magnetically is placed in parallel to the less permeable surrounding tissue, thus forming a preferential path for magnetic field lines to travel from a generation location to a target location.

The same is in principal true for thermal conduction where thermally conducting material is used instead of electrically conducting material to form the conducting (heat transferring) element-to-element bridge throughout the mixture which thermally is placed in parallel to the less thermally conducting surrounding tissue, thus forming a preferential path for thermal conduction from a heat generation location to a target location (for heating; for cooling then the heat conduction is preferentially from a target to a heat drain location).

The same is true for vibratory conductive materials which transmit mechanical waves through a preferential patch from a vibration source to a target.

The present invention, comprises a variety of material specific physical parameters including, without limitation, curing inside or outside the body with the ability to adopt and retain the shape of a specific bodily optimal interface form, from flexible to stiff and/or rigid post cure, with different conductivities and the ability to mechanically interface with nearby locations within the body next to the target organ to have additional stress and/or strain relief on both, an organ and on a cured electrode post placement.

As disclosed herein, the needle-based and laparoscopic approach to placing liquid mixture 1 resulting in a cured electrode allows for a dorsal surgical approach to electrically (or by other means than just pharmacologically) connect to organs in novel ways, similar to the ability of connecting to intercostal nerves and ganglia of the autonomic nervous system, as further described herein.

Porous electrodes disclosed herein are highly advantageous for kilohertz frequency alternating current (“KHFAC”) and non-destructive DC nerve block, i.e., charge-balanced direct current (“CBDC”) nerve block. Recent preclinical studies with focus on reversible electric nerve block have shown that KHFAC nerve block can lead to DC contamination which may be more of a problem if an electrode's charge injection capacitance Qᵢₙᵢ is small. Other recent preclinical studies with focus on reversible electric DC nerve block have shown that a short-term nerve block using DC waveforms of several seconds in length is possible as long as the DC is injected as capacitive displacement current of the Helmholtz double layer at the electrode-to-electrolyte interface. Materials of large surface roughness such as Platinum Black have a larger charge injections capacitance Qᵢₙᵢ and thus allow a DC-nerve block to be applied for longer than with a material that has a smaller Qᵢₙᵢ (such as Platinum).

Advantages of porous metal electrodes vs. planar metal electrodes include (1) larger charge injection capacitance Qᵢₙᵢ allowing longer duration DC injection without incurring nerve damage, (2) relatively easy to manufacture via laser patterning, sputtering or chemical plating of conductive elements 6 that are then mixed with a liquid nonconductor 9 (plus potential additional additives) and thereby allow the forming of an electrode as described herein, (3) a volume effect vs. a surface effect may provide a large increase in charge injection capacitance. Using the entire electrode's volume as interface to the electrolyte in the body provides a huge charge injection capacitance.

In addition to forming a large electrode-to-electrolyte contact area throughout much of the volume of the liquid mixture with the approaches described herein, the surface area of the electrode on the outside of the volume can be made porous as well by lightly modifying the approaches described (using a variety of sizes for components that can be resorbed by macrophages) with the goal to create a surface porosity that promotes adhesion of advantageous cell types and minimizes the adherence of non-advantageous cells. This improves the modification of the fibrous tissue encapsulation 52 to create either thicker or thinner layers of connective tissue around the cured electrode, aiding with the mechanical integration of the electrode within the target tissue or the surrounding tissue near a target as well as with the option to aid with a modification of the electrochemical effects caused during an application of electrical energy to a target via the cured or curing mixture placed into the body.

In contrast to prior art electrodes 40, whose microscopic surface structure and macroscopic shape is formed ex vivo, the cured electrode 1 disclosed herein receives both its microscopic surface structure (the way all the elements within the mixture are aligned with each other at time of cure) and macroscopic shape (of the overall mixture) in vivo: by forming a “negative impression” of the target similar to how a cast forms as a mold around an arm or leg. This is achieved by one or more processes of manufacturing the electrode in vivo either inside a living organism or on the outside of a living organism. Although the electrode can be formed inside the body fully or in part, it may also be formed on the outside without touching the target tissue, but instead by adhering the electrode mixture to an electronic lead wire, with or without additional supportive structure, with the intent to modify the final electrode interface from a lead wire to an optimized neural interface.

### Placement and Connection to a Tens Electrode

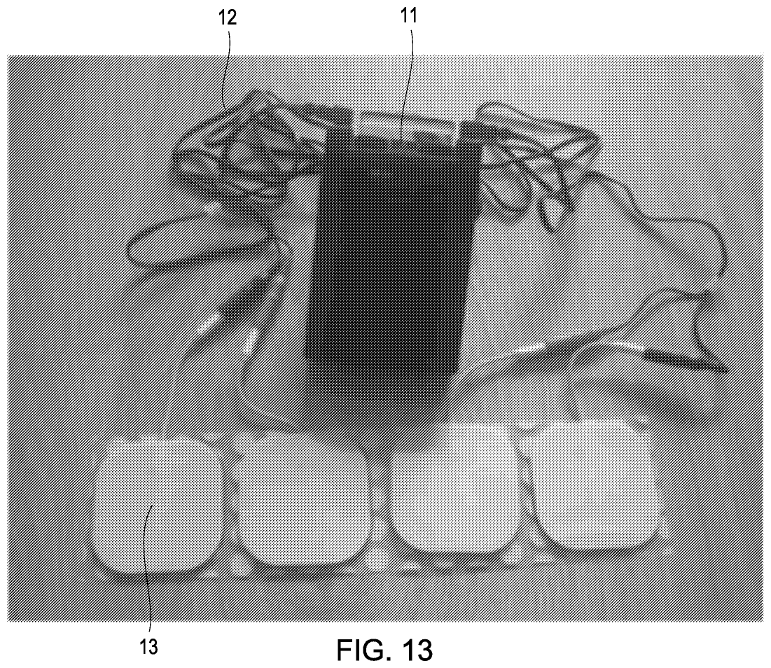

A Transcutaneous Electrical Neural Stimulation (TENS) system includes a signal generator 11, a least one cable 12 and a TENS pad electrode 13 (also 13A-13B), as shown in FIG. 13. TENS is often used for rehabilitation purposes or to provide non-invasive neuromodulation. TENS electrodes 13 are placed onto the skin and attempt to push enough current through the skin to a subcutaneous nerve that is close enough to the electrode to be depolarized, leading to an action potential. Unfortunately, densities of the current passing through the skin are dependent on the contact area (thus increasing when the electrode partially detaches) and it is often observed that stimulating a subcutaneous nerve requires current densities at the nerve which evoke action potentials in nerves and other sensory cells inside the skin between the electrode and the subcutaneous nerve which is the target 5, sometimes generating unpleasant sensations that limit the applicability of TENS to few neuromodulation situations where there is very little movement between a shallow subcutaneous nerve and an electrode placed on the skin to increase the chance of repeatable stimulation of said nerve via TENS. Even then, there may be a considerable spread in efficacy between patients and on the same patient between application days, which in turn may lead to poor patient compliance in the clinical reality. Advantages of TENS include the ability to electrically stimulate subcutaneous nerves that are within the proximity of the TENS electrodes placed onto the skin. There is no need for surgery to electrically stimulate these nerves in close proximity to the skin. Disadvantages include paresthesia and pain felt in the skin as side effects of the neural stimulation and loss of stimulation effects on the actual target nerve. Primarily, the current density in the skin underneath the TENS electrodes, especially in the skin at the edge of the electrodes placed on the skin, are significantly larger than the current densities near a targeted nerve, even at a depth of 0.5 to 1 cm for a target, and even more so 1 to 2 cm in depth away from the electrodes placed on the skin (distance measured perpendicular to the TENS electrode placed on the skin). The problem is that current densities at the level of the skin need to be increased to a level that causes the sensation of paresthesia or even pain in order to have large enough current densities (or voltage differentials) at a location deeper inside the body (i.e. 0.5 to 2 cm away from the electrode on the skin).

A low-impedance path for the TENS current to pass just below the skin while potentially not or only partially passing through the cells that sense paresthesia or pain in the vicinity of the outer layers of the skin avoids this problem, by means of the liquid mixture/cured electrode disclosed herein. One embodiment of the cured electrode comprises a contact pad 14 (just below the last layer of live skin as disclosed herein) to make a good connection to the TENS electrodes which is then connected through channels to a lower deposit of liquid mixture (uniting all the channels) which then is connected to a wire or another line of liquid mixture to reach a nerve with high current densities right away. This embodiment may further comprise an outside layer of liquid nonconductor/nonconductive layer around the deposit deep inside the skin.

Furthermore, placing an electrode via injection around a neural target and stimulating said electrode with electrical fields applied from the outside of the body to evoke action potentials (or even to cause a temporary temperature increase interrupting nerve conduction) near a cured electrode offers advantages over the prior art. The present invention's minimally invasive delivery, combined with other abilities like providing electric field shaping or guiding towards a target, (or away from an unwanted side target nearby), provide an advance over the prior art. The ability to be close to the target nerve offers the advantage of being able to activate or block or generally modulate said structure with small current amplitudes or voltage thresholds. Being able to guide the electrical energy from a contact pad 14 in subcutaneous tissue to the target location at e.g. 0.5 to 2 cm deep, or even deeper, by offering the current a path of <10Ω (or even <1Ω) means that current densities passing through the skin can be so small to cause no or only minor perceptions of paresthesia or pain during their passage through the outer layers of the skin. The present invention has the advantage of being able to more reliably activate neural tissue in close or far proximity to the outer skin of a person without intense or completely without the side effects of unwanted perceptions of paresthesia or pain in the skin near the TENS electrode. The present invention has the ability to further guide current around off-site targets that are not to be engaged with. Examples are multiple nerves running nearby and only one nerve to be engaged with from the surface of the skin or a nerve running near a ganglion where either the nerve or the ganglion is to be stimulated (i.e. electrically/magnetically) but the other one (ganglion or nerve) is to be not stimulated at the same time. Guiding the (i.e. electric/magnetic) field lines to the intended target and shielding them away from or guiding them around an unintended target are advantages of the present invention.

“TENS electrode” includes, without limitation, an electrode with a wire embedded in a hydrogel that separates the wire mechanically from the skin but provides an electrical connection to the skin. The electrical connection may further be emphasized by smaller sized TENS electrodes (size modification), change of materials (graphene, metals, or metal composites), different optimizations of the geometry of the subcutaneously placed contact pad 14 (one line wire, plus sign, double cross #, C-shapes, O-shapes, circles, ovals, partially or fully filled, entire networks or mashes formed from liquid mixture to ensure good contact to an outside TENS electrode. The TENS electrode may further incorporate small needles that penetrate the outer layer(s) of the skin and establish an even more directed energy path to the formerly injected cured mixture, further reducing energy requirements on the signal generator side as well as potential side effects from off-target stimulation as paresthesia, pain, etc. (caused from high voltage drop across the outer layer(s) of the skin) are minimized or completely avoided. The small needles may further aid with the anchoring of the TENS electrode in a specific location and add mechanical/locational and rotational stability of the TENS electrode on the outside of the body with respect to the user's body, even in a situation prone to sweat or movement.

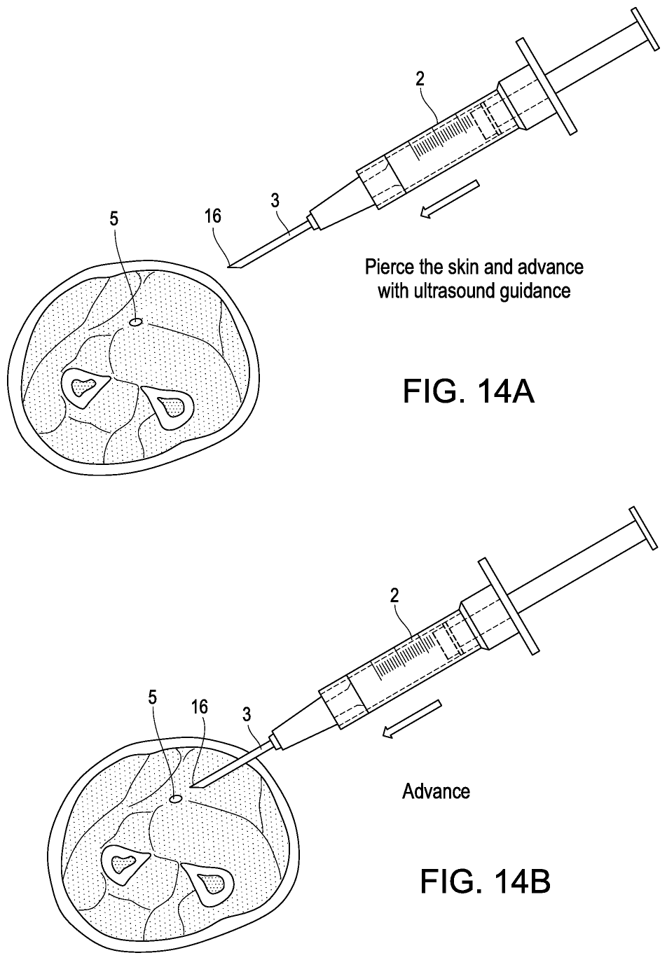

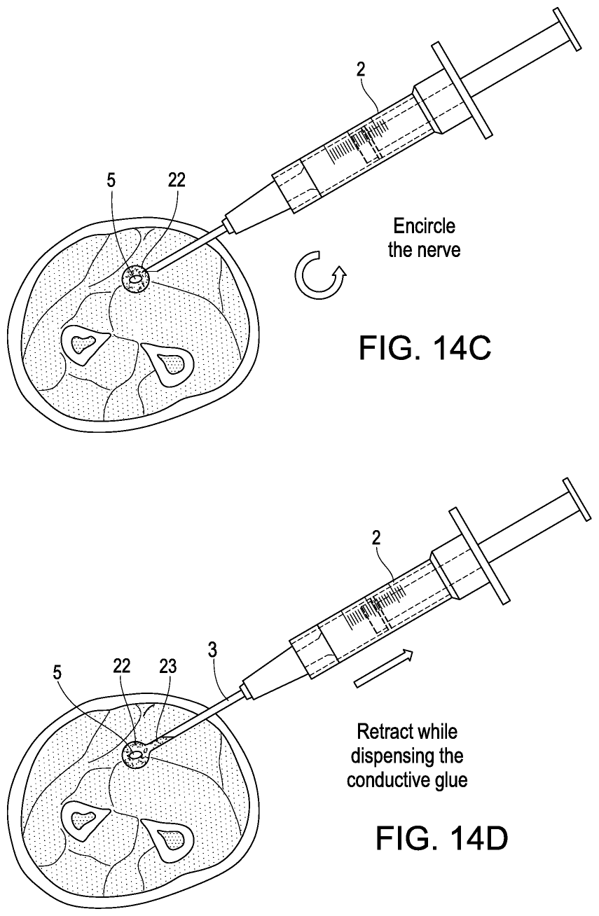

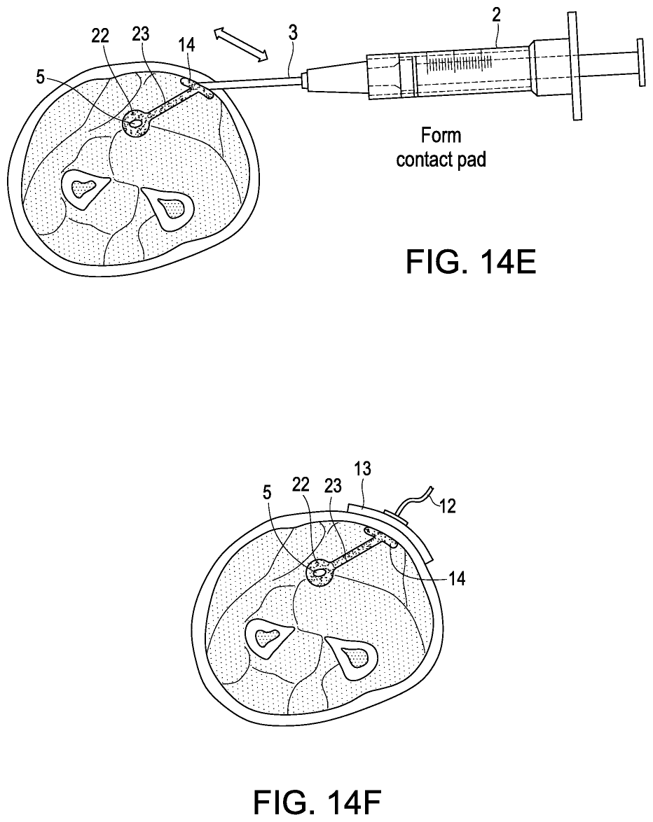

For example, a patient suffering from phantom limb pain after a traumatic injury (e.g., amputation) to the median nerve in the forearm is offered TENS stimulation to treat the pain. See FIGS. 14A-F, which are cross-section diagrams of the forearm. In order for the TENS signals to reach a PNS target 5 (here, median nerve) located in the deep tissue of the forearm, the present invention is injected around the median nerve and terminated just below the skin of the patient's arm to form a conductive pad. The procedure is conducted as outpatient procedure with localized anesthesia and in a 10 minute injection time frame. In one embodiment, the liquid mixture cures within 30-900 seconds of injection to form a mechanically compliant material with high electric conductivity form just below the surface of the skin to the target nerve but without an opening of the skin once that the opening has healed. This is different from percutaneous wires that are placed to remain reaching from the outside of the skin to a target nerve, creating a path for bacteria to follow from the outside of the skin to the nerve. As the present invention is completely implanted, there is no bacterial path from the outside of the skin to the nerve. By placing a TENS electrode on the skin at the location of the contact pad 14, and using a TENS electrode 13 connected to a stimulator 15, the patient is able to achieve pain relief by stimulating this deep tissue nerve with a surface stimulation technique. As the current is routed from just below the skin to the nerve of interest. The present invention has additional advantages for the usability and practicality in activities of daily living: If the dimensions of the TENS electrode on the outside of the body are larger and thus overlapping the dimensions of the contact pad 14 of the cured electrode, then moving the TENS electrode relative to the contact pad 14 does allow for current to reach the nerve even when the outside TENS electrode and the implanted pad are not 100% concentric in alignment. As the outer layer of skin is able to move with respect to the underlying structures, it is not uncommon to have a perfectly placed TENS electrode lose contact with a target nerve beneath the skin once that a person moves, bends or stretches as the electric field emanating into the body from the TENS electrode may be changed significantly by the bent, moved or stretched skin between the nerve and the TENS electrode on the outside. By providing a low impedance path from just beneath the skin to a neural target of interest, the electric field lines are still able to primarily take the path to the nerve even with movement, bend and stretch of the skin present as long as an injected (cured) mixture electrode is providing the low impedance path.

More specifically, the liquid mixture is injected all around the median nerve to form the neural interface as well as a conductive path similar to a metal wire. FIG. 14A is a diagram of a cross-section through the middle of the forearm with a dispenser 2 (here, a syringe with a needle 3) containing liquid mixture prior to injection targeting the median nerve. FIG. 14B shows the dispenser advancing to the target. Following the application of localized anesthesia, the dispenser is advanced (optionally under ultrasound, angiography or other visual guidance) to the target 5 median nerve buried deep inside the tissue. The proximity to the nerve can be verified by applying electrical pulses from an electrical stimulator 15 the dispenser's tip 16, as discussed herein, which is the only de-insulated part of the dispenser) in the range of standard neurostimulation pulses (if the connection target is a nerve but larger or stronger reactions of organs, such as muscle tissue), connected distally to the nerve are to be seen as the dispenser tip 16 comes into closer proximity with the target. If the electrical stimulator activates the nerve target, then the physician has confirmed electrical contact has been effected with the nerve. The lowest stimulation threshold driving the desired activation of the nerve (“activation threshold”) confirms that the deinsulated tip of the needle's cannula is in close proximity of the nerve. If the whole cannula of the needle is completely insulated along the way then the surgeon can pre-fill the needle with the conductive mixture and achieve the conductive path at the tip of the dispensing needle through the conductive mixture that fills the inside the of the cannula and is just about to emerge from the tip. Optimal location for the electrical connection to the target can be further confirmed by visualization such as ultrasound, x-ray, angiography or MRI where applicable, aided by the skilled physician's careful palpation of the anatomy in the vicinity of the target. FIG. 14C is a diagram showing dispensing of a ring-like portion 22 of the liquid mixture/cured electrode around the target. The dispensing can itself may be used to bluntly separate the target 5 from the connecting tissue or the liquid mixture can be dispensed in a cavity formerly formed around the nerve by blunt dissection. FIG. 14D shows dispensing the liquid mixture/cured that will form the cured electrode 1 forming a wire-like portion 23 of the cured electrode from the target 5 to the skin that provides an electrically conductive path from the neural target to the skin surface. FIG. 14E depicts dispensing the liquid mixture to form a contact pad 14 in the subcutaneous area which, in one embodiment, is formed by criss-crossing several lines of liquid mixture just below the skin. In this embodiment of the cured electrode, the ring-like/disk-like portion 22 is electrically connected to the wire-like portion 23 which is also connected electrically to the contact pad 14, such that the cured electrode may receive electrical current from a TENS electrode on the surface of the skin. Alternatively, the liquid mixture/cured electrode can be connected to neural signal generator 17 (“signal generator”) including without limitation an implantable pulseform generator (“IPG”) also implanted in the forearm located for example just below or in close proximity to the skin. FIG. 14F depicts utilization of a TENS electrode 13 applied to the skin at the approximately location of the contact pad 14 to drive electrical current to a deep tissue target 5 such as the median nerve.

In one embodiment, the present invention undergoes a phase change inside the body at body temperature, with or without the presence of air, water, and optionally cured by exposure to forms of energy such as ultrasound, UV or visible light, and radio frequency waves to form a partially solid, flexible or inflexible, or hard material. The carrier material 7 itself may solidify with or without the addition of air, water, energy, and it may release energy during the solidification process of forming a full or partially solid material. Conductive elements 6 to enable electrical conduction, and nonconductive elements to add to dielectric strength, are added. Hemostatic agents may be added in another embodiment. The present invention optionally may have a property to provide visualization inter-operatively via fluorescence, ultrasound or radio-/angiography, either as an inherent property of the liquid mixture, or through the addition of specific audio-, video-, mechano- or radio-opaque agents. Radio-opaque materials include, without limitation, platinum micro- or nano-elements.

In one embodiment the invention is a eutectic system comprising a liquid phase prior to injection and cures to a solid phase at or below body temperature, even under anaerobic conditions, the entire mixture forming a cured electrode upon solidification that provides impedance levels below 100Ω, in some instances below 1Ω, per mm of length and 1 mm² in diameter.

The present invention also comprises dispensers and systems that support the injection process by assisting a physician in finding the target (e.g., ability to electrically stimulate a nerve or sense neural responses) as well as by dispensing the liquid mixture or nonconductor.

The injection of the present invention enables formation of an insulated or uninsulated wire-like structure in one embodiment, having some similarities to (1) a bare wire in that a cured electrode 1 may not comprise a nonconductive layer 9 or (2) an insulated wire by the cured electrode optionally comprising and being at least partially surrounded by a nonconductive layer. Such a cured electrode comprising a nonconductive layer may be injected in its first liquid phase optionally through a multi-chamber dispenser 2, e.g., a first chamber 18 containing liquid mixture and the second chamber 19 containing liquid nonconductor. Or, in another embodiment, liquid mixture may be injected through one or more dispensers and the liquid nonconductor may be dispensed through at least one dispenser separate from the dispenser containing the liquid mixture.

Disclosed herein also are dispensers for pellets or capsules which are filled with liquid mixture or nonconductor, allowing the delivery of materials of different types at the same time, or to achieve curing which is delayed compared to liquid mixture or nonconductor not contained in pellets or capsules.

In one embodiment, a liquid mixture (and the resulting cured electrode) comprises resorbable materials 20 (e.g., FIGS. 12A-C) interspersed in a nonresorbable carrier material including, without limitation, sugars, amino acids, proteins and biodegradable materials which macrophages are able to consume any time after injection and within a period of 100 days, while leaving the external dimensions of the cured electrode intact, thereby creating pores 8 that the body is able to fill with interstitial fluid, connective tissue and other cells. These pores increase the electrode-to-electrolyte surface area as compared to a smooth surface of, for example, a traditional wire or metal contact, providing means to increase a cured electrode's charge injection capacity as the cured electrode “ages.” The cured electrode 1 may further comprise pre-cured components that are manufactured outside the body with materials in an already pre-cured component that facilitate partial resorption. An implementation may be an already porous structure, itself electrically conductive, that may be seeded with cells, nutrients or other eutroph factors that attract the in-growth of connective and/or neural tissue (as well as neural support tissue such as glia cells and the like), that is electrically connected with the cured electrode. Similar to the implementation with non-resorbable carrier material used as described, a version with resorbable components such as PEG is feasible in a combination with sugars, amino acids, proteins and biodegradable materials which macrophages are able to consume any time after injection and within a period of 100 days, while leaving the external dimensions of the cured electrode intact, thereby creating pores and having both, the biodegradable additives as well as the PEG be replaced by connective tissue and other bodily cells during the inflammatory and encapsulation process. To retain conductive properties for the energy modalities in question, the ratio of anon-conducting carrier to conducting components of 50% or less is required to ensure that the in-growing/invading cells and the surrounding cells forming the connective tissue (bio-fouling) do not severely degrade the charge injection properties as seen in chemically roughened electrode surfaces.

In one embodiment, the invention is capable of supplying an anodic current during the insertion of the dispenser (e.g. needle, cannula, auger, and the like) into the tissue and/or during the extraction of the dispenser from the tissue in order to achieve electrically mediated vasoconstriction. Anodic (positive) current activates a process leading to the constriction of blood vessels, reduces the probability of small vessels being ruptured during insertion and reduces bleeding time from small diameter vessels. Anodic current contracts blood vessels via the release of nitric oxide. This may be used in combination with the other modalities of energy injection into the body described in the present invention to reduce blood supply during i.e. the injection of a nerve block, be it via thermal, electrical (i.e. DC) or other modes as the restriction of blood flow to a set of arteries providing oxygen to a nerve is able to provide a temporary nerve block via ischemia. Alternatively, this approach of combining modalities may be used during a tissue ablation procedure to minimize pain within a region, organ or specific location of the body.

In contrast to using anodic current alone, higher-level (10V to 50V amplitude voltage controlled cathodic first, symmetrical charge balanced pulse trains at approximately 10 Hz) are capable of stimulating the muscle tissue of blood vessels directly and causing blood vessel contraction. This approach may be utilized to reduce bleeding not only during the placement process but also, in one embodiment, restricts blood flow to an organ. This may be used in combination with the other modalities of energy injection into the body described in the present invention to reduce blood supply during i.e. the injection of a nerve block, be it via thermal, electrical (i.e. DC) or other modes as the restriction of blood flow to a set of arteries providing oxygen to a nerve is able to provide a temporary nerve block via ischemia. Alternatively, this approach of combining modalities may be used during a tissue ablation procedure to minimize pain within a region, organ or specific location of the body.

Achieving a lower access resistance to a nerve in comparison to a traditional electrode put next to, adjacent or around a nerve. The access resistance to a nerve is directly related to the amount of charge that may be wasted while a nerve is to be stimulated: The closer an electrode is to a nerve, and especially the more tightly it wraps the nerve in the form of a cuff, the smaller a nerve activation threshold may be. See Plonsey/Barr discussed herein.

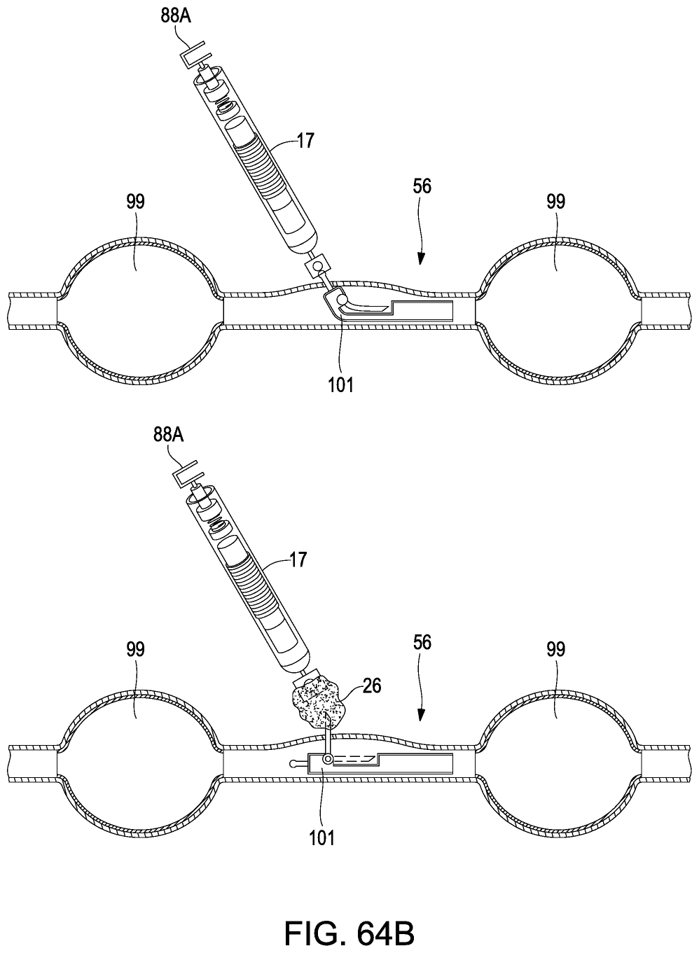

The cured electrode may be placed into, near, or around a blood vessel to be able to electrically stimulate, or block signal transmission in the blood vessel's cell wall. The liquid mixture may be injected around the outside of a blood vessel to stimulate arterial constriction or relaxation and thereby help to regulate blood flow into an organ a cell mass, the skin (to improve blood flow or reduce it to conserve body heat). The present invention may, in another embodiment, be placed around blood vessels to a tumor to prevent or reduce blood flow to a cancerous or unnecessarily growing or self-replicating site inside the body, thereby occluding blood supply and thus reducing the availability of nutrients and oxygen, leading to a reduction of the unwanted growth. Organ or tumor growth may be reduced or reversed (facilitating an intended cell/organ atrophy as medical treatment). For that, the liquid mixture may be injected by a dispenser comprising a catheter (FIGS. 64A/64B) from the inside of a blood vessel towards the outside of the blood vessel, either injecting it into the wall of the blood vessel or outside to the blood vessel so as to electrically contact the blood vessel's outside to an implanted wire 10. Alternatively, another component of the cured electrode may be injected to the outside of the blood vessel with an approach that comes from further away from the blood vessel and comes closer to the blood vessel. The liquid mixture may be injected as a ring around a blood vessel by injecting it through at least one needle that pierce the blood vessel wall from inside to outside and create either an interrupted (but overall connected) or continuous ring around the blood vessel outside. Such a ring-like shape portion 22 of the cured electrode may then be contacted by a wire-like portion 23 of the cured electrode to facilitate the electrical connection to a blood vessel to a specific location inside the body or just below the skin of a patient. The wire-like portion 23 is located from outside the blood vessel from a separate injection.

Utilizing different activation thresholds for nerves and blood vessels helps to separate the two when closely aligned or nearby: While nerves will likely be depolarized at stimulation current amplitudes of 1 mA stimulation current applied for a 200 μsec pulse width, in a symmetrical, cathodic first waveform, blood vessels will more likely not react until about 10 mA++ are applied based on the fact that blood vessel walls are lined with smooth muscle cells whose activation thresholds are at least about an order of magnitude higher than that of axons in nerves. It is feasible without a nonconductive layer being placed to stimulate and activate a nerve next to an artery, but enables stimulation of only the blood vessel (e.g., to contract) but not depolarization of a nearby nerve through combinations of various stim and block waveforms.

In one embodiment, a cured electrode can be placed on the outside of a tumor to ablate existing blood vessels and newly growing blood vessels that may regrow nearby the old (ablated) ones with the tumor trying to replace the ablated ones. With an existing cured electrode already present only a small incremental amount of cured electrode is needed to fully encase new blood vessels the tumor might have grown, ablation may be used to heat up the newly and previously placed cured electrode when only one point of the entire cured electrode network around the tumor is touched.

By injecting angiographic contrast agents to blood arterial vessels that supply cancerous tissue, a tumor can be visualized against the surrounding tissue with increased contrast compared to the surrounding tissue, the contrast being increased if combined with other modalities such as contrast assisted PET and CAT scan of the cancerous tissue. Once the cancer and its margins have been visualized, a needle based delivery of a liquid electrode mix is possible under angiographic visualization. This allows the physician to place the liquid electrode in the border region of the cancerous tissue. If fluoroscopic contrast agents are used to further illuminate cancerous tissue under i.e. UV light, then a laparoscopic approach may be utilized to aid the physician in guiding the needle used for the delivery of the liquid electrode. The physician may aim to (a) inject the liquid electrode mix into the cancerous tissue itself at one or more locations (if i.e. an ablation of the tumor from the inside outward is intended), or (b) inject the liquid electrode mix into the cancerous tissue on the margins between cancerous and healthy tissue at key locations such as near vital arteries or veins that a tumor may not be easily resected from (if e.g. an ablation of the tumor at that barrier region is required to avoid spreading or prepare a later surgical removal of the tumor following some recovery time between ablation and resection as dead tumor tissue may be more easily resected from said vital tissues or organs), or (c) inject the liquid electrode mix into the cancer margins between cancerous and healthy tissue meaning injecting it into healthy and cancerous tissue (i.e. to ensure a wider ablation region and increase the probability of stopping the spread of any cancerous tissue), or (d) inject the liquid electrode mix around the entity of the cancerous tissue just outside the tumor margins (i.e. with the intent to ablate the entire outside and most of the inside of the tumor as well as blood vessels supplying it with nutrients, or (e) inject the liquid electrode mix around the blood vessels supplying the cancerous tissue with nutrients (i.e. with the intent of utilizing ablation of the blood vessels to starve the tumor from nutrients without the risk of bursting the blood vessels as the cured electrode may be heated in a more controlled manner than a traditional ablation electrode approach would allow).

The present invention, in some embodiments, may be placed into, near, or around an organ, especially specific structures of an organ such as internal blood vessels or neurons, or an inside or outside wall of the organ to be capable of electrical stimulation, or blockage of signal transmission, in the organ, the innervation or the blood supply of the organ, for example, the bladder. Organ activity can be changed by increasing or decreasing neural communication into and out of the organ, and some organ growth and activity can be up- or down-regulated by allowing more or less blood enter the organ, such in the case of the gut, the liver, the lungs or the kidney which are exchange systems for the body, utilizing a fine mesh of blood vessels intertwined with other vessels who either add or extract chemicals in the form of dissolved gasses or liquids. The present invention allows for an efficient way to contact an organ, such as by injecting the liquid mixture to the outside wall of an organ near an innervation point. The conductive elements may in such case comprise a biocompatible mesh (not pictured but well known in the art) attached via a liquid mixture and/or sutures to the organ, the electrical conduction between mesh and the organ being accomplished or improved by the liquid mixture.

Nerves close to the surface of the body that have been shown to respond to current injection by thin needle (i.e. electro-acupuncture and similars) can be targeted more reliably with a TENS unit once a cured electrode has been placed into and/or around the target nerves close to the surface. The physician first verifies the efficacy of neural stimulation of a specific nerve via thin needle (i.e. acupuncture), then map the nerve's dimension with the needle in the specific location (looking for smallest activation threshold), which may be assisted by ultrasound or angiography visualization. The physician may choose to only place a cured electrode into the nerve sheath, or the physician may choose to place a cured electrode as a partial or full ring around a nerve target of interest. Then the physician may choose to extend the liquid mixture from the nerve target as he/she is retracting the needle towards the skin, thereby forming a bulge or a wire-like extension from the bulk of the cured electrode near/around the nerve. This extension may be just 1-2 mm in length or it may be 10 mm in length or more with the intent to guide electrical field lines in an anatomically preferential path to the target nerve, the best path electrically not always being the shortest path mechanically. By terminating the extension of the liquid mixture near or just below the outer layers of the skin the clinician ensures that the tissues surrounding the nerve and the skin remain fully supply with blood and thereby nutrients, oxygen etc. while allowing a more reliable and interface to the nerve formerly only activated with needles from the outside of the body.

The mixture may further contain components that provide a magnetic interface effect, allowing an easy way to find a subcutaneous cured electrode with a magnet placed on the outside of the skin. This approach may aid the patient in placing the TENS electrode (then potentially with an added magnetic component via i.e. rare earth magnets) always in the correct spot and possibly, if alignment is important, in the proper alignment as long as the liquid mixture in the subcutaneous tissue has either two magnetic poles, or two locations that are able to interface with magnets (e.g. ferro- or ferrimagnetic elements). Furthermore, an electrically nonconductive layer 9 but magnetically active mixture may be placed into the subcutaneous tissue secondarily to the initial placement of a cured electrode. One such configuration is an M-E-M (magnetic-electric-magnetic) design where the electrical interface is centered between the two magnetically interfacing cured electrodes. The corresponding TENS electrode (or a TENS electrode placing device) may utilize two rare earth magnets to align the TENS electrode with the center, electric, interface by magnetically aligning with the two outer M cured electrodes. This may greatly enhance user friendliness for finding the subcutaneously placed cured electrodes and always optimally placing the TENS electrode on the outside of the body.

Nerves may further be visualized by angiography and injection of angio contrast agents into the arterial blood supply of the neural target. The liquid mixture may contain contrast agent added to the mixture (same agent as injected arterially, platinum components, etc.) to aid with the visualization during cured electrode placement. With both, the liquid mixture/cured electrode and the nerves showing sufficient contrast against the surrounding tissue, angiography may be utilized in very similar ways as done during the placement of a stent during a cardiac procedure.

The biocompatible liquid mixture comprises conductive elements and nonconductive carrier material and optionally other elements (affecting curing times, integration with the body, inflammatory response, etc.) which is mixed together by the physician shortly before placing it inside the body (or thawing it shortly before placing it inside the body) and which cures and functions as a conductor inside the body, i.e., an aqueous environment with or without the aid of additional energies. The liquid mixture has great mechanical stability and homogeneity even though, prior to curing in the body, it may flow as a liquid, gel or paste. After curing, the cured mixture has conformed to the bodily structures against which it was formed. The resulting cured electrode has resistance <10 ohm for a shape of 1 mm diameter and 1 mm of length (meaning a volume impedance of 10 Ohm*mm) (−). The present invention is not intended as a thin film, and this application specifically disclaims any aspect as a thin film manufactured outside the body.

### Carrier Materials

The carrier material 7 provides the capability of being injected because it comprises first a liquid phase and then it cures to a solid phase and, as such, the liquid phase carrier material allows injection of the conductive elements 6 which are interspersed in the carrier material 7. Although curing may begin outside of the body, at least some of the curing process is capable of occurring inside the body, distinguishing the invention from prior art electrodes which are pre-configured prior to implantation. The carrier materials include hydrogels, elastomers, tissue glues, protein glues tissue adhesives other than glues, tissue sealants, coagulants, cyanoacrylates, bone cements, dental resins, and dental amalgams. If powders are part of an embodiment, then the powder's dispenser allows the formation of a mechanical structure (with or without the addition of other materials) that becomes a less pliable structure after curing. Powders akin to some of the powders used as coagulants can form the non-conductive mechanical support structure by first coagulating bodily fluids and tissues in place co-located with the conductive carriers, while limiting the production or aiding with the transmission of excess heat away from sensitive tissues such as the neural target tissue.

Fast curing is often optimal, for example, a range of 1 to 5 seconds as the body is constantly moving with heart beats, breathing, pulse even in distal arteries, moving muscles; in other embodiments it is preferred for the curing to take no longer than 900 s. Although the curing time for a specific implementation may exceed 15 minutes (900 s) of time to reach the solid phase, a curing duration of less than 15 minutes is better in a surgical implementation than a duration of longer than 15 minutes. This curing duration does not include the encapsulation by the body or the partial dissolution and/or resorption of components or materials included as part of the embodiment of the invention in its liquid phase. Slow curing also has specific application for better long term integration to the surrounding tissue. Forming a good mechanical bond to the biological tissue is optimal. In the liquid phase the carrier material is dispensed via injection. The carrier material may have gel-like property as long as it is capable of curing further into a more stable form retaining the shape of contours of the target around which it is injected and molded against the contours of the target. The carrier material may be a putty-like, amorphous material (similar to “Sugru Mouldable Glue” in its mechanical behavior but, in contrast to Sugru, biocompatible; and curing fully without the release of toxic or partially toxic gases and other substances) that may cure inside the body, retaining some mechanical flexibility post curing or not. The carrier material may comprise a eutectic paste. The carrier material may be doped with the body's own cells to better integrate. The carrier material may also be doped with stem cells from the patient or other living organisms. It may be doped/mixed with radio-opaque elements or dyes (for example to allow the verification of the placement of the carrier material in its liquid phase around the nerve or through tissues as well as the ability to detect breaks in the cured electrode after years of wear and tear). It may be doped with sugars or other resorbable materials 20 which the body's macrophages resorb in order to change the injected liquid mixture into a porous structure (FIGS. 12A-C) as time passes and the body partially digests the blob, thereby increasing the active surface area to the embedded conductive elements. The carrier material also may comprise fluorescent elements or dyes that allow the verification of placement around the nerve or through tissues intra-operatively by shining a UV light onto it that does not cure the carrier material but instead makes it glow in the dark of the cavity and around the nerve or, if injected into the nerve, makes it glow from inside the nerve. The carrier material may also comprise pharmacological agents to produce short-term or sustained drug-delivery that have complementary action to the cured electrode (e.g. lidocaine to reduce pain from operation and/or produce local anesthesia, or other nerve-block agents or other pain-alleviating agents that may ordinarily be injected near a neural target).

The viscosity of the liquid mixture affects how readily it will flow and distribute itself within a created body cavity. Lower viscosity liquid mixtures will flow more easily than higher viscosities, but higher viscosities have greater ability to stick to a specific placement location and to hold a specific space filled without flowing to unintended spaces.

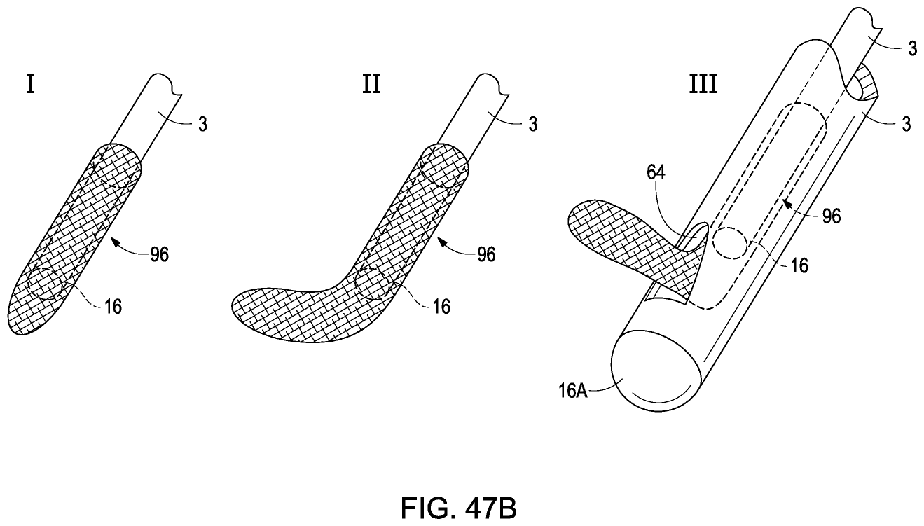

A low viscosity liquid mixture has an advantage in its greater capability to be injected behind or below a nerve but in some embodiments may be used with a pre-formed mold (e.g., FIGS. 47A and 47B) to be inserted at the target to hold this space open during the injection or other placement process. Higher viscosity affords a greater capability for the liquid mixture to resist forces from the surrounding biological tissue to be pushed out of the cavity, thereby retaining a minimum ring-like portion 22 around a nerve when injected without a pre-formed mold.

Among other advantages discussed elsewhere, higher viscosity carrier materials have the following advantages in aiding: (1) with combatting separation of conductive elements from the carrier material as the liquid mixture passes from the larger inner diameter of a dispenser to a smaller diameter needle; (2) with dispensing as the thicker material sticks in place; and (3) with surgical integration as the more viscous liquid mixture may be shaped in place, holding its form and shape to a certain degree before curing. Differences in viscosity are primarily achieved by changing the ratio of conductive elements vs. silicone carrier material. A secondary way of changing the viscosity is by adding surfactants, thickening or thinning agents. Thinning agents may be selected from a group including water, PEG solutions, glycerine, and other inactive excipients commonly utilized in the pharmaceutical industry found at https://www.accessdata.fda.gov/scripts/cder/iig/index.cfm. Thickening agents may be selected from a group including inactive polymer powders such as polyethylene glycol (“PEG”) powder, peptide powders, starches, sugars, silica powder, and additional metallic and non-metallic fillers that may or may not add further elements of high conductivity (graphene being one of them).

A comparison of the hydrogel PEG, fibrin glue and cyanoacrylate as a carrier material is useful. PEG becomes mechanically flexible in the solid phase after curing with medium to high water content. When PEG is hydrolyzed it dissolves, and its stability depends on crosslinking, and dendritic structures create higher cross linking. It may be polymerized or cross-linked to the solid phase by different mechanisms. Fibrin glue is also mechanically flexible as a solid having a medium water content. It can degrade enzymatically in vivo and its stability depends on crosslinking. Fibrin requires frozen storage and it may be stored up to two years, and it requires thawing before use. Cyanoacrylates have low water content and variable rigidity. Cyanoacrylates are very stable and hydrolyze over time, though the average time for a cyanoacrylate to hydrolyze is to be expected longer than the time needed for the body to take over the mechanical stabilization before the cyanoacrylate has substantially weakened. If the intended location in the body is anticipated to be under significant physical stress/strain, e.g. near contracting muscles or joints, longer hydrolysis times, at least greater than the time it takes to form a stable fibrous capsule around the implant, are desirable. The rate of fibrous capsule formation itself may be variable depending on location in the body, and is likely a function of tissue vascularity. Higher vascularization means a higher mobility of fibroblasts and macrophages to the site of implantation and thus a higher rate of scar formation.

### Hydrogel

A hydrogel is a network of hygroscopic (water-absorbent) polymer chains swollen with water. Hydrophilic gels that are usually referred to as hydrogels are networks of polymer chains that are sometimes found as colloidal gels in which water is the dispersion medium. One definition of a hydrogel is that of a water-swollen and cross-linked polymeric network produced by the reaction of one or more monomers. Another definition is that of a polymeric material having the ability to swell and retain a significant fraction of water within its structure, but not dissolve in water. Hydrogels also possess a degree of flexibility similar to natural tissue because of their large water content. The ability of hydrogels to absorb water arises from hydrophilic functional groups attached to the polymeric backbone, while their resistance to dissolution arises from cross-links between network chains.

A form of hydrogel, cross-linked gelatin forms a cohesive matrix with tunable post-curing viscosities. Gelatin easily flows at temperatures exceeding 50 degrees C. and undergoes a reversible transition from solid to gel under specific conditions. Gelatin is a naturally occurring, and generally well-tolerated biomaterial. Gelatin is an irreversibly hydrolyzed form of collagen. It is an animal collagen thermally denatured with a very dilute acid, with many glycine residues (almost one in three), proline and 4-hydroxyproline residues. A typical structure is -Ala-Gly-Pro-Arg-Gly-Glu-4Hyp-Gly-Pro. While the basic building blocks of gelatin and collagen are the same, collagen retains more of its tertiary fibril structure. Conductive elements may be mixed with gelatin above its gelation temperature (temperature threshold for the formation of a thermoreversible gel), injected into the body, and allowed to cool. The resulting cured gel containing conductive elements will be electrically conductive. Furthermore, gelatin comprises the processed form of collagen. Gelatin can be ground up, mixed with conductive elements (and optionally a surfactant and other additives) and then added to the carrier material to form a paste that undergoes the phase change in the body, and immediately after curing begins a process by which the body's inflammatory response starts to exchange, digest, or replace the gelatin based elements with the body's own cells, thereby growing into the cured electrode or partially digesting the cured electrode, which leaves pores inside the remaining cured electrode bulk, thereby creating a porous interface of much larger surface area compared to a smooth surface of the same outer dimension.

PEG is a hydrogel, and it has many advantages as a carrier material for the liquid mixture and the cured electrode. Hydrolysis of 20 kDa cross-linked PEG is approximately 4-8 weeks. Higher molecular weight or higher cross-linking density may achieve longer hydrolysis times. PEG is hydrophilic and will therefore adsorb proteins during and after implantation to the surface, without greatly denaturing them. This increases biocompatibility and adherence to surrounding tissues compared to silicone and other hydrophobic surfaces. PEG has much greater replacement by the body than silicone. PEG provides a regenerative growth substrate for repairing damaged neurons/axons. PEG's repeating ethylene glycol units provide ample opportunity for hydrogen bonding, particularly with carboxylic acids in microenvironments above their pK (˜4.5). Importantly, PEG can act as a chelator or buffer for bicarbonate, which can locally decrease the pH or presence of carbonic acid in the microenvironment which has demonstrated benefits for wound healing. A PEG based liquid mixture/cured electrode may be manufactured with an intentionally higher impedance than other carrier materials, by adding non-conductive materials, elements or elements to the mixture. The resulting insulating PEG cured electrode may be used to restrict electrical current flow from certain areas, or as a liquid nonconductor it may be used to achieve an insulation around the liquid mixture/cured electrode. In some embodiments PEG may be made more nonconductive by adding elements that make the final cured PEG more attractive to in-growth of fibrous tissue thus increasing insulation with the body's own fibrous tissue, in comparison to the PEG based cured electrode that is intended to remain conductive (with conductive elements) as the PEG is replaced by the organism.

In yet another embodiment, the addition of gelatin to carrier materials such as PEG hydrogels or silicones, is used to intensify the body's inflammatory response, on a continuous scale according to the concentration of gelatin added, thereby increasing the amount of encapsulation 52 that is formed by the body around the cured electrode. The cured electrode may also comprise gelatin to thicken encapsulation, for example, to keep the cured electrode in place and prevent conductive elements 6 from flaking off, or it may be applied as a second layer on the outer aspect of the electrode formed next to, or around, a nerve, to ensure a thicker encapsulation to increase the electric impedance towards the outside of the cured electrode with the goal to have a low impedance (i.e. thin layer) encapsulation on the inside of the ring-like portion 22 that touches the nerve and a large impedance (i.e., thick layer) encapsulation 52 on the outside of the cured electrode against the surrounding tissue. This approach may be used to interface selectively with various nerves running in parallel or it may be used to minimize muscle fiber activation or simply reduce current outflow out of the cured electrode wherever it does not do any work stimulating the target. The control of the encapsulation, and thereby the electrical interface impedance between the cured electrode and the surrounding tissue aids in constructing a lower side-effect and more energy-efficient neural interface that saves on battery lifetime for signal generators.

PEG is a carrier material which comprises a liquid phase to which conductive elements may be added or attached. PEG hydrogels are biodegradable and are resorbed by the body after injection and after curing to the solid phase of a cured electrode, thus allowing the formation of pores.

PEG is a polyether compound and is also called polyethylene oxide (PEO) or polyoxyethylene (POE), depending on its molecular weight. The structure of PEG is commonly expressed as H—(O—CH2-CH2)n-OH. PEG, PEO, and POE refer to an oligomer or polymer of ethylene oxide. The three names refer to the same compound, but historically the term PEG is preferred in the biomedical field, whereas the term PEO is more prevalent in the field of polymer chemistry. As used herein, PEG or polyethylene glycol means any compound comprising the general structure X—(O—CH2-CH2)n-Y where n is a variable number of repeat units and X and Y are functional groups at the terminal ends. If X=Y, then the PEG is called a “homo-bi-functional PEG.” If X does not equal Y, then the PEG is called a “hetero-bi-functional PEG.” If X or Y=—OH and is therefore unmodified, then the PEG compound is a “monofunctional PEG.” Because different applications require different polymer chain lengths, PEG has tended to refer to oligomers and polymers with a molecular mass below 20,000 g/mol, PEO to polymers with a molecular mass above 20,000 g/mol, and POE to a polymer of any molecular mass. PEGs are prepared by polymerization of ethylene oxide and are commercially available over a wide range of molecular weights from 300 g/mol to 10,000,000 g/mol. In one embodiment, the PEG suitable for the carrier material is within a range of 1000 g/mol-50,000 g/mol.

While PEG and PEO with different molecular weights have different physical properties (e.g. viscosity) due to chain length effects, their chemical properties are nearly identical. PEGs/PEOs come in a variety of molecular weights, with varying degrees of polydispersity. Furthermore linear PEG chains may be initiated and terminated by different functional groups, e.g., —CH3, —OH, —COOH, —SH, depending on the initiator, capping agents, and polymerization process used.

PEGs are also available with different geometries. In order to facilitate efficient crosslinking, a branched structure is desirable for a carrier material herein. The two market leaders for PEG products, Coseal and Duraseal, use 4-arm PEG which are suitable as carrier materials CoSeal has a MW of 10 kDa and DuraSeal has a MW of 20 kDa. Hyperbranch also provides a dendritic PEG adhesive with much higher branch numbers which are suitable. Branched PEGs have three to ten PEG chains emanating from a central core group. Star PEGs have 10 to 100 PEG chains emanating from a central core group. Combination PEGs have multiple PEG chains normally grafted onto a polymer backbone. The numbers that are often included in the names of PEGs indicate their average molecular weights (e.g. a PEG with n=9 would have an average molecular weight of approximately 400 daltons, and would be labeled PEG 400.) Most PEGs include molecules with a distribution of molecular weights (i.e. they are polydisperse). The size distribution may be characterized statistically by its weight average molecular weight (Mw) and its number average molecular weight (Mn), the ratio of which is called the polydispersity index (Mw/Mn). MW and Mn may be measured by mass spectrometry or by gel permeation chromatograhy. All the above configurations of PEG are suitable as the carrier material for the present invention.

PEG is soluble in water, methanol, ethanol, acetonitrile, benzene, and dichloromethane, and is insoluble in diethyl ether and hexane. It is coupled to hydrophobic molecules to produce non-ionic surfactants. If inadequately purified or characterized after synthesis, PEGs may potentially contain toxic impurities, such as ethylene oxide and 1, 4-dioxane. Ethylene Glycol and its ethers are nephrotoxic if applied to damaged skin. It is therefore important that the source of PEG materials be rigorously quality controlled, as has been accomplished by a number of other manufacturers having FDA-approved PEG adhesive formulations on the market.

PEG and related polymers (PEG phospholipid constructs) are often sonicated when used in biomedical applications. However PEG is very sensitive to sonolytic degradation and PEG degradation products may be toxic to mammalian cells. It is, thus, imperative to assess potential PEG degradation to ensure that the final material does not contain undocumented contaminants that may introduce artifacts into experimental results.

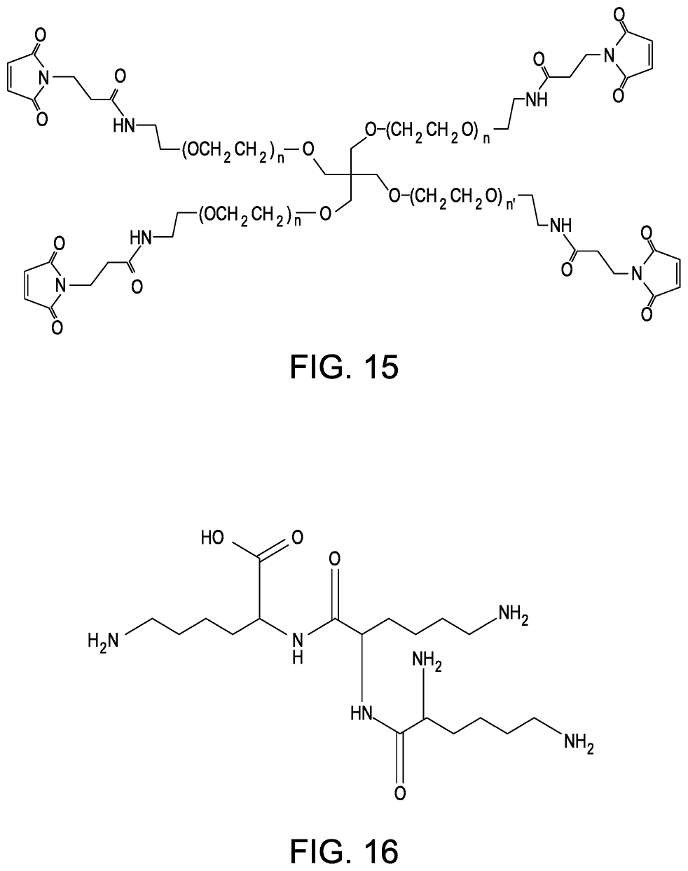

An example of a hydrogel which can be used as a carrier material in the mixture is a PEG tissue sealant commercially available called DuraSeal. It comprises a 2-part solution system that when mixed forms a synthetic hydrogel coating that is biocompatible and degraded in the body over 4-8 weeks. More specifically, it comprises (1) a 20 kDa, 4-arm Branched PEG, terminated with NHS-ester-activated functional groups, (2) a trilysine crosslinker, and additives including (4) a preservative: BHT (butylated hydroxytoluene), (5) Dyes—help to ensure mixing is complete, FD&C Blue, (6) Buffers—sodium phosphate for PEG, and (7) Buffers—sodium borate for trilysine. The PEG is dissolved at a concentration of 0.5 g in 2.5 ml of sodium phosphate buffered saline (20% w/v or 10.0 mM). The tri(L-lysine) acetate is dissolved at a concentration of 10.5 mM in 2.5 ml 75 mM sodium borate decahydrate. FIG. 15 is a diagram of the chemical structure of PEG in DuraSeal. FIG. 16 is a diagram of the chemical structure of Trilysine in Duraseal (showing 4 primary amines, as well as 2 secondary amines that are not reactive with NHS, which is an abbreviation for N-hydroxysuccinimide).

TABLE TWO
Total and relative amount of ingredient
per single dosage of Duraseal (~5 g).
Per Dosage Delivery

| | g | mg/kg |
|---|---|---|
| 4-arm PEG-NHS | 0.5000 | 7.143 |
| Trilysine | 0.0106 | 0.151 |
| Sodium Borate 75 mM | 0.0640 | 0.914 |
| Sodium Phosphate | 0.0027 | 0.038 |
| FD&C Blue | 0.0005 | 0.007 |
| BHT Preservative | 0.0001 | 0.001 |

TABLE 2
Cross-linking ratios of Trilysine:PEG

| | | | | Ratios |
|---|---|---|---|---|
| PEG | | Trilysine | | Trilysine:PEG |
| 0.5 | g PEG | 0.01057 | g Trilysine | 0.021 |
| 20000 | MW PEG | 402.53 | MW Trilysine | 0.020 |
| 0.000025 | mol PEG | 0.0000263 | mol Trilysine | 1.050 *** |
| 0.0025 | L Buffer | 0.0025 | L Buffer | 1.000 |
| 0.010 | M PEG | 0.0105 | M Trilysine | 1.050 *** |
| 4 | NHS/PEG | 4 | Primary NH2 | 1.000 |

Note:
*** 5% Excess of Trilysine: in order to ensure full consumption of NHS sites during reaction.

The above formulation of the PEG sealant is an example of a carrier material for use in the liquid mixture, with the addition of conductive elements at high enough concentration to create a continuous distributed network of separate conductive elements (described herein) such that the impedance measures below 100 ohm/cm for the purpose of curing to a solid electrode in vivo.

The PEG branching structure may be varied by changing the polymerization conditions during preparation of the PEG precursor in order to change the reaction kinetics and the ultimate hydrogel mechanical properties. The prototypical PEG used in commercially available PEG sealants is a 4-arm branched structure. The PEG structure of the present invention's carrier material may include, without limitation, any of the following structures:

(a) Linear—homo-bifunctionalized PEG provides two reaction groups and is the minimum required to form a continuous interconnected polymer hydrogel network. However, given the competing hydrolysis rate of NHS or other activated end groups, there will be some terminal PEG molecules, such that the network is likely to have some discontinuities in its structure. This may yield a low degree of crosslinking, and hence a less stiff or cohesive gel. For temporary cured electrodes or for anatomies that are particularly sensitive to stiff materials this may be a particular benefit.
(b) Branched multi-arm—The most common single-order branching structures of PEG are 3-arm, 4-arm (pentaerythritol core), 6-arm (dipentaerythritol core) and 8-arm (hexaglycerol or tripentaerythritol core). Due to multiple binding sites, the multi-arms are more likely to form an interconnected network upon curing than linear PEG, and the multi-arm structure is highly suitable as the carrier material. The increased number of binding sites will decrease polymer network mobility and increase stiffness and strength. (c) Multi-level branched (stellate/star)—the most common PEG dendrimers are generation 1, 2, 3, and 4, and yield 2\hat{ }(1+generation) potential functional —OH groups available for reactions. Certain dendrimers with particularly high cationic surface charge yield toxic side effects upon degradation, disrupting biological membranes and resulting in hemolytic toxicity.
(d) Random hierarchy—randomly branched PEG or “hyperbranched” PEGs are synthesized by random anionic ring-opening multibranching copolymerization of ethylene oxide with glycidol as a branching agent, leading to poly(ethylene glycol) structure with glycerol branching points. The benefit is a higher degree of branching and easier rate of manufacture. However, the downside is a stochastically formed polymer, which may lead to inconsistencies in polymer viscosities in batch-to-batch processing.

Ingredient concentrations in precursor PEG solutions may be varied by increasing or decreasing the molarity of the solutions and these variations will change the reaction rate and system viscosity. For example, increasing the concentration of PEG will increase precursor solution viscosity. Increasing crosslinker concentration relative to PEG will yield faster curing rates. It will also affect swelling characteristics. Swelling of Duraseal is ˜98% by volume. Increasing or decreasing the viscosity of the precursor solutions has advantages in getting selective or consistent suspension of conductive elements. Viscosity also largely determines the pressure required to deliver the solutions through syringe/needle devices. Lower viscosity solutions (lower concentrations) will mix more easily than higher viscosity solutions. Lower concentrations will also cure slower compared to higher ones according to a molecule-molecule interaction (collision theory).

The PEG molecular weight may be varied by changing the polymerization conditions, (e.g., the use of varying monomer feed-rates, feed-ratios, catalyst choice, catalyst ratio, duration of polymerization as well as the use of capping agents to quench the reaction) during preparation of the PEG precursor changes the reaction kinetics and the ultimate hydrogel mechanical properties as well as the viscosity of the precursor PEG solution to enable selective suspension or precipitation of conductive filler elements. Suitable PEGs for the present invention are in the range 5 kDa, 10 kDa, 20 kDa 4-arm branched structure. Higher molecular weight PEG will take longer to degrade and therefore have longer time for clearance in renal system. A hydrogel carrier material of 30-50 kDa is suitable for the present invention. At some point >50 kDa, the rate of dissolution of the lyophilized PEG powder with the diluent will be a limiting factor. E.g., 100 kDa PEG is likely to take over 15 minutes to reconstitute in aqueous diluent buffer without applying additional heat or solvents. This would make clinical implementation challenging.

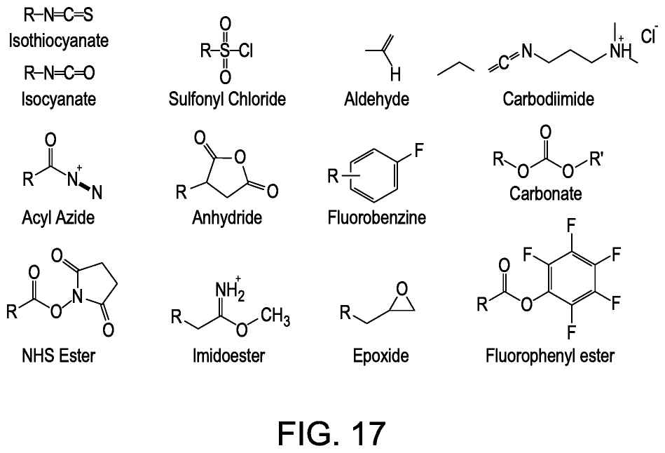

The amine-reactive functionalization chemistry may be varied by changing the active leaving group, for example from NHS to others listed in FIG. 17 in order to optimize reaction kinetics to allow for slower/faster curing times and/or lower toxicity of reaction byproducts. The change may also resolve compatibility issues in the presence of the conductive elements if the conductive elements negatively interact with the crosslinking chemistry (e.g. catalyzes undesired reactions)

The amine-containing crosslinker may be varied from trilysine to other multi-amine containing molecules selected from a group containing higher order poly-lysines (quad-lysine, pentalysine) polyamines selected from the group containing putresceine, spermindine, or spermine, and other branched polyamines selected from a group containing Tris(3-aminopropyl)amine and tetrakis(3-aminopropyl)ammonium. These crosslinkers may optimize the reaction kinetics to allow for slower/faster curing times and/or better mechanical properties of the final cured system. Furthermore selection of a different amine-containing crosslinker may enable different viscosities, allowing for better or more stable suspension of the conductive elements. The crosslinker itself may become a surface-modified conductive element. See herein re covalently bonded agents.

Additives for the PEG hydrogel may also be varied. Other preservatives, such as BHT, sucrose, trehalose, glycerin, sodium citrate, poloxamer, CTAB may be added to help stabilize the conductive element suspension or resuspension. Dyes may be added to allow ultrasound, MRI, or CT imaging, as well as buffers to change the reaction kinetics, e.g., high or low pH phosphate or boron buffers (e.g., 50-100 mM) as well as other ionic buffers (e.g. hypotonic, isotonic, or hypertonic saline, depending on desired swelling properties).

Conductive elements may be surface-modified by covalently conjugating (or otherwise associating chemically) moieties on the surface or in order to improve chemical or mechanical integration with the carrier matrix material.

A liquid nonconductor which cures in vivo to a nonconductive layer is also disclosed, using the same PEG hydrogel as used in the liquid mixture, described herein. As described herein regarding the carrier material for the liquid mixture, the PEG branching structure may be varied by changing the polymerization conditions during preparation of the PEG precursor in order to change the reaction kinetics and the ultimate hydrogel mechanical properties in different configurations: (a) Linear, (b) Branched multi-arm, (c) Multi-level branched (stellate/star), and (d) Random hierarchy.

The liquid nonconductor may also vary the ingredient concentrations in precursor solutions by increasing or decreasing the molarity of the solutions so that it will change the reaction rate and system viscosity. Higher molarity means more viscous. Different ingredient concentrations will also affect swelling characteristics. Swelling of “Example Commercial PEG Sealant” is ˜98% by volume. A higher initial ingredient molarity (e.g., hypertonic with respect to physiological conditions), will encourage more water ingress to attempt to balance the ionic and solute gradients, increasing the post-cure swelling.

Varying the PEG molecular weight of the carrier material by changing the polymerization conditions during preparation of the PEG precursor in order to change the reaction kinetics and the ultimate hydrogel mechanical properties as well as change the viscosity of the precursor PEG solution to enable selective suspension or precipitation of conductive elements. The selective suspension or precipitation of conductive elements may be used to create a phase-separated electrode, in which conductive elements sink to the bottom of the electrode solution confined in a volume, creating a conductive interface at the bottom, leaving a non- or less-conductive interface at the top. A lower viscosity suspension that would take longer to cure allows for conductive elements to sink to the bottom due to gravity if surgery/injection is done such that the nerve is lower or against a specific location then one can have a higher density filler against the nerve and lower density filler region away from the nerve—thereby creating an insulating layer on the top.

As with the liquid mixture described herein, it is possible to vary the amine-reactive functionalization chemistry by changing the active leaving group in order to optimize reaction kinetics to allow for slower/faster curing times and/or lower toxicity of reaction byproducts, for example from NHS to other compounds in FIG. 17. The change may also resolve compatibility issues in the presence of the conductive elements if the conductive elements (e.g. hypotonic, isotonic, or hypertonic saline, depending on desired swelling properties) negatively interact with the crosslinking chemistry (e.g. catalyzes undesired reactions).

To store a dry PEG carrier material mixture, mix dry PEG powder with conductive elements, then mix it with solvent when ready for use/injection. Mixing may include rapid shaking by a machine akin to a dental amalgam shaker.

Likewise, the PEG carrier material for the liquid nonconductor may vary the amine-containing crosslinker from trilysine to other multi-amine containing molecules, in order to optimize the reaction kinetics to allow for slower/faster curing times and/or better mechanical properties of the final cured system. Furthermore selection of a different amine-containing crosslinker may enable different viscosities, allowing for better or more stable suspension of the conductive elements.

Changes in additives may be made such as preservatives (listed herein) for better stability, dyes—allowing Ultrasound, MRI, or CT imaging to change the reaction kinetics. Glycerine/glycerol slow down the reaction kinetics and lengthen the curing time, as shown herein.

Another hydrogel suitable for the carrier material herein are hyaluronic acid gels which comprise hyaluronic acid, comprising a chemical formula of C₂₈H₄₄N₂O₂₃, and a molecular weight of 776.651 g/mol. It is a natural high-viscosity mucopolysaccharide with alternating beta (1-3) glucorinide and beta (1-4) glucosaminidic bonds. It is found in the umbilical cord, in vitreous body and in synovial fluid. Hyaluronic Acid is a glucosaminoglycan consisting of D-glucuronic acid and N-acetyl-D-glucosamine disaccharide units that is a component of connective tissue, skin, vitreous humour, umbilical cord, synovial fluid and the capsule of certain microorganisms contributing to adhesion, elasticity, and viscosity of extracellular substances. (https://pubchem.ncbi.nlm.nih.gov/compound/3084050#section=Top)

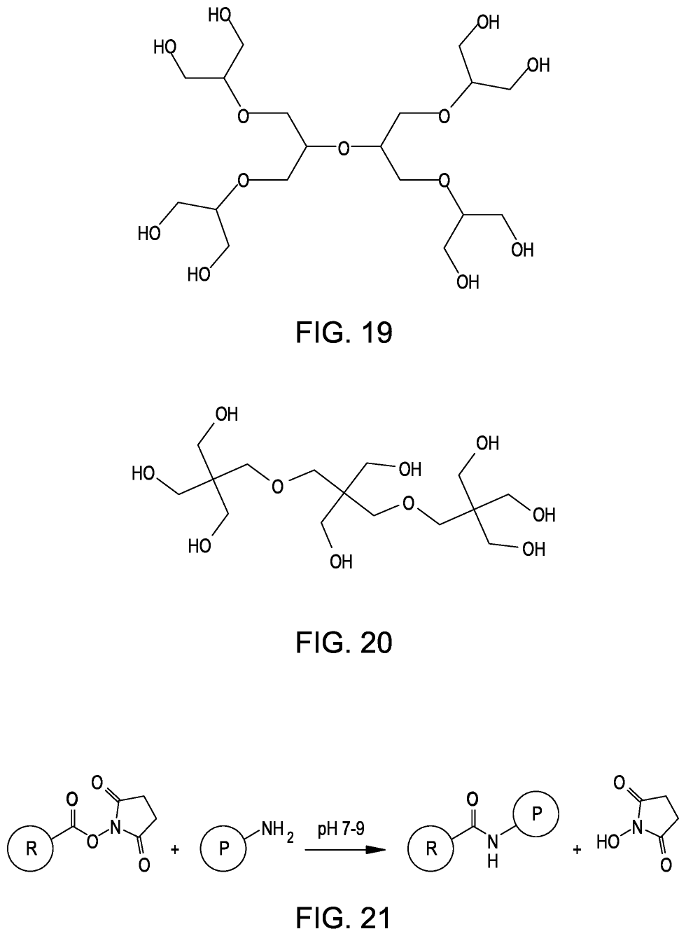

Variation of the PEG branching structure alters the rate of curing of the PEG hydrogel carrier material. “Example Commercial PEG Sealant” is a 4-arm PEG, but a 2-arm, 3-arm, 5-arm, etc. are suitable structures for the PEG carrier material by synthesizing or obtaining PEGs generated with varying core structures with the advantage being an increase or decrease in the number of potential cross-linking sites. This allows a change in the reaction rate and the strength of the polymer, the approach being focused especially on slowing curing and thereby allowing the physician to modify the liquid mixture for optimal fit in the body while or before curing in part or completely. FIG. 19 is a diagram of the chemical structure of a PEG with a Hexaglycerol core (8-arm). FIG. 20 is a diagram of the chemical structure of a PEG with a Tripentaerythritol core (8-arm).

Dendrimers are a versatile polymer structure that have been utilized in the field of drug delivery, in particular, used for improving solubility and bioavailability of poorly soluble drugs. Dendrimers have potential downsides resulting from biological toxicity related to their degradation byproducts or their cationic surface charge. Several strategies to counteract this toxicity have been employed including selection of biodegradable cores and other easily metabolized branching units, as well as by masking the surface charge by appending a neutrally charged group (e.g., PEG, acetals, carbohydrate or peptide conjugation). Such modified dendrimers do not exhibit the same degree of biological toxicity as their unmodified counterparts.

An example of variation of the ingredient concentrations in precursor solutions is a PEG concentration of 20% w/v or 10 mM and a Trilysine concentration is 10.5 mM. Examples of variation of the PEG molecular weight are disclosed as follows. Higher and lower viscosity mixtures are possible to enable homogeneous or heterogeneous suspensions of conductive elements. For example a 10 kDa PEG (20% w/v) may have an optimal viscosity to homogeneously suspend gold nanowires, however, gold microelements may sink to the bottom of the solution. On the other hand, a 100-300 kDa PEG solution (20% w/v concentration) may be optimal for fully suspending gold microelement and short microwire segments. “Example Commercial PEG Sealant” is a 20 kDa PEG. Two other PEG-based hydrogels that are legally marketed surgical sealants are suitable hydrogels: FocalSeal by Genzyme and CoSeal by Cohesion Technologies. FocalSeal has a molecular weight of 31,500 Da.

The amine-reactive functionalization chemistry may be varied. NHS-Ester activation chemistry converts hydroxyl (—OH) or carboxylic acid (—COOH) groups that normally terminate linear or branched PEGs into NHS-ester leaving groups that may react with amine (—NH2) functional groups. In the case of “Example Commercial PEG Sealant”, the PEG molecules are first modified to —COOH terminal groups using succinic anhydride, the intermediate is then reacted with sulfo-NHS, EDC, or DCC activators.

FIG. 21 contains diagrams showing steps of amine reactive crosslinker chemistry delivering stable conjugates and NHS. (Source: https://www.thermofisher.com/us/en/home/life-science/protein-biology/protein-biology-learning-center/protein-biology-resource-library/pierce-protein-methods/amine-reactive-crosslinker-chemistry html) Hydrolysis of the NHS ester competes with the primary amine reaction. The rate of hydrolysis increases with buffer pH and contributes to less-efficient crosslinking in less-concentrated protein solutions. The half-life of hydrolysis for NHS-ester compounds is 4 to 5 hours at pH 7.0 and 0° C. This half-life decreases to 10 mins at pH 8.6 and 4° C. The extent of NHS-ester hydrolysis in aqueous solutions free of primary amines may be measured at 260 to 280 nm, because the NHS byproduct absorbs in that range. NHS-ester crosslinking reactions are most commonly performed in phosphate, carbonate-bicarbonate, HEPES or borate buffers at pH 7.2 to 8.5 for 0.5 to 4 h at room temperature or 4° C. Primary amine buffers such as Tris (TBS) are not compatible, because they compete for reaction; however, in some procedures, it is useful to add Tris or glycine buffer at the end of a conjugation procedure to quench (stop) the reaction.

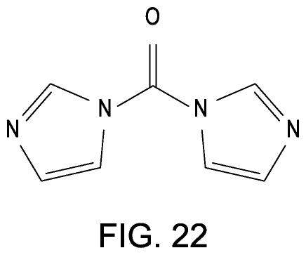

Other amine-reactive functional groups may be substituted for NHS-ester chemistry. A table of several examples is provided in FIG. 17. Furthermore, other chemistry linkage types may be substituted, including thiol-based (e.g. maleimide —SH), click-chemistry, or other common bioconjugation techniques known in the art. FIG. 17 depicts the chemical structures of examples of other chemistry linkage types. Carbonyldiimidazole (CDI) chemistry is another strategy for linking a carboxylic acid or hydroxyl group to a primary amine. The byproduct of the conjugation reaction is a urea, which possesses relatively low toxicity and readily cleared by the body. FIG. 22 is a diagram of the chemical structure of carbonyldiimidazole zero-order cross linker. An additional advantage of CDI is that the hydroxyls of the PEG may be directly activated as opposed to requiring prior conversion to carboxylic acid as with NHS chemistry. The coupling reaction of CDI-PEG proceeds much slower than that of NHS-PEG, such that the curing time of the electrode may be increased. At low temperature (4-deg-C), reaction rate may be extended up to 48 hours. CDI activation must be carried out in organic solvents, and the coupling reaction is most efficient in alkaline environments (or ˜1 pH above the pK value of the amine to be coupled). CDI-PEG remains active for years if stored in a properly desiccated environment. Imidazole carbamates (the reactive intermediate formed with CDI to PEG) have longer half-lives in water. Whereas the half-life of NHS-PEG in water is on the order of minutes due to hydrolysis. The half-life of the imidazole carbamate is on the order of hours. The rate of hydrolysis must be balanced with the rate of the reaction. If hydrolysis occurs too rapidly once reconstituted, it may be impractical for use. If hydrolysis is too slow, it may increase the risk of toxicity side effects.

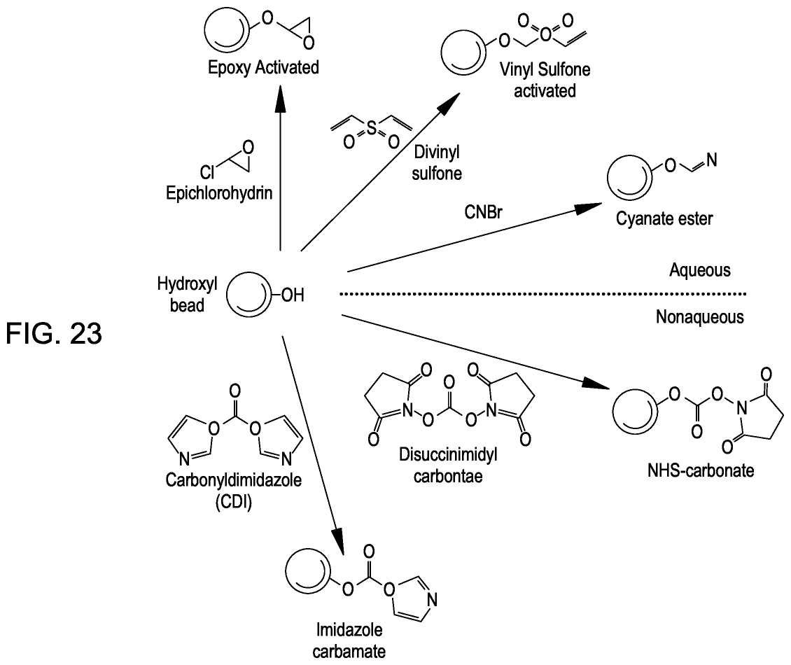

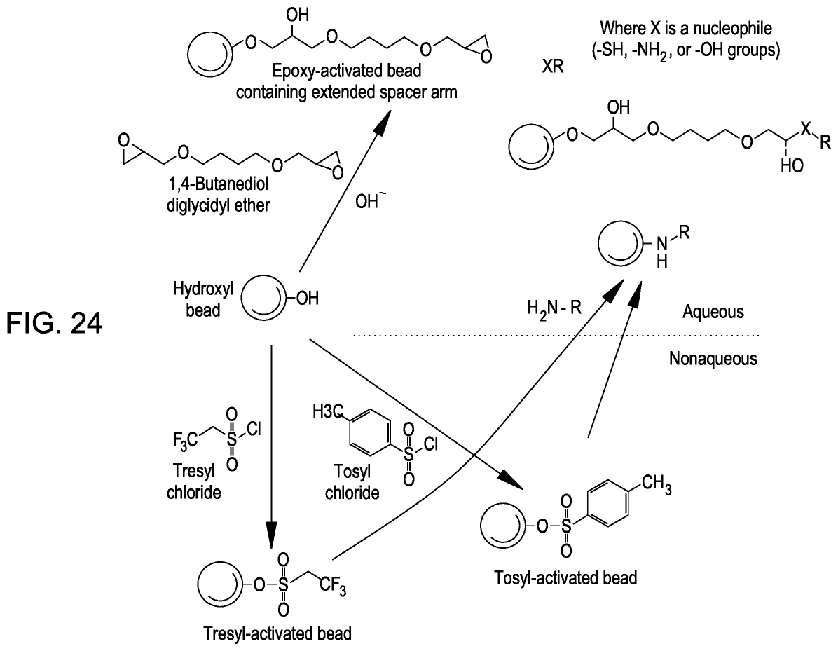

Hydroxyl-containing elements can be activated for coupling ligands using a number of strategies, which involve either aqueous or nonaqueous reactions. Epoxy and vinyl sulfone activation procedures provide reactive groups able to couple with amine-, thiol-, or hydroxyl-containing ligands. Cyanogen bromide activation and the CDI and DSC methods provide reactive groups for coupling amines. FIG. 23 is a diagram showing hydroxyl-containing elements use. (Source: Hermanson et al Bioconjugate Techniques). Additional hydroxyl element activation methods include bis-epoxide modification, tosyl activation and tresyl activation methods. The tosyl chloride and tresyl chloride activation procedures must be carried out in dry organic solvent, but the coupling of an amine-containing ligand can be performed either in organic solvent or aqueous buffer. FIG. 24 shows these additional hydroxyl element activation methods.

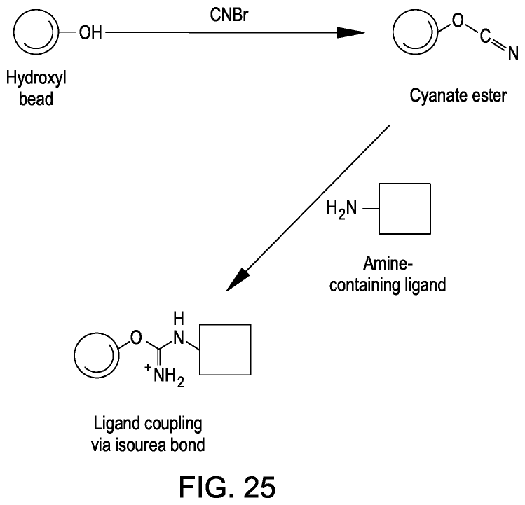

(Source: Hermanson et al Bioconjugate Techniques). Cyanogen bromide can be used to activate a hydroxyl element to a reactive cyanate ester, which can then be used to couple amine-containing ligands. FIG. 25 illustrates cyanogen bromide use. (Source: Hermanson et al Bioconjugate Techniques)

It is possible to change the amine-containing crosslinker from lysine by selecting a molecule from the group consisting of quadlysine, pentalysine, Lys-tryp-lys, Polylysine, and Polyarginine. These poly-amine containing molecules may be used as a crosslinking agent. Other poly-lysines may be used as a substitute for trilysine (e.g. poly(lysine)n where n maybe be any number greater than one. Other multi-amine valent peptides may also be substituted including poly(arginine)n. Poly peptides with primary amine functional groups (e.g. lysine or arginine) may also include patterned or randomly distributed spacer peptides (e.g. glycine, tryptophan, etc.) so as to reduce stereotactic hindrance of amine-crosslinking. Besides polypeptides, other polyamines may be used, including multi-arm or branched PEGs terminated with amine groups, micro- or nano-elements with surface modified amine presenting groups, or other polyamine molecules where the presentation of amines make them available for crosslinking.

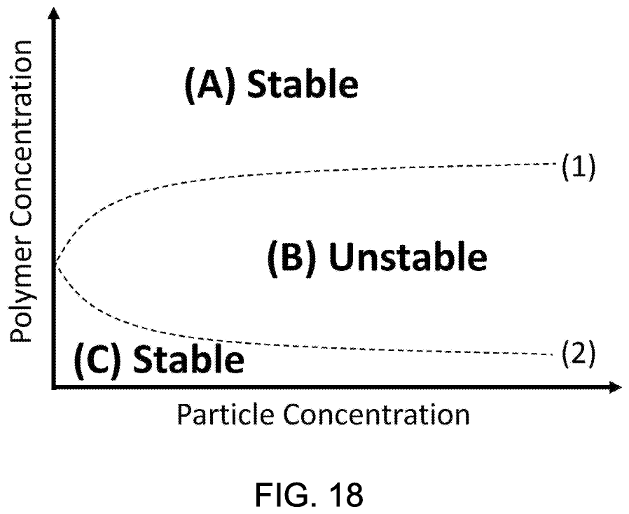

Preservatives may be added to PEG carrier material to achieve better solution stability, particularly surfactants for colloidal (re)suspension. FIG. 18 depicts the stability of PEG gels based on the concentration of preservative used. Hydrophobic elements have a higher propensity for aggregation in aqueous solutions and will shift the threshold (1) upward. Threshold (1) shifts downward on the y-axis with polymers of higher inherent viscosities, or with the use of surfactants that stabilize the elements in suspension. Threshold (2) shifts downward with larger or more hydrophobic elements. It shifts upward with the use of surfactants. At a greater polymer concentration (A) the suspension of elements is stable due to high viscosity of polymer (e.g., PEG solution). At a decreased polymer concentration (B) the suspension of elements is unstable as the polymer solution is not viscous enough to prevent element aggregation and/or settling; elements settle on bottom of container. At a concentration of polymer even further decreased (C), suspension of elements is stable due to low concentration of elements, thereby limiting chances for element aggregation to occur; this region is only of considerable relevance when elements are small (e.g., less than 100 microns). Macro-sized metallic elements (e.g. greater than 100 microns are unlikely to exhibit much stability in this region without the use of surfactants or other viscous stabilizers.

Buffers may be modified in PEG carrier materials, particularly increasing or decreasing the acidity or ionic concentrations of the buffers to change the reaction rate kinetics. Phosphate buffers and borate buffers, among others, in the range of pH 6-8 could may be used.

DuraSeal (Confluent Surgical, Waltham, Mass.; Covidien), is a 4-arm 20-kDa polyethylene glycol cross-linked with trilysine, used to prevent leakage of cerebrospinal fluid from dural sutures during spinal surgery; it is hydrolyzed and absorbed over a 4-8 week period. A newer formulation using a lower molecular weight polyethylene glycol, DuraSeal® Exact, has been reported to provide a tighter hydrogel matrix with less swelling than the original formulation. It is degraded by hydrolysis and reabsorbed over a 9-12 week period. In both cases, the hydrogel is believed to adhere to tissue by mechanical means.

CoSeal (Angiotech Pharmaceuticals, Vancouver, BC; Baxter), is a mixture of a 4-arm PEG tetra-hydroxysuccinimide ester and a 4-arm PEG tetra thiol, each of approximate MW 10 kDa, used for arterial and vascular reconstruction. The resulting gel comprises thioester linkages that are hydrolytically labile, resulting in eventual gel degradation and resorption. Tissue adherence is provided by reaction of some of the reactive hydroxysuccinimide esters, and possibly some of the thioester groups, with protein amine groups in the tissue. CoSeal is reported to remain effective at the application site for 7 days, and is fully degraded after 30 days. It is a synthetic, translucent gel for cardiovascular and peripheral vascular surgery applications. It consists of two PEGs that rapidly crosslink with proteins in the tissues, forming a covalent bond. Also mechanically adheres to synthetic graft materials. Intended for adjunctive use to seal areas of leakage.

Progel (Neomend, Irvine, Calif.), is a hydrogel which is human serum albumin cross-linked with a bifunctional hydroxysuccinimidyl-polyethylene glycol (U.S. Pat. No. 6,899,889 B1), used for intraoperative sealing of pleural air leaks. It is a hydrogel sealant made of human serum albumin and PEG. A formulation using a recombinant albumin, Progel Platinum Surgical Sealant, has been developed. Progel AB is a hydrogel adhesion barrier sealant that may be sprayed onto general visceral organs during surgery to help prevent postoperative adhesions. Approximately 60% of Progel is degraded after 1 day, and complete degradation is observed after 2 weeks

FocalSeal-L (Genzyme, Cambridge, Mass.) is a mixture of a polyethylene glycol capped with short segments of acrylate-capped poly(L-lactide) and poly(trimethylene carbonate) with a photoinitiator, eosin Y, and has been used to limit air leak after pulmonary resection. The solution polymerizes upon exposure to blue-green light to form a thin film hydrogel. The sealant does not bond covalently with tissue, and expands upon contact with bodily fluids over approximately 24 hours. Hydrolysis of the lactide and carbonate linkages allows for gel degradation and resorption. FocalSeal has been used as a tissue adhesive.

Adherus Dural Sealant and Spinal Sealant (HyperBranch Medical Technology, Durham, N.C.), a mixture of poly(ethylene-imine) cross-linked with a bifunctional PEG-hydroxy-succinimidyl ester, used in cranial and spinal surgery to prevent cerebrospinal fluid leakage and dural adhesions. Polyethyenimine can take different structures including linear or branched, with the general formula X—(CH₂—CH₂—NH)ₙ—Y, where X and Y may be primary amines, methyl or hydroxyl groups, and where branching may occur off the nitrogen groups, forming a tertiary amine structure. Molecular weights that may be used in such applications may range from 1,000 Da to 50,000 Da.

OcuSeal Liquid Ocular Bandage (HyperBranch Medical Technology, Durham, N.C.), a synthetic hydrogel that is applied directly to the ocular surface as a liquid, using a brush applicator.

Metro hydrogel glue utilizes a modified protein to form a UV-cross-linking adhesive. The protein-glue in this way, is similar to fibrin glue, and may be used as non-conductive carrier but different in that curing is controlled by UV light. A simple way of manufacturing a protein glue with similar characteristics as metro is using a poly-l-lysine modified in the same standard way as described in earlier literatures and Irgacure 2959. While this may not have the same elastic properties as MeTro glue, it is possible to use the same cross-linking mechanism, which allows the application of Polylysine, a more standard ingredient.

There are synthetic hydrogels, some PEG based, that are approved for use in the clinic. A combination with electrically conductive elements, wires, strands, meshes, fibers, one or more of them being optionally surface modified, and or optimized for a heightened mechanical integration with the synthetic hydrogel provides the electrical conductivity needed for them to be applicable in the field of neuromodulation.

The modifications described herein focus on ease of use for the physician user who places the liquid mixture with an emphasis on work time (may be as short as seconds or may be as long as tens of minutes), viscosity to allow optimal access around or into various target structures of interest, mechanical strength and ability to integrate with the surrounding tissue, degradability optimized for the specific tissues the liquid mixture is placed (i.e. injected) into or around or next to, as well as other factors of interest.

Other modifications of the synthetic hydrogel focus on achieving and retaining a homogeneous suspension of the electrically conductive elements in the PEG base by optimizing the viscosity of the PEG solution prior to (and/or during a beginning) curing process. This further facilitates reproducibly homogeneous conduction of energy, especially electrical energy, across the liquid mixture or the cured electrode and ensures an optimal connection between an active implantable device and a target in bodily tissue.

Another aspect is the modification of the synthetic hydrogel to be resorbed at a rate that is most optimal for the specific placement location. While a nerve in a location that is not subjecting the injectable electrode to shear forces may allow for a faster resorption time, most applications will require an injected electrode to be mechanically stable (cohesive) for a period of at least two weeks, and most applications for at least four to six weeks until the tensile strength of the encapsulating tissue is able to provide structural support. Faster resorption can be accomplished by using a lower molecular weight PEG. For example the 10 kDa, 4-arm PEG used in tissue adhesive/sealant applications degrades over 4-8 weeks. A reduction in molecular weight to 5 kDa can reduce resorption time to 2-4 weeks, whereas an increase in molecular weight to 20 kDa can increase the resorption time to 8-12 weeks. A liquid mixture may be injected at locations where shear forces are present or may be expected. By providing a cured electrode with higher tensile strength, these shear forces may be resisted better while the body absorbs and/or remodels the PEG/hydrogel by replacing it with connective tissue, fibrous tissue and or other tissues that may take up the forces.

Combinations of PEGs and Cyanoacrylates may be used to allow for a porous structure that i.e. binds temporarily to bony tissue or other tissues of higher tensile strength in the body, while providing the means for the body to grow into the structure and replace an overwhelming amount of the total volume of the porous structure with its own cells, or the porous structure is filled by interstitial fluid, thus adding to the surface area of the conductive elements, as described elsewhere herein.

An example of a PEG based cured electrode is as follows. A ˜1 mL volume nanowire-based liquid mixture has the nanowires suspended non-covalently in a PEG hydrogel matrix. Part A and Part B are mixed in a 1:1 ratio, and allowed to cure to form a PEG hydrogel-based cured electrode.

### Part A:

A. Carrier Material Part A: 0.1 g 20 kDa 4-arm PEG-NHS at 20% w/v (0.5 ml total solution) in sodium phosphate buffer, pH 7.4
B. Conductive elements mixed with Carrier Material Part A: Gold conductive elements (˜2 nm diameter, ˜5 μm length) at 25-50% weight % (with respect to PEG+carrier solution, e.g., 50% would be ˜0.5 g gold nanowires to ˜0.5 g PEG solution).

### Part B

A. Carrier Material Part B: 10 mM trilysine (0.5 ml total solution) in 75 mM borate buffer
B. Conductive elements mixed with Carrier Material Part B: Gold conductive elements (˜2 nm diameter, ˜5 μm length) at 25-50% weight % (with respect to trilysine+carrier solution, e.g., 50% would be ˜0.5 g gold nanowires to ˜0.5 g trilysine solution).
   The resulting mixture of Part A and Part B will form a cured electrode in 1-5 minutes. The conductive elements provide additional mechanical strength.

An example of a liquid mixture, comprising micrometer size elements+PEG based is described herein. A ˜1 mL volume gold powder-based liquid mixture that has gold powder/grains covalently bound in a PEG hydrogel matrix. Part A and Part B are mixed in a 1:1 ratio, and allowed to cure to form a PEG hydrogel-based cured electrode.
Part A: 0.1 g 100 kDa 4-arm PEG-NHS at 20% w/v (0.5 ml total solution) in sodium phosphate buffer, pH 7.4
Part B: 10 mM trilysine (0.5 ml total solution) in 75 mM borate buffer, or Modified Conductive filler Mixed with Carrier Part B: Gold conductive elements (˜100-500 μm major axis width, with aspect ratio 1-5) at 85-99% weight % (with respect to trilysine+carrier solution, e.g., 99% would be ˜0.99 g modified gold elements to ˜0.01 g trilysine solution). The elements will be themselves modified on the surface with a 5 kDa linear PEG terminated at one end with a thiol (—SH) and at the other end an amine functional group (—NH2). The thiol binds and forms a stable bond to the surface of gold, exposing a free primary amine that may itself react with the PEG-NHS carrier in Part A.
   The resulting mixture of Part A and Part B will form a cured electrode in 1-5 minutes. The element covalent bonding provides additional mechanical strength. The higher molecular weight PEG provides additional viscosity allowing the elements to become fully suspended to form a homogeneous mixture during the curing process. The conductive elements, having free amines are initially only suspended in Part B, which has the potential additional benefit of preventing unwanted reaction of the NHS with the metal surface which may or may not act as a catalyst for hydrolysis during storage.

Another example of a cured electrode comprises PEG and gold conductive elements, at least a portion of which form covalent bonds with one another when mixed, forming a higher degree of crosslinking between polymer and conductive elements, improving the mechanical/chemical interface characteristics.

Part A comprises PEG-NHS+Gold-NHS

Part B comprises Trilysine or PEG-NH₂+Gold-NH₂

Another PEG matrix cures rapidly and suspends gold conductive elements in solution—pre-cured. This allows gold conductive elements to coalesce and covalently or ionically interact during hydrolysis of hydrogel matrix. During hydrolysis, the gold conductive elements coalesce and cross-link
Part A comprises PEG-NHS+gold conductive elements with short (di)sulfide bridges that will react with the gold wires from Part B to form stable bonds.
Part B comprises trilysine+gold conductive elements

The impedance of several PEG-silver carrier materials using CoSeal were tested, with the silver conductive elements comprising aspect ratios of approximately 2:1 to 3:1 on average, with major axis as high as 6 microns, and the data is in Table Three.

TABLE THREE
Impedance of PEG-Silver Liquid Mixture/Cured Electrode

| | | | | | Impedance |
|---|---|---|---|---|---|
| | Ag | PEG | Glycerol | Ag-PEG | (ohms, 1 kHz) |
| Mix | (mg) | (μL) | (μL) | % | Pre- and Post-Cure |
| One | 800 | 200 | — | 80.0% | <1 |
| Two | 800 | 200 | 200 | 66.6% | 10-50 |
| Three | 800 | 200 | 100 | 80% | <1 |

Mix one was 80% silver, 800 mg silver and 100 μL each of part A and part B. Mix two was 66% silver with 16.6% glycerol, 800 mg silver, 200 μL glycerol and 100 μL each of part A and part B. Mix three was 73% silver with 9% glycerol, 800 mg silver. 100 μL glycerol, and 100 μL each of part A and part B. The curing times were: Mix one cured almost instantaneously, with in 3 to 5 seconds; Mix two cured over a long period of time, getting tacky within 30 to 45, and fully curing within 10 to 15 minutes; and Mix three became tacky within 10 to 15 seconds and fully cured within 45 seconds to one minute. The mechanical properties observed were: Mix one was brittle, chalky and had the most flaking of the three; Mix two was sticky/slimy, very flexible and Jell-O like once cured. There was some but not a large amount of flaking; Mix three was similar to mix two, being flexible, sticky and Jell-O like in consistency. Similar to mix two, there was not as much flicking in this formulation compared to silicone. Tearing force was relatively strong for a gel, breaking around approximately 3 to 5 g force. Mixes two and three were both still electrically conductive after stretching twisting and bending. Mix three was placed in water after cured. Its initial weight was 710 mg. After three hours of soaking, it had swollen to a mass of 1.2 g. A small amount of flaking was observed at the bottom of the beaker, but not a huge amount. The gel was removed from water and it was still conductive and mechanically cohesive.

An alternative method of delivering a PEG plus optional additives (cells, sugars, . . . ) plus conductive elements (e.g. gold) in a mixture is achieved by first hydrolyzing the PEG, then freezing it as one of the components and then mixing the frozen components in their respective ratios (ratios mentioned above in this section). One method of freezing the liquid components is to supply hydrolyzed PEG under moderate pressure in a heated syringe with heated nozzle in a freezer (temperature of negative 20 degrees C. or colder) with the effect of forming PEG snow which deposits on a tray within the freezer. This PEG snow must not be compacted to retain proper mixing ratios later in the mixing procedure. Similarly, any optional additives may be provided in i.e. aqueous solution to allow the generation of optional additive component (OAC) snow on a second tray (either within the same freezer but different compartment or same freezer). Likewise, conductive element (i.e. gold) powder of the chosen grain/particle size is cooled to the same temperature as the PEG and OAC snow and provided to a third tray within the freezer. A manually controlled, or semi or fully automated manipulation unit then collects the appropriate volumes of PEG snow, OAC snow and conductive particle powder, and uses the measurement of each of the component's weight to control the future properties of the mixture. All three components are supplied to a blender which may use a rotational motion, planetary mixing or spatula to blend the three components to a homogeneous mixture. Once a homogeneous mixture has been achieved, it is partitioned into syringes or other delivery devices, all of which are pre-cooled to avoid any unintentional melting of either of the snow components. The syringes may be stored within a freezer (−20 degC or cooler, better is −80 degC) or stored and/or shipped on dry ice (temperature approximately −78.5 degC) until the liquid electrode mixture is desired. Once the liquid electrode is desired, the cold syringe with cold contents may either be heated in a warm water bath for a duration lasting from seconds (thin syringe, cold temperature −20 degC) to a few minutes (thick syringe, cold temperature −78.5 degC). The temperature of the water bath may be between 15 and 42 degreesC, colder temperatures offering a slower fibrin formation and thus longer work time for the mixture prior to achieving full cure. Alternatively, the syringe may be heated in a purpose built heating device that measures the temperature of the mixture inside the syringe during the application of heat, reporting on the rise in temperature and reaching the mixing and later the dispensing temperatures. The purpose built heating device may furthermore provide a countdown that indicates the amount of time available until the mixture inside the syringe is beginning to harden by itself. Optimal blending of the mixture may further be achieved by agitation of the syringe via US, mechanical vibration or by using a mixing nozzle that forces the liquid mixture through channels inside the needle, leading to an increase in homogeneity of the liquid mixture just prior to injection/placement. The advantage of mixing frozen components is to retain the maximal curing time for the physician in the OR, and ensuring fresh mixtures of reproducibly high quality.

### Silicone

By combining vinyl terminated siloxane and a polyfunctional silicon hydride with a catalyst, silicones may be achieved that do not require moisture to cure, as follows:

Si—H+CH₂=CHSi->SiCH₂CH₂Si

The typical by-products of the condensation of such a silicone curing process is a small amount of hydrogen gas that may easily dissipate and not cause acute or chronic inflammatory responses in stark contrast to industrial silicones that create either alcohol or acetic acid as by product of curing. FDA has approved food grade silicones for chronic contact with food, and these cure around food and are known to not leach significant amounts of toxic by-products into the food before, during or after curing near or around food items intended for human consumption. The curing time may depend on the utilized catalyst and platinum has been shown to provide advantageous curing times (<5 minutes) while not causing a heightened chronic inflammatory reaction.

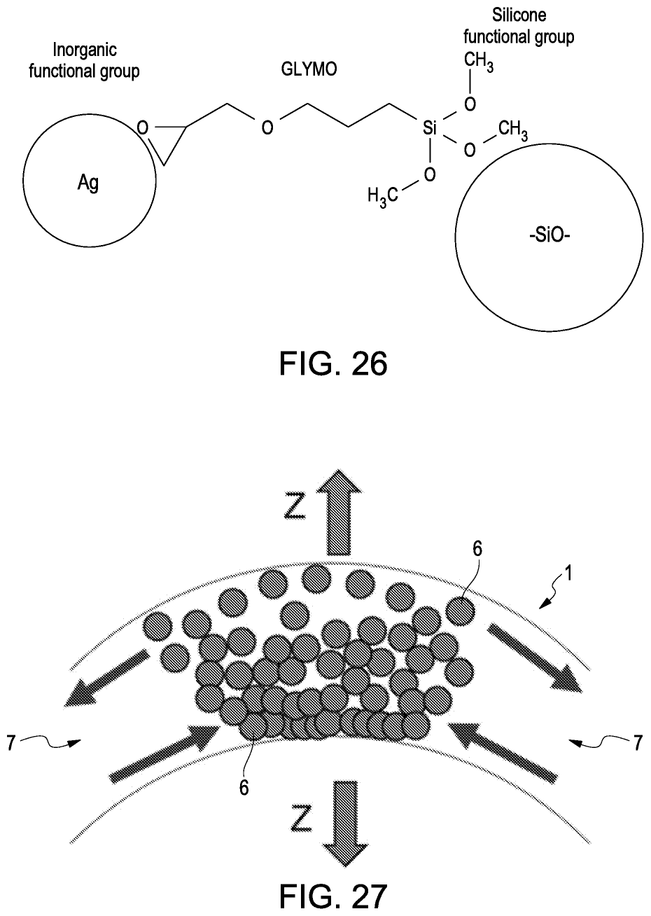

Silicone is also used in implanted medical devices, including breast implants, wire leads, and device components. It is tough, flexible, soft, and highly elastic. By itself it is an electric insulator, but it can be mixed with conductive elements as described herein to form a liquid mixture which cures upon injection into a bodily tissue. During polymerization it is very self-cohesive and tends to encapsulate conductive elements leading to non-percolation of the bulk composite. Addition of a surfactant (e.g., 3-Glycidyloxypropyltrimethoxysilane, herein “GLYMO”) helps to interface the metallic (inorganic) mixture with the polymer (organic) phase as shown in the diagram which is FIG. 26 which is a diagram of the chemical structure of bonds between, on one hand, GLYMO and a silicone as the carrier material and, on the other hand, GLYMO and silver as the conductive element. Thus, GLYMO as surfactant prevents the silicone from completely engulfing the conductive element, thereby preserving the liquid mixture/cured electrode's low impedance.

With two part curing silicone systems, it is possible to incubate the GLYMO with conductive elements first, ensuring a full and homogenous coating of the conductive elements. With silver (e.g., grains sized 50-200 um), the weight % (GLYMO/silver) in one embodiment is approximately 5-15% weight % of final electrode (e.g., 5% GLYMO, 75% silver, 20% silicone) to achieve uniform coating of the conductive elements with GLYMO. Beyond a level of approximately 50% weight % of the weight of the entire mixture (silicone-silver-GLYMO) the final silicone-GLYMO-silver element mixture was no longer electrically conductive, thereby suggesting an upper boundary of approximately 50% weight over which the GLYMO fully coats and electrically isolates the conductive elements.

The GLYMO-silver mix may then be mixed separately with part A and part B silicones. In one embodiment the silver-GLYMO-silicone mix required to achieve electrical percolation was measured to be at least 65% (silver/silicone) to achieve impedance values below 10Ω for the overall mix. Longer whisker metal elements (aspect ratio at least 5:1, or within a range of 5:1 to 10:1) allowed lower volume/weight percentages of silver to be present (such as 50-60%) to still provide sufficient conductivity (Z<100Ω) for a liquid mixture/cured electrode to be able to connect to a nerve at lower impedance values than the surrounding tissues. One embodiment achieving suitable conductivity comprises 200 mg silver, 50 mg GLYMO, and 100 mg silicone (50 mg part A, 50 mg part B). The precursor materials are mixed as such, where the mixing operations within the parentheses are performed first.
Step 1: (100 mg silver+25 mg GLYMO)+50 mg Part A silicone
Step 2: (100 mg silver+25 mg GLYMO)+50 mg Part B silicone
Step 3: mixture from Step 1 is combined and homogenously mixed with mixture from Step 2
   Different colored dyes are added to Part A and Part B silicones to allow for visual confirmation of mix homogeneity. In another embodiment, the silicone used may be a one-part room temperature vulcanization (“RTV”) curing system, although for biomedical applications, there are typically concerns over acetic acid buildup as a result of the condensation reaction during curing but with small amounts injected (e.g., 10-50 μL), the amount of acetic acid is low.

Table Four is a comparison of Silicone based cured electrodes outside the body utilizing gold and silver as conductive elements in various concentrations. All impedances were measured with sinusoidal waveforms at 1 kHz, and may be understood as volume impedance.

TABLE FOUR
Silver-Silicone Impedance

| | GLYMO | Silicone | Ag-Silicone | Impedance |
|---|---|---|---|---|
| Ag (mg) | (μL) | (μL) | % | (ohms, 1 kHz) |
| 100 | 50 | 100 | 50.0% | — |
| 150 | 50 | 100 | 60.0% | — |
| 200 | 50 | 100 | 66.7% | 2.4 |
| 250 | 50 | 100 | 71.4% | 2.0 |
| 300 | 50 | 100 | 75.0% | 1.9 |

Silicone-Gold Impedance

| | GLYMO | Silicone | Au-Silicone | Impedance |
|---|---|---|---|---|
| Au (mg) | (μL) | (μL) | % | (ohms, 1 kHz) |
| 200 | 50 | 100 | 66.7% | <1 |
| 150 | 50 | 100 | 60.0% | 2.5 |

Silicone has an advantage of high flexibility, and it can withstand elastic strains up to 50-100%. Due to this flexibility and bendability, the cured silicone may bend at very low radii. While cured silicone can withstand this bending strain, the cured electrode will undergo compression and tension at the inner and outer aspects of the bend, respectively. If the conductive elements comprise a low concentration or have a low aspect ratio, the resulting bend may yield a non-conductive surface on the outer aspect (Z increases), while the inner aspect may decrease in impedance. FIG. 27 is a diagram of the mechanism of a cured electrode 1 with low aspect ratio conductive elements retaining similar impedance during bending: as the convex top is bent and elements move apart slightly, elements at the concave bottom are pressed together. While locally the impedance at the top or bottom aspect may change during bending, the bulk conductivity along the axis remains relatively consistent.

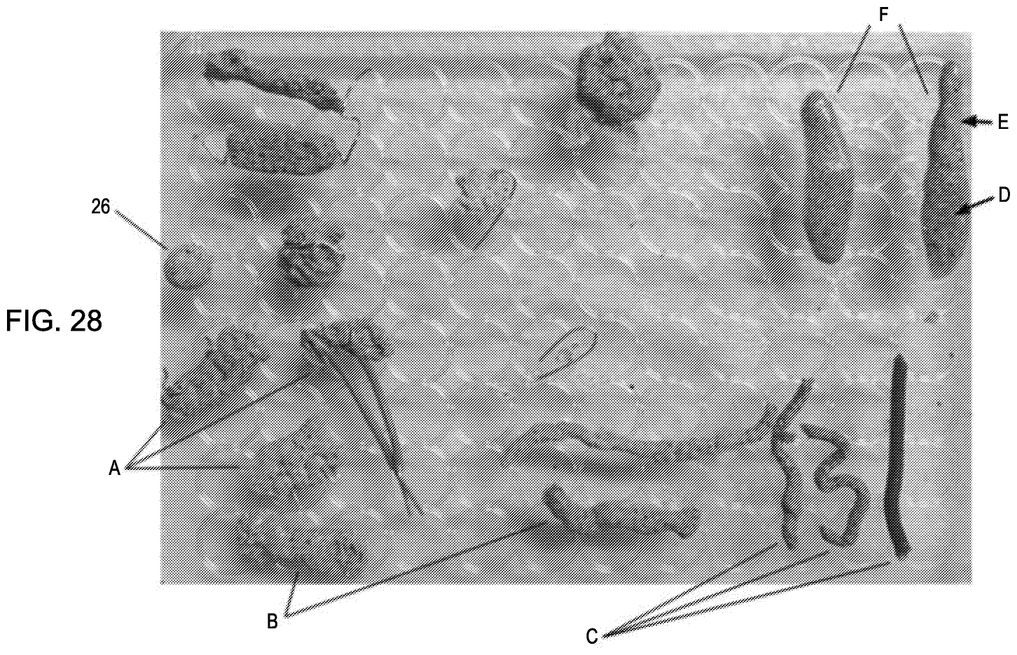

FIG. 28 is an image of a collection of different curing capabilities based on varying viscosities of a silicone carrier material. Pictured is a blob portion 26 of a cured electrode. Reference A shows cured electrodes with embedded wires. Reference B shows cured electrodes of high viscosity of 4-6 mm in diameter. Reference C shows cured electrode of high viscosity of 2 mm diameter. References D and E are for one cured electrode with a conductive element % weight which are low and high respectively. F also shows a low viscosity cured electrode. Differences in viscosity are primarily achieved by changing the ratio of conductive elements vs. silicone carrier material. A secondary way of changing the viscosity is by adding surfactants, thickening or thinning agents. Cured materials in FIG. 28 are all silicone based and retain their flexibility post-cure.

Other surfactants besides GLYMO include the IV injectable PEG Vegetable Oil (FDA CAS number 8051352), PEG-40 Castor Oil (61/791,126), Soybean Oil (FDA CAS number 8001227), PEG-60 Hydrogenated Castor Oil (61/788,850) and optionally the IM injectable Sesame Oil (CAS NUMBER 8008740), Polyoxyl 35 Castor Oil (61/791,126). Vegetable Oil and Sesame Oil were tested and proved to provide conductive silicone/silver mixtures at oil to silicone ratios of 1 to 2 and 1.25 to 3 with impedance values <10 Ω cm for the cured electrodes measured at 1 kHz.

An alternative method of delivering a Silicone+surfactant+optional additives (e.g., cells, sugars)+conductive element (i.e. gold) mixture is achieved by proving the silicone components A and B as frozen granulate, the surfactant as frozen granulate, likewise any desired optional additives as granulate of frozen carrier with additives and the conductive particles in cooled form. Alternatively, the conductive elements may first be mixed with a surfactant or an oil (to prevent surface interactions with the silicone during the melting and mixing procedures), then freezing this mixture and breaking it up into smaller parts to form a granulate of conductive particles covered in surfactant. This frozen granulate is then kept cold and stored in a third tray within the freezer, the two respective silicone granulates being kept in tray one and two. A manually controlled, or semi or fully automated manipulation unit then collects the appropriate volumes of frozen silicone part A granulate, frozen silicone part B granulate and conductive particle in surfactant granulate, and uses the measurement of each of the component's weight to control the future properties of the mixture. All three components are supplied to a blender which may use a rotational motion, planetary mixing or spatula to blend the three components to a homogeneous mixture. Once a homogeneous mixture has been achieved, it is partitioned into syringes or other delivery devices, all of which are pre-cooled to avoid any unintentional melting of either of the snow components. The syringes may be stored within a freezer (−20 degC or cooler, better is −80 degC) or stored and/or shipped on dry ice (temperature approximately −78.5 degC) until the liquid electrode mixture is desired. Once the liquid electrode is desired, the cold syringe with cold contents may either be heated in a warm water bath for a duration lasting from seconds (thin syringe, cold temperature −20 degC) to a few minutes (thick syringe, cold temperature −78.5 degC). The temperature of the water bath may be between 15 and 42 degreesC, colder temperatures offering a slower fibrin formation and thus longer work time for the mixture prior to achieving full cure. Alternatively, the syringe may be heated in a purpose built heating device that measures the temperature of the mixture inside the syringe during the application of heat, reporting on the rise in temperature and reaching the mixing and later the dispensing temperatures. The purpose built heating device may furthermore provide a countdown that indicates the amount of time available until the mixture inside the syringe is beginning to harden by itself. Optimal blending of the mixture may further be achieved by agitation of the syringe via US, mechanical vibration or by using a mixing nozzle that forces the liquid mixture through channels inside the needle, leading to an increase in homogeneity of the liquid mixture just prior to injection/placement. The advantage of mixing frozen components is to retain the maximal curing time for the physician in the OR, and ensuring fresh mixtures of reproducibly high quality.

### Cyanoacrylate

Cyanoacrylate based materials are also a carrier material for inclusion in a liquid mixture. Although offering less flexibility in comparison to silicone based mixtures, cyanoacrylates as a carrier material have a variety of advantages, such as more ability to resist stress and strain, and excellent integration with bone and other tissues. They offer the ability for immediate coagulation and control of bleeding under surgical conditions. There are surgical cyanoacrylate variations available as FDA approved surgical glue that function as more viscous and less viscous carriers in gel form. The gel variety has advantages for delivery via small diameter needles where the gel may help with keeping the liquid mixture with e.g. metal elements more uniform when subjected to the stress due to passing from large inner diameter syringe into small inner diameter needle. Once the cyanoacrylate-surfactant-conductive element mixture has been injected as a gel mixture and is allowed to cure inside the body, the carrier portion gel polymerizes and forms a solid that is able to provide structural stability to the cured electrode 1.

Certain cyanoacrylates are safe for biomedical application, including injection into the body as blood-contacting implants. These comprise a first liquid phase which is fast-curing within several seconds of application, in particular on contact with water. As cured in a second solid phase, it is significantly stiffer than soft tissues. It bonds mechanically strongly with biological tissues, including nerves, skin, muscle, fat and bone. By itself it has a high impedance and acts as an insulating material. When combined with conductive elements at high concentrations, the resulting liquid mixture/cured electrode is conductive.

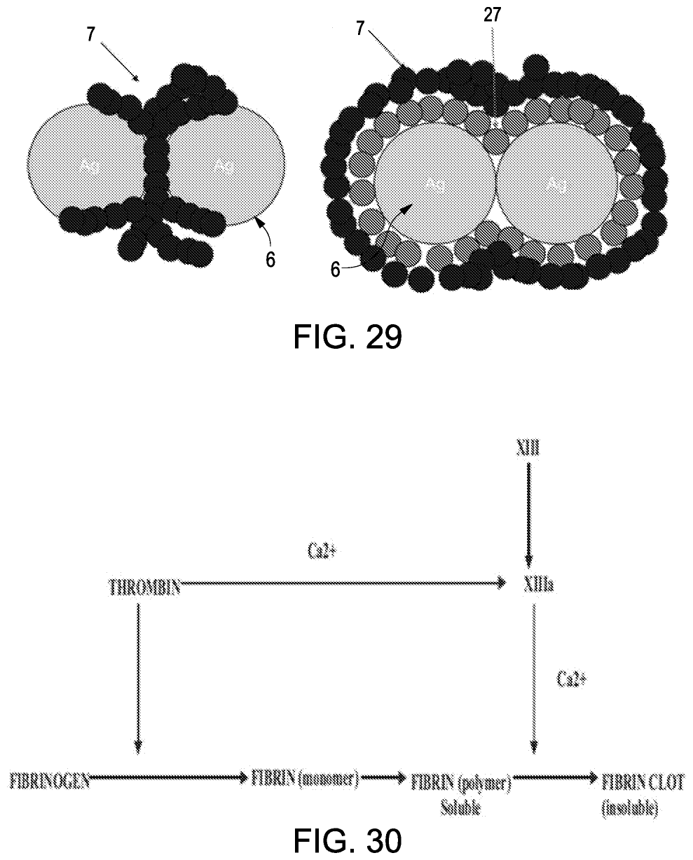

The conductive elements must be mixed with the cyanoacrylate solution in an ultra-dry environment. The conductive elements may be pre-treated with inert gases (e.g. Argon, Nitrogen), or dry solvents (e.g. isopropyl alcohol) to dry it fully. The cyanoacrylate may be mixed homogenously with the conductive elements. However, the conductive elements may also be intentionally separated into distinct high and low concentration regions by use of a thin (low viscosity) cyanoacrylate solution, in which the heavy conductive elements selectively sink to the bottom. This may be used to achieve low electrical impedance at the bottom interface, while achieving high impedance at the top surface after curing, and this process applies to all carrier materials and especially for those that are insulative: silicone, cyanoacrylate and dental resin. Furthermore, since cyanoacrylate has a high cohesive property while curing, it may cure all around the conductive elements 6 leading to complete isolation from one another. To overcome this, a surfactant 27 may be added, such as water and/or ethanol. Alternatively a 95% ethanol solution mixed with the conductive filler material appears to be sufficient to allow for electrical percolation. Ethanol is mixed with the conductive elements, and then immediately mixed with cyanoacrylate to prevent significant evaporation of ethanol. The mechanism of enabling electrical percolation is by coating the conductive elements with an ethanol/water layer, leading to condensation/polymerization of the cyanoacrylate at the water interface coating the conductive element rather than at the metallic interface itself, thereby allowing metal-metal contacts to persist throughout the curing process. Water alone initiates polymerization of the cyanoacrylate and is not as effective as alcohol to yield this effect. Ethanol dissolves the cyanoacrylate and reduces the rate of polymerization. FIG. 29 is a representation of the function of the water/ethanol surfactant 27 in a cyanoacrylate based cured electrode with silver conductive elements 6. Without any surfactant present, cyanoacrylate creeps between the conductive elements. If the conductive elements have been pre-wetted with surfactant, then the strong bonds between cyanoacrylate are not able to pull the conductive elements apart and isolate them. As a result, the overall liquid mixture/cured electrode that includes the surfactant remains conductive.

The most commonly used forms of cyanoacrylate for medical applications include n-butyl cyanoacrylate and 2-octyl cyanoacrylate, each of which has some flexibility after curing. Both are suitable for the liquid mixture herein. In some embodiments, cyanoacrylates is functionalized with chemical sub-groups that allow the carrier medium itself to become conductive, for example PEDOT:PSS. In that case a placement of the liquid mixture may be accomplished by spray-on similarly as liquid band aid is dispensed on an open wound, this time the liquid mixture being sprayed laparoscopically on a nerve, with spray channels both, a functionalized, electrically conductive cyanoacrylate as channel 1 and an electrically non-conductive cyanoacrylate as channel 2.

To achieve electrical percolation with silver conductive elements (in one embodiment, ˜50-200 micron size distribution) and n-butyl cyanoacrylate, over 85% weight % (silver/cyanoacrylate) was required, with the silver elements produced by a dremel. Lower silver weight % may be attainable with the use of additional surfactants or the ethanol (and resulting water phase separation) method discussed herein. Furthermore, the use of other high aspect ratio conductive elements, such as microwire rods or whiskers, allow electrical percolation to occur at lower conductive element weight % concentrations.

Omnex (Ethicon, Somerville, N.J.) produces a mixture of 2-octyl cyanoacrylate and butyl lactoyl cyanoacrylate which is used in vascular reconstructions, and which is suitable for the liquid mixture/cured electrode herein. Omnex degrades by hydrolysis over approximately 36 months. While cyanoacrylates have also been used as tissue adhesives, for example DermaBond (Omnex), their use is limited by toxicity, such as tissue necrosis at the site of application.

Cyanoacrylate (CA) and its possible derivatives may be processed similarly (as described above) in a frozen environment to form a CA-conductive particle granulate mixture that is transported under dry ice to the application location where heating allows for easily reproducible work and curing times for the physician.

### Fibrin Glue

Fibrin glue (also called Fibrin sealant) is a formulation used to create a fibrin clot. It comprises fibrinogen (lyophilised pooled human concentrate) and thrombin (bovine, which is reconstituted with calcium chloride) that are applied to the tissue sites to glue them together. Thrombin is an enzyme and converts fibrinogen into fibrin monomers within 10 and 60 seconds giving rise to a three-dimensional gel. In some embodiments, fibrin glue may also contain aprotinin, fibronectin and plasminogen. Factors that influence dimensional structure of fibrin gel giving rise to fine or coarse gel: (1) changing concentration of fibrinogen, (2) changing concentration of thrombin increasing concentration increases ultimate tensile strength and Young's Modulus of gel, (3) changing concentration of calcium, (4) pH, and (5) temperature.

Fibrin glue is a human-derived tissue adhesive used for hemostasis and sealing of tissues. This biological glue can be manufactured from clotting factors taken from donor plasma (fibrinogen, cryoprecipitate and thrombin) or made intraoperatively out of fibrinogen coming from the patient's own blood. A mixture of thrombin and fibrinogen to enhance local surgical hemostasis (arrest of bleeding) and to provide effective tissue adherence has long been explored, in 1998 a commercial product (Tisseel) was approved by FDA. Later, a number of other fibrin glue products have been developed commercially. (i.e. FloSeal). Also, many fibrin pads, bandages and patches have become available that help arrest bleeding.

Intraoperatively, making fibrin glue, has become state-of-the-art with the development of devices for the harvesting of platelet-rich plasma (PRP). Medtronic's Magellan, Cell Factor Technologies's GPS System, Interpore Cross's AGF Processor and Harvest's SmartPReP are some examples of new technologies available intraoperatively to process PRP for tissue adhesives, which can be employed to prepare an autologous fibrin glue for incorporation as a carrier material into the liquid mixture/cured electrode of the present invention.

Fibrin glue is derived from two components. The first component contains human fibrinogen and coagulation factor XIII and varying amounts of other plasma proteins such as fibronectin and plasminogen. The second component contains thrombin (of either bovine or human origin). In Europe, both components are human derived and supplied in commercial fibrin glue “kits”. In the United States, only the bovine thrombin component is commercially available, but commercially manufactured human thrombin and fibrinogen preparations are currently under development.

The elastic property, tensile strength, and tissue adhesiveness of plasma fibrin glue or sealant has made it an important adjunct in microsurgical techniques, conventional hemostasis, cardiopulmonary bypass surgery, colostomy closure and splenic injury repair. On cosmetic surgery, tissue fibrin adhesives have been used in lieu of sutures to reduce scar formation and in aiding skin graft fixation in burn patients. In various microsurgical techniques, fibrin sealants have been used not only to achieve adequate hemostasis but also to attain a fluid or air barrier, to maintain tissue adhesiveness and as adjunct in bone and cartilage repair.

When human tissue is injured, bleeding ensues and then ceases due to formation of a blood clot. This is the initial mechanism of natural wound closure. A clot is formed as a product of the final common pathway of blood coagulation. Fibrin glue mimics this coagulation cascade resulting in its adhesive capability.

Once the coagulation cascade is triggered, activated factor X selectively hydrolyses prothrombin to thrombin. In the presence of thrombin, fibrinogen is converted to fibrin. Thrombin also activates factor XIII (present in the fibrinogen component of the glue), which stabilizes the clot, by promoting polymerization and cross-gluing of the fibrin chains to form long fibrin strands in the presence of calcium ions. This is the final common pathway for both the extrinsic and intrinsic pathways of coagulation in vivo, which is mimicked by fibrin glue to induce tissue adhesion.

There is subsequent proliferation of fibroblasts and formation of granulation tissue within hours of clot polymerization. Clot organization is complete two weeks after application. The resultant fibrin clot degrades physiologically.

The two components of fibrin glue can either be applied simultaneously or sequentially, depending on the surgeon's preference.

Generally, the two components of the fibrin glue may be mixed with the conductive elements right before injection/placement into the patient; or one of the two components may be pre-mixed by the manufacturer, thereby providing a situation for the physician where to mix two components together (“component A,” by way of example only, being a 15% fibrinogen, 70% gold element mix; “component B” being the remaining 15% thrombin of the weight of the total volume of 100% liquid mixture). In another embodiment, “component A” may contain a 15% thrombin, 70% gold element mix; “component B” being the remaining 15% fibrinogen of the weight of the total volume of 100% liquid mixture. These ratios may be skewed more towards a 25% fibrinogen, 25% thrombin, 50% gold (or other metals) liquid mixture ratio or other ratios as needed to be thin enough to be dispensed by the means applicable and able to provide sufficient levels of conductivity inside the body once cured.

When simultaneous application is preferred, both the components are loaded into two syringes with tips forming a common port (e.g., Duploject syringe). When injected, the two components meet in equal volumes at the point of delivery. The thrombin converts the fibrinogen to fibrin by enzymatic action at a rate determined by the concentration of thrombin. The more concentrated thrombin solution, thrombin 500, produces a fibrin clot in about 10 seconds and the more dilute thrombin solution, thrombin 4, results in a clot in about 60 seconds after glue application to the surgical field. As mentioned earlier, both the extrinsic and the intrinsic mechanisms of blood coagulation are bypassed but the physiological final common pathway of coagulation is replicated. Factor XIII (present in the fibrinogen component of the glue) cross-glues and stabilizes the clot's fibrin monomers while aprotinin inhibits fibrinolytic enzymes, consequently resulting in a stable clot. The final common pathway of coagulation cascade is represented by the diagram in FIG. 30.

For sequential application, thrombin is first applied to the area of interest, followed by a thin layer of fibrinogen. In a minute or two, coagulation starts and by two or three minutes, polymerization is complete.

Alternatively, when apposition is required between opposing surfaces, thrombin solution may be applied to one and fibrinogen to the other surface.

In all of these cases, prior to application of the glue, the surgical field must be dried meticulously. After application, the tissue is pressed gently over the glue for 3 minutes for firm adhesion. At the end of the procedure, pad and bandage is applied after instillation of antibiotic drops.

Fibrin glue prepared from a donor is as safe as other tested blood products. Most but not all viruses can be inactivated by solvent or detergent treatment.

The alternative approach to ensure that fibrin glue is virus free is by preparing it from homologous fresh frozen plasma from donors in whom current tests for viral markers are negative for at least six months after the donation. This simple accreditation measure excludes the theoretical possibility of the donors having been in the “window period” when they donated blood or plasma. To further ensure its safety, most of the proteinaceous products are sterilized by gamma irradiation.

If autologous serum is utilized to produce the liquid mixture, then manufacturer provided conductive elements may be mixed in pre-determined ratios by weight to provide e.g. gold-based conductive elements with the patient's blood to form the conductive fibrin glue. Such autologous serum based may be very well tolerated by patients and, not utilizing ingredients such as grass-fed beef, it is vegan and thus applicable to a larger patient population.

Fibrin glue reduces the total surgical time because time required to place sutures is saved. The use of glue has been found to lower the risk of post-operative wound infection, contrary to conventional suturing. This can be attributed to accumulation of mucous and debris in sutures which may act as a nidus for infection. However, there is no data available to substantiate the low incidence of post-operative reaction and infection.

Mixtures of fibrin glue and antibiotics are being used for local delivery of antimicrobial activity. It is well tolerated, non-toxic to the tissue wherever it is applied and has some antimicrobial activity. The smooth seal along the entire length of the wound edge results in a higher tensile strength, with the bond being resistant to greater shearing stress. Fibrin glue is also a useful adjunct to control bleeding in selected surgical patients. It has a low incidence of allergic reactions. However, anaphylactic reactions following its application have been reported. This reaction has been attributed to the presence of aprotinin in fibrin glue.

Fibrin glue encourages the formation of adhesions when applied to contaminated tissues. Its use in infected wounds has been reported by two authors. This may be possible due to presence of aprotinin which possesses some antimicrobial activity. Chen et al. Curr Pharm Des. 2002; 8(9):671-93, however, reported that fibrin glue failed to demonstrate any bacteriostatic effects to either Gram−ve or Gram+ve bacteria by verifying the size of the bacterial growth inhibition. They also detected minimal cytotoxic activity but this was not found to be significant clinically.

Collagen, proteins and other patient-provided, animal-sourced, other-patient-sourced or synthetically gathered components may further be part of the mixture to advance with tissue integration and wound healing.

Biodegradable (mesh/suture) strips that have a glue (dispensed) on them to be able to attach to themselves in the wound and be filled or coated on the other side with conductive, biocompatible mix (fibrin glue and other carrier materials with conductive elements).

Hemaseel (Haemacure Corporation, Montreal, CA), is a fibrin-based sealant used between skin grafts and wound sites, and is suitable for use as the carrier material in a liquid mixture. As used currently, the use of the fibrin sealant between the skin graft and the wound bed interface provides adhesive qualities allowing fixation of the graft without the use of staples or sutures and seals the tissue bed layer, thereby inhibiting seroma or hematoma formation without compromising the healing process, resulting in a higher percentage of graft take with a more acceptable cosmetic outcome than using mechanical fixation.

As with two part silicones, each part of the fibrin glue (e.g., thrombin-containing crosslinker and fibrin) may be separately mixed with conductive elements and then mixed via a dispenser at the time of application. To improve the chemical and mechanical characteristics of the conductive element integration within the fibrin matrix, the conductive elements may be surface modified with a tri-amino acid sequence, arginine-glycine-aspartate (“RGD”) peptide or other functional group that improves the interface between the two materials. With conductive elements consisting of gold, the surface may be modified through disulfide chemistry with the gold surface.

The process of injecting a liquid mixture from autologous ingredients includes the following steps (1) blood is drawn to extract serum; (2) the serum is processed to extract the ingredients to form fibrin glue or a likewise structure to form the carrier medium of the liquid mixture, and (3) the carrier medium is then mixed with conductive elements to form the liquid mixture to form a cured electrode.

An alternative method of delivering a fibrin+thrombin+conductive particle (e.g. gold) mixture is achieved by first freezing the components and then mixing the frozen components in their respective ratios (ratios mentioned above in this section). One method of freezing the liquid components is to supply liquid fibrin under moderate pressure in a heated syringe with heated nozzle in a freezer (temperature of negative 20 degrees C. or colder) with the effect of forming fibrin snow which deposits on a tray within the freezer. This fibrin snow must not be compacted to retain proper mixing ratios later in the mixing procedure. Liquid thrombin is processed similarly to collect thrombin snow on a second tray (either within the same freezer but different compartment or same freezer). Likewise, conductive particle (i.e. gold) powder of the chosen grain/particle size is cooled to the same temperature as the fibrin and thrombin snow and provided to a third tray within the freezer. A manually controlled, or semi or fully automated manipulation unit then collects the appropriate volumes of fibrin snow, thrombin snow and conductive particle powder, and uses the measurement of each of the component's weight to control the future properties of the mixture. All three components are supplied to a blender which may use a rotational motion, planetary mixing or spatula to blend the three components to a homogeneous mixture. Once a homogeneous mixture has been achieved, it is partitioned into syringes or other delivery devices, all of which are pre-cooled to avoid any unintentional melting of either of the snow components. The syringes may be stored within a freezer (−20 degC or cooler, better is −80 degC) or stored and/or shipped on dry ice (temperature approximately −78.5 degC) until the liquid electrode mixture is desired. Once the liquid electrode is desired, the cold syringe with cold contents may either be heated in a warm water bath for a duration lasting from seconds (thin syringe, cold temperature −20 degC) to a few minutes (thick syringe, cold temperature −78.5 degC). The temperature of the water bath may be between 15 and 42 degreesC, colder temperatures offering a slower fibrin formation and thus longer work time for the mixture prior to achieving full cure. Alternatively, the syringe may be heated in a purpose built heating device that measures the temperature of the mixture inside the syringe during the application of heat, reporting on the rise in temperature and reaching the mixing and later the dispensing temperatures. The purpose built heating device may furthermore provide a countdown that indicates the amount of time available until the mixture inside the syringe is beginning to harden by itself. Optimal blending of the mixture may further be achieved by agitation of the syringe via US, mechanical vibration or by using a mixing nozzle that forces the liquid mixture through channels inside the needle, leading to an increase in homogeneity of the liquid mixture just prior to injection/placement. The advantage of mixing frozen components is to retain the maximal curing time for the physician in the OR, and ensuring fresh mixtures of reproducibly high quality.

### Protein Glues, Amino Acids, Arginine, Polyamine, and Other Ionic Conducting Polymers

Protein glues are suitable carrier materials. One example of a protein glue suitable as a carrier material is transglutaminase, also called meat glue that provides a carrier medium for the liquid mixture. Transglutaminase is an enzyme that stimulates a bonding process at the cellular level with the amino acids lysine and glutamine in proteins. It is a protein present naturally in both plant and animal systems. The product used in kitchens is created from natural enzymes using a fermentation process. The preparation of the liquid mixture may require further processing to ensure proper human biocompatibility.

Generally, transglutaminase may be any of various enzymes that form strong bonds between glutamine and lysine residues in proteins including one that is the active form of clotting factor XIII promoting the formation of cross-glues between strands of fibrin.

By doping the protein glue with conductive (biocompatible) materials before or during the curing process, electrically conductive tissues may be built inside the body to form a liquid mixture.

### Dental Resins, Cement and Amalgam

Dental resins are nonconductors by nature, biocompatible, malleable when placed and may be cured with the application of UV or blue light in-vitro. While many applications for the liquid mixture/cured electrode may require a flexible electrode, there may be situations where an inflexible cured electrode is advantageous. For these applications, resins that are mixed with a conductive element in appropriate mixture ratio (e.g., 70% mixture, 30% resin; or 50% mixture, 50% resin; etc.).

Dental composite resins are types of synthetic resins which are used in dentistry as restorative material or adhesives. Synthetic resins evolved as restorative materials since they were insoluble, aesthetic, insensitive to dehydration, easy to manipulate and reasonably inexpensive. Composite resins are most commonly composed of Bis-GMA and other dimethacrylate monomers (TEGMA, UDMA, HDDMA), a filler material such as silica and in most current applications, a photoinitiator. Dimethylglyoxime is also commonly added to achieve certain physical properties such as flow ability. Further tailoring of physical properties is achieved by formulating unique concentrations of each constituent.

Many studies have compared the longevity of composite restorations to the longevity of silver-mercury amalgam restorations. Depending on the skill of the dentist, patient characteristics and the type and location of damage, composite restorations can have similar longevity to amalgam restorations.

As with other composite materials, a dental composite typically consists of a resin-based oligomer matrix, such as a bisphenol A-glycidyl methacrylate (BISGMA), urethane dimethacrylate (UDMA) or (semi-crystalline polyceram) (PEX), and an inorganic filler such as silicon dioxide (silica). Compositions vary widely, with proprietary mixes of resins forming the matrix, as well as engineered filler glasses and glass ceramics. The filler gives the composite wear resistance and translucency. A coupling agent such as silane is used to enhance the bond between these two components. An initiator package (such as: camphorquinone (CQ), phenylpropanedione (PPD) or lucirin (TPO)) begins the polymerization reaction of the resins when external energy (light/heat, etc.) is applied. A catalyst package can control its speed.

A hand-held wand that emits primary blue light (λmax=450−470 nm) is used to cure the resin within a dental patient's mouth and may be used similarly near neural structures in the patient's or proband's body without risk to the neural structure during light application. An example of a dental resin liquid mixture comprises (1) Bis-GMA or other dimethacrylate monomers (TEGMA, UDMA, HDDMA), and (2) Ag or Au. It is injected in its liquid form and then cured in the body with blue light in the way that it is dispensed around a target, then cured, then dispensing continues, then curing continues. This process continues alternating the dispensing of the liquid mixture and the curing as needed to mold the desired shape of the electrode around or near the nerve.

A range of resins and cements exists, providing different levels of mechanical hardness and stability.

Glass ionomer cement (“GIC”)—composite resin spectrum of restorative materials used in dentistry. Towards the GIC end of the spectrum, there is increasing fluoride release and increasing acid-base content; towards the composite resin end of the spectrum, there is increasing light cure percentage and increased flexural strength.

Resin electrodes might allow an integration of the liquid mixture which then cures into a bone, mechanical fixation around, near or into a bone, as well as the formation of mechanically stiff cured electrode able to resist muscle forces where needed.

GICs are hybrids of glass ionomers and another dental material, for example Resin-Modified Glass Ionomer Cements (RMGICs) and compomers (or modified composites). These materials are based on the reaction of silicate glass powder (calciumaluminofluorosilicate glass) and polyalkenoic acid, an ionomer. Occasionally water is used instead of an acid, altering the properties of the material and its uses. This reaction produces a powdered cement of glass elements surrounded by matrix of fluoride elements and is known chemically as Glass Polyalkenoate. There are other forms of similar reactions which can take place, for example, when using an aqueous solution of acrylic/itaconic copolymer with Tartaric acid, this results in a glass-ionomer in liquid form. An aqueous solution of Maleic acid polymer or maleic/acrylic copolymer with Tartaric acid can also be used to form a glass-ionomer in liquid form. Tartaric acid plays a significant part in controlling the setting characteristics of the material.

Fissure sealants, which involve the use of glass ionomers as the materials can be mixed to achieve a certain fluid consistency and viscosity that allows the cement to glue into fissures and pits located in posterior teeth and fill these spaces which pose as a site for caries risk, thereby reducing the risk of caries manifesting.

Cermets are metal reinforced, glass ionomer cements and they improve the mechanical properties of glass ionomers, particularly brittleness and abrasion resistance by incorporating metals such as silver, tin, gold and titanium. The use of these materials with GIC increases compressive strength and fatigue limit as compared to conventional GIC, however there is no marked difference in the flexural strength and resistance to abrasive wear as compared to glass ionomers. This means that there are some processes of mixing the dental cements with metal elements in place and a substitution of the currently used metals (aimed at mechanical stability) for a metal aimed to increase conductivity is desirable (Ag, Au, etc. as well as non-metal mixtures such as graphene, carbon nanotubes etc.)

Eutectic systems, for example dental amalgams, are metal compositions that are composed of metals in powder form and at least one metal in liquid form at the time of formation. Dental amalgam is one example of such a eutectic system, where mercury provides the flux (ability to flow and react) for the said metals to form a eutectic structure in an exothermic reaction that creates a hard, durable and electrically conductive medium. A cured electrode formed as a eutectic system does not necessarily need another carrier medium, as the metallic components of the eutectic system provide high levels of electric conductivity.

As amalgam assumes the mechanical properties of a paste prior to curing, so a simple syringe/needle system may not be sufficient for delivery/injection, especially a small gauge needle. In these cases, the needle/syringe and the amalgam column inside is vibrated at frequencies of 600 to 60,000 Hz. Vibrating the dental amalgam can allow more viscous material to achieve a lower effective viscosity (similar to how sand can flow similar to a liquid when vibrated). Vibration may be used to assist in delivery of amalgam and also any other liquid mixture.

Dental amalgam is a liquid mercury and metal alloy mixture. Low-copper amalgam commonly consists of mercury (50%), silver (˜22-32%), tin (˜14%), copper (˜8%) and other trace metals.

Basic constituents include (1) Silver, to increase strength and expansion, (2) Tin—to decrease expansion and strength, and to increases material setting time, (3) Copper—to bond to tin, reduce tarnish, corrosion, creep and marginal deterioration and increase strength; (4) Mercury—to activate reaction of the material; (5) Zinc—to decrease oxidation of other elements, increase clinical performance, and produce less marginal breakdown; (6) Indium—to decrease surface tension, reduce amount of mercury necessary, and reduce emitted mercury vapor; and (7) Palladium—to reduce corrosion. Being electrically conductive, amalgam does not need conductive elements to increase its conductivity.

Amalgam may not be applicable for all potential applications, though there are locations where high tensile strength, shear strength, or mechanical stiffness may be advantageous or not considered a problem. Examples of such implant locations are the leg stump of an amputee where there is no muscle activity or where a nerve is running very close to a bone and there is little or no lateral motion between the nerve and the bone. Placing the amalgam partially into the bone (optionally, after creating a hole in the bone) allows for a stable attachment of the amalgam in one location. A dispenser may employ a small drill to provide an anchor point for the cured electrode in which the carrier material is dental amalgam.

A variation is a cured electrode of dental amalgam which is both soft and hard. One part is hard and e.g. anchored into a bony tissue near the nerve to be stimulation to eloquently hold it in place, while the contact to the nerve is established though a soft portion which is glued (electrically conductive or non-conductive) to the hard portion, allowing for mechanical stability of the entire system and increased flexibility of the connection to the nerve.

An amalgam cured electrode, when encased in a nonconductive carrier material during or post curing/setting of the amalgam may further provide a variety of applications to conduct electrical current inside the body without unintentionally stimulating nearby tissue or losing currents to crosstalk and parallel pathways.

In yet another embodiment, a cured electrode of amalgam may be placed without anchoring it into a bone to be able to move with the surrounding tissue. As long as the relative motion between a nerve and an amalgam cured electrode is minimal, such as not more than 0.5 mm co-axially and not more than 0.2 mm radially to the nerve, then such a cured electrode may be applicable in a location where there is very little or no muscle and/or skin movement near the cured electrode.

### Bone Cement

In another embodiment bone cement, or poly(methyl methacylate) (“PMMA”) based materials, may be used as the liquid carrier material. PMMA bone cement has been used extensively as an implantable biomedical material. It is a rapidly curing polymer and may be mixed with conductive elements to yield a liquid mixture. A cold-cure system typically consists of a powder, a cross-linking agent, and an accelerator that is typically integrated with the solvent (e.g. N,N-Dimethyl-p-toluidine). A conductive filler may be combined homogenously throughout the powder (PMMA+crosslinking agent) which is then combined with the solvent and accelerant solution to initiate polymerization.

PMMA mixtures likewise may be processed similarly (as described above) in a frozen environment to form a PMMA-conductive particle granulate mixture that may be transported under dry ice to the application location where a heating allows for easily reproducible work and curing times for the physician.

### Conductive Elements

Very high intrinsic electrical conductivity is the primary property for the conductive elements, although intermediate conductivity levels are useful when resistivity is to be exploited to form electrodes of varying impedance levels. In one embodiment element sizes tested are in the μm range (in a preferred embodiment approximately 10 to 300 μm) as produced by filing metal with a conventional metal file and most filings had a diameter of approximately 100 to 200 um. The nanometer range shall be avoided for conductive elements as metals in the nanometer range have been reported to show characteristics (such as toxicity) which are not observed in micron and macroscopic levels. Considering that the conductive elements are applied as a device to conduct electricity and not as a “drug” to kill cancer cells which can be observed with gold elements in the nanometer range, conductive elements have at least one dimension which is one micron or more, and in some preferred embodiments the conductive elements are in the range between approximately 10 and 300 μm. Different conductivities are desirable to enable resistive as well as well conducting lines in parallel. This partially-conductive material may comprise a conductivity between the most conductive and the most insulating material. Innate biocompatibility of the conductive elements mixed with the carrier medium is advantageous, but not absolutely necessary.

The conductive elements herein may comprise dental amalgam (comprises Ag, Ni, Cu, Hg). Although dental amalgam includes elemental mercury (approximately 50% of the material content as measured by weight in dental amalgam is Hg), the Hg is bound so well in the eutectic structure of the amalgam, that the amount of Hg leached even when mechanical forces (biting) and chemical solvents (in saliva as well as acids in fruit and other food) are applied in combination, the contamination of the human eating with a Hg-containing tooth filling in their mouth is considered safe. Innate biocompatible materials are gold in pure and in alloyed form, titanium (pure or alloy), platinum (pure or alloy) and others. The conductive elements comprise a metal with appropriate properties selected from a group consisting of gold, vanadium, niobium, iron, rhodium, titanium, tantalum, gallium, arsenic, antimony, bismuth and platinum. While some of the alloys and pure forms of these metals possess innate toxic properties, limiting the metal's bioavailability is key to its use as implanted materials. The conductive elements also may comprise a carbon-based conductive material from a group consisting of graphite, graphene, diamond and carbon nanotubes. While diamond is an insulator, graphite and graphene show highly conductive properties for electrical current. Another metal with high conductivity is aluminum (Aluminium internationally). Carbon Nanotubes, nanometers in diameter but micrometers in length, are highly conductive for electricity and are very biocompatible, biotolerable or bioinert in chronic implantation in both, preclinical and clinical studies and applications. Stainless steel is used widely in medical implants and is electrically conductive. Alloys such as nitinol (51% nickel, 49% titanium) are being used in heart valves, ocular applications and other implant locations of the body. While a patient may have an allergic (or otherwise unwanted medical) reaction to a compound (e.g. nickel) of an alloy, patient tolerance to the alloy as a whole is significantly improved. While toxic to bacteria and living tissue (such as cells of animals or humans), copper and silver (in pure form as well as their alloys) are highly electrically conductive and can be implanted when bioavailability is limited. Copper (II) ions are toxic for biological systems and it is important to shield copper metal from dissolving and its ions being able to diffuse or otherwise travel away from the implant location, thereby becoming bioavailable. If on the other hand copper (and copper alloys, as well as similar metals considered harmful to biological tissue) are coated with another metal that does not dissolve in the biological environment under chronic conditions, then copper can be used. Corrosion resistance to the chronic implant location is important, not necessarily for the pure metal as such, but for the implanted system as a whole: While aluminum itself is highly reactive with oxygen, it is the oxides of the metal that allow aluminum to be practically inert in nature, making it attractive for many industrial applications. Furthermore, many metals are present in the human body in bound form (referred to as “biometals”), meaning that the human body is able to process metals in solution to a certain extent, especially when they are present in chemical compounds inside the body. One example of a metal alloy is bronze.

TABLE FIVE
Conductance For Metals And Carbon Based Substances, In Ohms:

| Material | Conductivity (1/(Ωm)) |
|---|---|
| Au (gold) | 6 × 10⁷ |
| Ag (Silver) | 6.29 × 10⁷ |
| Ti (Titanium) | 2.4 × 10⁶ |
| Ni (Nickel) | 1.43 × 10⁷ |
| Pt (Platinum) | 5.5 × 10⁷ |
| Cu (Copper) | 5.95 × 10⁷ |
| Al (Aluminum) | 3.77 × 10⁷ |
| Fe (Iron) | 1.03 × 10⁷ |
| Carbon nano-tubes | 106 to 10⁷ |
| Graphene | 2.9 × 10⁷ |
| Conductive polymers | 104 × to 10⁷ |

The conductive elements may comprise high aspect ratio materials, although all conductive elements need not have a high aspect ratio. As used herein, aspect ratio of the conductive elements refers to the ratio of the maximum dimension of the element compared to the minimum dimension. A sphere, by definition has an aspect ratio of 1, where as a rod with diameter 1 micron and length 10 microns has an aspect ratio of 10. High aspect ratio conductive elements have the advantage that they may achieve electrical percolation throughout a composite matrix at a lower weight percentage than lower aspect ratios.

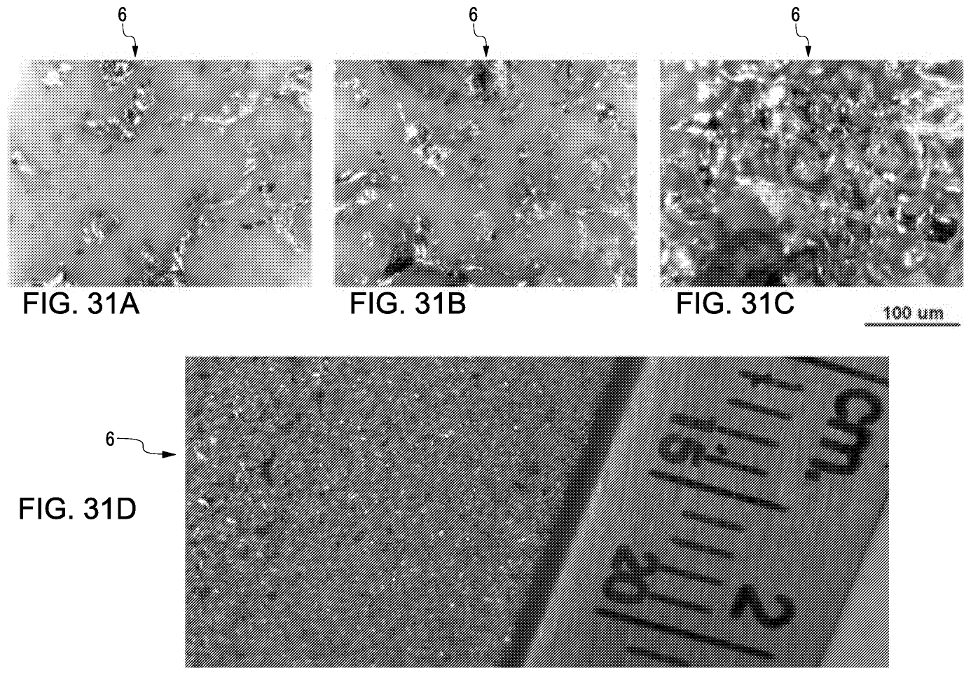

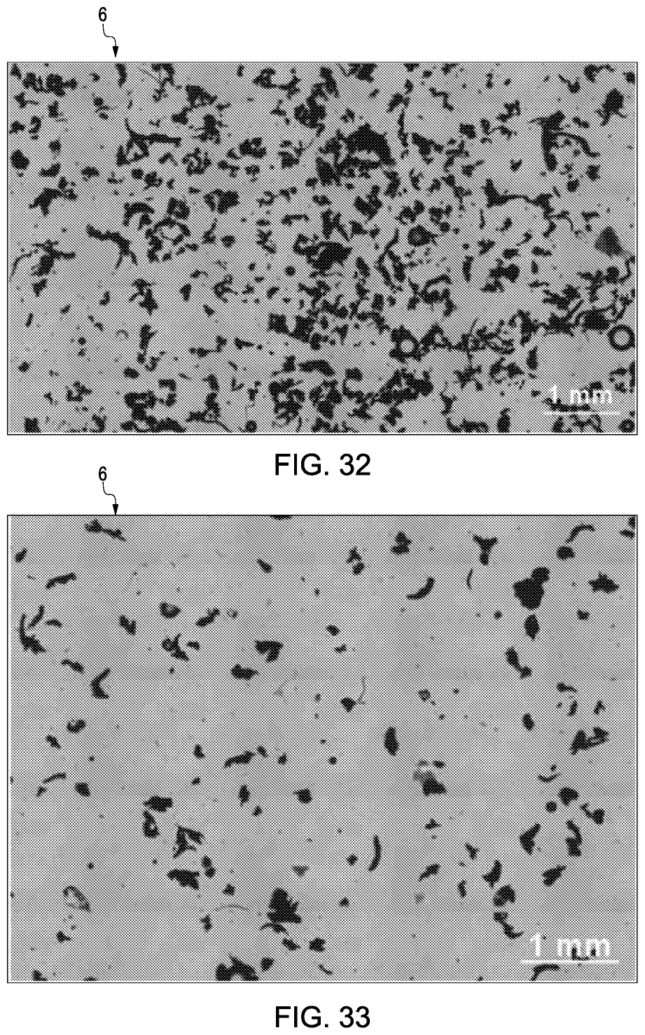

FIGS. 31A-D are images of silver flakes manufactured with various grain size sand paper wheels using a Dremel tool. FIG. 32 is another image showing the same high-aspect ratio silver filings as shown in 31A-D. FIG. 33 is an image of gold flakes of various aspect ratios produced with a Dremel tool.

A portion of the metal flakes produced by grinding in this fashion comprise shapes that may interlink as a hook and loop fastener holds on and bonds through many small connections for both electrical conductivity as well as mechanical stability of the cured mixture. Thus, the production of flakes through grinding via dremel produces inherently non-uniform, high aspect ratio, bent and pointy metal elements. Another method of producing conductive elements is use of wet and dry sand paper of 600 pitch grain, which produces conductive elements affording significantly increased the flow rate through a thin needle for liquid mixture/cured electrodes of the same weight percent as compared to the non-sandpaper-post-processed material. In addition to the interlinking properties of metal flakes described herein, the conductive elements, metal or otherwise, may be manufactured with specific features selected from a group consisting of hooks, loops and coils, so that these features can interlink with one another, thus improving the connectivity and durability of the network of conductive elements. In one embodiment the conductive elements are small bits cut from a conductive material comprising fibers of a shape found in a steel wool.

For metal flakes and other conductive elements an aspect ratio of 5:1 and up to 1000:1 is desirable, although an aspect ratio as low as 2:1 is acceptable depending on the application. Conductive elements of less than 2:1 are capable of conducting current, but the percent weight of the conductive elements within the mixture (comprising the carrier and the conductive elements) would increase. The aspect ratios stated herein may be, but are not necessarily, uniform throughout a liquid mixture/cured electrode. The conductive elements, as described herein, maintain connectivity even under mechanical deformation that a flexible carrier material can withstand.

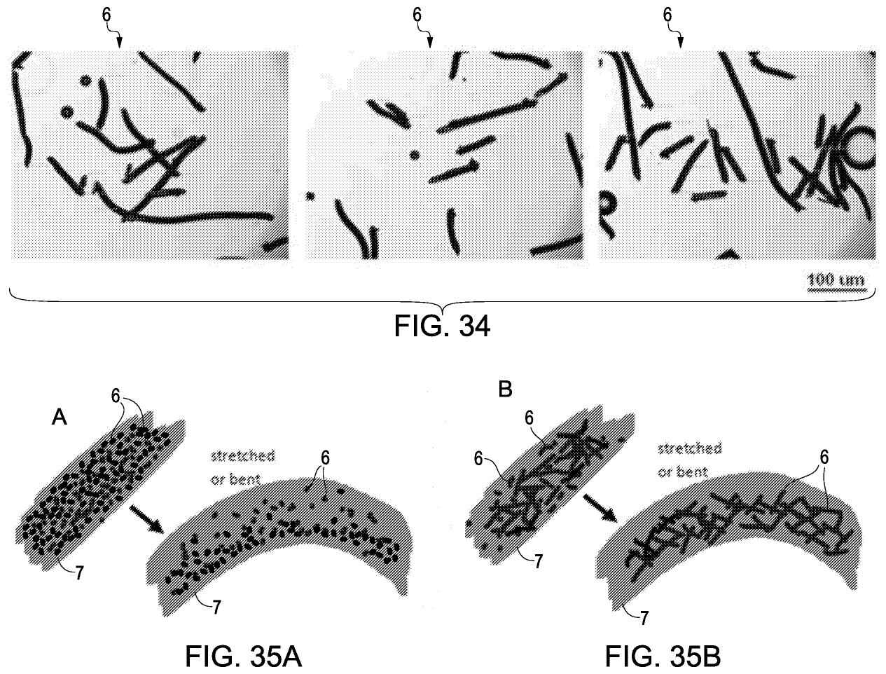

In one embodiment, gold bonding wire, as used in the semiconductor industry, is used to manufacture conductive elements is a suitable source for the conductive elements. Gold bonding wire—describe diameter/width and any other relevant information such as a product or manufacturer name. In one embodiment, the gold bonding wire may be cut into bits comprising lengths of 10 μm to 900 μm, three images of which are shown in FIG. 34. The shorter wire bits (approximately 10-60 μm) are better for fitting through a tight needle, with a maximum of 20 gauge, and preferentially 22-26 with <10 micron conductive elements, improve conductivity even when the cured electrode is stretched or bent. FIGS. 35A-B are idealized section views of a cured electrode in its original shape and a subsequent bent position showing how, after bending, the high aspect conductive elements FIG. 35B maintains connectivity compared to lower aspect ratio FIG. 35A. The mechanism of action of longer bits providing better conductivity at lower weight percentages, especially when non-uniform, bent and with the ability to interconnect is shown in low aspect ratio FIG. 35A conductive elements are more likely to lose connection to neighboring conductive elements when the cured electrode is bent, not so high aspect (FIG. 35B) versions.

When connective elements of high aspect ratio are used, such as fibers, whiskers, bonding wire bits, flakes, then these elements can shift in two dimensions within the cured electrode without the loss of connectivity. Using the example of two bits of bonding wires, these may slide along their axes, twist against each other, and slide along each other's axis so that connectivity (by continuing to touch) is never lost. In contrast, if low aspect ratio (e.g., a sphere) is shifted against another sphere then connectivity is lost virtually immediately. In one embodiment, advantageous results are achieved with at least a portion of the conductive elements comprising an aspect ratio 2:1 to 20:1, comprising a diameter of 15-50 μm, and length 15 to 300 μm, maintaining a high likelihood of maintaining contact with movement in two of three dimensions.

Methods of manufacturing conductive elements include: (1) Laser cut: (plain cutting or with the goal to round off the cutting edges, essentially forming a small ball at each end of the wire; if the ball is larger in diameter than the wire and a mini barbell is formed, then these too may interconnect and interlock with each other, providing added mechanical strength, while minimizing the risk of puncturing the nerve with sharp edges as well as minimizing the electrical field density at the tip of the wire on the edge of the liquid mixture/cured electrode at the interface to the electrolyte). (2) Electrical cut: burning through the wire at specific points with high current; similar results to Laser cut are possible. (3) Scissor cut. (4) Cryo-Cut approach: encase gold bonding wires (or the like) into a matrix, then freeze, and use a sharp blade to cut the matrix, which increases the ability to mass-produce similar length wire bits. Microwires, such as gold bonding wire, may be incorporated in a cutting matrix such as an OCT (optimal cutting temperature) compound used for cryo-histology. The wires may then be cut using a precise microtome (or vibratome, cryostat) such that a reproducible length of conductive elements is produced, which are collected from the collection pan below the blade, and rinsed on a filter to remove the cutting matrix compound. (5) Shaving from a spool: using a file, a knife, an angle grinder—because of shaving from a spool, mass production is possible by essentially cutting through the spool.

Gold bonding wire, cut into various lengths as conductive elements, may be (1) uniform or varying length within limits (2 sigma within L=100 μm long, rest 50<L<200 μm), (3) with bending (intentionally) or without bending (intentionally), and/or (4) with or without tips rounded via electric zap.

Conductive elements of nitinol wire (or other shape memory electrical mixtures) may be processed with aspect ratio, in one embodiment, 10:1 to 30:1. When processed from an oblong spool, in one embodiment they can be programmed to be curved at the edges (hooks at both ends), and flattened during cold processing, then they may be injected (easily flow) at room temperature, and then heated above their return “shape memory” threshold such that they return to a “hook” shape and are more likely to cross and form interlocking features 28, such as coils and hook-and-loop-like structures, for conductive elements 6. In one embodiment, nitinol conductive elements change their shape from body heat when they are injected into a body. In another embodiment, they may be extruded as a straight wire and coiled afterward with cold-processing, and then cut into small segments such that they can be injected with low aspect ratio (coils that flow easier through a needle) and later be uncoiled into straight rods with high aspect ratio (better electrical percolation through the matrix, more mechanically encapsulated in the material. In another embodiment, they may be injected at room temperature as straight bits of wire then, from a body's normal heat, change into shapes which interlock and form a mesh. This allows for an injectable mesh, which is assembled by the body itself from the nitinol bits being exposed to body heat.

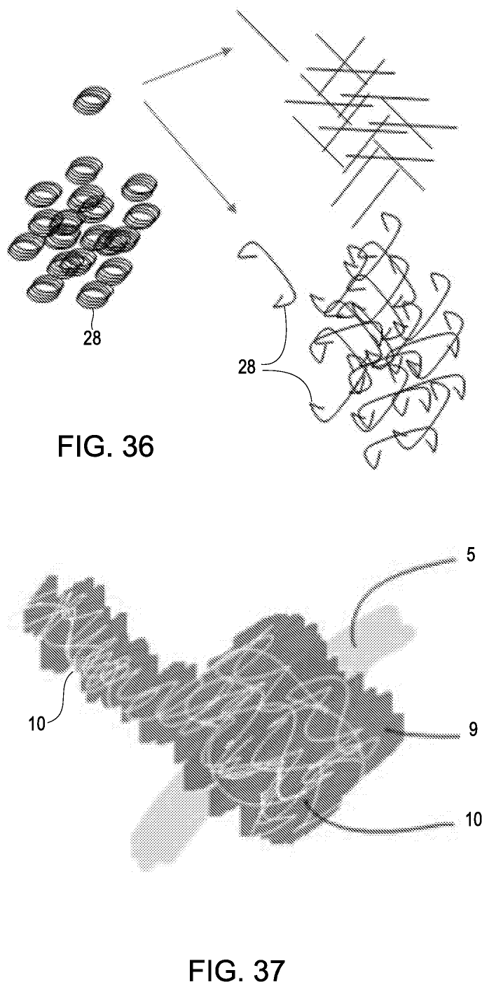

FIG. 36 is a diagram of a mechanism of action for NiTi wire conductive elements added to the liquid carrier material to provide a decrease in impedance. NiTi wires may come pre-coiled to a small diameter with the drive to straighten once subjected to body temperature (transition temperature about 35 degrees C.). The pre-coiled assembly facilitates the delivery of the small NiTi wire coils 28 through a smaller diameter bore needle or other dispenser, while the straightening of the wires (with or without ends remaining hooks) themselves interconnect within the carrier material once inside the body and heated to body temperature but before the carrier material of the carrier has cured. Once the liquid mixture has fully cured, a matrix of interconnected NiTi wires retains a low impedance value.

In yet another implementation, the wires are partially un-coiled for delivery through a small bore needle or other dispenser. At a transition temperature just below body temperature, the wires coil slightly. As the partially coiled wires link together more post-delivery (after injection) but pre-curing of the liquid carrier, the small coils 28 interconnect across the bulk of the mixture, forming an interconnected network from small formerly disconnected elements.

In another embodiment, high aspect ratio conductive elements with sharp tips have these advantages: (1) ability to penetrate the epineurium over time and provide a better SNR, (2) ability to make electrical contact with the entire nerve, (3) may be added with a liquid carrier material as “glue” to existing electrodes just prior to implantation to achieve better electric coupling to the nerves, and (4) may be used to integrate better into bone and other rough surfaces.

In another embodiment, low aspect conductive elements may be interspersed with those of high-aspect, so as to reduce irritation to tissue in some applications.

In another embodiment, the conductive elements may comprise a network of conductive mesh 24, forms or filaments of electrically conductive surgical suture; and mesh, forms or filaments of other conductive elements; the filaments may be made from materials/fibers such as conductive metals or carbon-based materials or biocompatible polymers with functionalized groups for conductivity. Alternatively, carbon nanotubes (of at least a micron in one dimension) may be used to create the mesh, or may comprise individual elements. One embodiment of such a mesh structure is to form it from one wire instead of dispensing a net of conductive elements. A dispenser 2 dispenses a thin (e.g. 15 μm diameter) bonding wire covered with surfactant. This wire 10, in one embodiment, may be dispensed as a continuous string through a dispenser comprising multiple chambers and controls on the dispenser: one dispenses carrier material alone, another dispenses wire alone and yet another dispenses carrier material 7 and wire at the same time. The wire may be dispensed through a multi-chamber dispenser, each chamber having its own exit point 29 near the dispenser tip 16, and the wire is pushed by rollers toward the exit point 29 of one of the chambers, as shown in FIG. 37. FIG. 37 is a diagram of a mesh of gold bonding wire continuous loops that interconnect with each other. Even though the wire is one continuous wire, the meshing and interweaving of the surfactant-covered bonding wire allows for many physical connections between gold wire loops. In case one of the wire loops breaks or loses connection to a neighboring loop, there are still many others conducting electricity to the target.

Another material which can be used as a conductive element is poly(3, 4-ethylenedioxythiophene) polystyrene sulfonate (“PEDOT:PSS”) which is a conductive polymer. It can be solidified and ground into elements and dispersed through a liquid carrier material.

In another embodiment the conductive elements comprise surface-covered Si2O grains using chemical vapor deposition (CVD) or physical vapor deposition (PVD) to deposit diamond. This conductive diamond covered sand is electrically conductive and may be used as conductive elements.

In another embodiment, the surface of conductive elements such as gold may be functionalized with a sulfo-PEG-X or disulfide-PEG-X where X is a —OH, —COOH, —NH2 or —SH group. Surface functionalization may covalently interact during cross-linking, e.g. amine functionalized with NHS-PEG, and it may act as a surfactant or allow chain-chain interactions with the carrier material (e.g. PEG-PEG or PEG-PAA hydrogen bonding)

### Other Placement Methods and Devices

A significant advantage of the present invention electrode is that the entire procedure of finding the connection target (i.e., nerve, blood vessel, organ or alike), placing the electrode into, next to, around or nearby the connection target, laying the connection (similar to a “lead wire”) to the connection target and connecting to another target (biological or non-biological such as a signal generator)) can be done minimally invasively.

This means that the entire procedure may be done through one small “keyhole” incision of <1 cm in length, or even without an incision if a dispenser comprising a needle is used in conjunction with a non-invasive visualization: ultrasound is able to visualize both, organ walls, blood vessels and often nerves that generally run alongside an artery. Furthermore, ultrasound is able to visualize tissue plains to some degree and separated tissue plains easily, and it can visualize a metallic needle or other dispenser for the mixture in the first (liquid) phase placed into bodily tissue. Ultrasound is furthermore able to visualize live, without the need for additional contrast agents and it may visualize the dispensed liquid mixture material around, inside and near a target, especially metallic conductive elements.

In addition to ultrasound, angiography (radiography, video- and still X-ray) may be used to visualize the target, a dispenser advancing to the target, injection of the liquid mixture at the target, as well as any other structure in the area such as a previously implanted electrode or lead wire. Furthermore, visualization after post-chronic encapsulation by fibrous tissue may easily be achieved months and years post-implantation especially when metals are used as the conductive elements, such as silver and platinum. If desired, visualization may be improved by adding some platinum or silver powder in sufficient quantity (i.e. 5 to 20% by weight) as both are very radio-opaque. A continuous insulation of a cured electrode may be visualized post-operatively if the mixture uses platinum elements on a nano-scale level as long as these do not become bio-available. Although these kind of nano-elements may not intrinsically provide an improvement of conductive properties, they may not significantly increase impedance either. Even elements that are naturally electrically conductive on the micrometer scale, tend to be completely surrounded by the carrier material in such a way that the carrier medium interrupts continuous electrical connections. Surfactants may be used to aid with the assurance that sufficient direct mechanical connections between the conductive elements exist in order to facilitate for the whole network of conductive elements to possess an overall low mixture impedance as described above. Platinum powder on the nanoscale level provides visualization because of its radio-opaque character, while providing sufficient insulation through the carrier and absent electrical connections between the nanoscale elements. Visualization may be further improved by utilizing contrast agents that are injected into blood vessels near the target (i.e. an artery next to a nerve of interest; an artery providing oxygenated blood to the bladder for electrode placements near or on the bladder wall) as angiography is used in cardiac and neurosurgery.

Furthermore, in one embodiment the dispenser itself has the ability to electrically stimulate when an insulated wire is included in the dispenser which is capable of providing current through a de-insulated tip in contact with the injected liquid carrier material around, at, inside or near the connection target. Finding a nerve is achieved by providing a repetitive (or intermittent) neurostimulation pulse (200 us pulse width, 1 mA current amplitude, cathodic first vs. distal return, symmetrical charge balanced for nerves being the connection target; other, likely larger current and time values for muscle stimulation, blood vessel stimulation or organ/muscle stimulation). As the dispenser's tip 16 (e.g., needle tip or exit point 29) comes in close proximity with the target, a response may be visible (e.g., muscle movement), measured (e.g., muscle EMG, change in blood flow distal or proximal to the stimulation location measured with Ultrasound-doppler) or otherwise verified. In one embodiment the invention has the capability to immediately visualize a functional response following the electrical stimulation of the liquid mixture/cured electrode placed at, into, near, or around the target allows for immediate documentation of a successful placement. Furthermore, as the dispenser is retracted to form a wire-like portion 23 of a cured electrode, a successful continuous connection through the wire-like portion 23 can be verified by continued intermittent stimulation as only the intact connection placed by the electrode will provide conduction. A pressure sensor inside the delivery device measuring the pressure during injection/extrusion of the uncured material mixture may aid with the assessment of line continuity. Furthermore, by adding accelerometers or other types of positioning sensors to the delivery device with or without monitoring of or restraining of the target tissue, the relative position of the delivery device inside the body/tissue can be calculated by a processing system. This allows for relative motion between delivery device and various bodily tissues be used to drive the injection/extrusion of the uncured material mixture in relative manner to the motion of the delivery device. Such a computer aided delivery may utilize location information, pressure data, conductivity data and other information to assess the injection/extrusion speed, pressure and if pulsatile delivery is used, define the specific pressures and timing of injection/extrusion pulses. The automated injection/extrusion may be further correlated with expected blood vessel density in a specific tissue with the intent to seal off, glue or coagulate any blood vessels that may have been partially or completely severed during the insertion or manipulation of the delivery device into the body by injecting/extruding slightly more volume (5-15%) from the needle than the needle took up, thereby utilizing the aid of some residual pressure caused by the material left in the location the needle (or delivery device tip) took up when present in the body.

The combination of electrical functional testing, ultrasound or x-ray visualization and a general understanding of the anatomy allows a skilled physician to place a cured electrode at or around a target within five minutes or less measured from beginning of the injection to having the dispenser removed from the body of the patient.

The placement/injection of the electrode as herein may be accomplished under local anesthesia, disabling sensation in only one limb or even only a part of a limb. Avoiding general anesthesia means saving lives (local anesthesia has a much reduced risk profile in comparison to general anesthesia), cost, operating room time and personnel, and reducing recovery times. Conducting the placement of the electrode under localized anesthesia allows many interventions in bioelectronics that have previously required general surgery to become outpatient procedures.

The ability to place the entire electrode through a needle and without a large incision for surgical instruments to place a prior art cuff or electrode such with further need to secure it by sutures or encase the indwelling electrode with further reduces surgical risk of complications and reduces OR time.

An example of a patient-physician interaction for placing a neural connection would include: Office visit 1: finding the neurostimulation target 5, applying local anesthesia and verifying the best placement location via electrical stimulation through the dispenser 2. Placement of the liquid mixture/cured electrode 1 through a needle and verification of good connection from, e.g., a pad formed just below the skin to allow an interface for TENS electrodes later. Entire placement procedure done in <5 minutes. Office visit 2: One day to one week later, providing the patient with the TENS unit, verifying activation thresholds and best stimulation parameters. Patient takes TENS unit home; this TENS unit comprises very specific minimum and maximum parameters programmed into it to ensure that the TENS unit does not accidentally over-stimulate the nerve. Office visit 3: One month post implant: Verification of efficacy and safety, collecting patient feedback, verifying neural activation thresholds and adjusting waveforms as needed.

Visualization may be created or further improved by adding imaging contrast agents for use with MRI, CT/x-ray/angiography, and ultrasound, unless radio-opaque elements are added to form the PEG based liquid mixture, in which circumstance the metal component alone may be sufficient to increase visibility on MRI, CT/x-ray/angiography, ultrasound.

Visualization and other properties (stability of the suspension during the injection process, ease of injection, ease of placement, improved stickiness, ability to coagulate blood vessels and add the element of facilitating hemostasis during the injection process) may also be effected and improved by adding other polymers to the mixture. These may be in form of soluble materials or as insoluble suspensions. Soluble materials may be such as hyaluronic acid, PVA, PVP or other hydrophilic biomaterials. The insoluble suspensions may be elements of degradable and non-degradable biomaterials, including esterified HA, ceramics, polymeric suture materials and the like.

More than one of the cured electrodes of the present invention may be placed near one another for selectivity and specificity, to yield different nerve activations. If one cured electrode herein is placed near but not touching the other, then stimulating either one of these leads to an activation of a different set of nerve fibers. This is because the fibers within a fascicle as well as the fascicles within a nerve trunk as well as the nerves within a set of nerves are not stationary with their location relative to the epineurium, the outermost layer of dense irregular connective tissue surrounding a peripheral nerve. Fascicles change their relative position within a nerve trunk in relation to the others down the length of the nerve trunk. As the probability function for a nerve fiber to be activated is described by the second spatial derivative of electric field potential over time, and the electric field itself decays as function of the squared distance from its source, and the probability of axon depolarization is especially correlated with the distance of a given nerve fiber to a depolarizing electrode. Secondly, as nerve fibers are primarily activated at the nodes of Ranvier, and the fact that the nodes of Ranvier for various fiber diameters do no not necessarily line up the same way within the distance of a few (e.g., 5 to 10) millimeters, essentially the width of a given electrode placed around a nerve, it may be assumed that any single electrode's interface with a nerve might not line up with the same nodes of Ranvier each time.

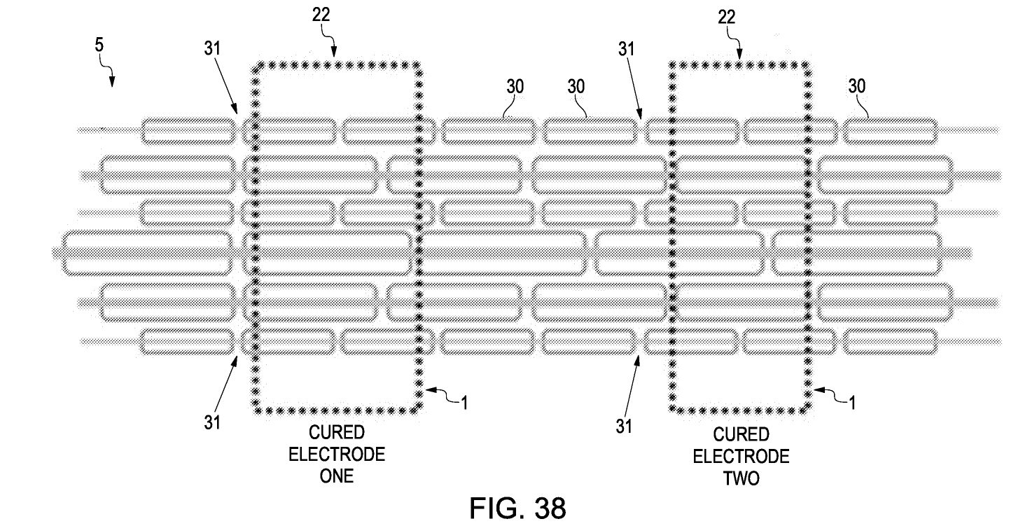

FIG. 38 is a depiction of two cured ring-like electrodes 22 on the same nerve fiber 5. While the interface of electrode one lines up well with all the nodes of Ranvier 31 between the myelin sheaths 30 of all fibers, this is not the case with for electrode two. As the change in electrical field density is the highest at the edges of an electrode, which is equally true for a cured electrode, it is the physical location of the cured electrode that defines which nodes of Ranvier will be depolarized at a given electrical field strength (i.e., stimulation amplitude in voltage or current). This causes a different activation threshold for the fibers of the whole nerve for electrode one in comparison to electrode two. Specifically, the activation thresholds for all nerve fibers, thin and thick alike, at once for this nerve will be the lowest for electrode one, while the expected stimulation thresholds for electrode two will be larger. This may be true for all fibers of this nerve and equally true for a subset of nerve fibers of a given diameter. Furthermore, not every cured electrode placed around a nerve is alike. Some might form a ring 22 around the nerve with a ring width of 2 mm (not the diameter, but the width of the ring formed by a 2 mm diameter injection needle). Some might form a 1 mm ring width. Some might form an oval shaped object. Some cured electrodes might be a thicker ring on one side of the nerve than on the other. Either way, placing several electrodes along one nerve and connecting them to different signal generators will likely lead to different nerve activation thresholds for each of the electrodes and thereby the option for selective neural stimulation by placing a multitude of electrodes on the nerve. Related concepts are presented herein.

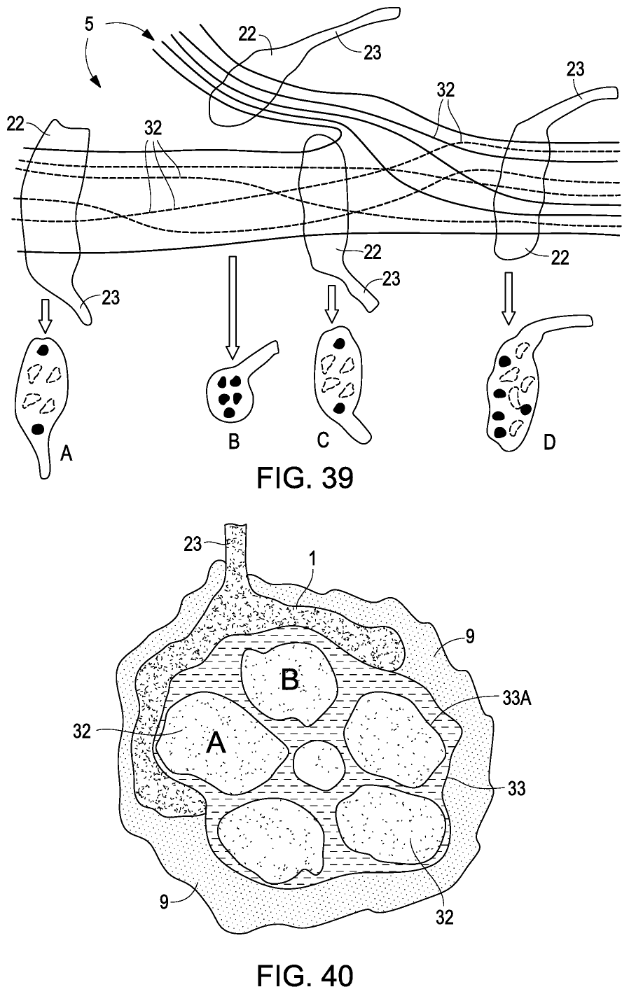

In one embodiment, a nerve fascicle may be selectively activated with a cured electrode injected along a nerve and at a nerve Y-junction, i.e., where a nerve branches. Fascicles inside a nerve do not retain their position along a nerve for an extended period of time. In fact, especially when nerves branch into two or more sub sections, a reorganization of fascicles inside a nerve takes place. This biological phenomenon can be utilized to interface with several cured electrodes, placed around the whole nerve at specific intervals, in order to achieve fascicle selective activation. This allows the specific activation of some fascicle with one electrode at one location placed more proximal along a nerve with respect to another electrode placed more distally, that cannot activate that fascicle within the same nerve at the same stimulation amplitude as the more proximally placed cured electrode did. This is especially apparent in cases where significant reorganization of fascicle locations occur such as around a Y-junction. FIG. 39 is a diagram showing four different electrodes placed at different locations provide means of fascicle selective interfacing with the present invention.

FIG. 39 depicts four ring-like cured electrodes 22 which have been injected along a nerve 5 with a Y-junction. The longitudinal view illustrates the location of the four electrodes along the nerve while the transversal (cut through each electrode across the nerve) illustrates the location of each specific fascicle 32 in relation to the electrode. Each distinct fascicle shape illustrates how the relative position of fascicles shifts throughout the nerve over distance. Similar shaped fascicles from one cross-section to the next are used to show how one fascicle may be right next to the outer rim of the nerve, meaning next to the epineurium of the nerve, while being more in the middle of the nerve in another location along the nerve that is surrounded by an electrode of the present invention. Proximity of a fascicle to the electrode may determine activation thresholds for that specific fascicle, providing a different fascicle selective activation for cured electrode “A” from that achieved with cured electrode “C.” As cured electrode “B” only surrounds the smaller nerve sub section, it will provide a different activation of fascicles too. The cured electrode, “D”, surrounding all fascicles of both nerve sub sections forming the Y-junction, has the ability to drive all fascicles.

In one embodiment a fascicle-selective electrode may be constructed by using liquid mixture 1 and liquid nonconductor carrier materials 9 to surround the whole nerve or only parts of a nerve. By surrounding only a part of a nerve's epineurium 33 with the mixture, fascicles 32 with closer proximity to the liquid mixture will be activated preferentially to fascicles more distant to the liquid mixture. The remainder of the nerve may be surrounded with liquid nonconductor or may be left uncovered. For example, using a two-chamber dispenser to deliver two separate carrier materials, one with conductive elements and the other without, the physician may selectively surround the nerve with either liquid mixture or nonconductor, while still providing a structure that encases the entire nerve and provides the mechanical stability and anchoring of the cured electrode around the nerve. FIG. 40 depicts a selective interface to two specific fascicles. FIG. 40 depicts a liquid mixture/cured electrode 1 and liquid nonconductor/nonconductive layer 9 surrounding a nerve with six fascicles 32. Only the two fascicles (A) and (B) are preferentially stimulated with this configuration. The depicted optional surrounding of the liquid mixture with a nonconductive layer provides additional electrical shielding against the environmental biological tissue such as adjacent nerves, connective tissue, blood vessels or muscle fibers.

In one embodiment, a nonconductive layer may be added to a cured electrode of the present invention after the cured electrode has cured in place at or around a target in bodily tissue. First, a liquid mixture is provided and mixed and loaded in a dispenser. Then the surgeon injects the liquid mixture in the first phase at or near a target in bodily tissue, and then withdraws the dispenser (needle) for at least 5-10 minutes to allow the liquid mixture to undergo a phase change. In one embodiment, a wire is embedded in the first injection. Then the surgeon opens the wound again and bluntly separates the cured electrode by vibration, pulsed air or a blunt needle tip from the surrounding tissue on the outside of the cured electrode (muscle, fascia, etc.) Next, the physician injects the liquid nonconductor of the same type as contained in the just-cured cured electrode. If a wire was encased earlier, the liquid nonconductor is placed around that wire, adding to the anchoring of the wire with the surrounding tissue. Optionally, the physician may make a loop or knot in the wire and embed that loop/knot near some structure such as a bone in the nonconductive layer. Next, the surgeon withdraws the needle and allows the nonconductive layer to cure around the cured electrode.

An example of the above paragraph is a liquid mixture comprising silicone as a carrier material and silver as the conductive elements, which is placed either by a needle or in a laparoscopic procedure around a peripheral nerve under ultrasound or angiogram visualization.

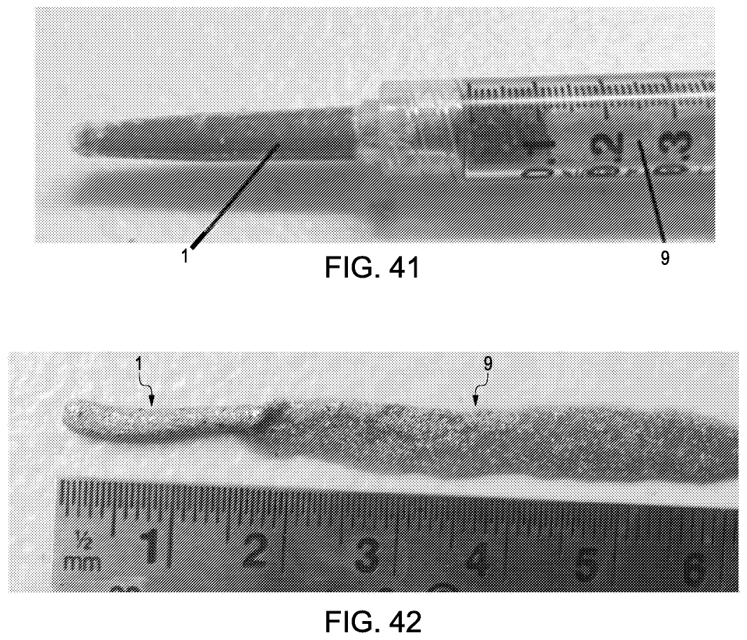

FIG. 41 depicts a method of loading the liquid mixture 1 and liquid nonconductor 9 in the same syringe 2, with the mixture in front (1st) portion and the nonconductor in back (2nd) portion. During the placement, the physician may choose to place the 1st portion at the neural interface, encasing the nerve and connecting a lead wire with the mixture as it cures, or just after curing. Immediately or after a short wait the physician may encase the cured electrode with the liquid nonconductor to add insulation as well as further improve mechanical attachment to the surrounding tissue in the formerly created cavity, but without the risk of introducing new connective points that are in any way connected to the cured electrode. This configuration allows dispensing liquid mixture and/or nonconductor around a target and wire, then after a brief pause (10 sec to 10 minutes) continue to inject from same syringe the back 0.5 cc to insulate the placed liquid mixture against the other bodily tissue and improve the mechanical attachment of the overall cured electrode at the injection location. FIG. 42 is an image of an embodiment of a low viscosity silicone and silver based cured electrode 1 injected through the arrangement depicted in FIG. 41. The darker portion closest to the needle, shown on the left side of the image, is the liquid mixture 1 and is highly conductive due to the high Ag content. The lighter portion is the nonconductive material or layer 9 on the right side of the image has only sparse amounts of Ag and is inherently insulating.

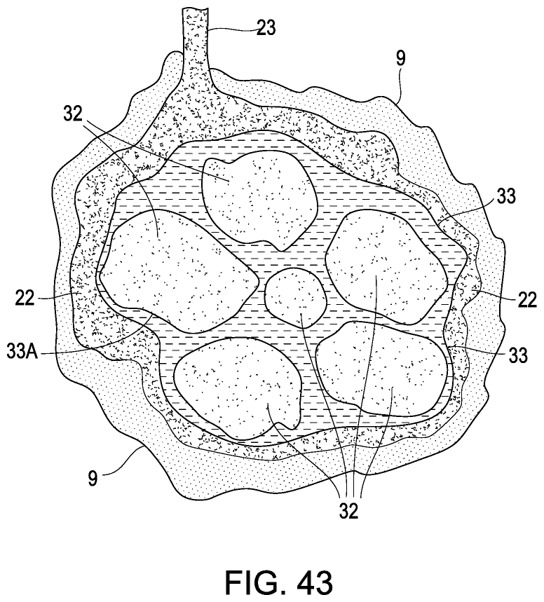

The present invention includes a method for minimizing “flaking” of the conductive elements from a cured electrode, and preventing mobilization of any flakes from the cured electrode over a period of chronic use in the body, by using chemical bonds to increase the cohesion of the bulk of the cured electrode. Such chemical bonds include, without limitation, valence bonds, Van der Waals bonds, hydrogen bonds, covalent bonds, and ionic bonds (between the conductive elements added to the liquid carrier material and specific functional side groups added to the carrier molecules or chains). Surface tension and/or the use of surfactants to cause the aqueous environment to drive the conductive elements toward a hydrophobic bulk material like silicone (i.e., drive toward lowest energy conformation) may be used (FIG. 43). Another method or configuration to minimize any mobilization of conductive elements that may have formed flakes and became mechanically detached from the bulk of the cured electrode, a nonconductive layer 9 may be dispensed to keep any flakes in place.

FIG. 43 depicts covering the liquid mixture/cured electrode 1 with nonconductive layer 9, an additional layer of mechanical stability may be provided to the cured electrode as a whole as well as any conductive elements. In some embodiments, the nonconductive layer 9 may be seeded with cells, other biological or non-biological components to produce a thicker encapsulation of the cured electrode on the outside, while the inside cured electrode (the mixture in contact with the nerve) remains encapsulated by a thin encapsulating layer of fibrous tissue 52. The fibrous connective tissue formed by the body as it encapsulates any foreign object such as the cured electrode will add yet another layer of mechanical stabilization and reduce the probability of conductive element mobilization. The thickness of the fibrous connective tissue may be modified intentionally by seeding the mixture or nonconductive material with elements, cells, other biological and abiotic components to enhance the inflammatory response of the body temporarily and cause a thicker outside encapsulation. Reduction of flaking may also be encouraged by the shape of the conductive elements themselves. Other embodiments for reducing flaking include, without limitation, conductive elements with a high aspect ratio, interlocking features 28 at either end (e.g., hook, loop or coil), or a coiled or similar structure throughout the length of the conductive elements to improve mechanical stability within the cured electrode. More solutions are described elsewhere herein.

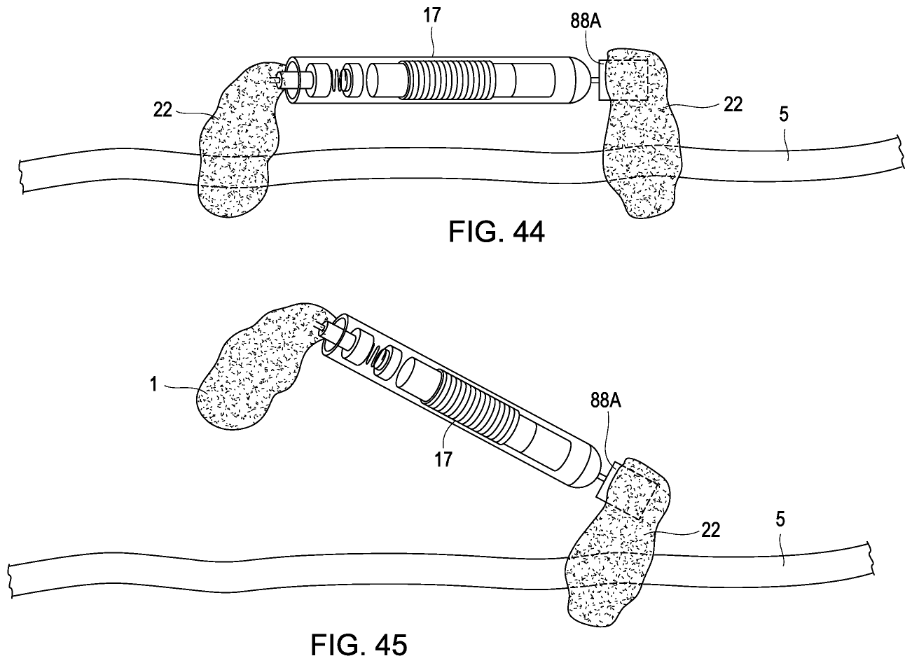

In one embodiment a signal generator 17, an IPG such as a miniaturized BION (e.g., Alfred Mann Foundation, Bioness, Advanced Bionics) or similar may be connected to a target in bodily tissue with a cured electrode at or surrounding the target. Some of these signal generators may be injected via a large bore needle and thus may relatively easily be placed into a patient's body without the need for a major surgery. The shortcoming of these very small signal generators is that they are not able to depolarize, address, stimulate, or block the whole nerve without the use of a cuff-like structure that encases the nerve. Any prior art small metal contact placed next to a nerve will not achieve a uniform field or an electrical field that is more or less of the same field strength all around the nerve, but the present invention may incorporate an IPG or other signal generator 17 to address these issues. (FIGS. 44 & 45). FIG. 44 is a diagram showing an embodiment of the present invention with each of two cured electrodes 1, at a first end of each cured electrode at a specifically adapted connector 88A, connected with a signal generator 17, and at the other ends connected to a nerve to provide a uniform electrical field for the whole nerve, not just a strong depolarization signal to the nerve fibers inside the nerve that are closest to the signal generator's contacts. Two cured electrodes may be placed, one on each contact of the signal generator to utilize two active cured electrodes, one cathode and one anode.

FIG. 45 shows how a cured electrode may also be placed on only one side to connect the signal generator to the nerve (active cathode), or may be placed at another location to provide a better electrical interface to the surrounding tissue at the location of the distal anode.

In another embodiment, the present invention has the ability to bluntly separate tissue plains. That is, it has the ability to be injected into spaces and crevices created by blunt dissection. This blunt dissection may be accomplished by traditional surgical means with forceps and scissors or it may be achieved by directing pressurized air, liquid, or a liquid mixture or nonconductor at an interface between two tissue plains to separate these two plains. A simple way to encase a nerve using the liquid mixture or nonconductor is to inject the material directly around the nerve at a 10 to 90 degree angle to cover (1) more nerve tissue longitudinally (using the 10 degree angle measured vs. the longitudinal axis of the nerve) or (2) a shorter distance along the nerve and place more of a thin ring around or at least a C-shape liquid mixture/cured electrode behind/next to the nerve (using an angle closer to 90 degrees as measured vs. the longitudinal axis of the nerve). In one embodiment of the present invention, there is a method of blunt dissection which may be aided by vibrating the liquid column inside the dispenser or by vibrating the tip of the dispenser or by vibrating both, the tip and the liquid column, using the vibration as a means to have short moments of higher and lower pressure gently move the tissue plains apart for the injection. The vibrating pressure may be applied in bursts or continuously, it may be directed in the same direction as the longitudinal axis of the dispenser or it may be directed orthogonally to the longitudinal axis of the dispenser. The vibration may be along one axis or it may be circular to cover a two-directional movement of the dispenser and or dispensed liquid material next to the two tissue plains intended to be bluntly separated. In yet another embodiment, injection of the liquid mixture itself allows the physician a method of blunt dissection of the tissue around the target, so that the liquid mixture itself aids in blunt dissection.

The present invention also has the ability to form an electrode-to-nerve interface in stages, in seconds to hours. Uncured liquid mixture, as long as it has not been contaminated with bodily fluids or tissue, may be added to a previously injected cured electrode of the same carrier material or, in some combinations, of a different carrier material of compatible chemical and mechanical properties. Cured electrodes, especially when fully or partially covered by biological tissue, may first require an optimized cleaning procedure (including mechanical cleaning and a chemical deep-clean or even roughening of the cured electrode surface) prior to continued electrode placement/molding/sculpturing in the patient.

A cured electrode in a cuff-like embodiment around a target may be injected as a continuous stream of liquid mixture, or in steps, to cover first the volume behind or underneath a nerve, before placing liquid mixture next to the nerve and on top of that nerve to close the ring-like portion 22 of a cured electrode.

A cured electrode also gives the physician the ability to go in a second time later and fix a sub-optimal prior art electrode or other device, or even a prior implanted cured electrode, without the requirement of explantation of the previously implanted device. In so doing, the cured electrode provides an opportunity to restore or supplement the function of a previously implanted electronic device.

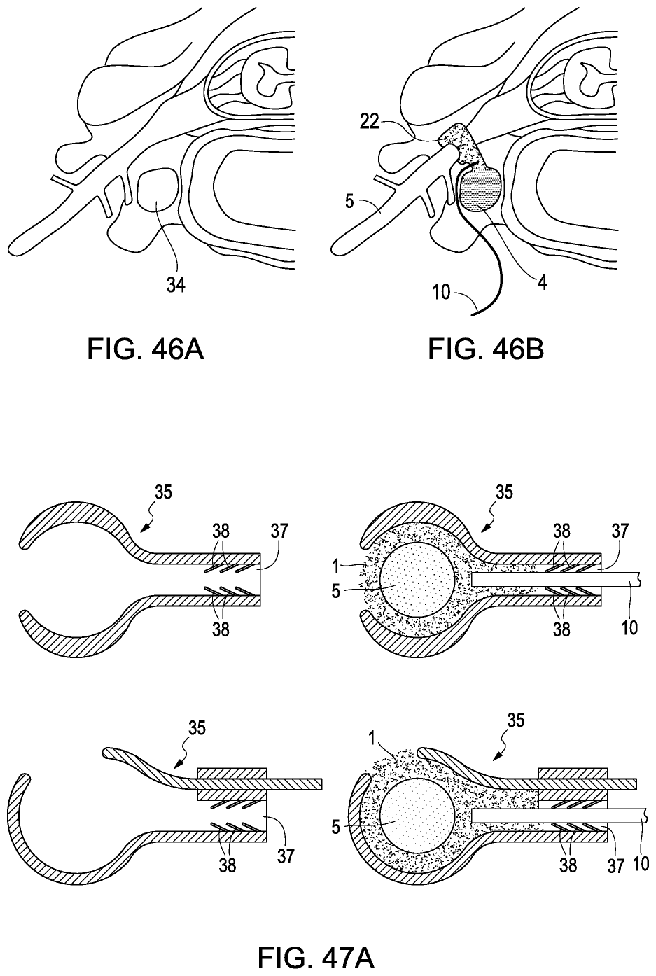

The present invention also has the capability to integrate with boney tissue. Nerves of the PNS often run close to bones and generally do not move significantly relative to these bones, and liquid nonconductor may be used to anchor a cured electrode used to stimulate a nerve in close proximity near a bone. For that, the bone itself may be encased in part, or completely with liquid material; or the bone skin (periosteum) may be lifted away from the bone at a location close to the cured electrode-to-nerve interface to allow the injection of liquid carrier material into a pocket between bone skin and bone; or the bone itself may be punctured or drilled to form an anchor point for a placement of liquid mixture; all of which may be done through a minimally invasive, laparoscopic approach (FIGS. 46A and 46B). FIG. 46A is a section diagram of a vertebra, and 46B is the same view after placement of liquid mixture 1 to encase a nerve 5 and attach a lead wire 10, then anchor the liquid mixture with liquid nonconductor/non-conductive layer in the foramen transversium 34. The anchor 4 for a cured electrode may be done in a hole drilled specifically for the purpose of providing space for an anchor 4, or a naturally occurring bony structure that may take up mechanical force may be used. An example of an anchoring point is a foramen.

Another embodiment of the present invention further comprises integration of a current-limiter within the cured electrode-nerve-interface. A significant danger to the nerve in the vicinity of the neural interface is current over-stimulation that may lead to temporary nerve damage or permanent nerve damage and scarring. The lead wire itself may comprise a fuse component included that may be glued back in place using the present invention if the fuse is blown by, e.g., a static shock, applied currents of unintentionally high levels, or a shorting caused by improper electrode injection/placement during surgery.

A pre-formed mold 35 may be used to hold the shape of the liquid mixture/liquid nonconductor temporarily or permanently during or after it is applied and cured in the body. The advantage of a pre-formed temporary mold: a specific shape for a cured electrode covering a specific volume may be created. The removal (including removal by biodegradation if the pre-formed material consists of a labile material such as), would then fully expose the cured electrode to the body tissues. A permanent pre-formed mold 35 may be used, in one embodiment, which is porous to allow free passage of ionic currents. This has the advantage of fully containing the liquid mixture or nonconductor during and even after curing. A permanent pre-formed mold 35 that still allows for proper functioning of the invention, has the advantage that it would ensure flakes of the conductive elements 6 do not migrate into tissues, and complete removal of the pre-formed mold-encapsulated device could be accomplished.

In one embodiment, a pre-formed mold 35 comprises the shape of hook 36 which may be fit loosely around a nerve with liquid mixture. Once the nerve is freed up from surrounding tissue (e.g., in a laparoscopic procedure), a mold in the form of a C- (as in FIG. 47A) or O-shaped hook may be placed around a nerve. In another embodiment, an injectable hook (not pre-formed) may be injected in liquid form to surround the nerve by 180, 210, 240, 270, 300, 330 or even 360 degrees. The pre-formed mold 35 may be in the form of a cuff that is sliced open. It may be in the embodiment of a hook comprising a slider to close the hook. These pre-formed molds, in different embodiments, are electrically conductive, but the injection of liquid mixture makes them conductive. The hook, in another embodiment, may also comprise a valley running inside the opening around the hook which is filled with liquid mixture to ensure a minimum thickness of liquid mixture around the nerve. In one embodiment the hook 36 comprises an opening 37 on the opposite side away from the nerve with means for securing the end of a wire, such as crimp hooks 38 to which a lead wire may be connected by just sliding it into the hook. The hook, in one embodiment, allows the inserted wire 10 to touch the liquid mixture that is injected into the opening between nerve and hook (either prior to putting the hook on the nerve or after the hook has been placed on the nerve), but the wire 10 is prevented from touching the nerve by having designed minimum separation distances between the nerve and the distal end of the hook, which will correspond with a measure on the lead wire that prevents an insertion which is too far from the cured electrode material. FIG. 47A is a diagram depicting two embodiments of the hook 36 which enable a complete covering of the nerve with liquid mixture. Liquid mixture 1 may be placed onto or into the hook prior to placing the hook on/around the nerve in a laparoscopy or other surgical procedure, or it may be injected into an opening 37 on the hook or in a gap between a loosely fitted hook and the nerve. The hook further ensures that the lead wire does not touch the nerve and that the lead is integrated with the liquid mixture. The hooks may be manufactured from a flexible or a more rigid material. The pre-formed mold may be left in place around the liquid mixture/cured electrode or, in another embodiment, it may be removed prior to the end of the procedure, once the cured electrode is complete. One means for removal comprises the pre-formed mold being made in multiple pieces which may be disassembled by the physician near the end of the procedure.

In other embodiments of delivery methods for cured electrodes for neuromodulation or ablation, a mechanical holder or “sock” 96 is provided. The sock has the ability to curve around a nerve as it is filled with the liquid mixture. The liquid mixture is mechanically stabilized by the sock-shaped mesh and utilizes the liquid nonconductor simply to aid with the transport of the conductive elements from a delivery device (i.e. syringe) through an applicator (i.e. needle) into the sock. This sock, in multiple embodiments, may comprise a pre-configured curvature and differing dimensions to aid with placing the sock in a particular location. The mesh openings of the sock 96 must be smaller than the conductive elements 6. In this way the sock functions akin to a filter, letting the liquid nonconductor material pass through but holding in place the conductive elements and filling into an optimal shape. The sock can be filled under sufficient pressure to retain a tension (sock filled to max), or it can be filled and remain flaccid (sock filled to i.e. 70% to 90% of max volume). FIG. 47B depicts different embodiments of the sock during filling process with conductive elements in a suspension by liquid nonconductor. A needle Version (I) is the straight sock, version (II) is the pre-configured curvature and version (III) is the sock able to extend at a perpendicular angle (or other angle if the directing opening has a specific different angle than 90 degrees.

In order for the “sock” to be biocompatible, the same materials as used in surgical meshes, such as for hernia repairs or reconstructive work in the body where mechanical tissue integrity and/or cohesion is improved by suturing a mesh to the bodily tissue. Materials applicable are silk mesh, polypropylene (PP) mesh, polyethylene-terephthalate (PET) mesh and polytetrafluorethylene (PTFE) mesh among others.

The sock can be used to create a neural interface for stimulation, partial or full block, temporary or permanent nerve block. It can be used for nerve ablation. It can be used to create a neural interface that extends perpendicular from a needle introducer 3, which is especially of interest in hard to reach locations behind anatomical obstacles or inside the CNS, such as when a DBS approach requires an electrode to be extending e.g. perpendicular to its initial insertion path. The introducer needle (one embodiment in FIG. 69B with a blunt end 16A and side opening 64) is inserted into the sock 96 and then the needle and sock are inserted into the body. For the sock shown in FIG. 47B III the introducer needle (exit point 29 at the tip) and sock are then placed into an outer needle (FIG. 69B). For version III, the sock 96 is pushed out of the side opening 64 by the liquid mixture extruded from the introducer needle 3.

In another embodiment, the present invention provides a method for repairing broken electrode leads for targets, i.e., the wire connections between an implanted signal generator and an electrode which is placed on a target. Sometimes these electrode lead wires break. This is a problem for neural and cardiac applications alike. In fact, one of the reasons for revision surgeries in cardiology is to replace broken cardiac pacemaker leads that do not deliver the signal from the signal generator to the stimulation location inside the heart. The liquid mixture material has the ability to “weld” or “glue” cardiac leads with a minimally invasive procedure. The main advantage of repairing instead of replacing a broken electrode lead is that the interface between the electrode at the end of the lead and the body's tissue does not need to be disturbed as is usually the case when a broken electrode lead is being removed: A typical technique used in the cardiac space is to simply pull out the electrode lead, which may lead to tearing and other unintended damaging of the heart muscle, the cardiac valves and other surrounding tissues. In contrast, by leaving the electrode lead in place and only fixing the break, the lead is allowed to stay in place and the electrode/tissue interface is not injured.

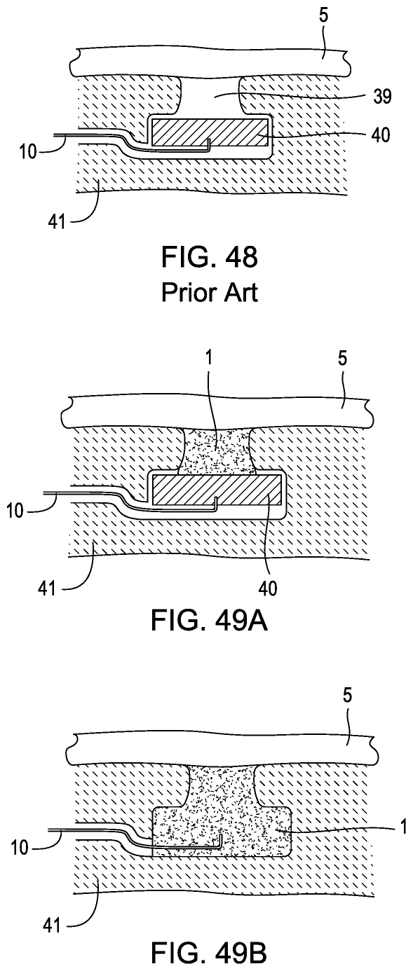

Another capability of the liquid mixture is to increase the contact area for prior art electrodes which have a limited contact area to the electrolyte as well as the target 5 in bodily tissue: most prior electrodes provide a planar interface which is not perfectly suited to interface with a 3D-object such as neural tissue in the body. In fact, most pre-configured cuff electrodes 40 implanted in the body have a pre-formed carrier 41 such as a strip of silicone (manufactured outside the body which holds the metal contacts (providing the electrode-electrolyte-interface) in place, but also causes the electrode contacts to be recessed into the carrier 41 (FIG. 48). FIG. 48 is a diagram showing a section view of a prior art electrode around a nerve, showing a void 39 between the metal contact of the prior art electrode 40 (e.g., platinum) and the nerve 5. This void 39 creates additional distance for the electrical current to pass (thus reducing stimulation capability) and also fills with fibrous tissue that causes a significant change (often 2-5× increase) in stimulation impedance. As a result of chronic encapsulation 52 over the time of a month or more inside the body, this void fills with connective tissue, increasing the electrode-to-nerve impedance significantly and causing a (sometimes large) portion of the current used to stimulate the nerve actually shunt around the nerve as the impedance in the interstitial fluid between electrode and encapsulation may be significantly smaller than the impedance electrode-encapsulation-nerve-encapsualtion-back-to-return-electrode. In contrast, the injectable liquid mixture 1 allows for a direct interface of the conductive elements 6 of the liquid mixture material to the electrolyte near the target nerve without leaving a void for encapsulation to build up. This results in a smaller electrode-to-nerve impedance for chronically implanted cured electrodes in comparison to prior art cuff electrodes.

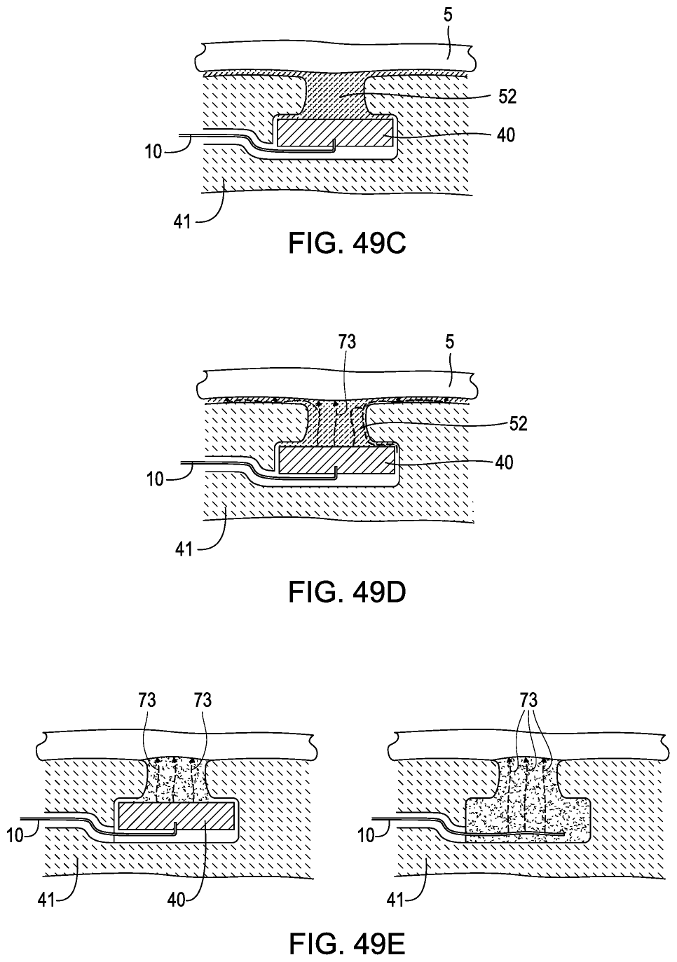

It is advantageous to cover prior cuff electrodes with liquid mixture at their respective electrode contact locations to fill the void 39 marked in FIG. 48. By using liquid mixture to fill this void prior to implantation, long term electrode-to-nerve interfaces may be provided that have smaller impedances, advantageous for both stimulation and sensing (FIG. 49A). FIG. 49A depicts the same prior art cuff electrode as in FIG. 48, but also shown is the void 39 in FIG. 48 having been filled with liquid mixture prior to implantation, so that only a thin film of fibrous tissue may form between the cured mixture material and the nerve, providing a better long term chronic interface. The liquid mixture may be injected/extruded into the void 39 above the original metal contact 40 (FIG. 49A) or may replace the metal contact entirely, providing the connection from the lead wire directly to the nerve, as in FIG. 49B.

FIG. 49C depicts how fibrous tissue fills the void 39 in FIG. 48 between the metal contact and the nerve in a traditional electrode. FIG. 49D shows how electrical field lines 73 spread because of the fibrous tissue encapsulation 52 being thick and filling the gap between the electrode contact as well as lining the inside of the cuff electrode. FIG. 49E shows how by filling the void between the Pt contact bonded to the lead wire, or just filling the void 39 from the lead wire 10 (replacing the Pt contact), with liquid mixture 1, the electrically conductive cured electrode may allow for much higher field densities and further concentrate the electrical field lines 73. By choosing conductive materials such as gold, platinum, platinum-iridium, titanium, graphene, carbon tubes potentially with the additional placement of local anti-inflammatory medication that facilitate thin bio-encapsulation, the current spread may be further limited, thereby allowing smaller stimulation currents and a lower noise neural recording interface.

In another embodiment, the liquid mixture or nonconductor may be seeded with stem cells, including the patient's own stem cells, or neurons, glia, astrocytes, red or white blood cells, tendon or muscle cells. The resulting cured electrode may chronically form a thinner encapsulating layer as well as a spongy bulk form, allowing for better integration with the surrounding biology of the cured electrode recipient. Thicker encapsulation between the cured electrode and the non-target bodily tissue is desirable, whereas thinner (preferably, none) encapsulation between the target and the cured electrode is desirable

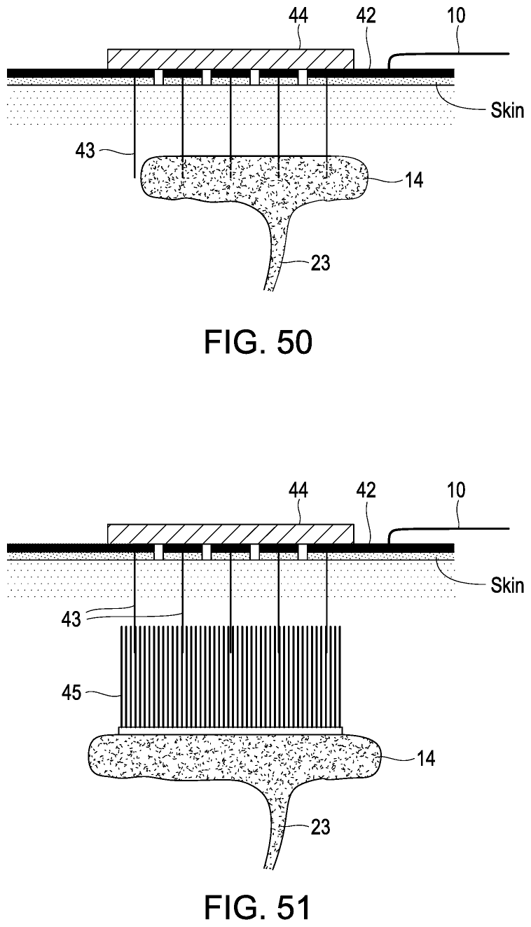

In another embodiment, needled skin patch electrodes 42 may be placed on the skin outside the body. In order for a skin patch electrode to make a continuous contact to a deep tissue nerve 5, a continuous electrical connection of low impedance throughout is advantageous. The skin provides an impedance of about 500 to 1000Ω transcutaneously (depending on thickness, sweating) produces a large voltage drop if not compensated appropriately. Although the approach of placing a pad of liquid mixture/cured electrode subcutaneously in electrical communication with a TENS unit (e.g. FIGS. 14A- to 14F) may overcome the skin impedance, other embodiments of the present invention provide additional solutions to the problem of skin impedance. One embodiment is a needled skin patch electrode 42 comprising small needles 43 which form a direct electrical connection to the contact pad 14 and thereby are able to reduce the transcutaneous impedance to levels below 10Ω. The needles 43 may connect to electronics 44 to test and report impedance, in order to determine the sufficiency of the electrical connection of the needles 43. The needled skin patch electrode 42 itself may or may not be conductive any more as the primary means of conducting the electrical energy is to pierce the skin with the needles to connect to the subcutaneous cured electrode. If the patch electrode is not conductive, then it is a sticky patch without any electrical hydrogel replaced with glue similar to that on band aids. For example, electrodes without hydrogel may serve as band aids with needles, or, a TENS electrode 13 with micro needles 43 to connect electrically to an electrical field connector 15. This allows the test between needles to verify successful integration into the contact pad 14, allowing the physician to confirm successful contact has been established. FIG. 50 is a diagram of a cross section of a needled skin patch electrode with test electronics 44 connected to a needle matrix 45 connected to a contact pad 14, here just below the skin.

Disclosed is a method of testing the needled skin patch electrode 42 embodiment of the present invention to verify successful connection through the skin. If needles 43 penetrates the skin to connect either to a cured electrode in the shape of a pad or to a fixture such as a needle matrix 45 (FIG. 51) embedded in a contact pad 14 in the subcutaneous tissue, then an impedance measurement may be used to determine the connectivity of the microelectrodes to the cured electrode. This enables the physician to ensure that only needles 43 which are in direct connection to the contact pad 14 or to a needle matrix 45 in one embodiment) will receive electrical energy. FIG. 50 is a representation of a cross-section of the needled skin patch electrode 42 with an implantable needle matrix 45 embedded in the contact pad 14, and the needle matrix 45 and the needles 43 from the outside electrode 42 are configured to make electrical connection with one another. The implantable needle matrix may take 1,000,000 needle injections and not bend, as in the Utah electrode array (FIGS. 2A, 2B) turned towards the skin and using spring action.

In FIGS. 50 and 51, a subcutaneous contact pad 14 of a cured electrode 1 may contact needles 43 inserted from the skin and this connection transfers current across the skin. If a set of needles 43 penetrates the skin to connect to either an contact pad portion 14 of a cured electrode 1 or a fixture such as a needle matrix 45 in the subcutaneous tissue, then an impedance measurement may be used to determine the connectivity of said needles 43 to the contact pad 14 or needle matrix 45. This ensures that only microelectrodes who are in direct connection to the cured electrode (or fixture) will receive electrical stimulation energy. A needled skin patch electrode 42 with hydrogel or with band aid glue and needled electrodes 43 will achieve good direct (continuous) electrical contact by, for example, the needles 43 (conductive core, partially insulated to pass through sensitive area of the skin) piercing the subcutaneously buried cured electrode pad inside the deep tissue. Needles 43 may come with or without insulation in different embodiments.

The cured electrodes disclosed herein may be used with a current limiter to avoid neural over-stimulation from static shocks or applied currents of unintentionally high levels. A current-limiter is embedded in the wire-like portion 23 of the cured electrode, or one current-limiter is added to each of the needles 43 to provide a safety feature for the nerve. That is, a current limiter is seated between two sections of the wire like portion 23, or at the beginning or end of each of the needles 43. The applications for the current limiter include post-surgical or post-operative pain treatment with self-dissolving cured electrodes that allow TENS treatment for a deep tissue nerve. The current-limiter is in the needle or in the wire leading to the electrode.

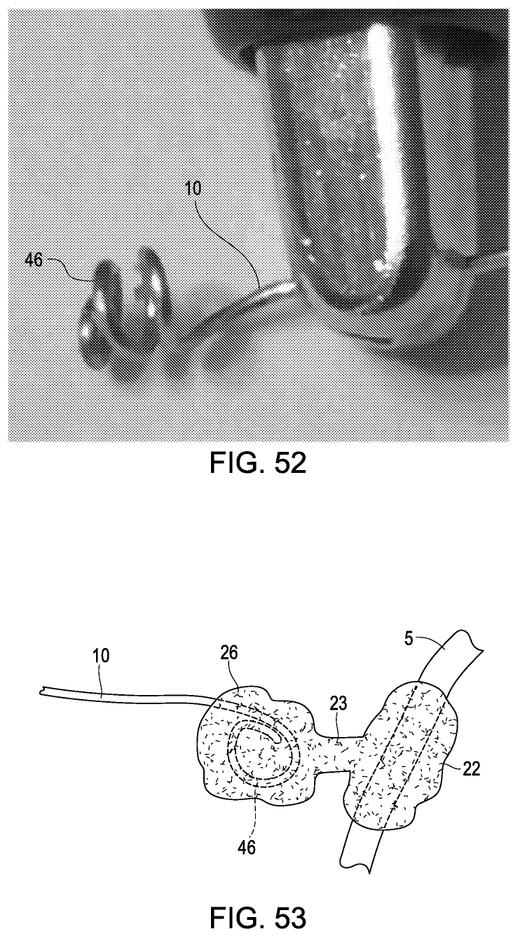

Leads, cables, or connecting wires are continuous metal connections, generally insulated for most of their length, which allow a direct metal connection between, for example, a signal generator 17 and a signal applicator. A typical signal applicator in the prior art is an electrode, or a metal connection to a target 5 with insulating components. In the present invention the wire 10 (i.e., cable or connecting wire) may form a direct (i.e. continuous or pure metal connection between, for example, a signal generator and the liquid mixture/cured electrode 1, which in turn connects to the target 5. A lead wire 10 may be a helical or double helical metal wire encased in silicone to provide insulation against the surrounding biological tissue. The function of the lead is to connect the electricity from, e.g., a signal generator to a nerve. In the case of the present invention, a prior art cuff electrode may be replaced by the liquid mixture/cured electrode. To achieve an optimal mechanical and electrical contact between the lead and the cured electrode, specific interfaces are described herein. One type of lead comprises a connecting feature 46 such as a helix, screw or other type of barb at the end (terminal) as interface to the cured electrode as shown in FIG. 52 which is an image of a helix screw (or, cork screw) interface with a cured electrode, held for display by an alligator clip. Another embodiment of a connecting feature resembles the shape of a bird's nest, or a mesh, to interface with the cured electrode. In one variation, the connecting feature 46 at the lead terminal(s) may be a crumbled up wire, similar to a bird's nest. This may be formed by continuously (or on button push) dispensing a gold bonding wire (that is optionally covered by a surfactant for good electrical conductivity that is not impeded by having the entire outside of the wire be covered by the carrier such as silicone, cyanoacrylate, fibrin etc. FIG. 37 is a representation of the “bird's nest” or mesh of gold bonding wire loops that interconnect with each other. Even though it is one continuous wire, it is the meshing and interweaving of the surfactant-covered bonding wire that allows for many physical connections between the gold wire loops. In case one of the wire loops breaks or loses connection to a neighboring loop, there are still many others conducting electricity to the nerve. Another connecting feature 46 for a lead wire 10 is a loop or a similar shape to increase mechanical adhesion, as compared to a linear shape of a wire, to connect to the cured electrode. In some cases, a loop may be the most advantageous connection as shown in FIG. 53, a representation of a wire loop 46 which is embedded in one portion of a cured electrode blob 26 which also comprises an interface molded and cured as a ring 22 around a nerve target.

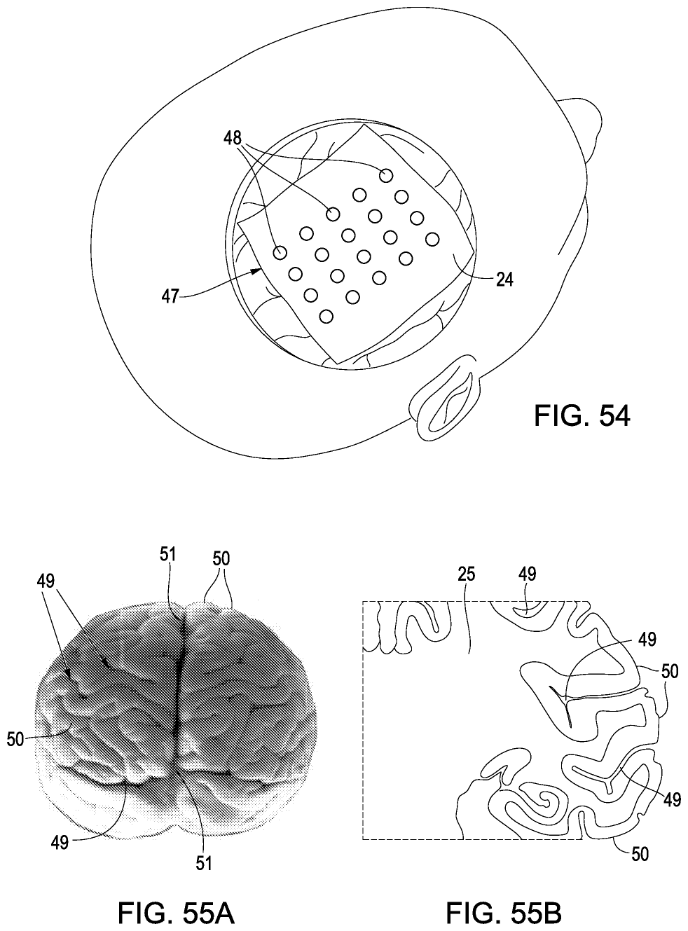

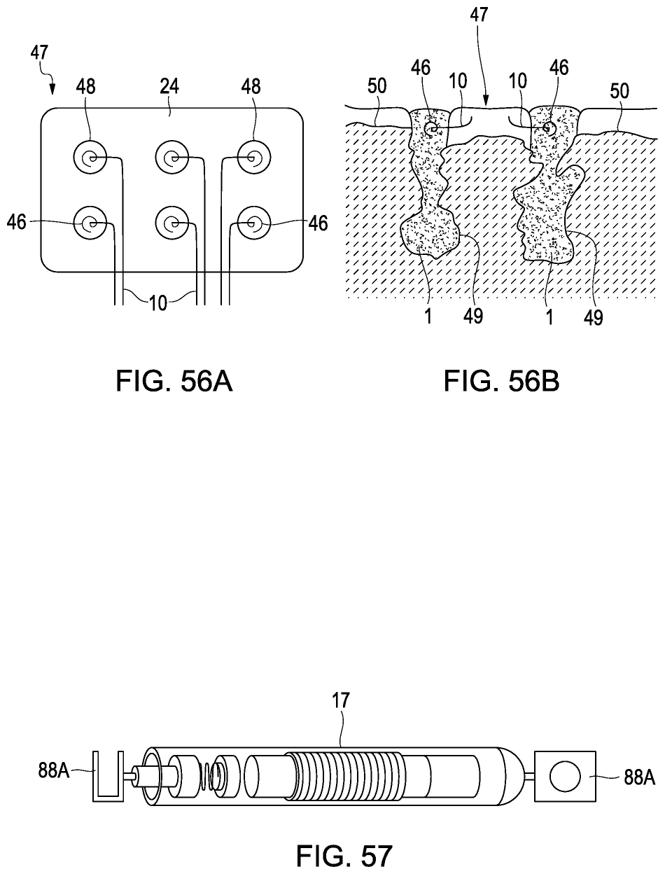

The cured electrode also has embodiments for cortical applications, connecting to sulcus and gyrus alike, as in FIG. 54 in an electrocorticography (“ECoG”] electrode matrix 47 where the liquid mixture 1 is pushed through at specific ECoG points such as holes 48, some embodiments of the holes comprising structures (e.g., the hole in embodiments in the shape of a frustum open at the smaller end nearest the brain, or as shown in FIG. 56B) with the connecting wire 10 of the lead exposed and the hole or conduit, in one embodiment the shape of a frustum 48, allowing for mechanical attachment and integration of cured electrodes in each of the holes. De-insulated tips of wires 10 (optionally with the connecting feature 46) are incorporated into the electrode matrix 47 and extend into the middle of each of the holes 48, for the liquid mixture to be injected into through, thereby making an electrical connection to the liquid mixture 1 injected to the gyms 50 or sulcus 49 underneath. A prior art ECoG electrode as placed subdurally and on top of the arachnoid mater is depicted in the perspective drawing which is FIG. 54. Prior art matrices are able to contact only the gyri 50 (hills) of the brain's cortex but are not able to penetrate into the sulci 49 (trench/valley) between two gyri. FIG. 55A is an image of a brain, of interest here more specifically the sulci of the cortex and the midline 51 which is the deep trench between the two hemispheres. The gyri and sulci enable the cortex to have a large surface area. FIG. 55B is a representation of a section of neocortex and the underlying white matter 25 showing the depth (and relative inaccessibility) of the areas within the sulci and midline, and how stimulation of gyri only through prior art electrodes is inadequate for any area of the neocortex not specifically on a gyms. Prior art ECoG electrodes do not reach into the depths of the sulci, but the liquid mixture 1 of the present invention can be injected into the sulci as shown in FIG. 56B, and without the risk of injuring the blood brain barrier as the liquid mixture 1 in one embodiment is formulated to be molded and cured as flexible and pliable against the soft neocortex. Injecting liquid mixture (deep) into the midline 51 allows mid and deep brain stimulation without injuring the blood brain barrier. FIG. 56A is a representation of a portion of an ECoG electrode matrix 47 from the top showing the matrix and holes with wires terminating in the holes where the wires make electrical contact with the liquid mixture which has been injected into the hole to make close with, and to mold and cure against, the neocortex underneath. The holes 10 allow the surgeon to place the liquid mixture material deep into the sulci (FIG. 56B). On one end the wires 10 at each hole 48 terminate in the open area of the holes and, on the other end, terminate at a signal generator 17 and, optionally, each wire may be activated separately from each of the other wires, by means of a controller inserted at the time of the procedure. In contrast to the traditional ECoG electrodes that only touch the tip of the cortex, thereby only getting a high SNR signal from gyri underneath the contact point of the ECoG electrode matrix, the combined liquid mixture-ECoG electrode is able to get signals from the sulci between the gyri by injecting the liquid mixture into a sulcus. The ability to interface with high SNR to both, a gyrus as well as well as a sulcus allows for better sensory and stimulating neural prosthesis. The advantage of being able to press the liquid mixture/cured electrode into the deep valleys 49, 51 and letting the liquid mixture mold against and retain the shape of the valley minimizes damage to the neocortex, assures a perfect fit, and allows for a high fidelity, low-risk neural interface into deep valleys of the brain which does not breach the blood brain barrier.

Aside from injecting the liquid mixture onto a gyrus or into a sulcus through the pre-positioned liquid mixture-ECoG matrix 47 sitting on the cortex, yet another embodiment uses a laser to display the most probable location of all (e.g., 20) contacts of a prior art ECoG electrode as they sit on the brain's cortex to allow the surgeon to place the liquid mixture on top of gyri and inject liquid mixture into sulci, followed by then placing the prior art ECoG electrode (without the holes described herein) onto the cortex. By having liquid mixture placed in contact with the cortex first at the specific locations that the ECoG matrix 47 of the present invention has its electrodes, a connection to a traditional ECoG electrode can be made with the advantage of being able to connect to the deeper structures within the sulci and a better direct interface with the gyrus directly beneath each ECoG electrode of the matrix.

These advantages put the liquid mixture-ECoG approach in a range of interface fidelity between that of a traditional ECoG placed on top of the arachnoid mater and that of a penetrating microneedle-based electrode system (e.g., FIGS. 2A and 2B) that breaches the blood-brain-barrier by injuring the arachnoid mater and the combination of neural supporting and vascular tissue beneath the arachnoid. The present invention allows a novel combination of the liquid mixture/cured electrode 1 with the ECoG electrode matrix 47 to provide the safety level of the traditional subdurally placed ECoG, while achieving a much higher SNR than the traditional ECoG array placed as a generally planar interface on top of a 3D-object such as neocortex. Additionally, as the liquid mixture-ECoG is mechanically adapted and to a certain degree mechanically integrated within each sulcus, SNR stays high even with brain movements present due to heart beat, breathing, and inertia moving cortical tissue during walking or other causes of (even abrupt) accelerations and decelerations of the brain or the skull. The liquid mixture/cured electrode makes not only a good electrical connection to the neocortex, but also a strong mechanical connection as the cured electrode in a sulcus acts to fasten the ECoG matrix electrode 47 in place. The liquid mixture which cures to a solid electrode allows for a more flexible neural interface with the cortex and thereby allows the physician to control the expected mechanical match between cortical tissue and cured electrode. Specific liquid mixture mixtures comprising hemostatic agents (described herein) further offer the ability to immediately stop any bleeding, making the liquid mixture an excellent choice for brain surgery with an open cortical wound where the blood brain barrier is already breached or, similar to a DBS electrode, the liquid mixture may be injected into the cortical (or deeper brain) tissue to connect to said structures while being able to stop bleeding at the source of the injury by using the liquid mixture as a blob 26 to glue any bleeding vessels and then, in so embodiments, supply current. Such a cured mass may be chosen as a liquid mixture/cured electrode or a liquid nonconductor in order to later connect to e.g. an electrical wire, allowing the liquid mixture 1 (initially used to stop a bleeding) to be used as electrode for neural stimulation, block or sensing applications.

In another embodiment the liquid mixture 1 may also be placed through a small skull bur hole in the skull through which a dispenser, e.g. a flexible tube, may dispense the liquid mixture, under ultrasound or angiographic visualization with the goal to form an contact pad 14 of liquid mixture 1 epidurally or subdurally. Such a contact pad 14, when stimulated by a signal generator 17, may be used to arrest seizure activity in patients. In contrast to other electrode technologies, the liquid mixture may be placed through a very small bur hole.

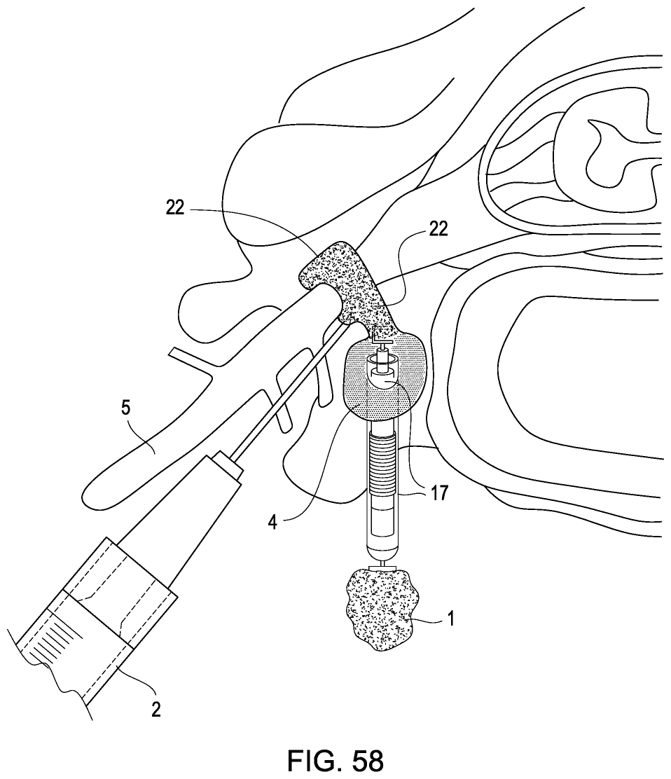

In another embodiment, the present invention comprises a specially adapted connector 88A (e.g., clip, hole, matrix, mesh, sponge) for attachment to the output(s) of a signal generator 17 to connect mechanically and electrically with the liquid mixture, a wire, signal amplifier or any other signal applicator. FIG. 57 is a representation of two types of specially adapted connectors 88A to enable an excellent mechanical and electrical connection to the cured electrode.

Another embodiment of the present invention's ability to encase other electrical components achieves a mechanically and electrically stable interface to a signal generator 17 in liquid mixture 1 and liquid nonconductor 9 as depicted in FIG. 58. FIG. 46A shows the anatomical structure including a foramen 34 before insertion of the signal generator and the liquid mixture and nonconductor. FIG. 58 is a representation of a signal generator 17 encased with liquid mixture 1 comprising a ring-like portion 22 and a nonconductor 9 as an anchor 4 in the foramen 34 (shown in FIG. 46A) for electrical and mechanical integration with the underlying neural tissue.

Incorporating signal generators as described herein provides yet another embodiment of a neural interface system, the signal generator providing the signal, and the cured electrode providing the mechanical and electrical integration with the anatomy and biology optimized during implantation for each specific patient. The present invention thus provides the capability of connection of a signal generator 17 to internal organs with highly flexible surfaces selected from the group consisting of bladder, stomach, gut, heart and liver as well as the ability to connect to neural plexi in the abdominal cavity and other locations of the body.

In another embodiment, the present invention comprises an electrically conductive mesh 24 wrapped around or covering a target. The mesh 24 is configured and shaped outside the body and does not require curing inside the body and the present invention also comprises an electrical and mechanical connection to a wire 10, allowing for an electrical interface to the target 5 encased in liquid mixture 1 insulated by the nonconductor 9.

Disclosed here are further advantages of the present invention comprising a liquid mixture injected into the body by optimizing electric lead-cell communication. The electrode-electrolyte-cell interface is established primarily between a liquid mixture and cured in the close vicinity of a bodily target. The electric signal of interest travels as an input or output in relation to a signal application This input or output, an electronic lead, is commonly made of metal or another highly conductive material, that passes through an opening in a perimeter which is the enclosure of a synthetic device capable of either generating an electric output waveform or capable of sensing an electric input waveform. As used herein, “waveform” means the change of voltage potentials of the lead vs. another lead or the outer shell or another distantly placed electrode (distant being a relative term, encompassing electrically the concept to be a location that is far enough to provide a common reference point to which an electronic signal may be measured against).

There is a difference in the environment in the body between an acutely and a chronically placed electrode lead (or other implanted synthetic material), and the environment changes over time to become more hostile the implanted object. Whenever an object is implanted into a living organism that the organism recognizes as a foreign object, the living organism will begin a process of attacking, concealing and expelling said foreign object. This process is a foreign body (object) rejection reaction and it incorporates an acute and a chronic inflammatory reaction of the living organism against the foreign object. As the foreign object is first attacked with macrophages and encased in fibrous tissue, the electrical interface impedance between the foreign object (i.e. an electrode, a lead, or the outside wall of a signal generator) and a stimulation target in the vicinity of the foreign object may increase.

As used herein, the electrode or the lead connected to a signal generator, other signal applicator or implant shall be sometimes referred to herein as an “electronic interface object” and sometimes as a prior art electrode, both being referenced as feature 40 herein. An increase in electrical impedance may result from (1) Encapsulation of the electrical stimulation (or sensing) sites on the electronic interface object with cells that form an added impedance between the e.g. metallic surface of the electronic interface object and the target; (2) “Walling off” of the electronic interface object by the body through growth of fibrous tissue, which in addition to the formation of cells, is further dried out by the body, further increasing the mechanical strength of the encapsulation while increasing the electric impedance between the cured electrode and the target interface cells of interest, or (3) Physically moving the electronic interface object by thickening the encapsulation, similar to the process of walling off, but with an active movement in one preferred direction and potentially away from the target interface cells of interest.

In one embodiment, the present invention enables extending an electronic interface object towards a cell (electrically and otherwise). As described herein, the cured electrode possesses the ability to change the path a neurostimulation current takes after an electrode has been in the body and the process of walling off has begun. The present invention allows the ability to correct bad electrode placement (such as in DBS or other rod-shaped electrodes for the PNS) by creating a better current path later on through the injection of the liquid mixture herein, for example, by placing a trace of liquid mixture on the opposite side of a stimulation site to re-route current to that site. Such an extension may be accomplished during the implantation procedure of the electronic interface object. Such an extension may be accomplished a day after the implantation procedure of the electronic interface object and thereby during the acute phase of the living organism's rejection (i.e. inflammatory) reaction. Such an extension may also be accomplished a few days to weeks after the implantation procedure of the electronic interface object and thereby during the beginning chronic phase of the living organism's rejection (i.e. inflammatory) reaction. Or, an extension may be accomplished at least three weeks after the implantation procedure of the electronic interface object and thereby during the stable chronic phase of the living organism's rejection (i.e. inflammatory) reaction. In fact, the extension may be accomplished even before the implantation procedure of the electronic interface object and thereby in preparation of the implantation of the electronic interface object.

In yet another embodiment, the present invention enables extending a chronically implanted electronic interface object towards a target 5. For this embodiment, the implanted electronic interface object shall be understood as having been placed several days to a few weeks prior with a stable inflammatory response having at least to some degree been walled off the implant from the surrounding environment. As a result of the beginning (or stable) chronic stage encapsulation, electric communication between the electronic interface object and the target is impeded or distorted in its communicated frequency components or otherwise changed from the level of communication quality that was present on the implantation day or potentially shortly thereafter. This loss in signal or communication quality may impact the amount of voltage a signal generator needs to provide in order to achieve a consistent or a predictable or a preferential response by the electrically interfaced target. This loss in communication quality may render the implanted electronic interface object useless or merely unreliable for its intended task. To address this problem, liquid mixture/cured electrode may be placed to extend the chronically placed electronic interface object electrically, mechanically (or otherwise) towards the target, either (1) by pushing the tissue formed by encapsulation closer to target, or (2) by breaching the tissue formed by encapsulation between the electronic interface object and the target, (3) by forming a bridge through (or across) the encapsulation between the electronic interface object and the target, (4) by pushing the target closer to the encapsulation formed around the electronic interface object, or (5) by two or more of the above combined.

In another embodiment, the invention enables reproducible stimulation, especially reproducible selective stimulation (i.e. by fiber type, fiber size or with effects of unidirectional activation) as well as partial and/or full nerve block by establishing a stable electrical interface between the electronic interface object and the target intended to be modulated with stimulation and/or block waveforms. In order to achieve an optimal electrical interface, a cured electrode may be placed by surrounding the nerves (axons, or nerve fibers as a whole) with liquid mixture in the PNS prior to placing a conventional lead (or conventional lead with conventional electrodes) next to said nerves, or it may be placed shortly thereafter. In order to achieve an optimal electrical interface, the liquid mixture may be placed to surround the target (axons, or nerve fibers as a whole) in the PNS after a conventional lead (or conventional lead with conventional electrodes) had been placed days or weeks, or months, or even years before next to said nerves. The liquid mixture may be placed in an open cut-down procedure, in a laparoscopic procedure, in an injection via syringe and needle or similar setup utilization based procedure, or otherwise facilitated by the liquid mixture.

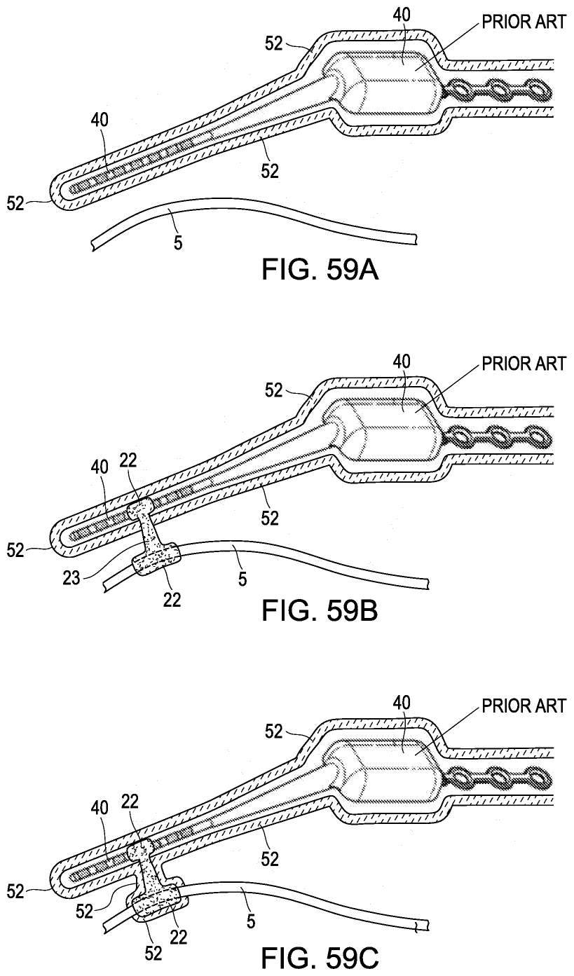

FIGS. 59A-C are representations of how a cured electrode can re-establish successful electrical connection between a chronically implanted electronic interface object 40 and a target 5, where the electronic interface object has been walled off by the body's encapsulation 52 by the body's fibrous tissue. FIG. 59A is a representation of an electronic interface object 40, here a prior art electrode from U.S. Pat. No. 8,494,641 B2 as shown in FIG. 61, surrounded by encapsulation 52. FIG. 61 is another example of a prior art rod-shaped electrode carrier/lead with disk electrodes as shown in U.S. Pat. No. 8,565,894 B2, which could also be encapsulated.

FIG. 59B represents a step in which a physician, in a revision procedure, has cut away the encapsulation 52, encircled each of the electronic interface object 40 and the target with ring-like portions 22 of a cured electrode and connected them with a wire-like portion 23, thus establishing a good electrical connection between the electronic interface object 40 and the target 5. FIG. 59C is the same as 59B, except that encapsulation 52 has now surrounded all the portions 22, 23 of the cured electrode and therefore the encapsulation 52 by the body's fibrous tissue has now provided insulation. The solution of placing the liquid mixture/cured electrode provides a means for the waveform energy to travel from the signal generator to the target nerve again using the path that the cured electrode provides.

Reproducible stimulation, especially reproducible selective stimulation (i.e. by fiber type, fiber size or with effects of unidirectional activation) as well as partial and/or full nerve block may require a stable electrical interface between the electronic interface object and the neural cells intended to be interfaced/modulated with stimulation and/or block waveforms.

The present invention has beneficial effects of increasing signal integrity and preservation. Based on the activating function developed by Frank Rattay, the further away an electrode (or open/uninsulated end on a lead) is from a target, the larger the voltage must be in order to electrically interface with the voltage gated channels of that cell. The activating function is a mathematical formalism that is used to approximate the influence of an extracellular field on an axon or neurons and is a useful tool to approximate the influence of functional electrical stimulation (FES) or neuromodulation techniques on a target. It predicts locations of high hyperpolarization and depolarization caused by the electrical field acting upon the nerve fiber. As a rule of thumb, the activating function is proportional to the second-order spatial derivative of the extracellular potential along the axon. By reducing the distance between an electrode (electrode contact/lead contact/exposed electrode) intended to electrically interface with a target, various advantages arise:

### (1) Signal Preservation

(a) Electrical signals traveling to and from the target are received with a reduction of distortion, at a higher SNR and likely of higher signal quality and integrity. This may be achieved with the aid of an additionally placed cured electrode.

(b) Signal strength may be preserved better with the aid of an additionally placed cured electrode.

### (2) Lower Current Densities

(a) Lower current densities at the electrode-electrolyte interface may be achieved with the aid of an additionally placed cured electrode which allows a reduction in voltage needed to convey the neuromodulation effect reliably, thus requiring lower voltages to be applied when the electrode is used as an output medium to transmit signals to a target.

(b) Lower current densities passing through tissue in the vicinity of the (conventional) electrode in order to reach the specific target.

(c) Lower compliance voltages may be needed by an output unit in order to drive the lower currents needed to achieve the reproducible neuromodulation effect, thereby further reducing the probability of high current densities either through tissue or at the electrode-electrolyte interface.

(d) Lower levels of charge per phase and lower levels of charge density per phase may be required if the cured electrode is placed to overcome a distance and/or encapsulation issue with chronically placed conventional electrodes/electrode-lead combinations etc.

(3) Smaller Battery Requirements, and Less Issues with Battery Life

(a) With a reduction in voltage requirements thanks to decreased electrode-cell (as well as variations thereof such as electrode-neuron, electrode-axon, lead-axon, or lead-neuron) distance, meaning thanks to an increase in proximity and/or thanks to a cured electrode bridging through (or across) one or more layers of encapsulating tissue that may have been in place between the electrode and the target (as well as variations thereof), there may be a reduced need for stored charge in a battery.

(b) This reduction in stored charge may enable the use of smaller batteries, it may enable longer discharging intervals and time spent before a battery may need to be re-charged and it may enable longer battery life before a battery reaches its end of life due to the overall number of charging/discharging cycles or due to the depth that a battery was discharged to (optimal charging levels for typical batteries used in implantable devices such as lithium ion batteries are often in the range of 70% to 30%, whereas charging them up to maximum capacity (95+%) or discharging them to being almost empty (down to i.e. 15% of capacity or less) may be damaging to the long term lifetime of the battery).

### (4) Smaller Coil Size Needed to Provide the Inductive Charge of a Transcutaneously Powered Device

(a) With a reduction in voltage requirements thanks to decreased electrode-cell (as well as variations thereof such as electrode-neuron, electrode-axon, lead-axon, or lead-neuron) distance, meaning thanks to an increase in proximity and/or thanks to a cured electrode bridging through (or across) one or more layers of encapsulating tissue that may have been in place between the electrode and the cell (as well as variations thereof), there may be a reduced need for electrical energy to be transmitted via coil.

(b) The reduction in electrical energy required to operate an implantable device (with or without the additional presence of a charge storage device on board such as a battery or a capacitor) may enable the use of smaller receiver (and/or transmitter) coils.

(c) With smaller coils being used to transmit the energy, smaller form factors may be possible for implantable devices. The key is a more efficient electrode-cell transmission.

### (5) Possibility to Retain Enough Power in a Capacitor to Drive Stimulation or Block

(a) With a reduction in voltage requirements thanks to decreased electrode-cell (as well as variations thereof such as electrode-neuron, electrode-axon, lead-axon, or lead-neuron) distance, meaning thanks to an increase in proximity and/or thanks to a cured electrode bridging through (or across) one or more layers of encapsulating tissue that may have been in place between the electrode and the cell (as well as variations thereof), there may be a reduced need for stored charge in a capacitor.

(b) Capacitors may be used instead of batteries to store the charge in an implantable device while retaining a long enough application of the device without the drawback of degradation of the charge storage over time to the same tune as is known from batteries.

(6) Transforming a Wire into a Cuff

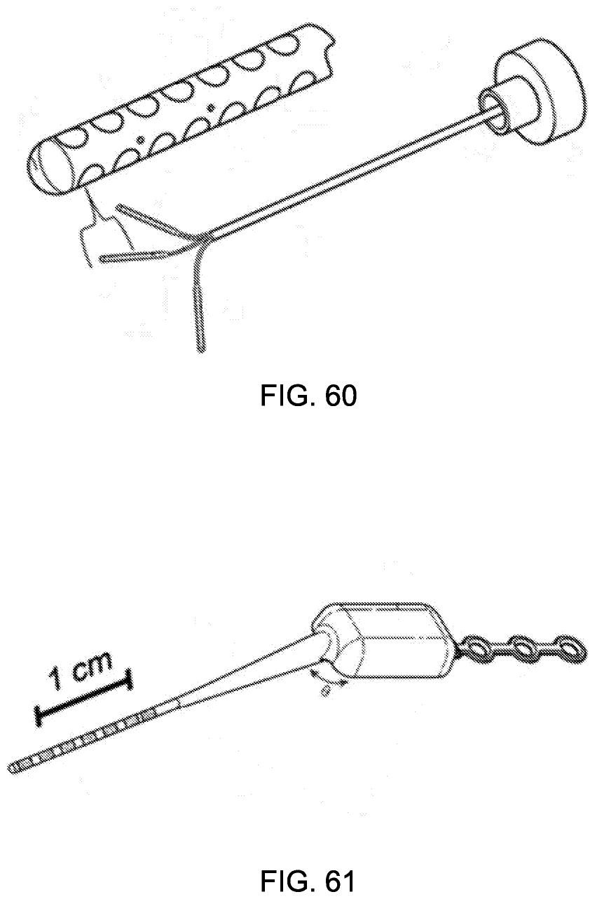

(a) Some implanted neuromodulation devices may utilize electrodes placed on the outside of a lead, showing the appearance of DBS-style electrodes with electrodes placed either as circumferential ring or as disk-shaped electrode next to other non-disk or non-ring shaped electrodes, e.g. faceted lead technology (FIG. 60-61).

(b) All of these electrodes still only sit on the outside of a rod-shaped structure.

(c) None of these electrodes are encasing, enclosing, cuffing, or otherwise surrounding a nerve (such as a cuff would around the vagal nerve for example).

(d) As these rod-shaped structures become encapsulated by the body's fibrous tissue, they may be physically moved, as the body first encases a foreign object and then contracts the cells while drying them out, thereby being able to mechanically express (expel) a foreign object from the body over time.

(e) All these encapsulation reactions have a high likelihood of decreasing the SNR, increasing the voltage requirements for a stimulation to occur reliably and potentially making it impossible for a successful neuromodulation to occur if the rod-shaped lead with electrodes on-board has been walled off sufficiently or moved away from the target (neural) cell or both in conjunction.

(f) Furthermore, the rod-shaped (lead with integrated electrodes) structure itself may have been placed sub-optimally with respect to the target (nerve) cells. It may have been originally too far away, it may be at an unfortunate angle, or it may be that movement of the body (of the implanted person) may impact the impedance between the electrodes and the target cells.

(g) In either way, the cured electrode herein may be placed via injection, open cut-down, via a laparoscopic procedure or otherwise to facilitate a low-impedance bridge between the electrodes and the target cells of interest. This cured electrode may be placed through encapsulating tissue and surround either the nerve, or the rod-shaped structure, or both, like a cuff

(7) Achieve KHFAC or Neuromuscular Junction (“NMJ”) Nerve Block with Help of the Cured Electrode

(a) While a rod-shaped electrode (FIGS. 60 and 61) is able to achieve a stimulation effect on a neural cell, it is not very likely to achieve a nerve block effect as the emitted electric field is neither homogeneous nor stable over time.

(b) By surrounding a nerve that is intended to be blocked with a cured electrode (i.e. via placing the cured electrode around said nerve), one is able to ensure that the electric field applied to the nerve is either uniform, or homogeneous, or relatively stable over time (less affected by the effects of encapsulation than a non-cuff approach), or two or all of these three in combination.

### (8) Interface Means for Neuromuscular Junctions (NMJs)

(a) NMJs generally require a wire to be interwoven (threaded) into the muscle in close proximity to where the nerve enters the muscle. By placing liquid mixture, which cures to an electrode, at that interface (with or without the threading of the wire being utilized) a better energy transfer may be achieved at lower amplitudes required to achieve neuromodulation. When the body's encapsulation has insulated a prior art implant's interface with the bodily target so much that electric stimulation waveforms generated by the implant do not achieve the desired response by the target, placing a cured electrode around or on the contact(s) of the signal generator (or generator's lead) and placing the same cured electrode around the target nerve, or just in the close proximity of the target nerve (a cuff may not always be needed) now allows for the electric stimulation waveforms generated by the implant do not achieve the desired response by the target nerve nearby. The newly forming encapsulation encases the cured electrode around the nerve and around the prior art electrode(s) of the signal generator without again adding so much insulation that communication were hindered sufficiently.

### Immunoreactivity

The body is constantly remodeling and therefore presents the unique challenge as well as opportunity for implanted materials to have differing properties over a specified time course to achieve different goals. Furthermore, with local release or modification of materials, it may be possible to achieve localized regional effects at different locations of the same cured electrode.

The cured electrode is designed to actively utilize the body's inflammatory response for an optimization of its properties. As such, various cells are being used based on their ability to grow into the cured electrode, grow around the electrode, encase, or even resorb parts or the entire cured electrode depending on whether the cured electrode is intended for non-resorbable (permanent) application or if it is intended to be in place only for weeks to months by being resorbable. In that regard, interactions with macrophages are very important as they are part of the innate immunity system. They are attracted to and phagocytose various types of foreign molecules. Proteins and protein fragments, or other macrophage chemo attractants such as endotoxins may be used to promote a macrophage response, which in-turn elicits recruitment of other scar forming cell types (e.g. fibroblasts) that remodel the surrounding tissue. By making proteins and protein fragments and other macrophage chemo attractants such as endotoxins part of the liquid electrode, the properties of the cured electrode in the body are modified, allowing for an enhanced the chronic encapsulation and porosity of the cured electrode, and on the other hand allows for an increase in porosity and for resorbable cured electrode an increase their resorption rate by the body.

For a cured electrode in one embodiment, it is possible to accelerate and increase remodeling of the local environment to produce or accelerate a fibrous encapsulation 52 around the cured electrode 1, thereby forming a naturally occurring insulating layer around the cured electrode to isolate it from surrounding tissues that may be activated as collateral during stimulation. It should be noted that the encapsulation of an electronic interface object from fibrous tissue does not inherently produce a high impedance, but rather it acts to physically separate the tissue from the electrode by a given distance, thereby decreasing the electric field by a factor of the inverse square of the distance. Thus, the present invention can produce a controlled inflammatory response (“CIR”), which term means an increase of inflammation leading to a predictable thickness of encapsulation.

The goal of mediating the inflammatory response may vary but can be used to 1) achieve encapsulation 52 for the cured electrode serving as a wire lead, 2) achieve encapsulation 52 for a cured electrode serving as an contact pad 14 so as to prevent collateral activation of nearby subcutaneous c-fibers during transmission from the electrical stimulus from an external stimulator, through the contact pad 14, 3) downregulate the inflammatory response at the intended nerve interface to prevent fibrous encapsulation between the nerve and the electrode, 4) for use with a biodegradable carrier system so as to cause a progressive “tightening” of the conductive elements 6 as the cured carrier material (e.g., hydrogel) degrades. The encapsulation 52 (i.e, scar tissue) thus squeezes the conductive elements of the cured electrode together.

Modulation of encapsulation may be achieved through the addition of cells and other inflammatory mediators selected from a group consisting of: (1) cells (e.g. mesenchymal stem cells that are known to secrete anti-inflammatory molecules), (2) inflammatory mediators (e.g., minocycline or dexamethasone, having precedence in the demonstration of lowering the glial scar formation with CNS implanted devices, (3) NSAIDs (non-steroidal anti-inflammatory drugs) and the like.

Nonconductive materials may be coupled to conductive materials, as described herein. Disclosed is a method of dispensing liquid mixture 1 around a target, followed by the dispensing of liquid nonconductor/nonconductive layer 9 with and without deploying anchors 4 is described, comprising: (1) Connecting the liquid mixture (which cures to an electrode) to a target, (2) insulating the liquid mixture or cured electrode, using similar material (silicone based liquid mixture is covered with silicone based liquid nonconductor; and the same is true for fibrin glue mixtures, cyanoacrylate glue mixtures and the like), and (3) optionally, the nonconductive layer may be used to further anchor the cured electrode to the target or to surrounding structures or just the local anatomy nearby the cured electrode.

The present invention, in one embodiment, may use current to change the carrier material of the liquid mixture/cured electrode, or the neighboring environment, as follows:

(1) Driving currents to cause partial dissolution of the material by means of:

(a) Material changes chemical composition, or

(b) Fast cycling with kHz frequency to not cause nerve activation but cause partial dissolution of the carrier

(2) Changing the thickness of the encapsulation with currents

(a) increasing or decreasing encapsulation with application of kHz frequency, or

(b) increasing or decreasing encapsulation with application of MHz frequency

### Powder Mixtures and Hemostasis

Combining a hydrophilic polymer and potassium ferrate can provide a mixture that is able to form a stable scab when applied into a wound first under pressure. When this mixture is combined with conductive elements a powder mixture results. These powders are available as prescription-free, over the counter solutions for small external cuts and bruises. Upon contact with blood (as well as chicken meat), the powder forms a sticky compound that keeps mechanically fused biological tissues mended as well as blood vessels coagulated. A mixture of hydrophilic polymer and potassium ferrate can also be added to another carrier material as a hemostatic agent.

Styptics cause hemostasis by contracting blood vessels. Anhydrous aluminum sulfate is the main ingredient and acts as a vasoconstrictor in order to disable blood flow. The high ionic strength promotes flocculation of the blood, and the astringent chemical causes local vasoconstriction. Anhydrous aluminum sulfate powder mixed with a conductive metal powder may be seen as yet another embodiment.

Chitosan hemostats are topical agents composed of chitosan and its salts. Chitosan bonds with platelets and red blood cells to form a gel-like clot which seals a bleeding vessel. Unlike other hemostat technologies its action does not require the normal hemostatic pathway and therefore continues to function even when anticoagulants like heparin are present. Chitosan is used in some emergency hemostats which are designed to stop traumatic life-threatening bleeding. Their use is well established in many military and trauma units.

Kaolin and zeolite are minerals which activate the coagulation cascade, and have been used as the active component of hemostatic dressings (for example, in QuikClot).

All of the above may be provided in solution or suspension or as powder and mixed with conductive elements.

As powders may express the mechanical behavior of a high-viscosity paste prior to curing, a simple syringe/needle system may not be sufficient for delivery/injection, especially when a small gauge needle is utilized. In these cases, the needle, the syringe, the powder column inside the syringe or needle may be vibrated at frequencies of 600 to 60,000 Hz. Vibrating the structure or the mixture can allow more viscous material to achieve a lower effective viscosity (similar to how sand can flow similar to a liquid when vibrated). This approach may be utilized for both, pure element mixture approaches as well as low-viscosity powder mixtures.

The dispenser may not be a basic syringe and needle system for such a powder based mixture, but the conductive material may instead come in small capsules that are opened at the target for connection or a vibration (similar to the one explained for the amalgam) may be utilized in a syringe based dispenser.

### Dispensers

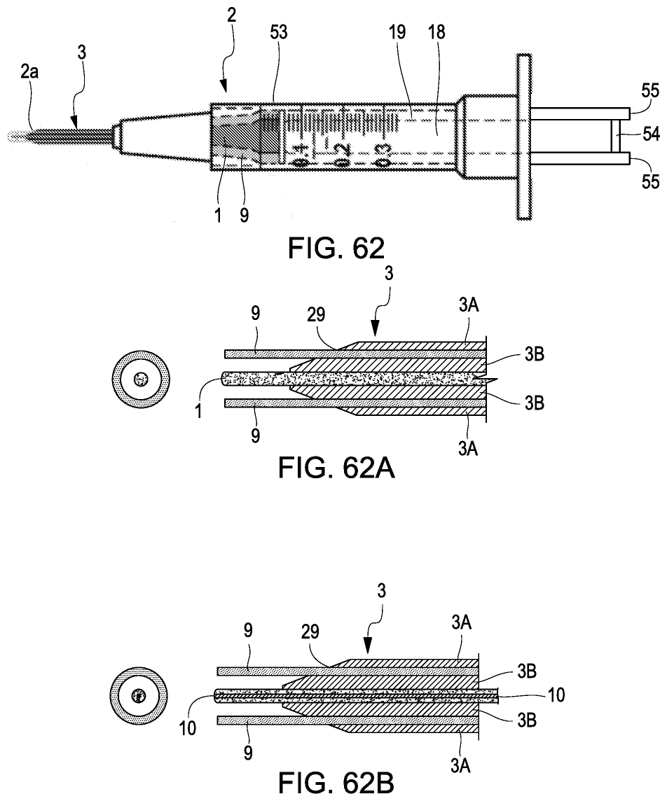

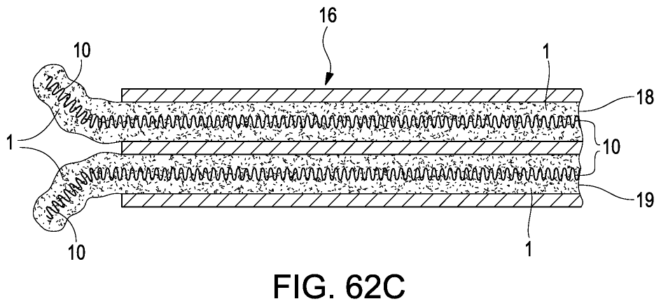

Placing an insulated wire-like structure is feasible with a multi-chamber dispenser 2 which can simultaneously or sequentially, or alternating between the two, injects liquid mixture and/or nonconductive carrier material and/or a continuous wire. FIG. 62 is a diagram of a two-chamber dispenser 2 comprising a syringe body 53 comprising two coaxial chambers 18,19, a first chamber 18 containing liquid mixture 1 and a second chamber 19 containing liquid nonconductor 9, said second chamber encircling said first chamber, a first plunger 54 fitted for the first chamber, and a second plunger 55 fitted for the second chamber, a coaxial needle 3 with an exit point 29 for both chambers. FIG. 62 is an enlargement of the coaxial needle 3 in cross section, showing the outer wall of the needle 3A enclosing an outer needle lumen containing liquid nonconductor and extruding it beyond the exit point 29, the wall 3B of the inner needle lumen extruding liquid mixture beyond the exit point 29. To the immediate left of the exit point in FIG. 62A is the pattern of extrusion of liquid mixture (inner circle) surrounded by liquid nonconductor (outer circle). 62B is the same as 62A, except that wire 10 is being extruded from the inner lumen. In one embodiment the inner chamber for the liquid mixture is surrounded by the chamber for the nonconductive carrier material, i.e., they are coaxial. In one embodiment, the dispensing needle comprises two channels which are coaxial, the inner lumen being for dispensing the liquid mixture and the outer lumen for dispensing the nonconductive carrier material, and the inner channel of the needle communicating fluidly with the inner chamber and the outer channel of the needle communicating fluidly with the outer chamber. Each plunger may be activated separately or they may be activated simultaneously. When the first chamber's plunger is activated separately, only the liquid mixture is injected into a bodily tissue and, upon curing, this material will be a cured electrode without exterior insulation. When the second chamber's plunger is activated separately, only the nonconductive carrier material will be injected and will cure as a nonconductive structure, such as for anchoring. If both plungers are activated simultaneously, then both chambers will dispense material and the liquid nonconductor will surround the liquid mixture and, when cured, will take the form of an insulated wire-like structure, having a conductive middle and a nonconductive outer covering.

In another embodiment, the present invention comprises a dispenser 2 comprising two separate chambers 18, 19, each chamber fitted for a plunger 54, 55 to dispense from one chamber a liquid mixture comprising a carrier material and conductive elements and, from the other chamber, a nonconductive carrier material which is an insulator. The two chambers can next to one another in any configuration or relation to one another.

In another embodiment, two separate syringes can be filled with different materials which can be extruded into a single lumen needle or into a separate coaxial needle like the one in FIG. 62-62B.

In another embodiment a wire is embedded in liquid nonconductor 9 in each of at least two chambers 18, 19 which are adjacent but not coaxial. Two or more electrically conductive wires are injected into tissue with a needle that possesses a bridge in the middle of the needle, as shown in FIG. 62C. Each wire 10 may be spiral as in FIG. 62C or insulated (or not). If the wire is insulated then the first few mm of the wire are not insulated to be brought into close contact with the neural tissue. In a placement in a brain sulcus 49, each wire is ejected from the needle simultaneously and optionally with its own angle, e.g., to left and right of needle towards the neurons within the depth of the sulcus. Once ejected from the needle and injected into the space near the neural tissue (or into the neural tissue), the wires are held in place more and more as the liquid nonconductor cures. If insulated wires (with the first few mm of the tips being de-insulated) are used then these wires criss-crossing within the sulcus is not a problem as the insulation prevents crossing current paths later for sensory or stimulation applications. FIG. 62C depicts a selective wire-based cured electrode for, e.g., sulcus interfacing.

In one embodiment, the dispenser further comprises the attachment of a fiber that conducts light to the injection site to provide illumination for the surgeon (during laparoscopy) or with a different light using e.g. blue or UV to cure the just dispensed liquid mixture or nonconductive carrier material.

Mixing fluorescent or radio-opaque dyes into the insulator enables the surgeon to verify that there is no breakage in the insulation around the mixture. Utilizing such fluorescent or radio-opaque dye in the liquid mixture can help the physician to verify proper application of the glue interoperatively, and even years post-op when radio-opaque dye or elements are part of the liquid mixture.

In one embodiment the dispenser is a device that holds the target in place or that is held against the nerve. The dispenser can inject the liquid mixture at predetermined angles and to predetermined depths into or near the nerve. The dispenser is further able to dispense from both the inner and outer chambers while the dispenser is being extracted from the bodily tissue, thereby sealing or coagulating any potentially formerly nicked blood vessels, and also creating a linear structure from the target to the subcutaneous region.

Needle sizes may vary based on the exact composition of the liquid mixture, e.g., viscosity and other physical properties of the liquid mixture such as the size and shape of the conductive elements, as well as the anatomical environment of placement. The needle may be designed to have a sharp edge to pierce the encapsulation that is present around a chronically implanted electrode, or electrode/lead combination, or electrode/stimulator, or electrode/lead/stimulator combination. Or, the needle may be designed to have a blunt edge to minimize risk of damaging vital anatomical structures. The needle may also have a retractable or otherwise moveable blade to pierce the encapsulation that is present around a chronically implanted electrode, or electrode/lead combination, or electrode/stimulator, or electrode/lead/stimulator combination. The needle may have an opening on the side to facilitate the placement of liquid mixture or nonconductor at an angle to the insertion tract of the needle. The needle may be formed as a continuation of the syringe, of the same material or of a different material and be or not be detachable. The needle may have elements of these points described above in combination.

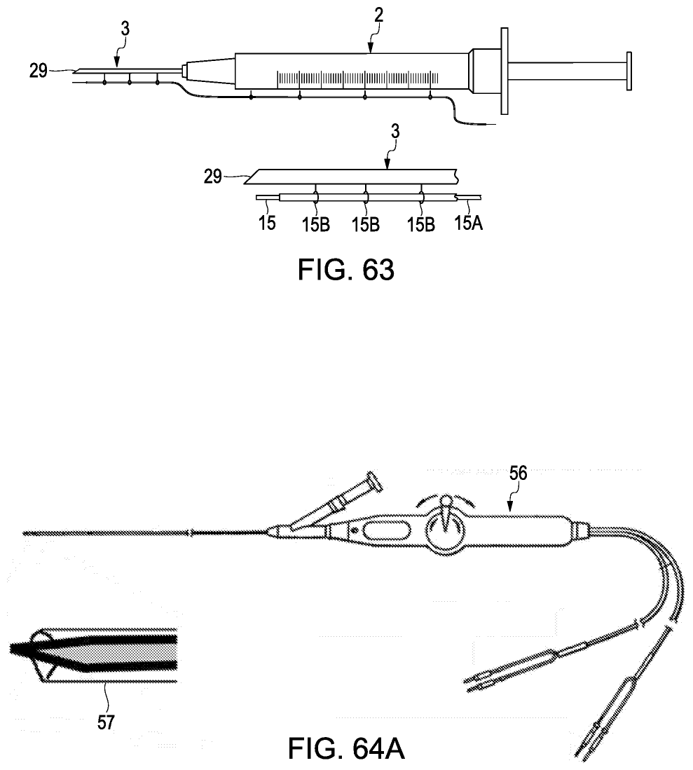

In another embodiment, the dispenser comprises an insulated stimulator wire 54 with an uninsulated electrical stimulator 15 which is near the exit point 29 of the dispenser as shown in FIG. 63. The stimulator wire's other terminal is connected to a power source. The liquid mixture and nonconductive carrier material are injected and the electrical stimulator 15 can provide electrical current to the liquid mixture or to the target 5, to determine if current flows to a target. The feeding of the wire 10 may be similar to a fishing rod feed the fishing line to the hook from a spool (FIG. 63). In this embodiment the electrical stimulator 15 may contact the blob 26 of liquid mixture or nonconductive carrier material which has been injected, formed and molded at or around the contours of the target. The liquid mixture establishes the connection of the wire to the target with a large surface are and good mechanical coupling. Using the dispenser, the liquid mixture and the stimulator wire can both be guided to the connection site at the nerve.

FIG. 63 depicts one embodiment of the dispenser with a stimulator wire, i.e., a syringe filled with liquid mixture with wire guides 15B attached. Wire 10 is threaded through wire guides to be able to contact the target directly or an electrode placed around or near the nerve that the wire is being contacted to. This provides the ability to test the electrical connection of the injected material with the target. The stimulator wire may then be similar to the traditional electrode lead.

Mechanical stability is provided by extruded wire (not the stimulator wire described above) while interface to the body is provided by the blobs 26 of liquid mixture or nonconductor (similar to how a spider dispenses a web) and then places liquid mixture on key points onto the web). Extruded wire with blobs is placed at various points, so that it approximates “blobs on a string.” Extruded wire can be fed external to the needle or through the exit point 29, thus part of the dispenser. Wire can be extruded through the same needle tip and only when the liquid mixture or nonconductor is dispensed, a blob is formed along the wire. Wire can be extruded through the needle of the dispenser, only pulling liquid mixture or nonconductor along when pushed out in parallel to the extruded wire. When the wire is pushed out alone then no liquid mixture or liquid nonconductor is placed around the wire. Liquid mixture or nonconductor is then only placed on locations were the wire is to be connected to the tissue electrically and mechanically.

The dispenser's electrical stimulator 15 is able to provide current in order to verify proper liquid mixture or nonconductor flow or placement either before dispensing, during dispensing or after dispensing. The needle itself may be conductive and be connected directly to a power source (FIG. 63). The liquid mixture itself may have a connection through the syringe/dispenser to verify that the dispensed liquid mixture is indeed connected and in electrical communication with the target. This allows the physician to prevent bad connections during the dispensing process. In one embodiment, the dispenser has the ability to verify correct placement location near or inside the target before and during injection. The dispenser in one embodiment thus has the ability to both sense as well as stimulate a target by connection to an amplifier, a display, and a signal generator. Optionally, a secondary electrode may be placed distally or proximally to the injection site to be able to listen to compound action potentials or single fiber activity at the injection site prior and during inject. In another embodiment the dispenser can deliver anodic current to contract blood vessels such as arteries and arterioles that respond to anodic current with contraction, thereby aiding in hemostasis during the needle (or other dispenser) injection and extraction process.

In another embodiment the dispenser automatically dispenses during retraction from the target. If the dispenser retracts in an automated fashion from the tissue into which it is injecting liquid mixture then the thickness of the blobs or strings (wires) of liquid mixture may be varied based on the retraction speed of the needle (or other dispenser tip) in proportion to the ejection speed of the liquid mixture. The retraction may be achieved by the following steps and configurations:

(1) The dispenser comprises a sensor for acceleration (e.g., accelerometer) and uses this information to predict extraction speed.
(2) The dispenser comprises a pressure sensor for measuring pressure applied during the injection of the liquid mixture while the dispenser is being extracted. The pressure information is used to predict extraction speed.
(3) The dispenser comprises a sensor (mechanical or via laser) to determine a distance to skin measurement to acquire the information to predict extraction speed.
(4) All or some of the above combined.
(5) A visualization system from the outside is capable of displaying an image of the liquid mixture comprising radio-opaque elements. This display may be used to determine injection speed to allow a sufficiently thick line. The display information may be fed to an analysis device running visual signal analysis (e.g. via ImageJ) able to determine the thickness of the injected liquid mixture. This information may be fed back into the dispenser automatically and control the injection speed.

If liquid mixture is being placed as the dispenser is being retracted a specific path the dispenser may be anchored at or near the location of dispensing to ensure that there is no relative motion due to pulsing tissue, heartbeat, breathing or any other movement. The physician may select the desired thickness of the blobs or strings of liquid mixture. The surgeon may provide the information about the tissue into which the liquid mixture is being injected. This matters because fatty tissue for example possess significantly less resistance than do tight connective tissue or various muscle tissues. In another embodiment, a component providing pressure measurement during injection is able to help with a heightened accuracy during the injection. Injecting liquid mixture into more dense tissue will give different pressure results during injection than will more soft tissues. The liquid mixture may be visualized via ultrasound, angiography, or MRI as applicable.

In another embodiment, the dispenser comprises a catheter 56 to inject the liquid mixture to a target. This embodiment of the invention comprises:

(1) A catheter with control rods (or other means) inside.
(2) Exit point 29 holds a retractable needle 57 (retractable; may be retracted into the shaft when not in use to dispense liquid mixture into target) to dispense the liquid mixture.
(3) An electrical stimulator 15 is located near the exit point 29 for verification of proper injection location as well as verification of successful modification of injection.
(4) Retractable needle 57 must be electrically conductive to verify correct injection location with the application of stimulation during the injection process and needle communicates with a power source and sensor in the body of the catheter.
(5) Catheter optionally has the ability to electrically stimulate the tissue prior to and during placement,
(6) Catheter optionally has means to inject liquid mixture or nonconductor and other additives such as resorbable materials, immunoreactive and hemostatic materials and the like.
(8) Catheter optionally has the ability to dispense a fluorescent or radio-opaque dye to improve visualization of correct injection location prior to, during and post injection.

FIG. 64A is a diagram of one embodiment of a dispenser as a catheter for dispensing liquid mixture or liquid nonconductor herein.

There is also a need to reach neural structures nearby blood vessels 100, specifically a need to reach neural structures in proximity to blood vessels but a few millimeters away from blood vessels. The cured electrode provides means to service this need. A system comprising a catheter 56 with balloons 99 to stop blood flow, a signal generator to receive RF signals and make contact with liquid mixture and then the cured electrode to (a) stop any bleeder post-surgery, and/or (b) seal the blood vessel to be able to conduct normal blood flow without leaking, and/or (c) provide a fixation, meaning mechanical integration, of the signal generator on the outside of the blood vessel, and/or (d) providing a better electrical interface to the surrounding neural and blood vessel tissue. Such a system is introduced in FIG. 64B, which shows a catheter 56 including an actuator 101 to deliver a signal generator 17 through a blood vessel wall and seal this wall post-delivery with liquid mixture and liquid nonconductor on the outside and, where applicable, the inside of the blood vessel post signal generator delivery to assure blood vessel tightness against blood leaking from vessel to surrounding tissue. This delivery system has certain advantages over the delivery of a signal generator as well as liquid mixture from the outside of blood vessels: It allows the signal generator and liquid mixture to reach a location that was formerly inaccessible or hard to access with conventional means where a traditional cut-down and spreading are needed to deliver said stimulator, or hard to access with a laparoscopic approach where e.g. the skull of a person would need to be opened in order for the signal generator to be delivered. This system enables the delivery of liquid mixture and a signal generator to the cortex of a subject or patient without the need to gain access to the delivery site by opening the skull of the patient.

In another embodiment the dispenser 2 uses vibration to aid with the dispensing process. Vibration is applied to the column of liquid mixture and/or nonconductor which allows the injection of higher density mixtures of liquid mixture. Vibration further helps to keep the liquid mixture more uniform provides finer or less fine elements during injection. The vibration can be applied throughout the entire dispenser, or just the needle, or just to the column of the liquid mixture (e.g. from the side or the back of a syringe). The vibration can be tuned to specific liquid mixture properties. The vibration, depending on the chosen frequency, can make the liquid mixture appear stiffer or more pliable during dispensing. Vibration allows a very fine needle to dispense rather highly viscous liquid mixture having large conductive elements. Vibration applied at the tip of the dispenser helps to achieve blunt separation of tissue plains.

One embodiment of the dispenser enables injection of liquid mixture or nonconductor into a nerve. An example of this embodiment uses a smaller diameter needle, e.g., 27 gauge (outer diam. ˜0.4 mm), to insert into and place material inside a nerve. In one embodiment, the dispenser comprises elements such as e.g. a rounded tip or a source for pressurized air for blunt separation of tissue. Another capability in one embodiment is a pressure sensor to measure the pressure applied during injection to ensure that the blood supply inside the nerve is not being obstructed as the injected material increases the pressure inside the nerve and any intra-neural pressure in the PNS above 60 mm Hg quenches off blood supply to the structures of the nerve that may be distally to the injection site.

In another embodiment the dispenser is enabled for the injection around a nerve, as in a larger diameter needle (see FIG. 14A-F) 12 gauge (outer diameter ˜2 mm), to inject liquid mixture around a nerve, especially for higher viscosity material. The dispenser also comprises elements for blunt separation of tissue. Such elements may be spreaders, blunted scissor tips that can be opened and closed with a by-wire mechanism (similar to elongated alligator slips)

An embodiment of the cured electrode is produced by dispensing and securing the liquid mixture/cured electrode to a target by covering the target in a crisscross fashion similar to how a spider attaches a web to a twig. Spiders need their webs to be attached to surrounding structures in a mechanically very stable way in order for the web to withstand forces resulting from wind on the web and the surroundings (twigs of a tree the web is attached to) as well as the force when an insect is caught and decelerated by the web. Spiders crisscross the twigs with their web. The present invention is dispensed in vivo to cure in the shape of a mesh, as in FIG. 37. The liquid mixture also may comprise a substance which “etches” an insulator off the wire so that the system itself becomes one fully insulated wire that then is only de-insulated where the blobs are placed.

In one embodiment the dispenser can dispense pellets or capsules of liquid mixture mixed on or near the target inside the body to have the ability to use materials that require very little time to solidify (or otherwise transform to form a mechanically more stable structure). One embodiment of the present invention provides a system that utilizes capsules or pellets that can be applied laparoscopically very close to the connection site. Pellets are loaded into a dispenser and then placed where needed. Capsules may comprise either one or two components, in the case of the latter having a separating wall in-between them and the wall may be crushed or pierced to initiate mixing. The pellets or capsules have application, for example, in the CNS, e.g., connecting to a DBS electrode sitting next to the stimulation target and is able to stimulate the target correctly when the pellets of liquid mixture connect the DBS electrode to the stimulation targets. They also have applications in the PNS (e.g. to form a cuff-like conductive structure around the nerve and behind the nerve or inside a nerve), or for placement in the abdomen in or near an organ by placing pellets or capsules next to each other that then form a conductive path to a wire, a signal generator or similar.

The dispenser also, in another embodiment, possesses the ability to provide UV or blue light for curing at the target in bodily tissue. If the material is a UV/blue light cured compound (like dental acrylic) then the dispenser may comprise a syringe with a UV/blue light LED on the top of the needle, and this can be coupled with visualization through an endoscope.

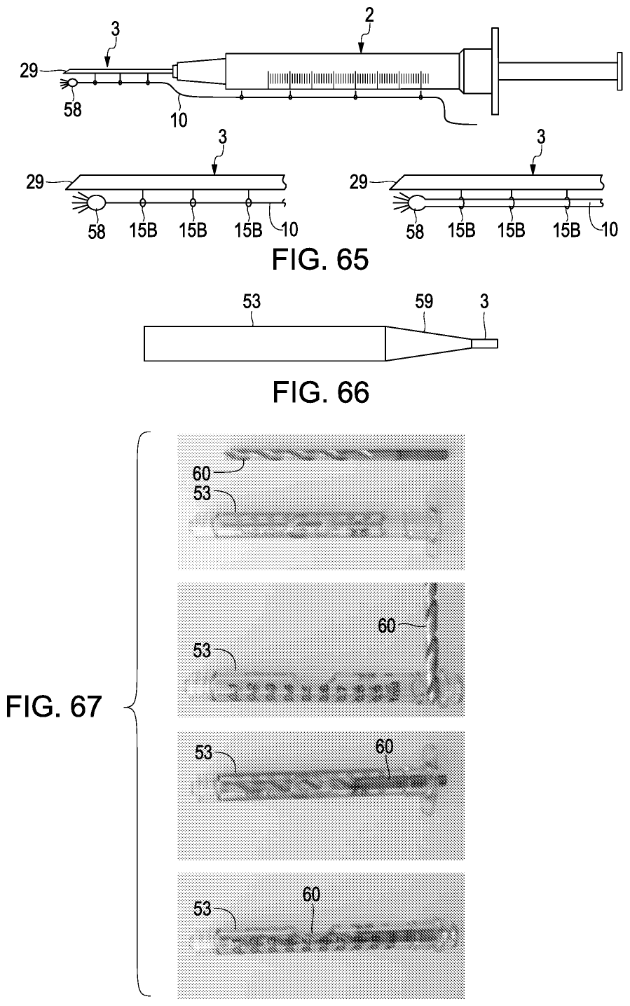

FIG. 65 depicts the dispenser 2 in one embodiment comprising a light 58 such as an LED attached to the needle 3. The light is positioned near the exit point 29 and can be connected to a power source by means of a wire 10 attached with wire guides 15B (similar to the manner as described regarding FIG. 63).

In another embodiment, the dispenser has the ability to provide blunt dissection, using either arms that can spread tissue or pressurized water or pressurized air to bluntly separate tissue near the tip of the dispenser and hold the tissue separated may be an advantage. The dispenser can comprise an element (e.g., rounded tip 16A) which can provide blunt dissection (thereby opening the path around the nerve) and an element that can keep a cavity open for the material to fill around a nerve (i.e., holds open a channel for the material to flow in around the nerve); the blunt dissection being provided by blunt tips like on blunt scissors. In another embodiment, instead of arms that spread tissue along tissue plains, blunt dissection may be achieved with pressurized saline or pressurized air. Once the dissection through muscle plains and other tissue plains reaches the nerve, the nerve can be freed from its surrounding tissues with such a technique without injuring the nerve. Another embodiment can create an air filled cavity near the target by pumping air out near the target and blocking the escape path out the keyhole incision with approaches such as a catheter 56 with at least one balloon 99. Such a catheter comprises a balloon a few (˜10 to 15) centimeters recessed from the tip of the catheter to be expanded and thereby block the artery or vein that it is passed inside. If the balloon inflates wide enough in a small keyhole incision that was created laparoscopically then it can hold air or saline inside the cavity injected from the tip of said catheter. Such a cavity created around the nerve the nerve to be freely suspended once freed from surrounding tissues and thereby provide an easy way to form a molded cuff from injectable material around the nerve.

Another dispenser embodiment comprises a syringe-needle-system with a conical frustum 59 near the end of the chamber of the dispenser transitioning to the needle 3. As the liquid mixture or nonconductor is being pressed out of a large diameter syringe into a smaller diameter needle, pressure points can arise at each location where the diameter of the dispensing column decreases. Such high pressure points may lead to a separation of the liquid carrier material and the conductive elements. Therefore, the ideal flow for most liquid mixtures and nonconductors involves plug flow, or uniform flow rate, across the entire cross-sectional area of fluid being delivered (i.e., flow rate at the wall is the same as that in the center). By gradually decreasing the diameter of the dispensing column from the syringe, or another version of the primary container during storage and/or dispensing, diameter to the needle diameter, steps between the various diameters are avoided. FIG. 66 is a diagram of a conical frustum 59 for graduated diameter decrease to a needle 3 for a syringe. The result is a typical decrease in diameter tested successfully at a decrease from 5 mm inner syringe diameter to 1.5 mm inner needle diameter over the distance of 1.5 cm, and other geometries are also available. The gradual decrease in diameter avoids the step function and dispensing of more grainy and thicker liquid mixtures is more easily accomplished. This method is further improved when ultrasound or mechanical vibration are added to the syringe, either to the column of liquid mixture or liquid nonconductor inside or to the syringe itself. Vibration makes the conductive elements behave more as elements of a liquid, allowing the entire composite to advance without separation from the large inner diameter needle to the smaller inner diameter tip of the needle and eventually the syringe.

In other embodiments the dispenser comprises means for vibration. Vibration has been tested and shown to aid in mixing the carrier material with conductive elements and keeping the carrier material mixed thoroughly with the conductive elements while the liquid mixture is in a liquid phase. Such mechanical vibrations may come from a sound transducer, an ultrasound transducer or a mass out of midline (balance) able to slightly move the carrier material or conductive elements at a relatively high frequency (more than 20 times per second, in one embodiment 50 to 100 Hz). This vibrating column is able to pass through smaller diameter needles and overcome larger changes in inner diameter over travel distance inside dispenser and has even been shown to overcome small step function changes in the dispenser chamber.

Controlling viscosity of the liquid mixture also has been shown to minimize separation of the conductive elements from the carrier material. As the liquid mixture is being pressed out of the larger diameter syringe into the smaller diameter needle, pressure points may build up at each location where the diameter of the dispensing column decreases. Such high pressure points may lead to a separation of the less-conductive carrier medium and the more conductive elements added to the carrier to increase conductivity of the overall mixture and increasing the viscosity of the carrier medium and/or other components of the liquid mixture.

In one embodiment, augers 60 (also called extruders) selected from a group consisting of a screw conveyor, screw feeder and auger drive. All of these systems use a screw inside a hollow tube (e.g., pipe, syringe or needle) that transports material along the axis of the hollow tube by turning inside the tube around the same axis, pushing materials with its threads. Auger based systems utilize any of: (1) A screw on the inside of a hollow tube. (2) A system of a guiding rod placed centrally inside a tube and a coil on the outside of the width of an outside tube providing the driving motion forward. (3) Two screws on inside of an oval shaped or somewhat eight-shaped hollow tube. Additionally, and optionally a forward-backward (or random directional) vibrating motion that may be employed to further avoid clog-up with the target to partially transform the transported material into behaving more like a liquid than a mixture of solids. FIG. 67 are images of an auger embedded in a syringe body 53 to provide a predictable forward motion of liquid mixture through the syringe and reduce the separation of large-grain elements from low-viscosity carrier media at the transition point between syringe and needle. By turning the auger, liquid mixture is transported from the entry-hole, located at the 0.5 ml mark, to the front end of the syringe. A liquid mixture based on silicone as well as metal and coagulant were dispensed from the syringe. The rotational speed determined the amount of material transported over time.

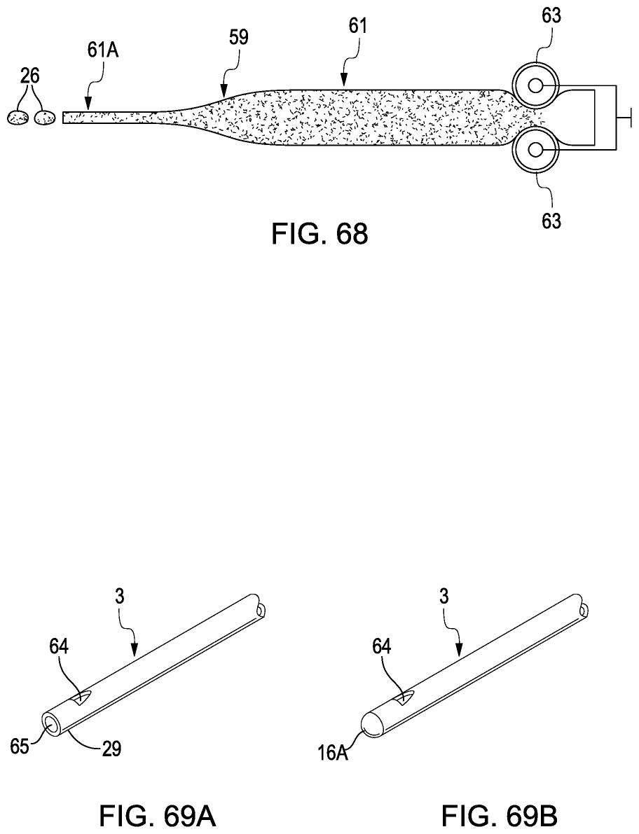

In another embodiment the dispenser comprises a tube 61 which may be rolled up from the rear to dispense liquid mixture from the nozzle 62, and note how the lumen of the tube narrows to the nozzle 61A in a manner consistent with, and for the same purposes as, the conical frustum 59 of FIG. 66. In one variation, the dispenser 2 relies on a tube filled with liquid mixture which is then compressed by rollers 63 that are applying pressure onto the tube starting from the back and moving forward. FIG. 68 depicts a rollable tube 61 embodiment of the dispenser comprising a nozzle on the front end and optional apparatus at the rear to facilitate the rolling of the tube to force the liquid mixture to the needle. In one embodiment, the tube is in the shape of a pipette, approximately 0.5 mm in inner diameter for the length of 10 cm, followed by a graduated tip of the length of 2 cm that ends at an inner diameter of 2 mm. In another embodiment, the tube is in the shape of a pipette, approximately 0.5 mm in inner diameter for the length of 10 cm, followed by a graduated tip of the length of 2 cm that ends at an inner diameter of 1 mm. The pipette-shaped tube is sealed at the back end and may be cut open before dispensing of the liquid mixture, causing any pressure that builds up on the inside of the tube by applying rolls perpendicular to the axis of the tube to force out liquid mixture at the front of the tube.

As the rollers 63 are compressing the back end of the pipette-shaped tube and move forward along the axis of the tube at a linear speed, contents of the tube are expressed at a speed which is linear correlated at the tip of the tube: the speed of liquid mixture dispensing is proportional to the speed of the rolls advancing forward (FIG. 68). In yet another implementation, a plunger is provided at the back of the tube instead of the rollers, utilizing more of a syringe approach to dispense the liquid mixture or nonconductor.

In another embodiment a dispenser comprises means for oscillating pressures and vibration that are at a continuous or variable rate. A continuously oscillating pressure has been investigated as a method of mixing and retaining liquid mixture mixed within the delivery. Furthermore, modulated amplitude vibrations have been investigated as a method of mixing and retaining material mixed within the delivery. Both methods allowed the liquid mixture to behave more similarly to a liquid than to a composite of dry elements, noted as effects equally for silicone and cyanoacrylate based carriers with silver and/or aluminum flakes, as well as dry silver flakes with coagulating powder mixtures. The oscillating pressure as well as the vibration by itself did not necessarily allow for a reliable dispensing of the liquid mixture by itself, but instead helped with a more uniform and linear flow from the syringe tip with the added benefit of a need for smaller pressures to be needed at the back end of the syringe to be applied at the plunger to drive liquid mixture from the dispenser.

In another embodiment a needle 3 of the dispenser comprises an exit point 29 on the side instead of at the front. The opening at the exit point 29 may be of any shape. In order to combat unwanted injury to the nerve or other tissues during the dispensing, different delivery needles were developed. One of these needle systems, depicted in FIG. 69A, utilized an open tip 65 at the exit point 29 at the needle tip as well as an open side port 64, to be able to dispense liquid mixture at both, at the tip and at the side port. Another embodiment of these needle systems, shown in FIG. 69B, utilized a closed and rounded needle tip 16A and relied only on the open side port 64 to be able to dispense only at the side port 64. The open and the closed (and rounded) tip allows a blunt dissection of the nerve with the ability to verify best needle location without unnecessarily high risk for injury to the nerve. Both needles may be insulated throughout except for the electrically conductive end at the exit point 29, as an alternate way to deliver current near the exit point, the wire to the needle may travel through the walls of the syringe body 53 or through the walls of the first chamber 18 in a coaxial dispenser. To be able to verify correct liquid mixture placement, needles may be insulated everywhere except at the exit point 29 or other location on the tip 16 in order to use electrical stimulation to determine proximity to the nerve. Alternatively, the de-insulated exit point 29 or tip 16 in one embodiment comprises a sensor to record electroneurography (“ENG”) signals as a method to locate a target nerve. When electrical stimulation was delivered with the needle placed blindly during surgery at a location assumed to be close to the target 5 then the activation thresholds for the target 5 were obtained and verified. The activation thresholds as smallest values that activate a sub-section of the nerve of interest provided the proper information about the likely best liquid mixture injection location.

For most injections of liquid mixture or nonconductor, a needle gauge smaller than 0.6 mm (>20 gauge) is desirable. The needle gauge can be modified to change the form in which it is extruded. In terms of characterizing the extruded liquid mixture or nonconductor, there is a mathematical relationship between the liquid mixture or nonconductor volume, the needle gauge, and the extruded paste length.

The smaller the needle bore, the longer the extruded material becomes, potentially making the electrode more porous too. The smaller the needle bore will also increase the force required to drive the material through.

TABLE SEVEN
Volumes of Liquid Mixture or Liquid Nonconductor per Needle
Extruded Length (cm)
Injected Volume μL

| | Inner | Needle | | | | | | | | |
|---|---|---|---|---|---|---|---|---|---|---|
| Nee- | Dia- | Vol/cm | | | | | | | | |
| die | meter | Length | | | | | | | | |
| Ga. | (mm) | mL/cm | 50 | 100 | 200 | 400 | 800 | 1,600 | 3,200 | 6,400 |
| 14 | 1.60 | 0.020 | 2 | 5 | 10 | 20 | 40 | 80 | 159 | 318 |
| 16 | 1.19 | 0.011 | 4 | 9 | 18 | 36 | 72 | 144 | 288 | 575 |
| 18 | 0.84 | 0.006 | 9 | 18 | 36 | 72 | 144 | 289 | 577 | 1,155 |
| 20 | 0.60 | 0.003 | 18 | 35 | 71 | 141 | 283 | 566 | 1,132 | 2,264 |
| 22 | 0.41 | 0.001 | 38 | 76 | 151 | 303 | 606 | 1,212 | 2,424 | 4,848 |
| 24 | 0.31 | 0.001 | 66 | 132 | 265 | 530 | 1,060 | 2,120 | 4,240 | 8,479 |
| 26 | 0.26 | 0.001 | 94 | 188 | 377 | 753 | 1,507 | 3,014 | 6,027 | 12,054 |
| 28 | 0.18 | 0.000 | 196 | 393 | 786 | 1,572 | 3,144 | 6.288 | 12,575 | 25,150 |
| 30 | 0.16 | 0.000 | 249 | 497 | 995 | 1,989 | 3,979 | 7,958 | 15,915 | 31,831 |
| 32 | 0.11 | 0.000 | 526 | 1,052 | 2,105 | 4,209 | 8,418 | 16,836 | 33,672 | 67,345 |

The diameter or width of the extruded material can be read from Table Seven under the “Inner Diameter” column. To fully form a ring-like portion 22 of a cured electrode around a large nerve in a human, approximately 400-800 microliters of material is required. For gauges 20-24, this yields extruded lengths of 141-1060 cm.

In one embodiment the dispenser is automated based on sensing neural activity: once the sensor is in proximity to the nerve, changes in impedance and electrical activity may be detected. The nerve may then be freed from surrounding tissues, with the position of the nerve being mechanically or electrically stored in memory. This embodiment may be integrated with ultrasound data as follows: the initial path of tool insertion may be predetermined from pre-operative ultrasound visualization, it may guide the tool path intraoperatively, or may be used at the tip of the tool to differentiate tissue types (e.g., nerve, muscle, fat, etc.) in proximity to the tool One sensor senses pressure during injection and extraction. Dispensing occurs at a pre-defined amount per second by actuation of a button allows a “3-D printing” of neural electrodes in vivo. Each actuation of a control may be graded: e.g., a volume of 1 mm3 is dispensed, or another kind of actuation dispenses every 0.25 seconds a volume of 1 mm³, optionally comprising a dial that selects the amount per click and the amount of time between click dispenses. In one embodiment, an auger system is used to dispense discrete amount with the button push.

In another embodiment a dispenser for use in general surgery combines the ability to throw stitches or place staples into (a) surrounding tissue, (b) the nerve itself, (c) an organ wall—with the goal to anchor the liquid mixture better to the organ wall, nerve or the surrounding tissue. This embodiment provides another method for long term attachment of the liquid mixture if general surgery is needed.

Dispensers may differ according to the type of material to be delivered to the target: (1) auger 60 (screw-in-needle system) to drive higher density/viscosity material, (2) syringe for lower viscosity material, or (3) tube 61 to dispense liquid mixture of medium viscosity. Herein, “high viscosity” is 100,000-10,000,000 mPa-s (e.g., toothpaste-like) and “low viscosity” is 1-100 mPa-s (e.g., water-like). “Mid viscosity” is 100-100,000 mPa-s (e.g., syrup-like).

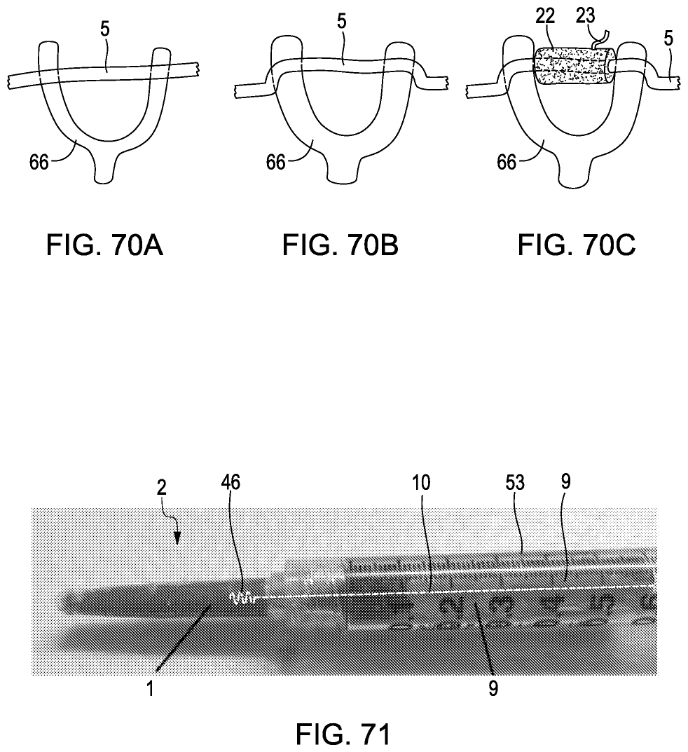

In another embodiment, pre-formed molds 35 may be used by the surgeon as stiff or as flexible devices, and may change in one or more dimensions. One such example is a balloon 66 for a mold that may be inflated when pushed as a “U” shape behind the nerve, then inflated in order to provide a specific cured electrode thickness between the nerve and the tissue behind the nerve (FIG. 70A-C).

FIG. 70A-C is a sequence of diagrams depicting, after a nerve has been bluntly separated from the underlying tissue, dispensing a liquid mixture or liquid nonconductor is possible but consistent thickness may not be easily guaranteed. By placing an uninflated U-shaped balloon 66 between the nerve 5 and the underlying tissue, as in FIG. 70A and then inflating the balloon 66, a uniform distance of the nerve to the underlying tissue may be guaranteed. Once this distance is established in FIG. 70B, the liquid mixture 1 may be safely injected below, behind, near and on-top of the nerve to form a ring-like portion 22 of a cured electrode of a guaranteed minimal thickness, as shown in FIG. 70C. The balloon is mechanically designed similar to a cardiac stent placement balloon: a u-shaped wire provides the mechanical stiffness and is covered with inflatable material, i.e., a balloon 66. When that material is filled with air or a liquid, it assumes a predetermined diameter. This diameter is equal to the separation distance between the nerve and the underlying tissue.

In another embodiment the dispenser comprises a magazine and the liquid mixture, already mixed, is loaded into the magazine. The dispenser is connected to a source of pressurized air, and pressurized air is used to propel small volumes of the liquid mixture from the magazine at a pressure that the physician can adjust to propel the liquid mixture. The pressurized dispenser allows an even or adjustable flow to the target site, and may also comprise a flexible hose for negotiating the tip of the dispenser into locations hard to reach by a straight device such as a needle, such locations as in the brain's midline and in cortical sulci. See FIG. 55A-B.

In another embodiment of the dispenser, an automated dispenser uses ultrasound and a Dispense-Jet, comprising (1) on the input side: (a) ultrasound to acquire a live data stream of the anatomical structure and any dispensed liquid mixture or pellets, (b) a graphical user interface that is part of input from the operator and part of output to the operator, that is, a display of the optimal placement at the target location and (c) a mouse or finger pointer to mark the optimal placement at the target; (2) on the output side: (a) a pressurized air dispenser to propel liquid mixture or pellets to a pre-calculated distance, and (b) a processor to determine the pressure and timing needed to dispense the liquid mixture or pellets at the optimal location.

In another embodiment shown in FIG. 7L an extruded wire 10 (represented in dotted line) is integrated within the dispenser, e.g., a syringe, so that the wire is coaxial with the liquid mixture. This allows liquid mixture to encase a nerve first behind the nerve and then, when the last ⅓ of the liquid mixture leaves the syringe the wire with anchoring leaves the syringe too. The liquid material behind the liquid mixture may be a liquid nonconductor 9 such as a biocompatible starch, cellulose or the like.

FIG. 71 depicts a syringe with a wire 10 with a connecting feature 46 at its forward most point embedded in the liquid mixture which enables forward motion with the viscous mixture. The wire begins in the second half to last third of the liquid mixture and continues to the end of the syringe (where the stencil is).

### Mixing

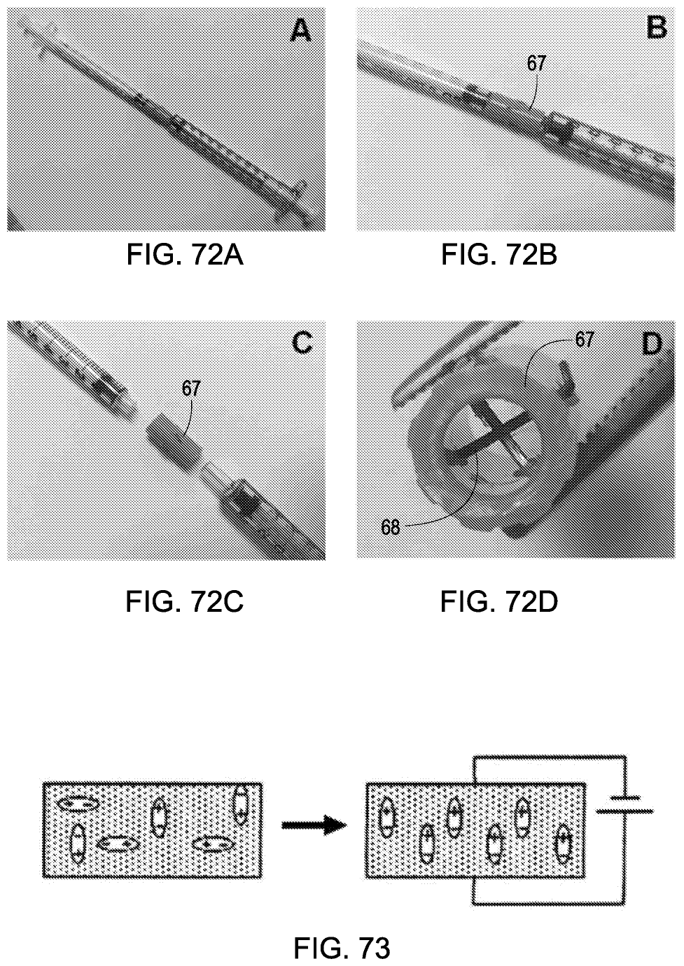

A mixer for the liquid mixture is also disclosed herein. For cases where four ingredients form the liquid mixture, an automatic mixer may be used to first mix components 1 and 2 together (such as conductive elements and a surfactant), then mixing components 3 and 4 together (such as in a 2-part silicone mix or fibrinogen mixed with thrombin to form the fibrin mix), followed by mixing the ½ with the ¾ mixtures. In different embodiments, the mixer may be part of or separate from the dispenser. The mixer may use a stirring, revolving or a shaking motion to mix components. In another embodiment the mixer uses manual action. In one embodiment the manual mixer is syringe based, with turbulence for improved mixing created in part by addition of at least one baffle 68 located within the lumen of a connector 67. Two syringes are joined with a connector 67 in the middle, with the connector comprising at least one internal baffle 68 to increase turbulence for material passing through the connector. Each syringe is filled with one or more of the components of the liquid mixture. The at least one baffle 68 causes an increase in turbulent flow and speeds up the mixing process as the liquid mixture components are being pushed from one syringe into the other and back a few times (FIGS. 72A to 72D). For example, a first syringe holds silicone part A and silver flakes that were formerly mixed with a surfactant such as PVA, and a second syringe holds silicone part B, and silver flakes that were formerly mixed with a surfactant such as PVA. FIG. 72A-D are four images of one embodiment of a manual mixer. Images A and B show two syringes without needles joined by a connector. Image C depicts the syringes and the connector prior to being joined. Image D is an image of the manual mixer comprising a baffle in the lumen of the connector.

In another embodiment, the liquid mixture or liquid nonconductor comprises polymers curing with radio frequency (“RF”) or other energy waves. The physician uses the dispenser to place this polymer (with or without conductive elements) which is subject to curing under a magnetic or RF field. Polar molecules will align themselves in the presence of an electromagnetic field. FIG. 73 is a schematic of dielectric polarization and heating brought about by RF waves.

### Surgical Modifications and Anchoring

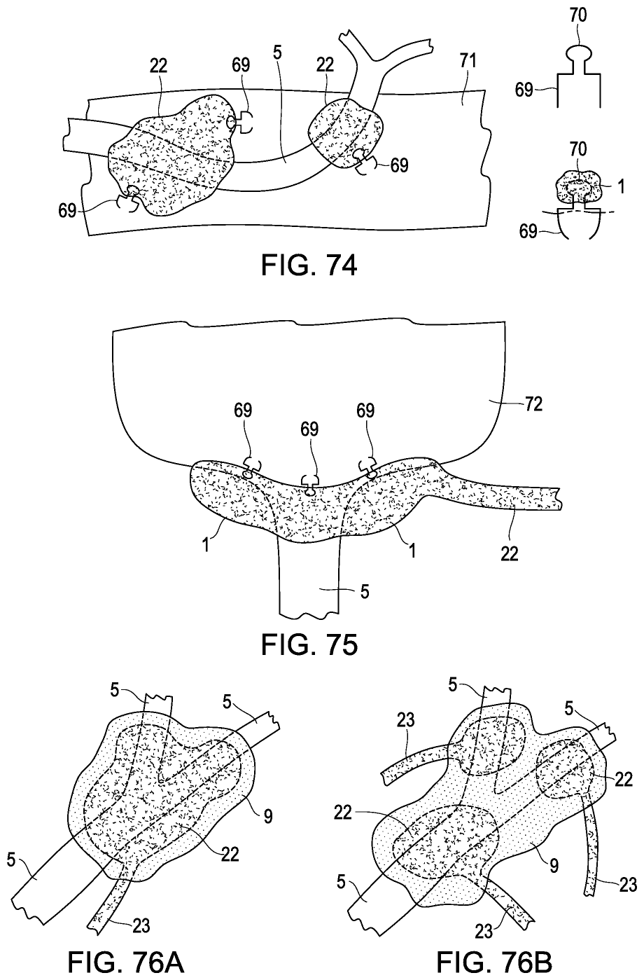

In some embodiments, additional surgical modifications and anchoring may be used with the liquid mixture and liquid nonconductor described herein. To ensure that the cured electrode is mechanically anchored well with the surrounding tissues near the target, additional structures may be used. These structures may be quick and easy to be placed surgically through a keyhole incision, require only very little time to be placed but may provide a significant increase in mechanical integration with the surrounding tissue. In one embodiment, prongs of staples 69 may be placed into the tissue next to the target, so that the stables provide a mechanical support (FIG. 74-75). FIG. 74 is a diagram of staples 69 inserted into a connective tissue plain 71 with the nerve target 5 running next to it. The staples have a connecting head 70 (akin to connecting feature 46) here in a mushroom shape which provides a better mechanical connection after being embedded in liquid mixture which cures. The connecting head may be any shape akin to a loop which creates additional friction to prevent the pulling out of the staple. The ring-like portion 22 of the liquid mixture/cured electrode 1 is anchored with a stronger mechanical attachment to the muscle using the staples. Staples 69 with a connecting head 70 are shown on the right side of FIG. 74: the upper view having straight prongs and mushroom-shape embedded in a cured electrode 1, the lower view with its ends crimped together post placement into e.g. connective tissue 71. FIG. 75 depicts staples 69 with a connecting head 70, the prongs of the staples crimped into an wall 72 of an organ (e.g., bladder), and the connecting head 70 embedded in the liquid mixture/cured electrode to ensure optimal mechanical integration with the cured electrode that is surrounding the bladder at a location of nerves 5 entering into or connecting with the organ wall.

Suture loops provide increased mechanical integration and, in one embodiment, suture loops may be placed similar to staples into the tissue near the target to provide a better mechanical integration with said locations. These sutures may be designed to have specific loops that are open for the liquid mixture to integrate with.

In another embodiment, injection around nerves at a Y-junction adds additional mechanical stability, connecting mechanically to at least one of several nerve branches as well as supporting structures nearby in the area. Placing the liquid mixture/cured electrode at a Y-junction of a nerve provides an excellent mechanical integration with the nerve, and additional advantages. There are several options. Placing the liquid mixture all around the connection point of the three side arms forming the Y provides a means to stimulate all nerve fibers entering and exiting the Y-junction as in FIG. 76A. A different option as in FIG. 76B is lacing ring-like portions 22 of the liquid mixture around each of the smaller side arms 5 as well as additional liquid mixture around the major remaining arm 5, then mechanically stabilizing these three placements with one liquid nonconductor/nonconductive layer 9 surrounding all of them allows for a selective stimulation of either one of the small side arms as well as the stimulating of all fibers by stimulating the major arm. One may further stimulate on one of the small side arms and block on one of the other two arms leaving the Y, either to block afferent or efferent activity directly or to suppress the resulting reflexive neural traffic coming from the spinal cord a few tens to hundreds of milliseconds post initial stimulation.

Blunt dissection of a nerve from surrounding tissue may be achieved by injecting the liquid mixture or liquid nonconductor. Blunt dissection provides ways to integrate the liquid mixture with the nerve but stay movable with the surrounding tissue, such as integration around a nerve Y junction secures it around the nerve but after encapsulation is somewhat movable against the muscle or fascia tissue around it. In another method for placing the liquid mixture, blunt dissection using pulsed air or water may be used to bluntly separate a nerve from its surrounding tissue. The air pressure is to be set to a level that does not overstretch the nerve in case the nerve is subjected to the full blast. Pulsed air as well as continuously flowing air were tested and pulsed air at approximately 2 to 10 Hz, meaning 2 to 10 air bursts per second, proved to be least destructive to the surrounding tissue as well as left the nerve intact, while separating the nerve from the underlying connective tissue. Pulsed water was tested at the same frequency bandwidth and proved to be efficacious. Water in contrast to air was able to “split open” muscle cells from each other, separating the strings of muscle cells, the open space between these muscle cells or strands remaining filled with water or air for seconds to minutes following the end of the pulsed water application. These gaps between the muscle cells, separated from each other but still intact longitudinally, may be filled with liquid mixture or liquid nonconductor injections, allowing a direct interface to muscle cells as well as the stretch receptors surrounding each of the muscle cells or cell strands. The pulsed air may be combined with the delivery of liquid mixture or liquid nonconductor: first a strong burst of air separates the tissues along their plains, then a less intense burst of air is used to shoot a small amount of liquid material into the void. The void is then extended by a stronger burst again, which in one embodiment is followed by an air delivered “pellet” of liquid mixture or liquid nonconductor. The process is continued until a nerve has been covered all around with liquid mixture or nonconductor.

Another aspect of the present invention is a cured electrode finder, say for example, a tool for use in revision surgery. A device may be used to find the extent to which cured electrode is spread below a tissue layer. While this may be done with an ultrasound machine or x-ray/angiography, there is the further option to use a needle-based system similar to the needled skin patch electrode 42 described herein that connect transcutaneously to the buried cured electrode and verify the existence of cured electrode in contact with two or more of the needles by measuring the impedance between the needles.

In yet another implementation, the method of measuring a change in capacitance at a distance of e.g. 2 cm may be utilized. The capacitance of biological tissue may here be understood as the background “noise” capacitance, which changes with cured electrode present within the vicinity of a capacitance reader. Such a capacitance reader may comprise an antenna connected to an output stage to send out an RF signal and connected to an input stage which is used to measure the wave reflected from the surrounding dielectric material. As a cured electrode with its relatively higher conductance will reflect RF signals differently from the lower-conductance biological tissue as well as air, the location of the cured electrode can be determined down to a sub-centimeter XYZ accuracy. When this RF based finder is combined with an accelerometer and moved across a likely cured electrode location, then a 3D-image of the cured electrode may be obtained using this device alone, without any ultrasound or X-ray use.

### Removal

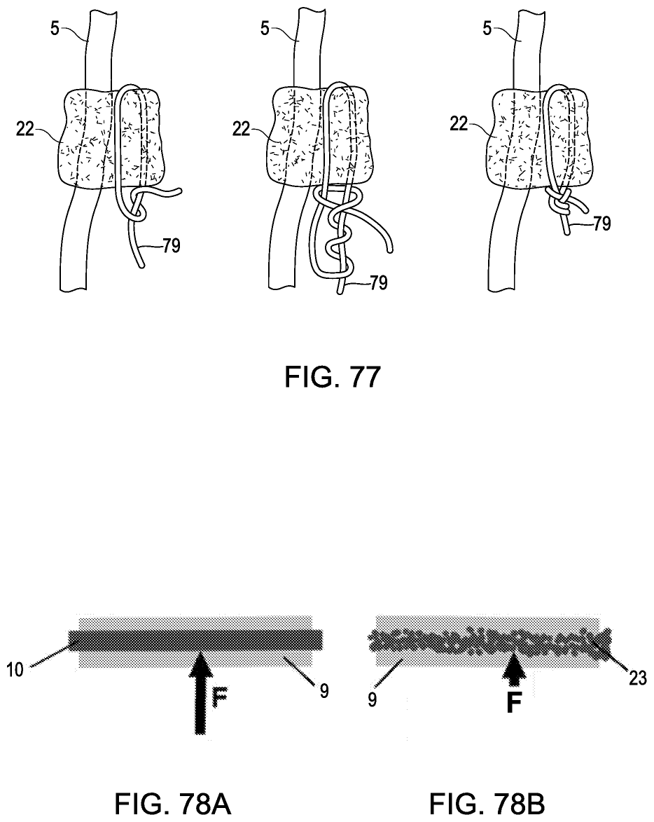

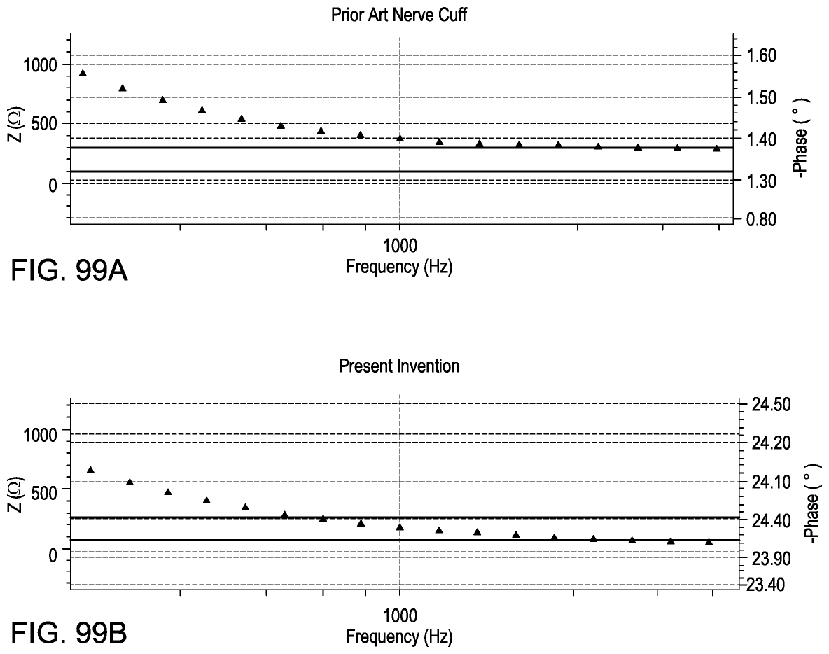

The present invention also comprises an integrated electrode removal system. Prior art neural electrodes do not incorporate a removal feature, so that removal requires the surgeon to cut into the connective tissue that surrounds any chronically implanted electrode followed by cutting the electrode itself. Disclosed herein is a break feature which, if activated, forces the electrode to break at a specific location. This aids with the removal of cured electrodes. A system has been developed and tested successfully to break a cured electrode, comprising a suture placed adjacent to the target before encasing both the target and the suture with liquid mixture or liquid nonconductor which is allowed to cure. Prior to encasement, the suture is tied in a knot which may be released later by pulling. Example knots are the adjustable grip hitch, the palstek knot and the like. FIG. 77 are diagrams showing steps of tying an adjustable hitch knot integrated with the cured electrode to allow breakage of the cured electrode by pulling on the loop to support easy removal of the cured electrode. The adjustable grip hitch knot allows for a tightening, thereby cutting through the cured electrode at a later point in time, even after years of chronic implantation. Also, see FIGS. 99B-C.

The temporary cured electrode (e.g. for DBS, SCS, PNS stim/block) is resorbable over the course of approximately 6 weeks by the body's regular processes, and it thus loses its mechanical integrity. The injectable electrode is placed minimally invasively in a first surgery using resorbable materials such as liquid carrier materials like fibrin glue, proteins, hydrogels and polymers that the body is able to digest, and mixtures of these. Conductive elements of iron, graphene and conductive polymers such as PEDOT:PSS should be sized no larger than 20 microns to allow resorption. Resorption speed may to some degree be controlled by the particle size, with mixtures utilizing particles at an average size of 1 um resulting in cured electrodes at higher resorption rates than mixtures utilizing particles at an average size of 10 um or even 20 um. Where there will be some resorption of mixtures utilizing particles at an average size of 50 or even 100 um, one shall expect mixtures of average particle sizes above 20 um to remain present whereas those below 20 um will over time be resorbed. Post injection, the electrode is used to e.g. test a neural stimulation target deemed likely to be the best location for a therapy. Two outcomes: either inject a liquid mixture designed to be permanent in the same location, or find a new location. In one embodiment the temporary cured electrode may comprise the patient's own cells integrated as part of the carrier material. The patient's own fat cells might be used to provide a partial resorption.

Any cured electrode is relatively easy to remove compared to prior art devices. The ring-like portion 22 of a cured electrode around a nerve is cut and removed. The carrier material for specific embodiments may be designed such that the electrodes can be more easily removed. The properties of the tensile strength of the mixture and the insulator materials chosen can be modified easier than those of standard silicone or polyimide used in traditional electrodes. Furthermore, by applying thicker injected electrodes around a nerve, a higher total tensile strength can be achieved while a thinner application of the material allows for a smaller tensile and shear strength. This means that the physician has a direct influence on the cured electrode's final tensile and shear strength during his or her electrode injection procedure. In contrast to prior art (Case spiral or Huntington spiral) cuff electrodes in which the carrier and metal connectors may be difficult to cut once they have been around a nerve and have become fully encapsulated, the liquid mixture and liquid nonconductor material can be one that has the mechanical tensile strength similar to silicone. Furthermore, there are continuous metal wire connections between the signal generator and the actual electrode contact in a prior art cuff. In contrast, the metal connections achieved by the cured electrode comprise many small elements requiring less force to separate than a continuous metal wire (FIG. 78A-B). The arrows labeled F in 78A and in 78B indicate the greater force necessary to break the prior art structure. The embodiment of the cured electrode may further utilize the body's encapsulation through formation of scar tissue to achieve mechanical stability. Without a prior art wire core (as in the prior art cuff) a cured electrode may be removed more easily, and less invasively.

The cured electrode can furthermore contain additional materials that allow for a long-term modification of the encapsulation. Such materials can be, but are not limited to, e.g., metals that cause a heightened buildup of connective tissue on the outside of the cured electrode (while the inside of the cured electrode next to the nerve is designed to have only a small encapsulation tissue thickness).

FIG. 78A-B are diagrams comparing the difference in tensile shear strength that can be achieved between traditional continuous wire-based conduction of electricity (78A) and the cured electrode (78B). The cut or shear forces for a solid wire connection are much higher and thus it is generally not possible to cut an implanted cuff inside the body that has been there for some time and has thus become encapsulated fully by the body. It is advantageous to be able to have specific wire like connections to and around a nerve that can be more easily cut by a surgeon.

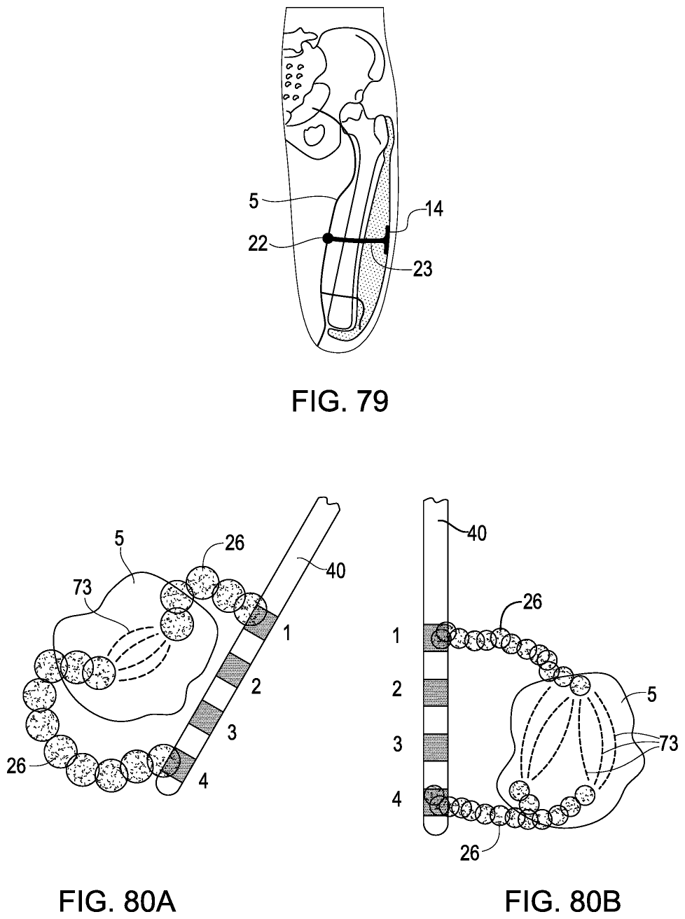

The present invitation may be used to relieve phantom limb pain, pressure, tickle or paresthesia after amputation. The remaining nerves can form a neuroma which can lead to phantom limb pain, the sensation that the amputated limb hurts. FIG. 79 is a diagram illustrating the location of the present invention in an above the knee amputation. A contact pad 14 under the skin surface collects signal (from a TENS electrode 11 as in FIG. 14F), and the current is transmitted on a wire-like portion 23 to a ring-like portion 22 around the nerve target 5.

In one embodiment the liquid mixture is dispensed as a rod-shaped cured electrode that may or may not be flexible post cure but will in every case be electrically significantly more conductive than the surrounding biological tissue of the limb. Instead of a portion of the cured electrode comprising a wire-like structure 23, the cured electrode may also comprise a contact pad 14 below the skin may terminate in a coil that may receive electrical energy via induction from a signal generator held against the skin from the outside, or outside the body from a TENS electrode.

Also disclosed is a method of repairing a broken electrode lead wire of a previously implanted electrode. Neural and cardiac stimulators often have the IPG in one location A and at least one of the stimulation or sensing electrodes in a remote location B. The connection between these two locations A and B is commonly achieved through a lead wire. If the lead wire breaks due to age, excessive movement, force or other causes, then the electrical conduction between point A and B is interrupted. Liquid mixture as described herein may be used to either contact the two ends of the wire directly at the location of the breakage, or it may be used in conjunction with a splitter that allows the surgeon to connect a multi-threaded wire to a connection board on one end and do the same on the other end.

### Connecting to a Prior Art Electrode

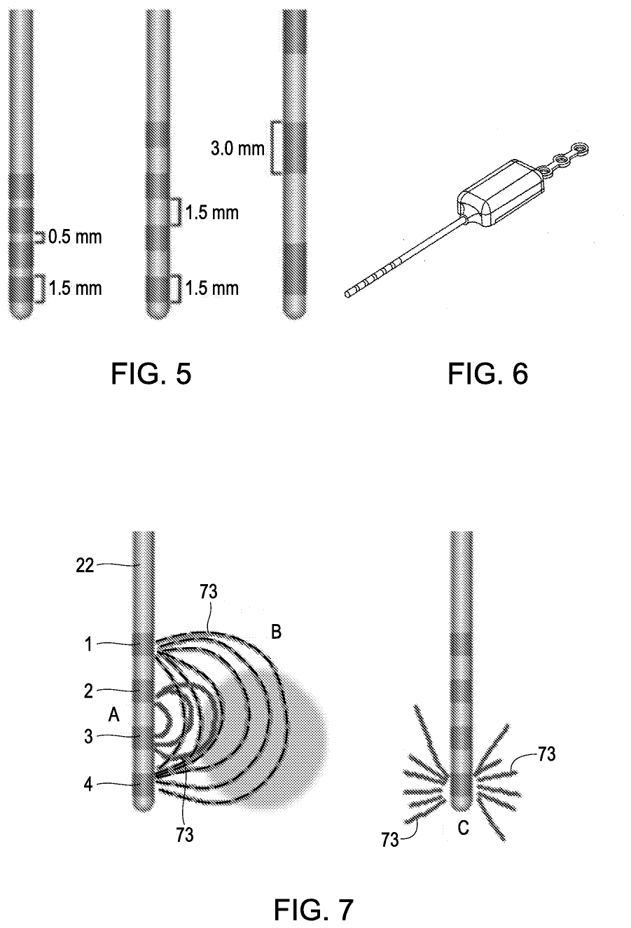

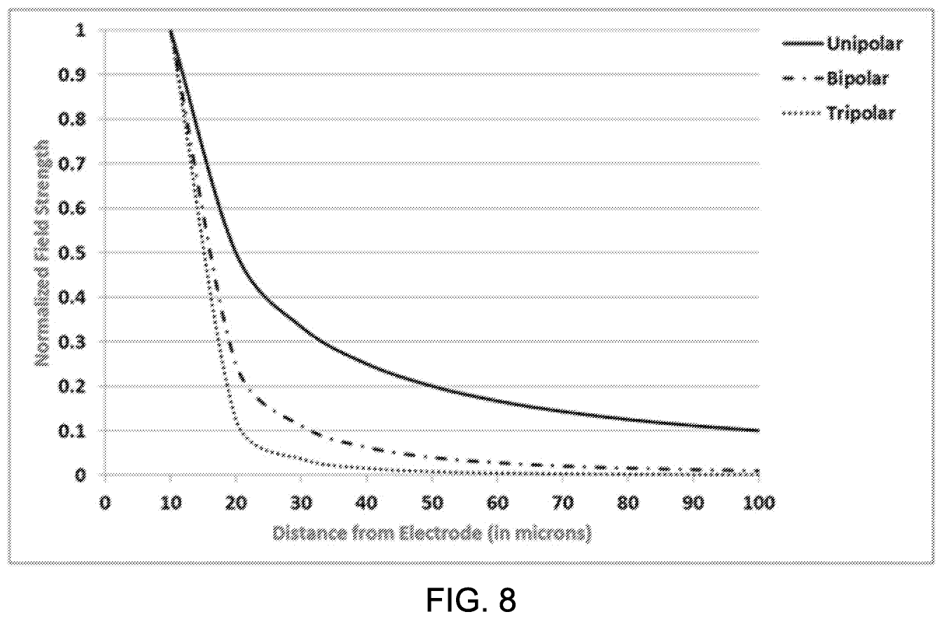

The present invention also comprises a method for electric field shaping to correct improperly placed electrode configurations, or ones which have deteriorated over time. As discussed herein, rod-like electrode configurations are utilized in the CNS for deep brain stimulation or in the PNS to stimulate neural targets from branches of the trigeminal nerve (FIG. 5 from US20110191275 and FIG. 6, from U.S. Pat. No. 8,473,062 B2) to ganglia such as the sphenopalatine ganglion. They are primarily used because of their ease of implantation. They have limited ability to steer the current field lines as each electrode contact is a “point source” from a field geometry perspective. It is hard to stimulate a structure near the rod without stimulating other adjacent structures unintentionally. The present invention incorporates methods and capabilities to combine rod-shaped electrode configurations with the cured electrode, including the ability to (1) change the path a current takes after an electrode has been placed chronically, (2) revise bad electrode placement (such as in DBS) by creating a better current path later on through the injection of liquid mixture, and (3) revise bad DBS electrode implants by placing a trace of liquid mixture on the opposite side of a stimulation site to re-route current to that site. The present invention also includes the capability to achieve a better fit for previously implanted prior art cuff electrodes and thereby increase selectivity.

The present invention includes capability to selectively stimulation and block of superficial nerves and thereby control muscles with surface stim selectively or block pain selectively that may otherwise not be possible with TENS surface electrodes. Selectivity is achieved through liquid mixture being injected into the nerve near specific fascicles. This reduces or eliminates pain formerly caused by high current densities in the skin.

Connecting previously implanted electrode wires to a nerve with a wire (e.g., a plain Pt wire) simply being injected with a 20 gauge needle, each end of the wire being connected to a liquid mixture: one blob 26 near (around) the nerve and a contact pad 14 in the sub-cutis. This allows the capability to stimulate deep nerves (in legs, in abdomen, etc.) with a surface-stim approach. The implant is only a wire 10 and two blobs of the liquid mixture. Materials needed include a very fine needle (micro-needle) for both, PNS and CNS applications, a syringe filled with liquid mixture, and a syringe filled with liquid nonconductor (chosen for high impedance). This approach includes (1), if a DBS electrode is too far from a neurostimulation target (as in FIG. 7 on the left side), the present invention provides the ability to guide the electric current to the proper location without a major revision surgery that requires the ejection and re-insertion of the DBS electrode, (2) using a micro-needle (of 8 to 10 cm length), that is attached to the syringe filled with liquid mixture, a current path can be injected into the brain through a series of “blobs” 26. FIG. 80A-B o are diagrams depicting examples of placement of liquid mixture “blobs” on prior art electrodes to align field lines through the target structure. That is, a string of blobs from contacts #1 and 4 makes contact with the target 5, and the electrical field lines 73 are centered on the target, unlike in FIG. 7, where the current fields A and B are not able to electrically stimulate the neural target structure (shaded area) with or without simultaneously activating other structures unintentionally. Thus liquid mixture may be dispensed to create a path from a prior art electrode to the neural target.

The present invention may be configured as a flexible DBS electrode. Materials needed include a long micro-needle, a syringe filled with liquid mixture (e.g. PEG carrier material mixed with silver conductive elements), and a syringe filled with liquid nonconductor (chosen for high impedance). This approach includes (1) use of a syringe, and a liquid mixture is placed into the brain from the GPI-STN as a string of conductive blobs 26 in the form of a track back out to the skull, where a contact point is made, and (2) (optionally) an insulator on the outside of the conductive track to avoid accidentally stimulating neighboring structures. An advantage is that this one cable, in form of pearls making the “cable” flexible, may stimulate the nucleus of interest in the brain. This DBS style design allows a more minimally invasive approach with the option to later correct the electrode placement by imply adding more liquid mixture at the correct location. The carrier material may be protein based with a matrix that holds the conductive elements (such as gold) in place, ensuring conductivity and keeping the flexible electrode in place. The mixture may be injected at the same or a higher rate than the injection needle may be extracted with the potential to chemically seal any bleeders that may arise from the injection of the needle into brain tissue. If the material is conductive from the point of injection onwards (meaning even before a curing period has passed), the conductive material may be used to apply an anodic potential that contracts small blood vessels in the vicinity of the injected electrode material. This approach is able to hold ruptured blood vessels shut for the first few seconds post injection and minimize bleeding into the wound channel, thereby reducing the expected neural scarring (glial scarring) at/near the injection site, thereby allowing lower neural stimulation thresholds and better SNR values for recording setups using the cured electrodes.

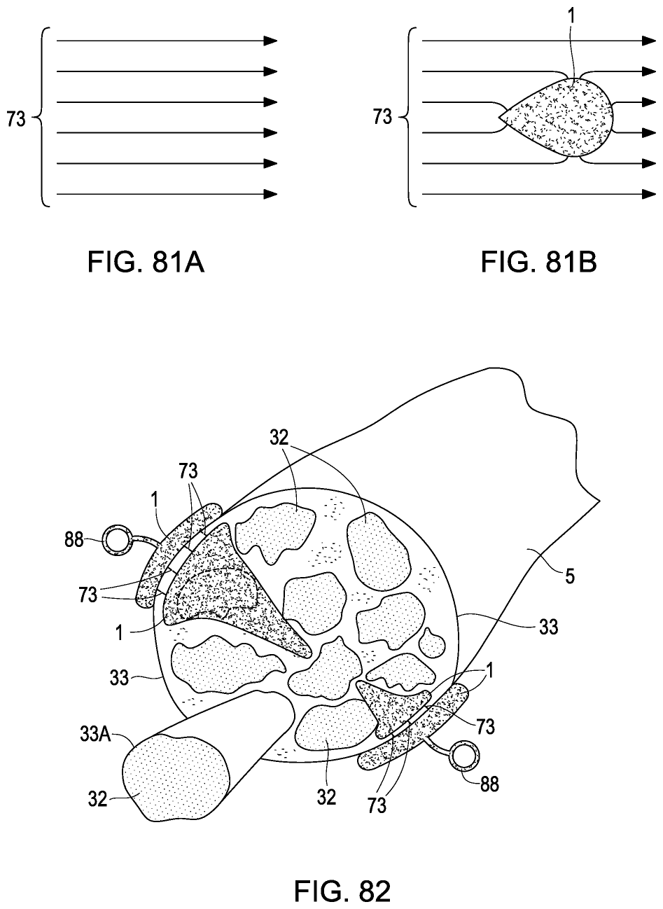

Electric field lines 73 using the present invention may be achieved, in one embodiment, by shaping by adding conductive material into the nerve. Using induced charge transfer to activate nerve fibers using kHz waveforms to stimulate, even a normal stim pulse of 200 μs cathodic and 200 μs anodic charge balancing will effectively be a 2.5 kHz signal for the moment of stimulation. The liquid mixture may be porous for maximal capacitance effects. Electrical field lines 73 pass preferentially through materials of low impedance. At the location of the interface of a good mixture to a bad mixture field lines are most dense. By injecting the conductive material into the nerve itself and without completely connecting the liquid mixture through the nerve's membrane, electric field shaping is possible as electrical field lines 73 follow the path of least electric resistance. FIG. 81A-B are diagrams showing how placing a material of high conductivity into a medium of lower conductivity with a homogeneous field that passes through the low-conductivity medium causes a distortion of the electrical field lines 73. In 81A there are homogenous field lines 73, but 81B depicts distorted field lines due to a placement of a liquid mixture into the electric field lines which are bent towards and into the medium of high conductivity. Field lines are able to pass through the medium of high conductivity in higher density. Thus, field lines in the medium of low conductivity may be bent towards the high conductivity medium, creating hot spots in the medium of low conductivity with locally heightened field densities. These higher field densities may be utilized by placing them near a stimulation (or block) target, i.e., placing a high conductivity liquid mixture blob 26 near a fascicle 32 with the fascicle in line with the liquid mixture blob 26 will cause higher field densities through that fascicle while blobs placed near a fascicle on an axis perpendicular to the field lines will reduce the field lines through that fascicle. The placement of liquid mixture blobs can change the probability for fascicles to be stimulated based on whether the blob is placed in line or perpendicular to the field lines.

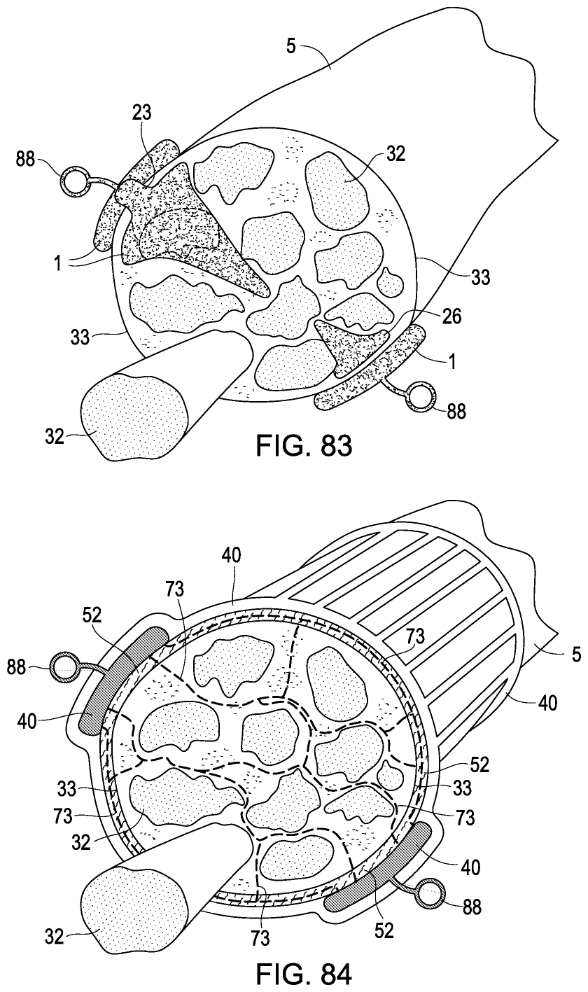

At least two configurations result from the foregoing. First the liquid mixture 1 may be placed inside a nerve 5 without an exit trace. FIG. 82 is a diagram showing liquid mixture blob 26 injected into the nerve 5 without leaving an exit trace through the nerve's epineurium 33, and the liquid mixture/cured electrode connects with two additional cured electrodes just outside the epineurium which in turn connect to other wires or devices at 74. Another option is to inject liquid mixture into the nerve 5 with a connection left across the epineurium. FIG. 83 depicts a liquid mixture blob 26 injected into the nerve while leaving a wire-like portion 23 of the cured electrode through the nerve's epineurium, here shown only on the left side but it is possible to do so on both sides. The liquid mixture 23 on the left side exits the epineurium to form a faradic bridge and the exit from the nerve can be at a 90 degree angle (perpendicular to the nerve) or at a very shallow angle leaving a comparably long trace inside the nerve. FIG. 83 shows the perpendicular exit of the wire-like portion 23 of the liquid mixture through epineurium. For these interventions in FIG. 82 and FIG. 83, the materials and approach include (1) a small diameter needle; (2) measurement of pressure during injection to avoid occluding blood supply to distal structures; (3) use of ultrasound or fluorescent dyes to verify injection into the nerve is successful, and (4) depending on a variety of parameters, the liquid mixture blobs 26 may be injected into the nerve without leaving a continuous stream through the epineurium utilizing capacitive displacement current and voltage field shaping for the intended effect. In FIG. 83, electrical field lines forming inside the nerve are changed from uniform lines to more compacted lines near the injected conductive blobs making up the cured electrode.

Electric field shaping may also be achieved by adding liquid mixture around or into the nerve. The current amplitude is always inversely proportional to the impedance of a current path. As there is generally more than one current path in a biological system, controlling current flow through optimal placement of low and high impedances (resistive and capacitive) becomes very important. A prior art nerve cuff electrode 40 shown in FIG. 84 (see FIGS. 4a-b) for example will rarely conform to the contours of a nerve optimally (i.e., without space between the outer cells of the nerve's epineurium and the cuffs inner diameter) unless the cuff is intended to reshape the nerve, thereby applying an intentional pressure to the nerve from the moment of cuff placement. This open space between the nerve and the prior art cuff will generally be filled with encapsulation 52 of fibrous tissue which is relatively dry and higher in impedance than the surrounding interstitial fluid as well as the neural tissue of the nerve to be stimulated. This means that some current (electrical field lines 73) will pass from one contact inside a cuff to another other within the same cuff without passing through the nerve (as shown by dotted electrical field lines 73 including those on the circumference of the nerve just inside the epineurium 33), even when two cuff electrode contacts 40 are diametrically on opposite sides of the nerve inside the cuff. FIG. 84 depicts field lines 73 through and around a nerve with two electrodes placed diametrically on opposite ends. Note how the shortest current path is through the nerve but some low impedance paths might be just outside the nerve and between the encapsulation 52 of fibrous tissue that has formed between the nerve and the cuff 40. Yet without a layer of insulation around the outside of the electrode contacts and the nerve, there is even more current spread which is why an insulating material helps to provide strong, more uniform electrical fields through a nerve instead of non-uniform fields around it, thus increasing the ability to stimulate or block the nerve.

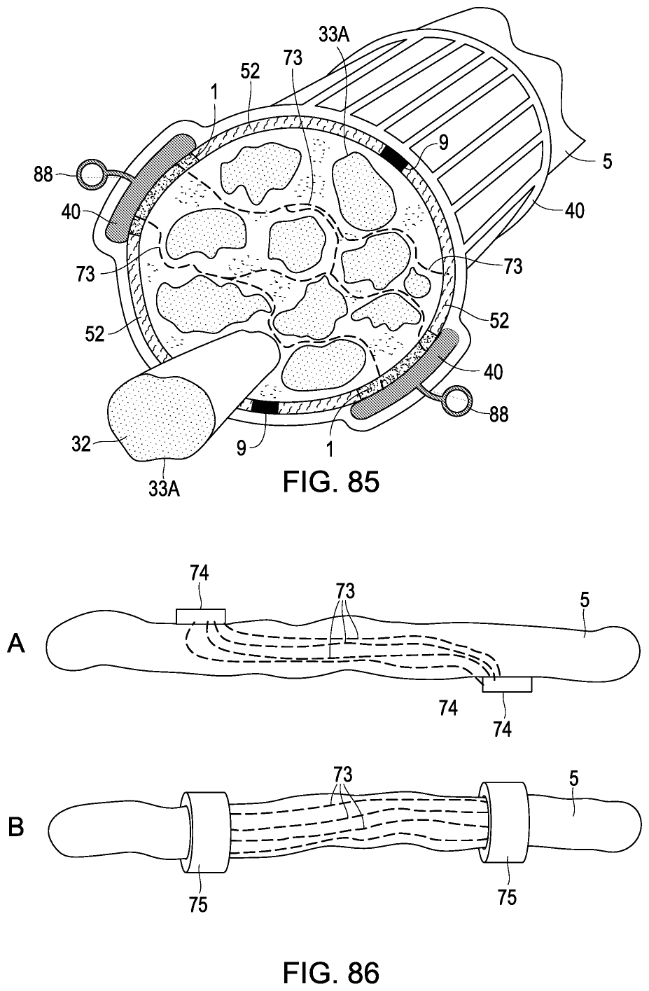

Current shunting around a fascicle is achieved in a manner similar to the method for shunting around a nerve when stimulation electrodes are outside the nerve or even when inside the nerve and the perineurium 33A around the fascicle 32 is too dense, so that injecting contacts next to the fascicle of interest can take care of that problem (FIG. 85). FIG. 85 is a diagram showing that field lines 73 (compared to FIG. 84) can be changed even in a chronic cuff electrode placement around a nerve 5 by placing liquid mixture 1 just underneath the two cuff electrode contacts on opposite sides of the nerve just inside the cuff electrode. Also, note that two insertions of liquid nonconductor 9 have stopped the electric field lines 73 from going circumferentially, as shown in FIG. 84, with the electrical field lines 73 concentrated in the middle of the nerve instead of scattered throughout or at the edge.

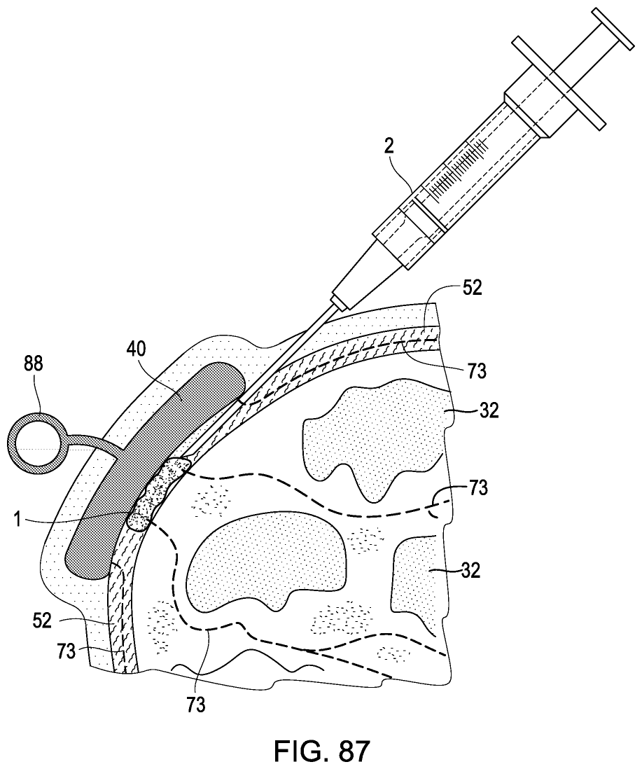

Another method allows shaping non-uniform electrical field lines 73 which current will follow. Another aspect of designing electrical fields 73 that depolarize all nerve fibers of a given fiber size within a nerve is to use circumferential electrode contacts instead of disc electrode contacts. Field lines 73 around ring electrodes 75 are not uniform: the closest field lines appear near the edge of the disc electrodes 74 that is facing the other electrode leading to higher current densities and thereby larger induced voltage differentials applied to nerve fibers at that location. As shown in FIG. 86, (a) disc electrodes represent a point-source electrically and allow higher selectivity through their ability of activating a nerve's fascicles with a higher probability in their proximity, and (b) ring electrodes 74 encircle a target provide more uniform electrical field lines and thereby more selectivity based on fiber size. FIG. 86 includes two diagrams showing the difference in electrical field lines between disc 74 (less uniform) and circumferential ring electrodes 75 (more uniform). These field lines can further be changed as needed by placing liquid mixture blobs 26 or rings 22 around, near or inside a nerve (or other target), as shown in FIG. 81B.

The present invention also allows a better electrical and mechanical fit for a prior art cuff electrode, thus modifying the electrical conduction between a conventional cuffs electrode contacts and the nerve. As indicated herein, cuff electrodes are often installed with a void 39 (see FIG. 48) between their electrode contacts and the neural target tissue. FIG. 84 is a diagram showing how encapsulation 52 with connective tissue grows in gaps between the electrodes and the neural target. As metallic electrodes often have recesses into the insulating carrier material (silicone, polyimide and others), connective tissue encapsulation 52 surrounds the nerve with a tight “wall” that is thicker at the location of the electrode (as it fills the void 39 between the recessed electrode and the nerve), thereby increasing stimulation thresholds and reducing SNR values for sensory applications.

FIG. 87 is a diagram showing creation of a gap in the tissue between the prior art cuff electrode's contact pads and the nerve and then injection of liquid mixture to fill that gap, and also a bridging of encapsulation. A liquid mixture 1 may function as a bridge between a prior art metallic electrode contact 40 and the nerve 5 if liquid mixture is placed onto the contact prior to implantation of the cuff, as in FIG. 49A. As shown in FIG. 87, this application of liquid mixture may also be placed post-implantation of the cuff if a fine needle is used to inject the liquid mixture 1 into and if the connective tissue right between the cuffs electrode contact and the nerve is removed by physical, biological or chemical means (FIG. 87). In FIG. 87 the electrical field lines 73 spreading inefficiently around the circumference of the nerve, will be redirected by a new application of liquid mixture added after original implantation jumps the void 39 and also cuts through the encapsulation 52.

The present invention may be used for re-establishing a cardiac conduction at locations where neural/muscle conduction of control signals to contract the heart is interrupted due to illness, injury or alike. A cardiac infarct can lead to the formation of scar tissue at a location that is required to transmit electrical signals from one location of the heart to another, thereby requiring the implantation of a cardiac pacemaker. The cardiac pacemaker senses the depolarization in one location of the heart (e.g. atrium) and then transmits this information to another location (e.g. the apex) that does not receive the command to contract any more due to injury, illness or alike. By injecting liquid mixture e.g. into the scar tissue within the septum that may conduct the control signal to the apex, the liquid mixture can reestablish the electrical conduction. Considering that cardiac pacemakers are more complicated than the re-establishing of conductive pathways, the injection of liquid mixture in the heart muscle provides a more reliable and efficient approach for patients than reinstalling a pacemaker.

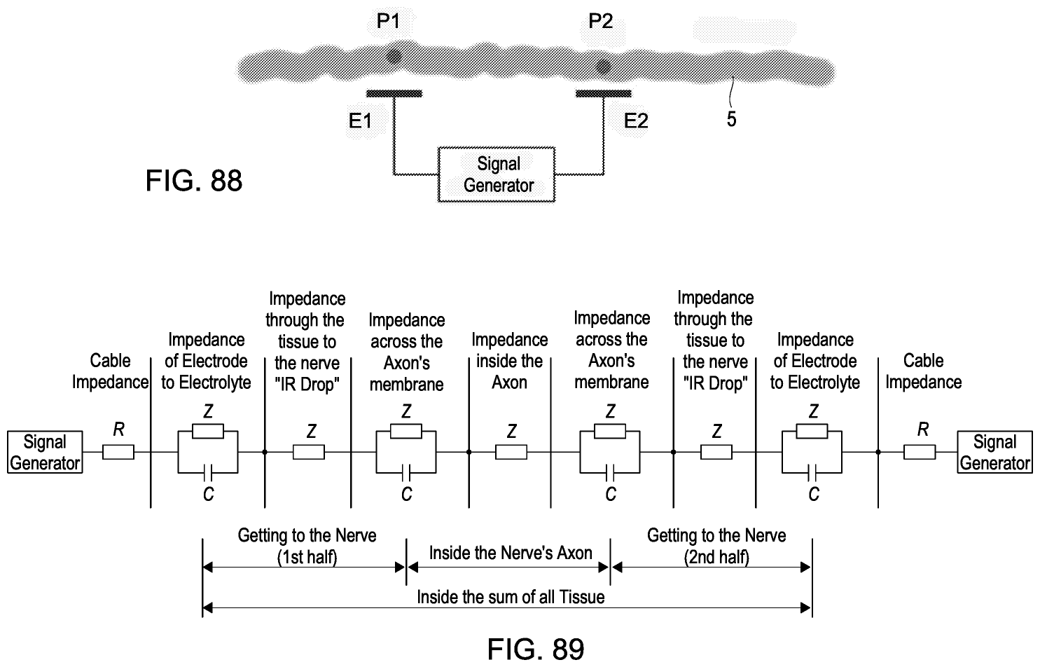

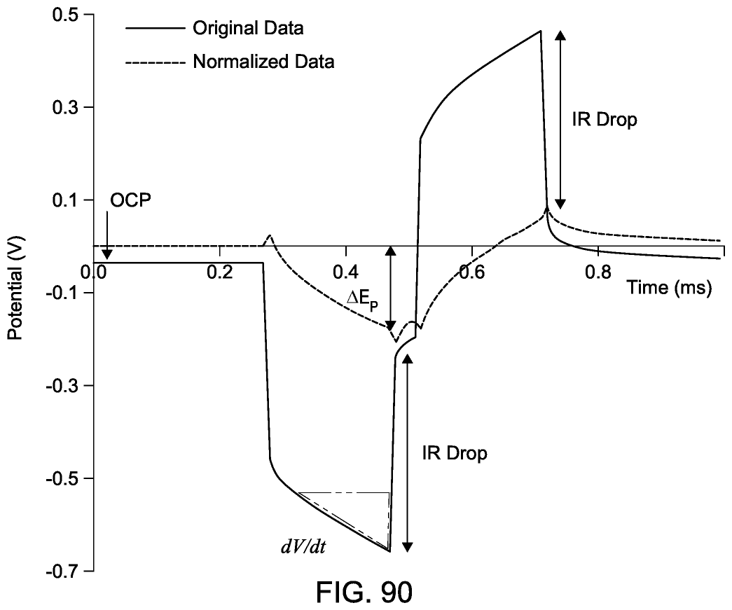

Reducing the IR drop is achievable with the present invention. In FIG. 88, it is assumed that two electrodes E1 and E2 from the signal generator are connected to the same signal generator and that a nerve is placed longitudinally between these electrodes. Of interest is the voltage between two points P1 and P2 inside the nerve, more specifically inside one of the axons of the nerve. FIG. 88 is a schematic of a nerve with two electrodes being placed along the nerve. When the voltage difference between P1 and P2 changes over a certain threshold at a specific (short) time then an action potential is evoked. In order for electric current to flow from point P1 to P2, an electrical difference in potential (voltage) must exist between P1 and P2 and a conductive medium must be present such as a metallic wire (electrons conducting) or an ionic liquid such as it is present inside a cell (ions conducting the electrical current). There are a several components to the final impedance from the signal generator to the electrode to the electrolyte, through the electrolyte to the nerve, across the membrane, inside the nerve, back across the membrane, through the electrolyte towards the electrode, the interface back from electrolyte to the electrode and from there back to the opposite end of the signal generator. FIG. 89 shows the total impedance from the electrical stimulator to the inside of the nerve and back. FIG. 89 is a schematic of resistive and capacitive impedance components on the path from one electrode through interstitial fluid to the axon within a nerve and back. In other words, of the total applied voltage from one side of the signal generator to the other side of the signal generator, there is a complex sum of impedances in the path. The largest purely resistive component of that path is the ionic conduction of current through the electrolyte and the tissue made up of connective tissue between the electrode and the nerve's axonal membrane. This more or less purely resistive component is captured in the “IR-drop” of an applied square wave current-controlled signal, shown in the solid line of voltage over time in FIG. 90 which is a schematic of the voltage curve measured during current controlled stimulation showing the resistive component (solid curve: vertical lines=IR-drop) and the capacitive component (dV/dt indicating the charging of surface boundaries). Source: http://iopscience.iop.org/article/10.1088/1741-2560/13/5/056011 via Google Images. If the voltage drop through the tissue and electrolyte were subtracted out, then the voltage measured to charge the electrode-to-electrolyte interface (and to a small degree the capacitance of the axon's membranes) is also shown in FIG. 90 as the dotted line.

### Follower Circuits

Follower-circuits may be used to pick up an electrical signal in the radio-frequency spectrum (e.g., 1 to 10 MHz) and they comprise a receiver coil, a diode, a transistor, a capacitor and a resistor, all of them passive components hermetically sealed, the product follower-circuit being encapsulated in silicone to provide some form of mechanical stability.

Instead of soldering the electronic components together, they may be glued together using liquid mixture, only to then be encased in liquid nonconductor to provide the mechanical stability. Such a circuit, constructed truly only from hermetically sealed components that are connected and encased only in liquid mixture or liquid nonconductor, offers the advantage of being more mechanically stable, less chemically valent (no solder means less metals that may form half cells inside an aqueous medium), and be mass produced outside the body as fully cured system that may then relatively easily be implanted and then secured inside the body using the liquid mixture while connections to nerves may be utilized using the same liquid mixture that was used to connect the hermetically sealed components earlier to form the follower-circuit. Aside from follower-circuits, other electronics such as for sensing, amplification and stimulation can be constructed using such manufacturing principles.

### Neurostimulation Studies

### Impedance—Tissue & Cadaver Study

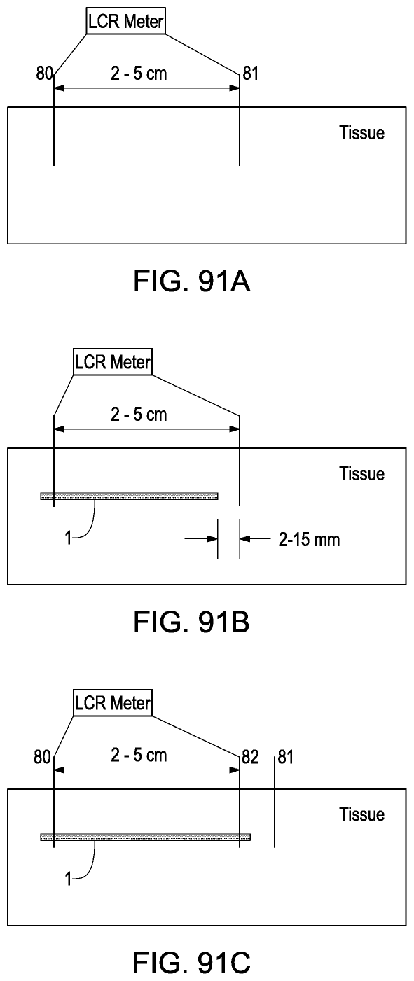

Tissue impedances of various samples were first measured without the present invention. Tissue impedances were measured with a LCR meter (DE-5000 Handheld LCR Meter; IET LABS, INC., Westbury, N.Y.) using a 1 kHz sinusoid by recording the impedance between two stainless steel wire probes 80, 81 inserted in animal tissue. Tissues examined were chicken muscle tissue, chicken sub-cutaneous tissue, pork muscle tissue, ham (processed pork), beef (muscle) and rat muscle tissue. First, stainless steel wire (SS 316L, 26 ga, Fort Wayne Metals) was placed into the tissue at a distance of 2 cm. The location was chosen such that the distance could be varied up to 5 cm. Caution was used to insert approximately 1 cm of wire into the tissue for repeatable metal to tissue interface areas. The LCR meter was connected to the stainless steel wire probes 80, 81 at distances between 2 and 5 cm apart. (FIG. 91A). Result: All impedances between 2 and 5 cm distance were determined to be between approximately 300 and 700 Ohms with the majority of tissue impedances recorded in the 500 to 700 Ohm range.

Next, electric field modification and tissue impedances were observed with the present invention. Cured electrodes 1 were placed into the meat by needle injection, originating from the location of one stainless steel wire probe 80 and bridging the distance to the second stainless steel wire probe 81 with varying gap distances between 2 mm and 15 mm of tissue left un-touched between the end of the cured electrode and the second stainless steel wire probe 81. (FIG. 91B). Result: impedances across the entire 2 and 5 cm distance were determined to be between approximately 150 and 270 Ohms and dependent primarily on the length of the gap between the end of the cured electrode 1 and the second stainless steel wire probe.

Finally, the cured electrode impedance was determined by placing a third wire probe 82 directly through the end of the cured electrode 1 closest to the second probe 81. The impedance of the cured electrodes did vary by length from about 0.25 to about 0.45 Ohms with smaller impedances correlating with shorter cured electrode lengths (FIG. 91C).

### Voltage Drop—Tissue & Cadaver Study

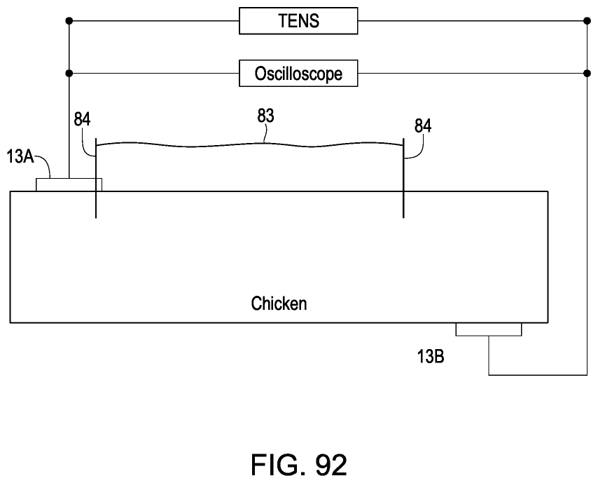

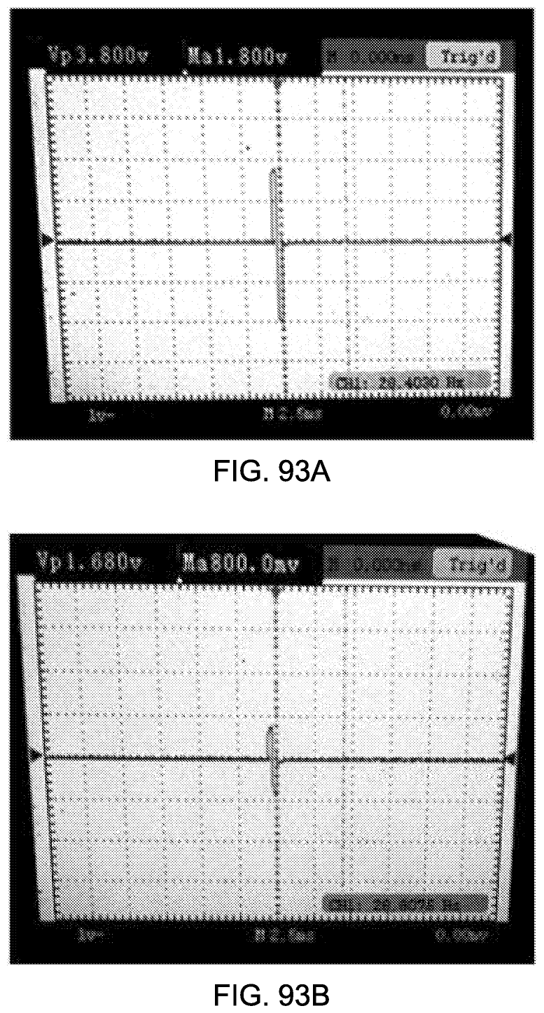

A voltage measurement was taken during TENS stimulation. A transcutaneous electrical nerve stimulator was applied with TENS electrodes 13 (cut to 1 cm square) to chicken meat (muscle, approximately 1 cm thick, 3 cm wide, 12 cm long). The electrodes 13 were placed approximately 8 cm apart and on opposite sides of the chicken meat. An oscilloscope was used to visualize the voltage needed to apply the current controlled biphasic stimulation waveform. A diagram of the setup is FIG. 92. The oscilloscope showed the voltage between the two TENS electrodes was 3.8 volts (FIG. 93A). The chicken tissue was wrapped into insulating foil to minimize dry out and parallel current paths through contacts on the table.

A 5 cm stainless steel wire 83 (line impedance <0.2 Ohm) with alligator clips was clipped to metal pins 84 and inserted through the short axis of the chicken and the wire placed into the chicken tissue. One pin 84 was placed in direct contact with one of the TENS electrodes 13A (“first electrode”), the other pin 84 was placed at varying distances along the long axis of the chicken tissue, but never the total distance to the second TENS electrode 13B. As the wire 83 produced a parallel low-impedance path along the long axis of the chicken tissue, the voltage measured by the oscilloscope dropped as driving the same current with the TENS unit was now possible through a lower impedance parallel path. The drop in voltage depended primarily on the size of the gap between the second TENS electrode 13B and pin 84 near it.

For gap distances larger than 50% of the distance (approximately 4 cm) between the two TENS electrodes, the voltage (e.g., 3.56 volts) needed to drive the current dropped some but not more than 30%. For small gap distances of about 1 cm of the distance between the second TENS electrode 13B and the nearest pin 84, the voltage (1.68 volts peak to peak) needed to drive the current dropped to values of about 50% of the total voltage needed if no shortening wire 83 was applied, as shown in the readout on the oscilloscope in FIG. 93B. For very small gap distances of <1 cm and especially <0.5 cm of the distance between the second TENS electrode and the pin, the voltage needed to drive the current dropped to values of about 20% of the total voltage needed if no shortening wire was applied.

A cured electrode 1 was placed by needle injection for the distance of approximately 3 cm into the chicken tissue and the outside TENS electrodes 13A, 13B were repositioned to allow a direct connection of the first TENS electrode to the cured electrode 1 while the second TENS electrode remained approximately 0.7 cm away from the cured electrode and the results were similar to FIG. 93B. The voltage needed to drive the same current through the chicken tissue dropped by about 65%. The voltage needed with the wire placed in parallel, shortening gap by approximately 90% resulted in a voltage drop of about 65% from the original value of 3.68 volts peak to peak.

### Rat Brachial Plexus

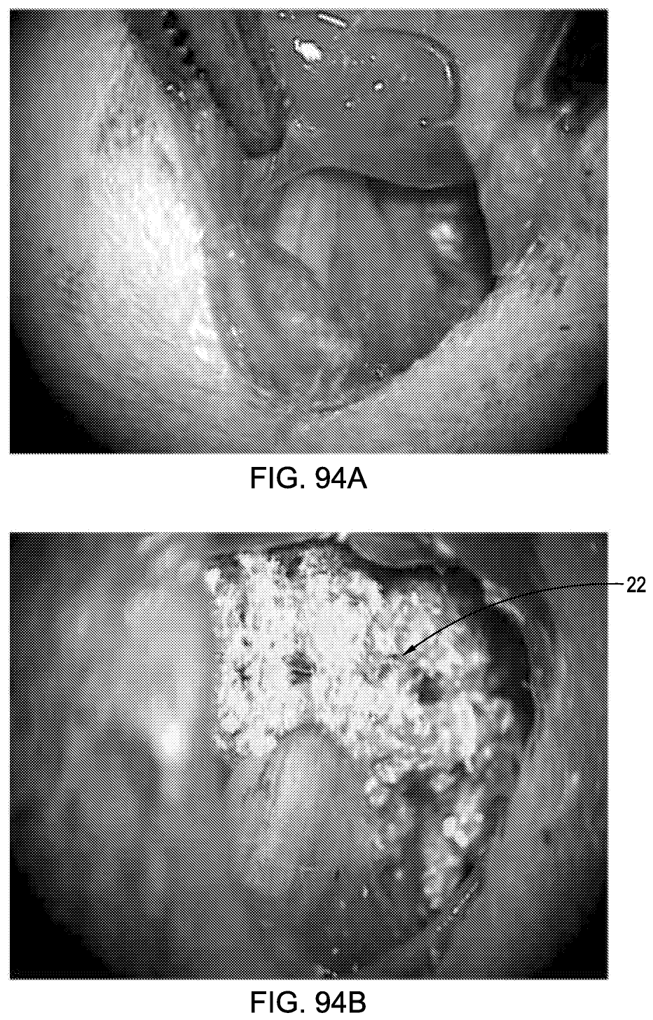

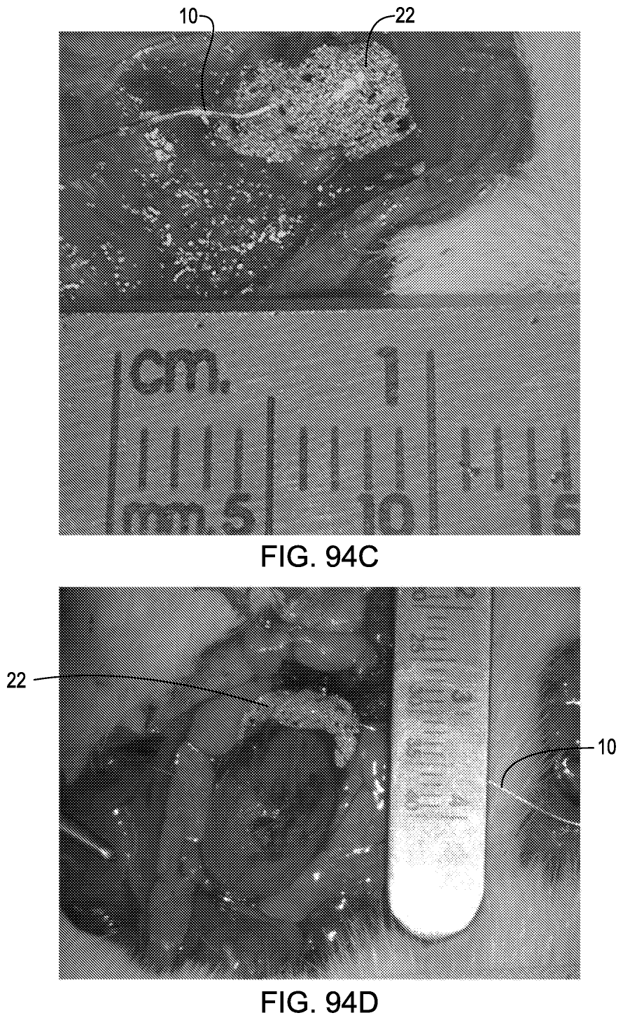

This study was performed on three rats, one animal at a time. Under deep anesthetic plane (anesthetic plane monitored using heart rate, breathing frequency, and paw withdrawal to toe pinch), the animal's left or right brachial plexus (sometimes “BP”) or both brachial plexi (left and right arm) were exposed with a small 5 mm incision (FIG. 94A). FIG. 94A is an image of obtaining access to the brachial plexus, with exposed nerves in the center. (If both BP were tested, testing was done sequentially to avoid tissue dry out.) Median, Ulnar and Radial nerve were freed from surrounding tissue but not from their respective nerve sheath; in one case two nerves ran together and it was not clear if median and ulnar had not yet separated at the surgical site. Cured electrode material (silicone and silver based, line impedance range from 0.2 to 0.4 Ohm*m) was injected behind and around the nerves, and the brachial plexus was surrounded as a ring-like portion 22 around the entire plexus (FIG. 94B). A lead wire 10 was sunken into the cured electrode material prior to curing (FIG. 94C). After the curing time of approx. 60 seconds, a signal generator 17 was attached to the wire 10 embedded into the cured electrode around the brachial plexus. A distal return electrode was achieved via a needle that was placed into the sub-cutaneous tissue near the lower back of the animal. Stimulation of the brachial plexus was achieved with signal waveforms of 1 Hz @ 0.5 mA, 30 Hz @ 0.5 mA, 30 Hz @ 1 mA, 30 Hz @ 2 mA, and 30 Hz @ 5 mA to differentiate various nerve fiber sizes. Nerve block was tested with 300 Hz ACh depletion block waveforms since the cured electrode provided a complete cuff. Parameters for block compared to stim were the same except for the frequency applied. To ensure stimulating and blocking all fibers, the parameters used were 30 Hz @ 5 mA for stimulation and 300 Hz @ 5 mA for block. Immediate block (onset duration <0.5 sec) was achieved successfully. In two animals, the incision was widened slightly and a second cured electrode was placed adjacent and more distally to the first one, about 1 mm away and without touching the first cured electrode placed earlier. A lead wire 10 was embedded before curing. Stimulation applied to the second cured electrode with the same as well as different parameters utilized for stimulation (1 to 30 Hz) showed different effects on lower arm, wrist, and paw movement. Stimulating both cured electrodes simultaneously provided combined movement resulting from the two cured electrodes. Applying stimulation waveforms to one of the cured electrodes while applying block waveforms (300 Hz at high amplitudes) to the second cured electrode led to flaccid paws and wrists as long as the block was applied.

### Rat Bladder Neck Study

Another neurostimulation study was on a rat cadaver performed with a cured electrode formed around the bladder neck (for access to nerves innervating the end organ) as shown before in FIG. 94A and after in FIG. 94B, In FIG. 94C and FIG. 94D, a lead wire was embedded in the cured electrode formed as a ring 22 and some more cured electrode material added for mechanical matching, by letting cured electrode material flow around a moment with slower curing time. The bladder neck is the primary path for nerves innervating (entering) the bladder tissue from the surrounding tissue inside the abdominal cavity. Placing a mechanically flexible electrode that conforms to the anatomical shape of the tissue of interest around the bladder neck provides a neural interface that can stimulate and block neural tissue in locations conventional electrodes do not conform to and thus do not perform well. The bladder was filled for demonstration purposes after curing of the molded electrode; the molded electrode remained in place and did not show major movement. This experiment demonstrated how a cured electrode may be placed around a flexible tissue composition or an organ at a specific target location in order to avoid having to manufacture electrodes outside the body and attempt to fit such a pre-manufactured electrode to a target tissue. The advantage of curing the electrode inside the body is to adapt to any anatomy of interest and, for specific mixtures, retain the ability to deform mechanically while retaining the ability to interface with the target tissue by means of energy injection (such as electrical current, thermal energy, light or others).

### Pig Brachial Plexus

After the rat brachial plexus data had been obtained, pig studies were also performed on the Brachial Plexus in three different designs. Materials used included: (1) Silicone electrodes: silicone, Kwik-Cast (World Precision Instruments), silver powder see powder specs below, and the surfactant, GLYMO; (2) PEG electrodes: CoSeal PEG 8 ml vial, Silver powder see powder specs below, and glycerol. Silver powder was the same as used in both formulations, conductive element sizes ranging from ˜0.6 micron to ˜6 micron, with aspect ratios ranging from ˜1 to 6 in a polydisperse system. Number average for the mixture was approximately between 2-3 aspect ratio, given the larger number of roundish elements seen. The silicone cured electrodes 1 comprised ˜73% wt % silver content: 200 mg Kwik-Cast (100 mg each of part A and part B), 800 mg silver powder, and 100 μl GLYMO. Silicone based cured electrodes were also provided a mixture with an added layer of Kwik-Cast added to the one surface to act as a selective insulator. The PEG cured electrodes 1 comprised ˜73% wt % silver content: 200 mg CoSeal PEG Reconstituted Using Supplied Syringe system (100 mg each of part A and part B), 800 mg silver powder, and 100 μl Glycerol.

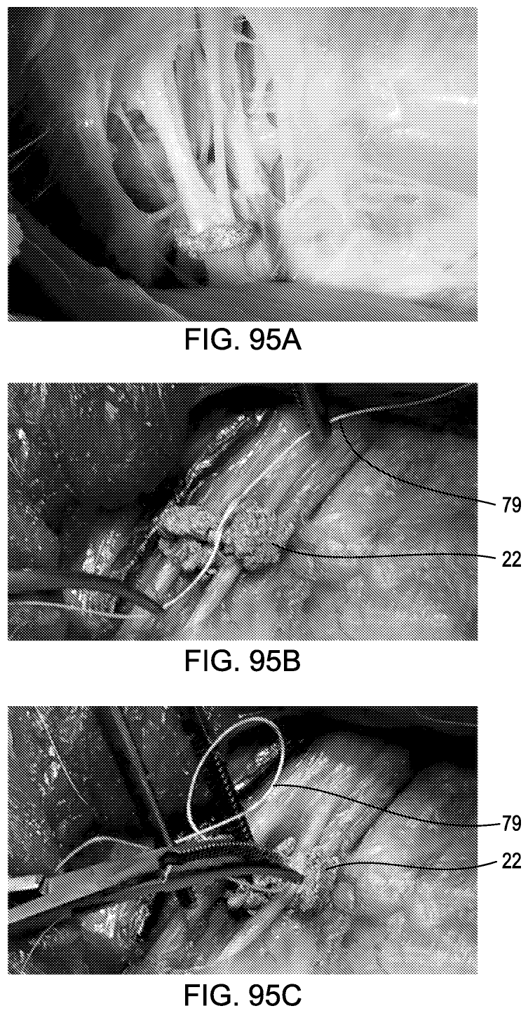

Study 1: This study was performed on 2 pigs, one animal at a time. The animals had just expired (defining the situation as tissue study) and allowed approximately 10 minutes of study time prior to ATP depletion. The animal's left brachial plexus (BP) was exposed with a large 10 cm incision to allow optimal visualization for documentation purposes. The nerves of the BP were carefully exposed and freed from the tissue underneath, and an electrode material mix was molded around these nerves to form a cured electrode. The ring-like portion 22 of a cured electrode was allowed to cure fully within 60 seconds. FIG. 95A. A handheld TENS signal generator was used to stimulate the nerves with current controlled biphasic, charge balanced waveforms. The TENS unit electrode contact associated with the cathodic first pulse of the waveform was used to temporarily touch the nerves of the BP as well as the cured electrode around said nerves, while the anodic first (TENS counter) electrode was placed as distal return by clamping it into the open cut down approximately 10 cm away from the cured electrode. Stimulation of the brachial plexus was achieved with signal waveforms of 2 Hz applied at a current amplitude that did cause the nerve to depolarize and arm muscles to twitch at 2 Hz when the cured electrode was touched, but not to depolarize when the nerve was touched with the probe contact coming from the TENS unit directly, either proximally or distally to the cured electrode. The study confirmed that the cured electrode is able to provide a low impedance interface and a concentration of the electric waveform energy to the nerve surrounded by cured electrode material and that activation thresholds are lowest with such a cuff configuration, lower than touching the nerve with the probe contact (probe tip surface area approximately 1 mm2). FIG. 95A is an image of the pig Brachial Plexus with the cured electrode molded during open cut-down. The proximal portion of the nerve is located south with respect to the cured electrode in the figure, the distal portions are north of it.

### Pig Cadaver Study

Following earlier benchtop studies and studies in chicken tissue, a study was performed on a pig cadaver to replicate the cutting of a cured electrode with a suture. FIG. 95B is an image of forming a knot with a suture 79 and pulling on the knot with two surgical clamps. FIG. 95C is an image of pulling on the knot with two surgical clamps and checking the path the suture took through the cured electrode. Note that pulling the knot split the cured electrode ring-like portion 22 into two sections, allowing this now C-shaped cured and cut electrode to be removed by grabbing it with tweezers and pulling it away from the nerve.

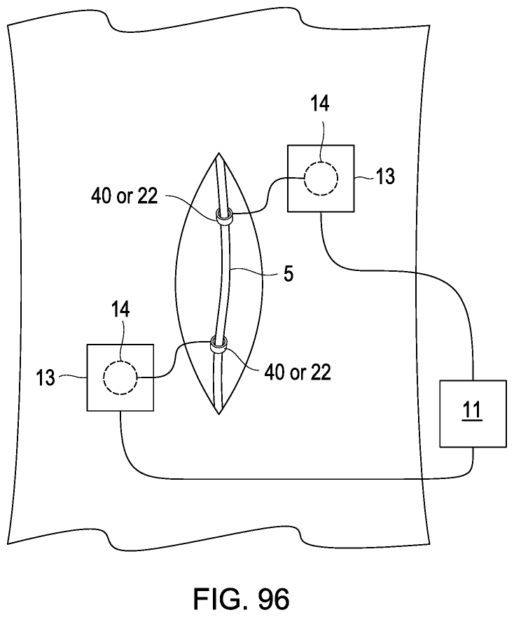

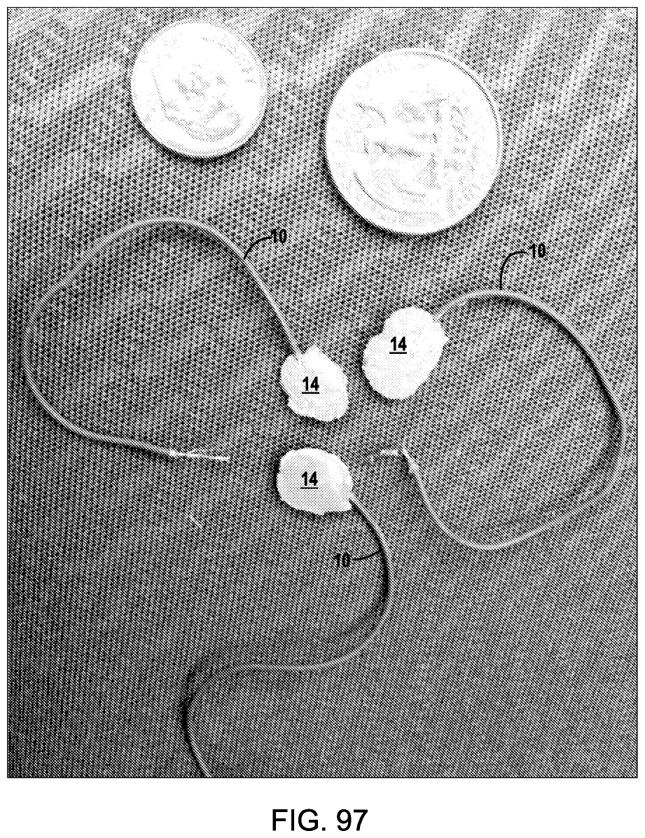

Following earlier studies on chicken (see above) and rat tissue demonstrating the ability to reduce voltage needed to bridge a current path through tissue by placing a cured electrode in parallel to the tissue, thereby shortening the distance between low-impedance elements of a circuit containing animal tissue in between opposite electrical potentials, a pig vagal study was performed in two pigs. This study was able to replicate the reduction of impedance by placing cured electrodes into the tissue, bridging distances to the nerve with low impedance materials (cured electrode and attached wire in this case). The study further demonstrated the ability to reduce Heart Rate with such a cured electrode and it demonstrated the ability to reduce Heart Rate with an external stimulator TENS unit that was never in direct contact with metal inside the animal. For the procedure, an animal on the table was placed into a deep plane of anesthesia. A vagal cut-down was performed to openly expose the vagus nerve. Two prior art cuff electrodes were placed around the vagus nerve and the lead wire from these cuffs was connected to a cured electrode placed into the sub-cutaneous tissue of the pig near the vagal exposure. TENS electrodes and a TENS stimulator were used to stimulate transcutaneously by electrically connecting to the subcutaneously placed cured electrodes through the skin (without a direct connection through the skin as the skin above the cured electrodes was never damaged) which in turn were connected to the cuffs around the vagal nerve. The study setup and cut down is diagrammed in FIG. 96 showing the elements internal to the animal with TENS patch electrodes 13 placed on the outside of the animal above the contact pad 14 just underneath, allowing vagal stimulation through the skin without damaging the skin. The two contact pads 14 (image in FIG. 97 next to coins) are placed subcutaneously, then connected to cuffs (either prior art cuffs 40 or formed as a ring-like portion 22 of a cured electrode live in pig). Each cured electrode and a corresponding TENS patch FIG. 13A/13B above is diagrammed in FIG. 96.

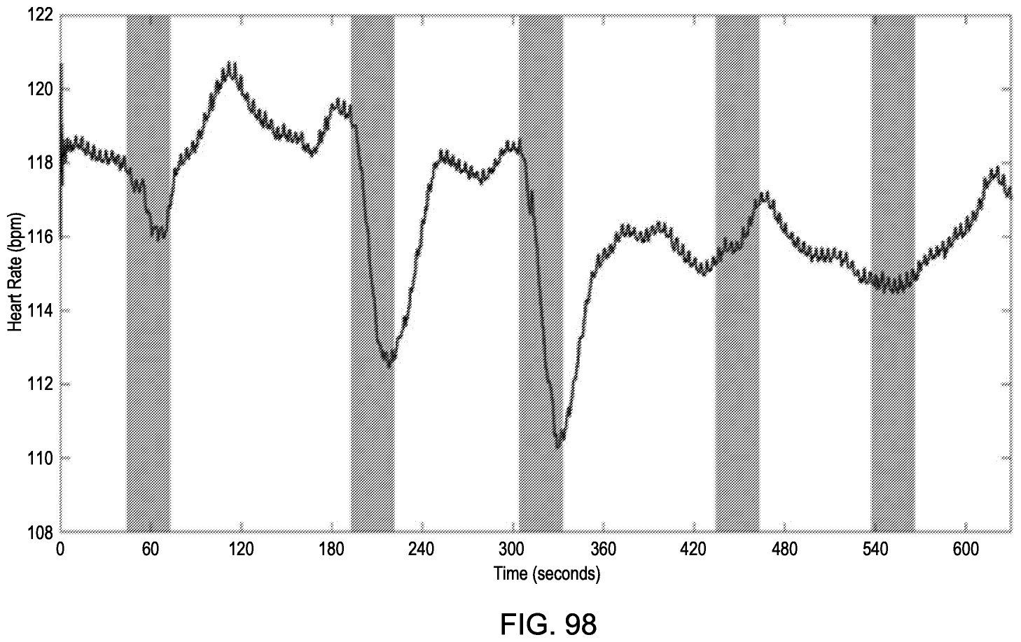

Five stimulation tests were performed: (1) low amplitude stimulation, (2) mid amplitude stimulation, (3) high amplitude stimulation, (4) removal of the subcutaneously placed contact pad 14 that connected to the cathode to test for leakage driving the HR reduction, with no leakage detected, and (5) removal of the subcutaneously placed contact pad 14 that connected to the anode to test for leakage driving the HR reduction, with no leakage detected. The results are shown in the chart, FIG. 98, which plots heart rate (bpm) versus time (seconds). The low amplitude stimulation (55 sec) provides a first response. The mid (200 sec) and large (305 sec) amplitude stimulation provide a strong HR reduction. Once the subcutaneously placed cured electrode under the cathodic TENS electrode was removed, stimulation did not result in changes to HR, indicating that the current flow had been via the cured electrodes and not via the open cut-down (430 sec and forward; control tests).

A comparison of electrodes was conducted for a Livallova prior art cuff (FIG. 99A) versus the cured electrode (FIG. 99B). The present invention had the larger capacitive charge injection capabilities. At frequencies of 2 kHz and above, the cured electrode was about ⅓ of the impedance of the Livanova cuff (100 ohms vs. 300 ohms), which saves battery energy for an implanted pulseform generator due to the lower voltage needed to drive the same stimulation current; one would expect a stimulator to require ⅔ less power to drive the same charge into surrounding tissue when using the present invention. The cured electrode demonstrated strong capacitive charge injection capabilities for the injection of current.

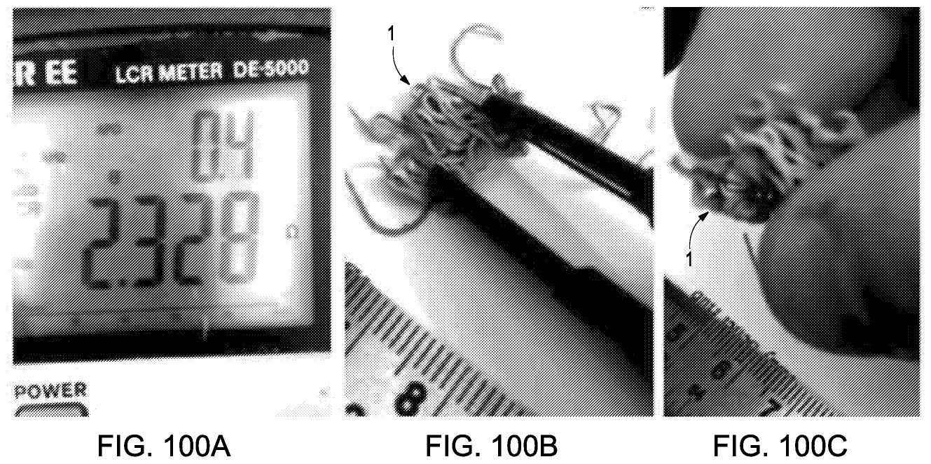

Very thin cured electrodes and wires (<1 mm) as extruded from a dispenser are shown in FIG. 100B and FIG. 100C. The impedance as shown on an LCR meter was 2.328 ohms, as in FIG. 100A, measured across the length of several turns and twists of the extruded electrode in the shape of a wire, further confirming that the impedance of each smaller section of the cured extruded shape is smaller than 1 Ohm.

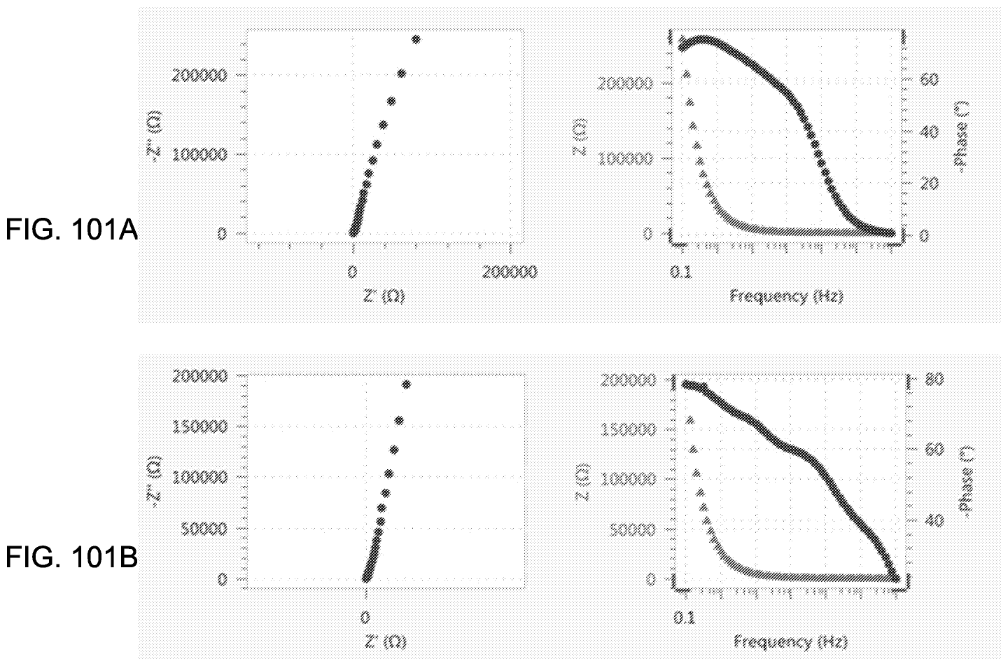

FIG. 101A reports Impedance Spectroscopy and a Nyquist plot for the prior art Livallova cervical vagus cuff electrode, and FIG. 101B reports Impedance Spectroscopy and a Nyquist plot for a silver/silicone (78% ag) cured electrode of same dimensions as the Livallova cuff. This data was recorded in PBS using an Autolab (AUT128N.FRA32M.v PGSTAT128N with FRA32M Module) for the commercially available Livallova cervical vagus electrode (101A) and compared to a silver-silicone (78% Ag) cured electrode (101B) of similar dimensions to further validate the AC and DC impedances measured in earlier experiments. The cured electrode showed comparable results to the Livallova cuff, though the recorded absolute impedance values for the cured electrode were lower (more favorable).

### Magnetic, Thermal, Vibratory and Optical

Energy is the property of matter and radiation (element/wave combination) that manifests as the ability to perform work. By transmitting energy from one source to a target location, work may be initiated or performed at the target location. If a target location is neural tissue, then energies may be transferred along a waveguide. The cured electrode 1 may be understood as exactly that, an energy wave guide cured in vivo at or nearby the target stimulation, block or ablation site. The conductive elements 6 may conduct electrical, magnetic, thermal, acoustic or vibrational energy, or combinations of these forms of energy, to transmit energy from a location to another one inside the body. Such a transfer may happen from a location at the surface or just beneath the surface of the skin to a location several millimeters or even several centimeters deep inside the body away from the skin. Such transfer may also happen from one energy signal generator to another energy transformer, which in turn may be connected to another energy transformer or a biological tissue inside the body. One or more than one type of energy waveguide may be used inside a body to achieve a modulation in organ activity, metabolic activity of tissue and other effects to change clinical and preclinical research and treatment paradigms.

In the case of electrical energy conduction, the cured electrode may comprise an electrically non-conductive material which is combined or functionalized with electrically conductive elements or electrically conductive functional groups which lowers the impedance of the overall mixture to allow the cured electrode to conduct electricity. This cured electrode optionally may be surrounded at least in part by a nonconductive layer, as an insulator, stabilizer or anchor.

In general, the term “electrically conductive” means impedance values (for a specific volume of e.g. 1 mm high by 1 mm width by 1 mm length) of <1 ohm for the electrically conductive elements themselves (meaning the additive that increases conductance for the combined mixture). A cubic volume (e.g. of 1 mm by 1 mm by 1 mm) “mixed or combined electrically conductive cured electrode” has an impedance value of <100 ohms as a sufficient value, <10 ohms as a good value and <1 ohm as an optimal value. This means that an optimal value material would have a volume impedance of <1 ohm*cm. Likewise, a cubic volume (e.g. of 1 mm by 1 mm by 1 mm) of an “electrically non-conductive” layer 9 or material 9A has a minimum impedance value >100 kilo-ohm for the “electrically non-conductive” carrier as well as, and preferentially >1 mega-ohm. The electrically conductive cured electrode may further provide a large capacitive and relatively small resistive interface to a saline electrolyte such as interstitial fluid inside a living organism or phosphate buffered saline (PBS) in a representative beaker. A magnetically conductive cured electrode comprises magnetically non-conductive (having low ability to form magnetic field lines within itself; being non-preferentially-permeable or non-permeable) carrier material which is combined or functionalized with magnetically highly-permeable (high ability to form magnetic field lines within itself) elements (e.g., iron), thereby providing a preferential path for magnetic field lines of any magnetic field applied from outside the cured electrode as well as if it were applied at least in part from within the cured electrode. There may also be a magnetically insulating version of the magnetically conductive cured electrode that disperses magnetic fields (diamagnetism for specific frequencies of changing magnetic fields) to provide a high magnetic impedance while providing similar mechanical features or an excellent mechanical (and or chemical, biological and/or biochemical) integration with the magnetically conductive cured electrode.

In general, the term “magnetically permeable”, or alternatively, “magnetically conductive” or alternatively, “magnetically guiding” means the ability of a material to conduct magnetic field lines (giving rise to magnetic flux) within itself for a specific volume (of e.g. 1 mm high by 1 mm width by 1 mm length) of a specific magnetic reluctance (akin to “magnetic resistance”). The magnetic reluctance of a volume (measured in 1/Henry) is dependent on its magnetic permeability which is the measure of the ability of a material to support the formation of a magnetic field within itself, especially when a magnetic field is applied from the outside, thus guiding the magnetic field lines through the said material. It is thus the degree of magnetization that a material obtains in response to an applied magnetic field. The absolute permeability of vacuum is μ_zero=4*pi×10-7 Henry/m which equals the relative permeability of 1. Any material of significantly larger permeability μ_N=N*μ_zero preferentially guides magnetic field lines through the inside of itself. Magnetically conductive elements providing the increase in permeability of the whole magnetic waveguide possess relative permeabilities of at least >=100 (such as carbon steel or nickel alloys), preferentially >=1000 (such as ferritic stainless steel or electrical steel) or in optimal cases values of >=10000 times the vacuum permeability (such as Adv. crystalline permalloys Ni₈0Fe₂0 and others).

The overall biocompatible mixture of magnetically liquid nonconductor (having a permeability close to vacuum permeability) and magnetically conductive elements (having a permeability several orders larger than vacuum permeability) offers a resulting permeability that is smaller than the permeability of the elements themselves but much larger than the permeability of the carrier or the biological tissue that it may be injected into/placed onto. The magnetically conductive elements may for purposes of increasing their biocompatibility be covered in part or completely in other materials that are not significantly affecting the overall permeability of the mixture, but shield the highly magnetic permeable material from the biological environment. One such embodiment are iron microelements that are coated in several nanometers of gold, the goal covering providing a bioinert interface for the cells of the body, while the iron core provides the increase in magnetic permeability of the composite element. These composite elements may then be suspended in a magnetically transparent (non-conductive) carrier such as silicone or PEG.

Examples for magnetic elements include, without limitation, 1) a sintered Nd₂Fe₁₄B compound of high saturation magnetization (Js ˜1.6 T or 16 kG), a rare-earth magnet, meaning a permanent magnet made from an alloy of neodymium, iron and boron to form the Nd₂Fe₁₄B tetragonal crystalline structure, 2) stainless steel with ferromagnetic iron components (primarily magnetic variants such as 440 or 420 stainless steel, and 3) ferrite elements in stainless steel. The following table is relevant here:

TABLE EIGHT
Magnetic Absolute Permeability and Relative Data For Selected Materials

| | | Relative |
|---|---|---|
| | Permeability, μ | permeability, |
| Medium | (H/m) | max., μ/μ0 |
| max. | | |
| Metglas 2714A (annealed) | 1.26 × 100 | 1000000 |
| Iron (99.95% pure Fe annealed in H) | 2.5 × 10−1 | 200000 |
| NANOPERM ® | 1.0 × 10−1 | 80000 |
| Mu-metal | 2.5 × 10−2 | 20000 |
| Mu-metal | 6.3 × 10−2 | 50000 |
| Cobalt-iron (high permeability strip | 2.3 × 10−2 | 18000 |
| material) | | |
| Permalloy | 1.0 × 10−2 | 8000 |
| Iron (99.8% pure) | 6.3 × 10−3 | 5000 |
| Electrical steel | 5.0 × 10−3 | 4000 |
| Ferritic stainless steel (annealed) | 1.26 × 10−3 − | 1000-1800 |
| | 2.26 × 10−3 | |
| Martensitic stainless steel (annealed) | 9.42 × 10−4 − | 750-950 |
| | 1.19 × 10−3 | |
| Ferrite (manganese zinc) | >8.0 × 10−4 | 640 (or more) |
| Ferrite (nickel zinc) | 2.0 × 10−5 − | 16-640 |
| | 8.0 × 10−4 | |
| Carbon steel | 1.26 × 10−4 | 100 |
| Nickel | 1.26 × 10−4 − | 100-600 |
| | 7.54 × 10−4 | |
| Martensitic stainless steel (hardened) | 5.0 × 10−5 − | 40-95 |
| | 1.2 × 10−4 | |
| Austenitic stainless steel | 1.260 × 10−6 − | 1.003-7 |
| | 8.8 × 10−6 | |
| Neodymium magnet | 1.32 × 10−6 | 1.05 |
| Platinum | 1.256970 × 10−6 | 1.000265 |
| Aluminum | 1.256665 × 10−6 | 1.000022 |
| Wood | 1.25663760 × 10−6 | 1.00000043 |
| Air | 1.25663753 × 10−6 | 1.00000037 |
| Concrete (dry) | | 1 |
| Vacuum | 4*pi × 10−7 (μ0) | 1, exactly |
| Hydrogen | 1.2566371 × 10−6 | 1 |
| Teflon | 1.2567 × 10−6 | 1 |
| Sapphire | 1.2566368 × 10−6 | 0.99999976 |
| Copper | 1.256629 × 10−6 | 0.999994 |
| Water | 1.256627 × 10−6 | 0.999992 |
| Bismuth | 1.25643 × 10−6 | 0.999834 |

A magnetically conductive cured electrode 1 may be interfaced with electromagnetically in order to enable mechanical (force) interaction between the curing or cured electrode and nearby biological tissue with the intent to compress, stretch or vibrate the nearby biological tissue, or other non-biological elements that in turn may convert mechanical energy to other forms of energy (such as piezo electronic elements that may be subjected to pressure changes to generate electrical differential potentials). A coil on the outside of the body is able to induce a electromagnetic field that may interact with the magnetically conductive cured electrode, setting it in motion and thereby transmitting mechanical forces via electromagnetic means. In one embodiment, the magnetically conductive cured electrode may be placed near proprioceptive sensory innervation of the skin to provide means of communicating with the tactile sensory system of the body by electromechanical means. In another embodiment, the liquid mixture may be deployed as an injectable that may pre- and/or post cure transmit mechanical forces to the surrounding tissue, in combination with a generator of a time-variant magnetic field (such as a coil supplied with a pulsed or an alternating current) and may be used to convey information to a person by providing an interface that allows location specific, amplitude specific and frequency specific means of information transfer. In one embodiment, a multitude of magnetically cured electrodes may be injected into the subcutaneous tissue just above the skull of a user to be able to utilize a multitude of coils placed in a helmet to transmit directional information, such as the information about oncoming traffic, a ball within a ball game or an approaching heat signature in the middle of the night. By driving larger or smaller sinusoidal currents through specific coils placed in a helmet, the location as well as the distance of an oncoming or leaving object may be conveyed to the trained user within a sub-second interval and without the need of the user to see the approaching or distancing object or subject. A helmet for such an application includes, without limitation, e a motorcycle helmet, an airline pilot's helmet, a construction worker's helmet, a police officer's helmet, a football player's helmet or alike.

Magnetically conductive elements 6 that are added to a magnetically non-permeable (magnetically reluctant) carrier may include ferrites (ferrite in ceramic form that by itself is electrically non-conductive but magnetically conductive), ferromagnetic elements, ferrimagnetic elements and other, highly permeable materials. Details of the magnetically conductive cured electrode and described below are embodiments in a helmet, a shoe, an example of underwear, a belt and other implementations. These mixtures (suspensions) of non-magnetically interacting bio-compatible carrier material 9 combined with magnetically interacting conductive elements 6 allow for the formation of in-body cured magnetically interacting composite mixtures (“cured suspensions”). Such a mixture may be injected via needle & syringe, then locally forms based on the local anatomy and may adhere to some bodily tissues providing added mechanical interaction and minimization of risk due to shear forces exhibited by the injected (placed) material.

Magnetically induced vibration of tactile/proprioceptive sensory tissues as they are present in the sole of feet, on hands, or for example in the vicinity of the anal or urethral sphincter may be utilized to provide strong sensory input to the patient with the intent to activate or interrupt reflexive behavior. One example is the placement of the magnetic liquid mixture 1 near (around/adjacent/into) the external anal sphincter muscle for the treatment of fecal incontinence. Another example is the placement of the magnetically conductive cured electrode near (around/adjacent/into) the external anal sphincter muscle for the treatment of urinary incontinence. The cured magnetically interacting composite mixture can be vibrated at specific frequencies (e.g. 10 Hz, 20 Hz, burst vibration at 50 Hz burst frequency for 1 second on/1 second off intervals) can be used to provide a patient with a proprioceptive input to activate or strengthen already present activation of the sphincter muscles and provide either anal or urinary continence or both. Placement of a magnetically interacting composite mixture around the anal sphincter may due to reflexive connections between the anal and the urinary system be used to provide not only anal continence but also urinary continence. The magnetically interacting composite mixture may be activated via coil(s) placed into a belt, underwear, outer wear or other devices with the ability to create a changing magnetic field (such as a magnet attached to a rod that is rotated by a motor) to induce the changing magnetic field that couples with the implanted magnetically interacting composite mixture placed in the vicinity of the respective sphincter.

Instead of adding elements aimed at the transfer of electrical current or magnetic flux, elements optimal for the transfer of heat may be combined with a biocompatible carrier medium that is optimal for curing inside the body. Such an energy waveguide for thermal energy may as a side effect also transfer electrical or other forms of energy besides heat, but the main focus is to transfer thermal energy. For that purpose, thermally conductive elements are added to an in-body curable carrier medium that by itself does not conduct thermal energy with the same high thermal conductivity.

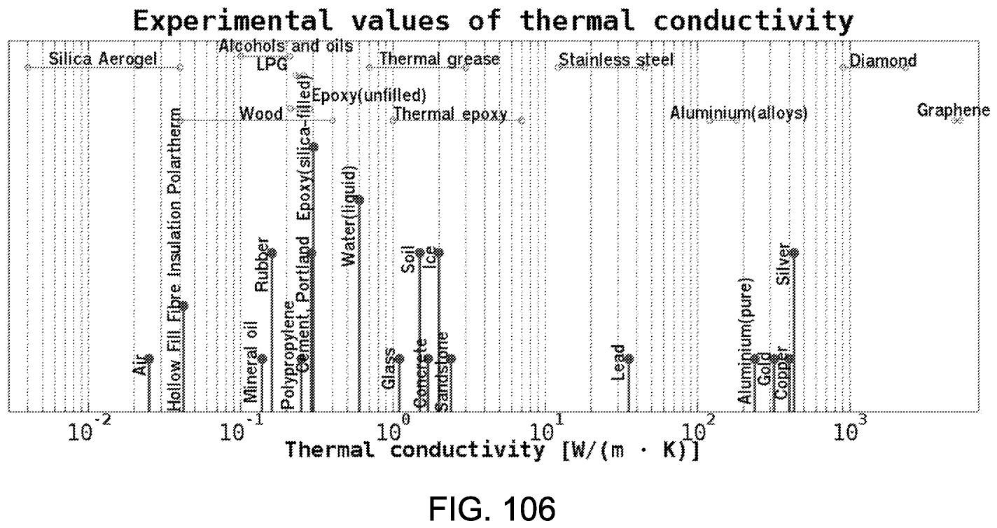

To achieve long-term stable thermally conductive cured heat waveguides, examples for elements added in i.e. powdered form may come in sizes of >1 um in at least one dimension (>1 um as a minimum, >20 um as an optimum for increased long-term stability as macrophages are less likely to engulf >20 um elements, for extreme examples even >100 um in at least one dimension further ensuring long-term stability). These elements may be composed of various biocompatible materials such as diamond or graphene, gold, platinum, titanium and other metals known to be stable and non-corroding in the body's environment. Examples for materials are shown in FIG. 106, the most thermally conductive being located upper and right.

Such a thermally conductive cured electrode may be used to transfer heat from one location inside the body to another location, or to transfer heat from one type of tissue or one organ inside the body to another type of tissue or another organ. The thermally conductive cured electrode may be used to transfer heat from the inside of the body to a location just below the skin of the body, allowing for a cooling of the inside of the body by dissipating heat to the skin of the body where the body's sweating process allows for a heat dissipation to the environment.

The thermally conductive cured electrode 90 may be used to transfer heat from an organ to a Peltier element driven at a direct current and thus operated as heat pump to drive heat from one side of the Peltier element to the other side. Such a Peltier element 90 is hermetically encased e.g. via ceramic can that may or may not have an increased heat conduction at specific contact points by employing thermal vias (metal bridges) that are soldered hermetically into the walls of the can in order to withstand the body's inner environment. The thermally conductive cured electrode 1 forms a heat bridge with a much higher thermal conductivity than the surrounding tissue, thus transferring heat from one location distant to the Peltier element 90 to the element on the cooling side of the Peltier element, and transferring heat from the Peltier element to one location distant to the element on the heating side of the Peltier element.

Thus, the cured electrode may function as a guide for thermal energy to conduct heat from one type of tissue to another. One embodiment conducts heat from one type of tissue to a Peltier element, which uses electricity to heat one of two places up while cooling the other plate down. This embodiment may be used to conduct heat from a Peltier element to an organ, or from an organ to a Peltier element. The thermally conductive cured electrode has embodiments which may facilitate applications that utilize the increase or decrease of metabolic activity in various tissues, provide neural block, or change the reflexive behavior of organs such as a bladder whose temperature receptors respond differently for cold urine than for warm urine. The thermally conductive cured electrode may be used to conduct heat generated (by e.g. a Peltier element) to a tissue inside the body that responds to heat treatment for the reduction of pain. The thermally conductive cured electrode may further be used to conduct cold generated (also by e.g. a Peltier element) to a tissue inside the body that responds to cold treatment for the reduction of pain. This cured electrode may contact the tissue directly to transfer heat (cold) or it may do so indirectly by directly contacting bodily vessels that transport i.e. blood (or interstitial fluid, or cerebro-spinal-fluid CSF, or other fluids) to the tissue that is to be heated (or cooled).

Thermal reduction of metabolic activity in cancerous tissue may aid with the reduction of cancer growth and inhibit the cancer's ability to spread via metastasis throughout the body. The effect utilized to cool an entire tumor is to cool blood vessels supplying a tumor, while sourcing heat from blood entering the tumor. The Peltier element may function as a heat pump with the cold side of the element placed near the blood vessels supplying the tumor. The hot side of the Peltier element is facing away from the blood vessels and thermally conductive cured electrode is used to increase the heat dissipating surface area and allows for interfacing with a variety of tissues and organs inside the body, which thereby function as drain for the collected heat pumped from the blood vessels supplying the tumor. Similar, the metabolic activity in organs may be reduced for medical purposes such as when localized cooling prevents organ damage (i.e. induced coma post cardiac arrest or post stroke to preserve healthy cardiac or brain tissue). Local cooling of blood vessels from one or more sides or even in the shape of a blood-vessel surrounding cuff may help to locally transfer heat out of the supplying blood, thereby providing organ or tissue cooling.

Thermal nerve block may be provided in a similar form to peripheral nerves by either cooling said nerves/ganglia/plexi directly or by cooling the blood supply to said neural structures. The cured electrode, in another embodiment, may function as a guide for optical energy, both in the visible and in the non-visible spectrum. This embodiment may be used to conduct light from one type of tissue to another. The light may be scattered within and to some degree out of the light guide the cured electrode provides and the light may be focused, or concentrated, close to or at the target tissue.

The cured electrode, in another embodiment, is conductive for acoustic, Ultrasound and Vibration energy. Ultrasound and sub-ultrasound waves may be transported (guided) preferentially along a cured electrode in order to concentrate the sound (vibrational) energy onto neural or other for activation or block of said tissue, or the cured electrode may conduct the sound waves to tissue that a patient is reporting as painful. Such tissue may be boney tissue, muscle tissue, cartilaginous or joint tissue, that responds to sound, vibration or ultrasound treatment but requires very high ultrasound, or sound energies at the outer skin level of a person to be effective, which is where the cured electrode helps to direct the vibration, sound or ultrasound energy to specific points and thereby allows significantly reduced energies to be applied on the outside of the skin to achieve the same sound/vibration/ultrasound energy densities at the target tissue as if the signal generator were much closer to the target tissue. The cured electrode may provide a focusing effect too.

The cured electrode, in another embodiment, is conductive for a combination of magnetic and vibration energy. A magnetic field may be used to mechanically actuate (“vibrate”) the cured electrode to magnetic fields. This cured electrode may be coupled to proprioceptive cells to signal information. This cured electrode may also be coupled mechanically to body tissue or to muscle tissue or other tissue to alleviate pain.

Vibration is often used in the clinic to temporarily block pain by providing a masking input to e.g. a muscle, together with proprioceptive sensory tissue in the muscle, the tendons and the surrounding tissue such as skin sends neural information back to the spinal cord or the brain, signals that travel on myelinated nerve fibers that are faster than c-fibers carrying pain signals, thereby masking the pain signal in the spinal cord of brain following the principal of the gate control theory of pain. The cured electrode, mechanically excited by magnetic or electro-magnetic stimulation or even vibration such as sound or ultrasound, may be used to generate such a vibration deep inside the body and thereby provide sensory input to the autonomic nervous system (changing the activity of reflexive circuitry), the proprioceptive system of the body or tissue innervated by both, c-fibers that sense pain and larger nerve fibers that sense motion, tickle, or vibration. The induced deep tissue vibration may be used to mask pain on demand for users that have reoccurring pain in specific regions that respond to vibration. Such a treatment may help to acutely reduce the sensation of pain as well as reduce the chronic perception of pain by reducing inputs to the spinal cord and brain that trigger heightened pain sensitivity with continuous presence of pain.

In another embodiment, the cured electrode is conductive for a combination of thermal and electrical energy. By combining materials and components conductive for heat and electricity, no-onset nerve block may be applied by first cooling neural tissue down, prior to applying electrical nerve block. The thermal nerve block may only be applied for a short period of time without unwanted side effects but long enough (in seconds to a few minutes) to allow for a fully established electrical nerve block that may be induced with KHFAC kilohertz waveforms, synaptic neurotransmitter depletion block waveforms or charge-balanced non-destructive direct current waveforms. The thermal nerve block and electrical nerve block may further be alternated to achieve a thermal nerve block during periods when the electrical nerve block is impossible or less likely, such as during an anodic recharging of the electrode-electrolyte interface that balances the introduced charges placed during the cathodic blocking period. Thermal and electrical nerve block may further be alternated to minimize unwanted side effects (such as remaining nerve block caused by electric means after applying the electric block too long) while retaining a partial or full nerve block. Both, thermal and electrical nerve block applications may be utilized fully as well as partially.

In order to achieve a repeatable partial cold nerve block with a cured electrode utilized primarily as a thermal conductor, two or more Peltier elements 90 are needed: one large active Peltier element to provide the heat transfer from a neural target, the neural target being connected to the cold side of the large active Peltier element via a cured thermal electrode, and a passive (and much smaller) Peltier element providing a measurement of the actual temperature of the cured thermal electrode 1 right next to the neural target. The passive Peltier element is connected to a reader (i.e. voltmeter) to determine various temperatures and correlate these with their specific nerve block effects: the temperature at which the first effects of a nerve block are noticed is considered the smallest thermal nerve block and may be achieved at temperatures of approximately 15 degrees centigrade, thereby recording the first point on a calibration curve. Then temperature at which the maximal effects of a nerve block are recorded as the maximal (100%) thermal nerve block and may be achieved at temperatures of approximately 5 degrees centigrade and less, thereby recording the second point on a calibration curve. While the temperature to block relationship for each patient may not be a linear but instead a sigmoidal one, it is important to record the temperature points that allow the desired nerve block effect, such as the reduction or even absence of a certain specific pain or spastic muscle contraction, specific organ activity or alike. This point, considered the active effect point, is the thermal block that must be measured by the small passive Peltier element in order to provide the patient with a repeatable nerve block experience.

The described partial thermal nerve block may be augmented by electrical nerve block, be it by providing a thermal block, then an electrical block and alternating between the two of them or by overlapping the two for a summation effect that may be more the sum of its parts (holistic effects of the system going beyond the sum of its parts).

The cured electrode 1 has properties which take advantage of the fibrous tissue encapsulation to mitigate component migration of the cured electrode. In simple terms, encapsulation is the body's response to an implanted object, and occurs in stages over time. Encapsulation begins within minutes of placing a foreign object into a living organism. A network of cells (i.e. platelets) and biological and chemical bonds, connections and elements (i.e. fibrin bonds) exert mechanical forces onto the foreign object as a whole as well as components of the foreign object as singular entities as well. The cured electrode 1 comprises conductive elements (such as i.e. micrometer and sub-micrometer (0.1 um to 0.99 um) size components of a cured electrode) for the application when its components are intended for a chronically un-stable cured electrode that may be processed in its entirety over an extended period of time of several months. Where the chronically stable electrode is to have only the carrier be fully or partially replaced, conductive elements and elements are to be from the small (1 to 10 um) to medium (10 to 20) and large (20 to 500) size micrometer range, the range of 20 to 100 um range elements being understood as too large to be processed and removed by macrophages, thus enabling long term stability without sacrificing large surface area mixtures that may be injected by small diameter (1 mm or less) needles. These elements are held in place post-injection by the body's own tissue which encapsulates these elements. The inflammatory response affects any size implanted object within the body with an exposed surface area to the biological tissue that initiates an inflammatory reaction leading to an encapsulation of the foreign object, such as the cured electrode. Encapsulation 52 creates within one week a network of mechanically robust fibers of sufficient tensile strength to hold micrometer size elements in place and prevent them from diffusing away from the implantation site. Encapsulation of the cured electrode may be enhanced intentionally by adding mechanical input to a cured electrode (e.g., small vibrations). Encapsulation may be enhanced by adding biological or chemical input to a cured electrode (such as cells and cell fragments enhancing the inflammatory response). The encapsulation ensures a reduced bio-availability of the foreign object within the body. The modulation (increase or decrease of the encapsulation) may change the bio-availability and chemical availability of the cured electrode as a whole or its constituents within the body. A thicker and/or denser encapsulation 52 reduces bio-availability, further limiting the body's cells being subject to unwanted interactions with the cured electrode, in whole or in part, over time. This process is discussed elsewhere herein.

The invention herein is highly scalable because its shape is conformable to any location in the body. Anatomy, the “art of how to cut properly”, is the scientific study of the structure of organisms that describes the “norm” of how biological structures such as organs, tissues, from organ subunits to conglomerates within a living body are shaped, aligned and connected. Even though there is a “norm”, there is great variation among individuals. In fact, even though one's left side is similar to one's right, anatomical substructures in something as simple as the neural structures in one's left arm are slightly different in shape from those on one's right arm. As a result, there is no optimal pre-shaped electrode that will fit on one person's nerve trunk X that will fit equally well in another person's nerve trunk X in the same location unless the pre-formed electrode (formed outside of the body) has enough tolerances built in to allow a sub-optimal fit on both nerve trunks. The cured electrode 1, being formed and cured (fully or in part) inside the body of the subject or patient in question, in contrast provides a perfect fit. This may be accomplished by flowing of the liquid mixture into place, massaging it into place, vibrating it into place, injecting it into place or pressing it into place. The shape of the liquid mixture 1 may be further altered prior to curing with the presence of spacers placed temporarily near the injection site such as inflatable balloons, partially or fully degradable spacers made from surgical sealants or surgical glue near the injection site, or by filling hollow permeable or semi-permeable flexible tubes (made from nylon, prolene, or degradable biodegradable material such as surgical degradable mesh) with pre-cured electrode material or with surgical sealants or surgical glue. This ability to adapt to any anatomical shape, be it the “norm” as described in anatomy or non-typical shapes, is one of the many advantages of the cured electrode improving the interface with biological structures with the aim to transfer various forms of energy, either to or from the biological tissue. Another major advantage of the cured electrode is its ability to scale from a small to a large nerve, either within the same individual or when scaling from a smaller individual of one species to a larger individual of the same species, or be it the scaling of the same or similar therapy approach from one species (e.g. mouse) to a larger species (e.g. rat, or rabbit, or pig) or to bridge the gap between small or large animal to human. In that sense, the cured electrode allows adaptability of the energy transmitting interface from one animal to another animal, as well as to humans and back to animals to facilitate evaluation of effects in one species as a result of an interesting observation in another species, for example. It enables the quicker evaluation of effects within one individual, within a species as well as between species.

The ability to combine various forms of energy transfer within one embodiment of the cured electrode (including, without limitation, heat and electricity, or light and electricity, or ultrasound and heat) allows experimentation with time courses such as sequential or parallel application of various forms of energy transfer. This includes without limitation the transfer of heat from a neural target while or just before (or after) transferring electricity to the same target. Combinations of energy transfer may be in the same direction (from one energy from abiotic to biotic and the other form of energy equally from abiotic to biotic) or in opposite directions (or in contrast, one form of energy from biotic to abiotic while the other form of energy is being transferred from abiotic to biotic simultaneously. The time courses for energy transfer may also be different, especially when different forms of energy are being transferred.

This transfer of various forms of energies may be scaled relatively easily by applying larger or smaller volumes of the liquid mixture at the specific anatomical interface target compared to another species. For example, the cured electrode at the plexus in the abdomen of a rat may be scaled upward or downward to repeat the same application of energy in a larger animal (e.g. rabbit, pig, whale) or smaller animal (e.g. mouse, fish, spider, sparrow bird), so there is no need to manufacture an electrode in a specialized facility (laboratory operating under sterile conditions) to fit a specific shape of the anatomical target outside of the body of the subject's/patient's body: (1) a smaller volume of liquid mixture applied to a specific structure (vagal nerve, brachial plexus, sympathetic ganglion) inside a small animal (e.g. mouse) may be optimal to encase said anatomical structure; (2) a larger volume of liquid mixture may likewise by needed to optimally to encase the same anatomical structure (vagal nerve, brachial plexus, sympathetic ganglion) inside a larger animal (e.g. rat, rabbit, pig); and (3) an optimal ratio of liquid mixture volume applied at a specific volume of anatomical or organ tissue. This ratio will be different for each specific target structure.

The liquid mixture 1 may further be easily sterilized with either water based steam, gamma radiation, ETO gas or other commonplace technologies depending on the liquid mixture in use. The primary requirement for choice of sterilization approach is that it does not disintegrate the liquid mixture or cause a rapid acceleration of the curing of the liquid mixture.

The components for the liquid mixture may further be easily sterilized with either water based steam, gamma radiation, ETO gas or other commonplace technologies depending on the components to form the liquid mixture in use. Such sterilization may take place at room temperature or it may be conducted below the freezing point of the components of the liquid mixture, such as below zero degrees centigrade. The primary requirement for choice of sterilization approach is that it does not disintegrate the components and their ability to later on react in the warm state of the formed liquid mixture or cause an unexpected (not reproducible) change in the expected curing time of the liquid mixture.

For the case where the components of the liquid mixture are first sterilized and then frozen or where they are first frozen and then sterilized, the then sterilized frozen components are to be stored in their frozen state for the foreseeable time to come. The frozen sterilized components may then be grated or otherwise broken up into smaller pieces of optimal diameter (generally: small (1-10) to large (20-500), the optimum being in the range of 20 to 100 micrometer in size), the entire process being conducted with clean and sterile equipment and in a clean and sterile environment. If desired, the grated frozen sterile components may be sterilized another time post grating. These graded frozen sterile components may then be mixed in the desired ratios to form the intended mixture, i.e. prior to dispensing them into a syringe, everything being done in a cold enough environment to keep all components frozen. It is much easier to properly mix and achieve a more homogenous mixture of all frozen components (in form similar to a granulate) than in the case where some components may be liquid and others may by in powder form. By combining all these frozen components to form the proper mixture ratio, a shortened and simplified mixing procedure may be achieved in small and large quantities, either in a specified mixture beaker or even in small volume syringes (1 cc in total or less). These fully mixed, homogeneous frozen granulates may, if so desired, be sterilized in their shipping container (and still under cold enough, meaning frozen condition). The sterilized fully mixed, homogeneous frozen granulates may then be shipped under frozen conditions (such as under dry ice) to the user who will may thaw the homogenous mixture i.e. in the syringe following a pre-set heating protocol prior to injection/placement of the then liquefied now warm mixture which during warming has formed the liquid electrode material.

Additional frozen components may be added to the mixture of frozen components such as steroids and non-steroidal anti-inflammatory medications to minimize the encapsulation thickness of the fibrous tissue to be expected around the chronically placed cured electrode. Such components may be mixed in at the appropriate ratio prior (e.g. e.g. below 1% to 10%) to the final mixture of the frozen granulate version of the future liquid electrode (liquid post warming).

The electrically conductive cured electrode 1 comprises a liquid nonconductor (backbone structure) that has been functionalized with electrically conductive elements, elements or ions to create an electrically conductive mixture that may be cured outside or inside the body. Electrical conductivity is function of capacitive, resistive and inductive elements. The electrical impedance Z is defined as a function of capacitive, inductive and resistive components to the overall impedance the moment that an AC signal is applied. Even for quasi-DC signals, the resistive and the capacitive components may vary, as the charge injection via capacitive displacement current is non-damaging to bodily (especially neural) tissue in the long run, while resistive injection of charge has been shown to induce irreversible chemical reactions and change pH levels in the vicinity of an implanted object (such as an electrode) that may damage biological tissue in the long run as well as change neural conduction in the short run, anticipated and intended or not and possibly noticed as a side effect.

To optimize neural stimulation via electrical current injection, the transfer of charge most optimal if conducted capacitively by e.g. charging and discharging the Helmholtz double layer at the surface boundary between the electrode and the electrolyte, and more specifically, between the highly electrically conductive (low impedance) elements of the cured electrode (e.g., metallic components) and the electrolyte bathing the electrode. As the thickness of the Helmholtz double layer is defined primarily by the materials used for the conductive elements of the cured electrode and the ions in the electrolyte solution, it is the amount of exposed surface area of the conductive elements of the electrode in direct contact with the electrolyte that defines the amount of volume available for capacitive charge injection into the Helmholtz double layer.

The electrically conductive cured electrode may thus utilize elements of various sizes and irregular or non-spherical shapes to increase the amount of surface area available for interaction between the conductive elements and the electrolyte to move fundamentally from a surface effect to a volume effect for charge injection where a large portion of the internal volume of the cured electrode allows for capacitive current exchange with the electrolyte, using surface areas inside the “blob” of liquid mixture formed around a neural stimulation target as well as the surface area in adjacent to the neural stimulation target itself

The magnetically conductive cured electrode 1 guides magnetic fields instead of electrical ones. A non-magnetic carrier 9 is supplied with conductive elements of high magnetic permeability in order to guide any applied magnetic fields within the cured electrode. This cured electrode then connects the magnetic field to a stimulation target or may guide the magnetic fields through a coil aimed to transform the magnetic energy passing through the cross-section of the coil (via a path the cured electrode takes through said cross-section) into electrical energy within said coil. The cured electrode thus helps to guide magnetic fields applied near the skin to deeper tissues to perform work on either the deep tissue itself or a transformer coil electrically providing to power a signal generator. The cured electrode further enables smaller, cheaper, and/or lighter coils to excite neural tissue as it helps to focus and further amplify magnetic fields locally. Thereby, the cured electrode may enable more mobile and cheaper approaches for transcranial magnetic stimulation (TMS) treatments.

In other cases, where electromagnetically induced electric field lines 73 may be used to stimulate neural tissue, the electrically conductive cured electrode 1 may be used to guide electrical field lines to nerves, then pass them through or into said nerves with the intent to activate (stimulate) said nerves. In yet other cases where the electrically induced currents are to be expected high enough to achieve a heating of either the cured electrode or the tissue between two nearby but discontinuous cured electrically conductive electrodes, these may be used to achieve a thermal ablation of the neural (or other target tissue) due to the current passing through the tissue and heating it due to resistive heating. In these cases a coil may be driven by an alternating current waveform, the coil to be understood as a primary coil in a transformer, and it may drive a current in the nearby tissue and any present electrically conductive cured electrodes placed formerly into the body. The induced currents in the tissue and the cured electrode (or electrodes) may be enough to electrically stimulate or heat tissue. For that application, the cured electrodes placed may be in circular or pseudo-circular (oval, rectangular, etc.) shape with continuity throughout the entire cured electrode except for the location where the nerve of interest is located, thereby generating voltage differentials between the two open ends of the cured electrode right at the location of the target tissue. These aspects are related to electromagnetic field lines, though they concentrate on guiding electric field lines that may be induced by an outside-the-body primary coil, utilizing the cured electrode as a guide for electric field line and thereby concentrating them at a neural or other tissue targets with the intent to either depolarize (stimulate/block) or thermally affect them.

In electromagnetism, permeability is the measure of the ability of a material to support the formation of a magnetic field within itself. Hence, it is the degree of magnetization that a material obtains in response to an applied magnetic field. It is also an indirect measure for a material to be able to concentrate magnetic field lines from an externally applied magnetic field. Magnetic permeability is typically represented by the (italicized) Greek letter μ. The opposite of magnetic permeability is magnetic reluctance. In SI units, permeability is measured in henries per meter (H/m or H·m−1), or equivalently in Newton per ampere squared (N·A−2). The permeability constant (μ0), also known as the magnetic constant or the permeability of free space, is a measure of the amount of resistance encountered when forming a magnetic field in a classical vacuum. The magnetic constant has the exact (defined) value of μ0=4π×10-7H·m−1≈1.2566370614 . . . ×10-6H·m−1 or N·A−2). A closely related property of materials is magnetic susceptibility, which is a dimensionless proportionality factor that indicates the degree of magnetization of a material in response to an applied magnetic field.

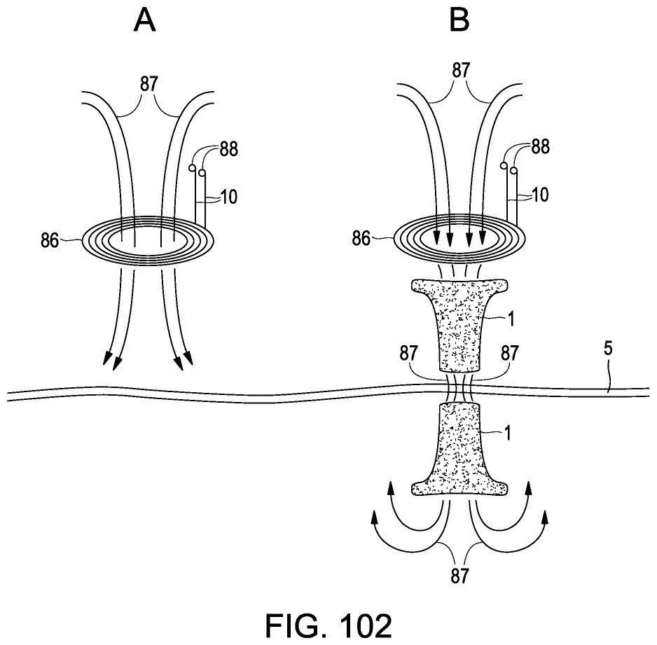

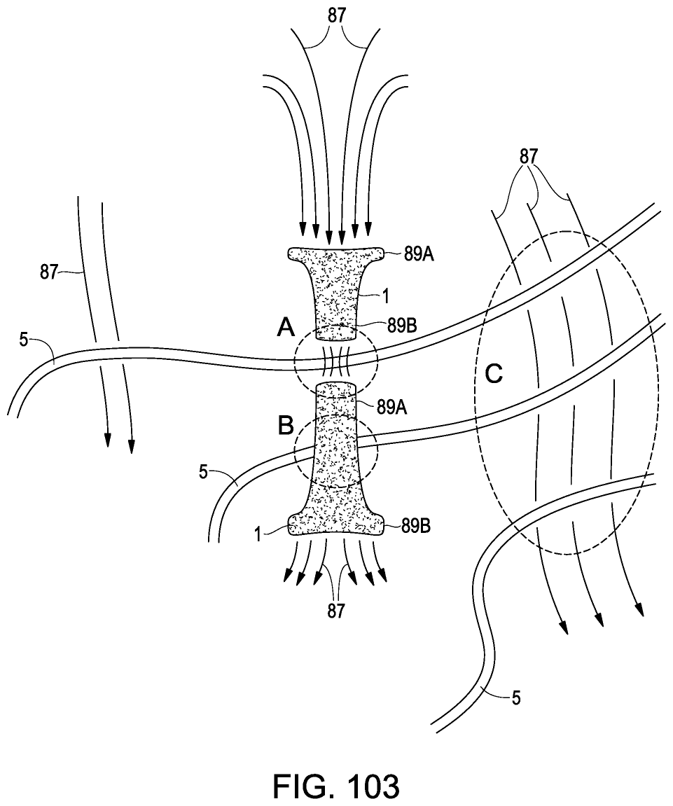

Guiding magnetic field lines 87 provides the ability to concentrate magnetic field lines. An iron core in a transformer guides magnetic field lines following the principle that magnetic field lines 87 concentrate when they enter the north- and south-poles 89A, 89B of a magnet and do not pass by in parallel to a magnet. This principle may be used to guide magnetic field lines from an external magnetic field to be entering and exiting from a magnetically conductive cured electrode 1 that is placed next to a target structure with its north- or south-pole as it is formed by an externally applied changing magnetic field. This allows the concentration of magnetic field lines right next to a neural target chosen for stimulation of block (or other target), which in turn experiences larger changes in magnetic field density and thereby intensity when the externally applied magnetic field is modulated. The magnetic cured electrode thus provides a concentration of field lines, effectively amplifying the magnetic field intensity right next to the cell membranes of the neural (or other) target, as in FIG. 102. In this figure, the cured electrode guides and concentrates magnetic field lines from a magnetic field inducing coil to a nerve. The magnetic field density to which a target tissue is subjected is directly affected by the distance between the coil 86 and that neural target tissue. When the magnetic field density/field strength is not enough to cause a desired neural response (i.e. depolarization) as a result of a changing magnetic field sent out by a coil (in A), injecting a cured electrode (in B) near the target aids in concentrating the magnetic field lines from the distant coil, guide them to the neural target tissue, focus them around the entire target tissue or focus said magnetic field lines to only a portion of the neural target tissue, thereby allowing a relatively small and distant magnetic field to depolarize the whole or selectivity only part of the neural target tissue. While this is described and depicted specifically for e.g. an axon of a peripheral nerve, the neural target tissue may equally be a ganglion, a plexus of nerves, a specific nucleus within the CNS or generally any bodily tissue within a human or animal, especially if said tissue has neural components within it similar to nerves innervating the heart, nerves innervating a gland, neuromuscular end-plates or even neural tissue in tendons to provide an access to the proprioceptive (or other sensory) portions of the body. The coil depicted in the figure may be an external device (outside the skin) or it may be implanted such in a subcutaneous or deep tissue location.

As the externally applied magnetic field (outside the body) is modulated (i.e. switched on and off, or sinusoidally modulated from one direction to the other, or increased and decreased in intensity, or simply modulated in direction), virtual north- and south-poles 89A, 89B are induced at the respective opposite ends of the injected magnetic cured electrode placed near the neural interface target, thereby concentrating the externally applied magnetic field lines near the neural interface target. This concentrated magnetic field is able to depolarize or hyperpolarize biological tissue, such as the neural stimulation target tissue, at a much smaller applied external magnetic field than without the presence of the cured electrode near the target tissue. As a result, lower external magnetic fields may be utilized to provide a neural stimulation effect to the tissue in close proximity (adjacent, or within 5 mm) to the cured electrode.

The magnetic cured electrode 1 furthermore is able to provide selective stimulation of neural tissue. Neural tissue not nearby an implanted magnetic cured electrode will perceive a much smaller changing magnetic field in contrast to neural tissue right adjacent to a virtual or real north- or south-pole of the cured electrode. Further selectivity may be achieved by placing a magnetic cured electrode as a “passing cured electrode” near a neural target that may not be magnetically stimulated (FIG. 103) as the magnetic field lines from externally applied magnetic fields are being concentrated through the cured electrode and away from the to-be-not-stimulated neural target placed near the cured electrode but far from its north- or south-pole. Placing the cured electrode can change the magnetic field density and field lines near or through neural target tissue, such as various nerves forming a neural plexus. Field densities may be increased dramatically by placing a magnetically conductive cured electrode close to a neural interface (i.e. stimulation) target, but leaving the said neural target in the “air gap” between the cured electrode, thereby forcing the magnetic field lines out of the upper cured electrode, through the neural target and back into the lower cured electrode (A). On the opposite end of the field shaping spectrum, magnetic field lines are forced around a neural target in order to minimize i.e. activation when magnetic fields are applied near a neural target. As such, the cured electrode functions as a shield, guiding magnetic field lines around a neural target that intentionally is not to be stimulated/blocked with the use of an applied magnetic field (B). By placing cured electrode far enough from other neural tissue, the effect on magnetic field lines is minimal (C).

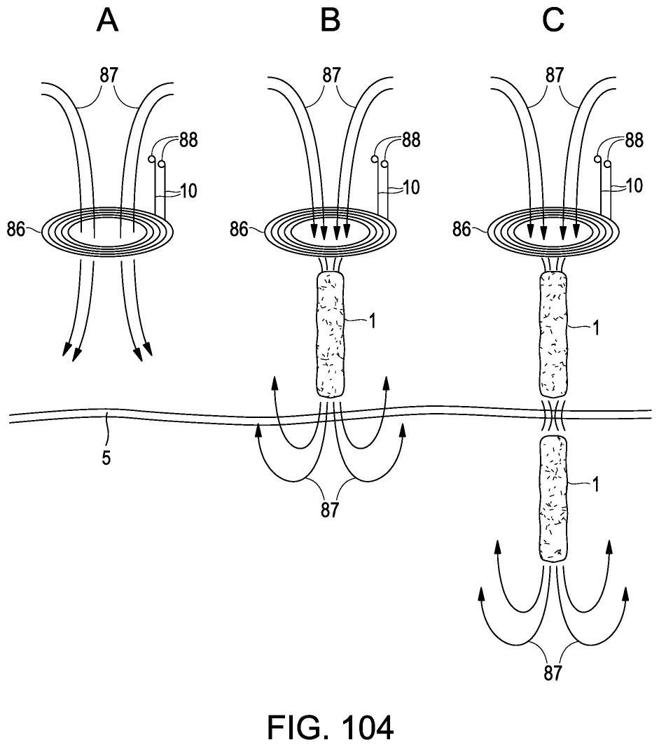

In order to guide, concentrate, direct, redirect or optimize magnetic field lines 87, thereby affecting the magnetic field densities that result from an applied magnetic field, a single magnetic cured electrode, or a plurality of them as a system (FIG. 104), may be used in different embodiments. In contrast to placing no magnetic cured electrode between a coil and the nerve, the magnetic field lines at the location of the nerve are distributed over a larger area, resulting in a small magnetic field density or magnetic field line density (dotted line circle A). By placing one magnetically conductive cured between the coil and the nerve, or behind the nerve on the axis coil-nerve, magnetic field lines near the nerve are concentrated at the location of the nerve (dotted line circle B). By placing two or more magnetic cured electrodes between the coil and the nerve, and behind the nerve on the axis coil-nerve, magnetic field lines near the nerve are further concentrated at the location of the nerve (dotted line circle C). FIG. 104 depicts two cured electrodes 1 placed in the vicinity of a nerve to concentrate the magnetic fields at the location of the nerve (or any other neural, glandular, muscular or otherwise bodily tissue of interest). In contrast to placing no cured electrode between a coil 86 and the target 5, the magnetic field lines at the location of the nerve may be distributed over a larger area, resulting in a small magnetic field density or magnetic field line density (A). By placing one magnetically conductive cured electrode between the coil 86 and the nerve 5, or behind the nerve on the axis coil-nerve, magnetic field lines near the nerve may be concentrated at the location of the nerve (B). By placing two or more magnetically conductive cured electrodes between the coil and the nerve, and behind the nerve on the axis coil-nerve, magnetic field lines near the nerve may be further concentrated at the location of the nerve (C).

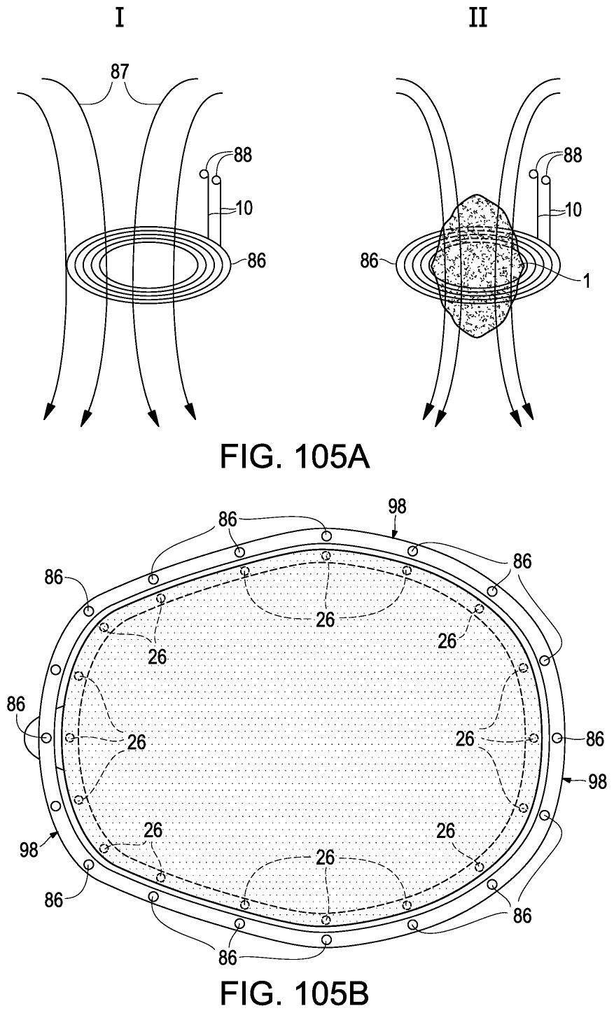

A magnetically conductive cured electrode 1 can also provide a material core for implanted coils. This core is used to guide magnetic fields preferentially to and through the cross section of an implanted coil that may be part of a signal receiver, power receiver, signal transmitter or similar within an implantable signal or waveform generator. On the one hand, a magnetically conductive cured electrode may be placed between the subcutaneous tissue or other tissue close to the surface of the skin as a starting point and the coil of said implanted pulse form generator (IPG) as the end point. On the other hand, magnetic liquid conductor may be enclosed in the encasing of the coil of the IPG to provide a better magnetic field path from the outside of the skin to the IPG and through the IPG with minimized “air gaps” describing the space of low permeability on the magnetic field path from the magnetic field generator (i.e. magnet or coil on the outside of the body) and the IPG. In yet another implementation, the magnetically conductive material is placed as a loop from the subcutaneous tissue to the IPG and back to the subcutaneous tissue in a loop like structure (FIG. 105A). By placing a magnetically conductive cured electrode on the inside of an implanted coil, magnetic field lines are concentrated and guided through a coil. This increases the coupling factor Q and thereby the coupling from an outside to an inside coil. This further allows for a coil with pre-cured magnetically conductive material on the inside to be magnetically connected to in an implanted location by adding more magnetically conductive material (perpendicular to the cross sectional area of the coil) and thereby achieve a better magnetic coupling between a magnetic field from the outside of the body to the implanted coil that may be placed deep inside the body. By pre-curing magnetically conductive material on the inside of the coil there is no “magnetic air gap” when the coil is being connected with magnetically conductive material during the surgery. Furthermore, the implanted IPG with coil does not need to be perfectly aligned with the outside magnetic field generator as the magnetic field lines are guided by the magnetically conductive cured electrode.

The method of placing such a magnetically conductive cured electrode 1 requires the implanted IPG with coil to have a passage within the coil that allows for a needle introducer to be passed through. Said needle introducer is passed through the opening (passage) in the co, then the pre-cured material mix forming the magnetically conductive cured electrode is being dispensed as the needle introducer is being retracted towards the skin. The dispensing may stop within millimeters or centimeters after the tip of the needle introducer passes the hole in the implanted IPG with coil (FIG. 105A-II), or dispensing may be stopped just below the surface of the skin, mere seconds before the tip of the needle introducer leaves the body of the patient. By providing a magnetic field guide from e.g. just below the surface of the skin to an implanted IPG using this approach, the IPG may be placed centimeters deep inside the body while retaining good magnetic coupling to an outside magnetic waveform generator to drive the implanted IPG.

The magnetically conductive cured electrode also allows smaller magnetic coils, both on the implanted side (the IPG) as well as the excitation coils on the outside of the body. As this cured electrode is able to channel (bundle & concentrate) magnetic field lines, magnetically conductive materials can be implanted into, for example, cranial locations to provide the patient with the means to achieve neural stimulation of deeply located neural tissue using a reasonably small external coil which may be fitted into a heat. TMS may be able to utilize smaller and potentially portable magnetic coils, lower currents to power these coils and still achieve a significant patient benefit by using the injected magnetically conductive cured electrode to bundle and concentrate an external magnetic field.

A variety of materials (ferromagnetic, etc.) are useable in a cured electrode to conduct magnetic fields. Ferromagnetic materials are Iron (including varieties in Steel etc.), Cobalt, Nickel, and obviously alloys made from these materials. Ferromagnetic materials themselves are magnetized by applying an external magnetic field and they possess the property to retain a magnetic state they were subjected in before. This persistence of a magnetic field is described in a magnetic hysteresis curve for the given material. In general terms, typical for ferromagnetic materials is that all of a material's magnetic ions or atoms may add a positive contribution to the overall magnetic field in order for a material to be considered ferromagnetic. These materials may have a strong remaining spontaneous magnetization. Amorphous (non-crystalline) ferromagnetic metallic alloys may be made by very rapid quenching (cooling) of a liquid alloy. These have the advantage that their properties are nearly isotropic (not aligned along a crystal axis); this results in low coercivity, low hysteresis loss, high permeability, and high electrical resistivity. One such typical material is a transition metal-metalloid alloy, made from about 80% transition metal (usually Fe, Co, or Ni) and a metalloid component (B, C, Si, P, or Al) that lowers the melting point. Elements (grains, pellets, “filings”, flakes) made from ferromagnetic materials, especially from amorphous (non-crystalline) ferromagnetic metallic alloys, potentially produced by quenching, are used to increase the magnetic permeability of a non-permeable carrier medium without significantly increasing the electrical conductivity to the overall material mixture. Such a magnetically conductive cured electrode, featuring a high magnetic permeability and low electrical conductivity has different applications from a magnetically conductive cured electrode that offers high magnetic permeability combined with high electrical conductivity, providing the user with the choice to include electrical neuromodulation with magnetic neuromodulation, or intentionally, minimizing any electrical conductivity, be it for reasons of safety. A magnetically conductive cured electrode close to the surface of the body which connects magnetic field lines to a location deep inside the body but does not provide a preferential path for electrical fields to travel increases the safety for the tissues and organs deep inside the body that would react negatively when a high electrical field were to be in their vicinity. Ferrimagnetic materials are materials that possess a major magnetic moment directed in one direction while spontaneously neighboring magnetic moments within the same material point in the opposite direction, resulting in a weaker remaining spontaneous magnetization. Ferrimagnetic materials have a specific temperature, the magnetization compensation point, at which the remaining magnetization becomes randomized and the material as such does not possess a remaining directionality for its permanent magnetic field. Heating the materials from below to above this temperature point may be used to “delete” a permanent magnetization. An implanted magnetically conductive cured electrode which is heated (i.e. with a changing magnetic field in a specific frequency bandwidth aimed not to use the magnetically conductive cured electrode to stimulate neural structures but instead to warm the magnetically conductive material) may be de-magnetized when ferrimagnetic materials are used. In certain cases, especially for chronically placed cured electrodes, the elements may be further bio-pacified by encapsulating them with bioinert or biocompatible materials such as gold, platinum, titanium or glass that allow for magnetic field lines to pass through the elements and be concentrated by the elements but also minimize the body's inflammatory response.

Paramagnetic properties describe the tendency of a material to enhance an external magnetic field. As such, a magnetically conductive cured electrode may be used to amplify a small magnetic field in addition to concentrating a magnetic field.

The present invention has the capability of enhancing TMS or other forms of magnetic stimulation of the body through focusing magnetic fields in its vicinity and thereby causing a change in direction for magnetic field lines (one embodiment in FIG. 104, here adopted for deeper tissue). Using this ability, the injectable capability of the magnetically conductive cured electrode allows the concentrated, preferential application of magnetic field lines to specific neural structures.

In one embodiment, the cured electrode acts as a magnetic wave guide for DBS applications. The outside is a coil which is implanted into or outside of the skull similar to a cochlear stimulation. The skull is sealed with a cap. Inside is either (1) a one channel (or multi-channel) cured electrode based on a coil embedded into the cured electrode which goes to a specific location within the brain, or (2) one channel only as a magnetic wave guide: ferromagnetic material guides the magnetic energy from beneath the skull to the location inside the brain that needs magnetic field densities that actually are able to depolarize a nerve or nerves.

In one embodiment, for e.g. DBS applications, ferromagnetic materials or ferrimagnetic materials may be mixed as powders with silicone or another liquid nonconductor prior to curing to encapsulate a coil that is used to couple in electromagnetic fields from an outside power- and control unit. One example of such simple coil- and passive components casted in silicone are implants to power electrodes placed on the cortex (vision to the blind) as well as electrodes driving sacral roots (bladder voiding), as shown in implants by Donaldson and Brindley. By providing a magnetically conductive cured electrode to the center of a coil of an implantable signal generator, a better focusing of the externally applied electromagnetic field is achieved. Generally, the magnetically conductive cured electrode may be part of an encasing that is formed around the coil and circuit components inside the body produces a nesting or conformity to the local anatomy as space is provided and to mechanically attach the entire circuitry with coil and other circuit components to specific anatomical structures such as vessels, fibrous tissue, or bones.

In one embodiment, the magnetically conductive cured electrode is formed outside the body when the circuit components and the coil are encased with the same or compatible silicone. In that case, the chronology of the cured electrode forming events is important: First, the magnetically conductive or permeable magnetically conductive material mix of the liquid nonconductor and highly permeable elements, e.g., iron powder (in one embodiment stainless steel such as CRGO steel) is mixed, then injected into the center volume surrounded by the coil (these steel elements may again be encapsulated by gold or platinum or glass or other bioinert/biocompatible surfaces to enhance their biocompatibility and long term life). A strong magnetic field is applied during the curing process to provide a pre-magnetization of the entire material inside the coil. Secondly, the coil and electrically/mechanically attached circuit components are encased in magnetically non-permeable or non-conductive liquid nonconductor material alone with an emphasis of not covering the cured electrode on the inside of the coil. This will later allow the direct attachment/contacting of the cured electrode with non-cured material during the implantation procedure to cast a channel of magnetically permeable material from e.g. a location of the subcutaneous tissue to the magnetically conductive material on the inside of the coil of the signal generator that may then be implanted deep inside the body but still receive a strong magnetic field when an external magnetic field is applied from the outside of the body and near the location of where the magnetically conductive material begins in the subcutaneous tissue.

First, the coil's wires are separately encased in a magnetic nonconductor (nonpermeable) silicone or other material, meaning for example just encasing the wires alone in silicone. Although optional, this step allows to confine the magnetically conductive cured electrode to be confined to a smaller inner diameter than the entire coil diameter if this may be desired.

A magnetically conductive cured electrode with or without a coil can be placed near material which is magnetically conductive (Fe, etc.) and molded into not magnetically conductive carrier to direct magnetic field from a location of magnetic field generation (coil) to a location of magnetic field application.

The cured electrode provides a more concentrated or a more homogeneous magnetic field in the CNS (by going into the sulcus of the brain) or in the PNS by surrounding the nerve. Also, the cured electrode conducts a magnetic field “around the corner” of anatomical structures and provides magnetic stimulation to locations hidden behind other anatomical structures or locations that would not be able to accommodate a coil for magnetic stimulation.

Magnetically conductive silk can be combined with a liquid nonconductor. Magnetically conductive silk may be produced similarly by making the silk worms eat ferromagnetic material such as Fe₂O₃.

Applying magnetic fields during the curing of the cured electrode increases magnetic permeability. CRGO steel elements align themselves in a strong, externally applied magnetic field. When such a strong magnetic field is applied as a non-changing magnetic field during the curing process while the liquid carrier is curing, potentially with the addition of vibration to further aid in the aligning of CRGO steel elements with the outside magnetic field, then permanent magnetization remains after the carrier material has cured. CRGO steel is only one example, and there are other materials of high magnetic permeability that may be used with such an approach as described herein.

A magnetically conductive cured electrode is able to bring magnetic field lines from the skin to an implant and connect it here, and the conductive elements may be embedded in the nonconductor-encased coil for the signal generator. This cured electrode can bring magnetic field lines from the skin to a target, powered by an external magnetic field generator. Optionally, this can be achieved by an energizer type device as a “Sandwich: cured electrode-nerve-cured electrode” (nerve in air gap) or as a “surrounding: cured electrode around a target, then another cured electrode

In one embodiment the invention acts as magnetic wave guide and concentrator for PNS applications. Outside, a coil is applied where stim is needed, as magnetic stimulation is not felt in the skin at the applied levels of EMF energy. Inside, either a wire-like portion of a cured electrode is placed as an injection of magnetically conductive materials (see inside a magnet in a radio), embedded in a magnetically-invisible carrier (such as hydrogel, silicone, fibrin and others). Or, optionally, crisscrossing said wire-like portion of a cured electrode below the skin to have a larger surface area interact with the electromagnetic-field and then guide the energy to the nerve or other target. In another embodiment a fuse or a max-field element is embedded in the path of the cured electrode to the nerve. The target nerve may be just touched or may be surrounded in a cuff-like fashion to improve the EMF stim and block effects. The cured electrode serves as an EMF concentration device.

In another embodiment, the magnetized ferromagnetic cured electrode is placed in a muscle to measure muscle activity, or in surrounding fascia without being anchored anywhere else. There is not necessarily any point of touching of the cured electrode with other implanted devices, though the cured electrode may be placed within the inside or center of an implanted coil. By placing a magnetized (ferromagnetic) cured electrode into a muscle it is possible to measure muscle activity by placing a coil in the vicinity of the muscle on the outside (or inside) of the body. For the greatest measureable effect, the cured electrode may be moved in a way that the magnetic field generated by the cured electrode or the magnetized non-ferromagnetic cured electrode or the magnetized ferromagnetic cured electrode is interacting at a 90 degree angle with the coil placed in the vicinity of the cured electrode in any of its several magnetically conductive embodiments. One such embodiment includes the coil to be placed around a muscle with the cured electrode being placed into the muscle and within the coil that surrounds the muscle, allowing the muscle to contract and move with respect to the coil. The coil 86 may furthermore be placed laterally at an angle of 90 degrees to the direction of the movement of the cured electrode within the muscle as the muscle is contracted, or at an angle of 0 degree or at any angle in-between 0 and 90 degrees. This allows for a way to measure muscle activity without having to rely on EMG as a way to determine contractile muscle activity presence (on/off) or quantity (off/weak/medium/strong/intense). In addition to measuring activity as one-dimensional data (pull back, release forward), by combining activity from various muscle groups that overlay in their directionality with angles unequal to zero (the muscles pulling in different directions and one muscle may displace another one partially in a non-coaxial manner) then multidimensional movement of a magnetically conductive cured electrode may be accomplished.

There is direct contact between magnetically permeable elements 6. Similar to the minimization of the air gap in iron cores in transformers, the magnetic field is contained best when traveling within a magnetically conductive cured electrode that features the magnetically permeable elements touching each other or being in close proximity to each other. Similar to the magnetic field within the iron core of a transformer, the magnetic field traveling within a cured electrode shows high magnetic potential differences across air gaps and may cause a concentration of magnetic field lines through pathways featuring a more dense and more continuous string of magnetically highly permeable elements.

In contrast to the iron core though, there is fundamentally no “air” gap between the elements that make up the high magnetically permeable feature of the cured electrode, but instead other low permeability materials such as interstitial or other bodily fluid as well as the carrier media of the cured electrode.

To allow for a magnetically conductive cured electrode 1 of a more densely packed high magnetically permeable material, a surfactant similar to the one in the electrically conductive cured electrode may be used as a “wetting” agent that allows the highly magnetically permeable elements to not be fully encased in the lower magnetically permeable carrier medium (such as silicone, cyanoacrylate, fibrin glue etc.), thereby having the magnetic domains of the highly magnetically permeable material elements physically touch each other. Similar to the crystals in a CRGO steel though, touching magnetic domains may only be providing a small increase over almost-touching domains, meaning that these cured electrodes offer almost the same overall magnetic permeability without a surfactant.

In one embodiment, a magnetically conductive cured electrode 1 is placed in body (i.e. subcutis) to convey information via vibration. It is placed as injection into the body of a subject as means to be used to utilize the generation of mechanical forces in one, two or more directions as well as repetitive forces applied along one or more axis to convey a perception of vibration or tickle to the cutaneous sensory nerves. Placing it into e.g. subcutaneous tissue allows the transmission of information as on/off signal, as frequency coded or amplitude coded signal by applying a magnetic field from the outside of the body that reaches the vicinity of the implanted magnetically conductive cured electrode. Such an implementation has the application of conveying a “feeling of music” instead of just hearing it but utilizing the body's proprioceptive sense of force, motion and vibration to perceive motion of an implanted permanent magnet on the subcutis or other tissue (such as muscles with stretch receptors, golgi tendon organs in tendons, or auditory sensory cells of the ear picking up any vibration that is transmitted to specific bones near the ear (such as the skull or foramen of the skull).

In addition to injecting a mixture of ferromagnetic (and/or antiferromagnetic) elements 6 mixed with non-electromagnetically permeable material, entire pellets or other shapes of rare earth magnetic materials (e.g. neodymium magnets) can be injected in one embodiment, themselves with a very high tendency to be only preferentially magnetized along a specific crystal axis, but being very difficult to be magnetized in other directions. Generally, one important aspect is the tetragonal Nd₂Fe₁₄B crystal structure having exceptionally high uniaxial magnetocrystalline anisotropy (HA ˜7 T-magnetic field strength H in units of A/m versus magnetic moment in A·m2) as seen in neodymium magnets. If ferromagnetic material of that or other kinds are implanted in order to be receivers of magnetic energy from the outside of the body, then carrier materials that cure inside the body may or may not be used to control optimal mechanical anchoring/hold, position, positioning, angle towards various elements of anatomy within the body, as well as help with the control of bleeding, the to be expected inflammatory response or other means of bodily reactions to placing a device such as a ferromagnetic element into the body.

In another implementation, the magnetically conductive cured electrode 1 may be placed in mechanical contact with bones of the body to convey information to the carrier/wearer. One of these bony structures may be the skull or may be the lower or upper jaw. By applying a sufficiently strong, variable magnetic field from the outside of the body, a perception of vibration at different frequencies and amplitudes may be conveyed to the carrier/wearer and function as means to transmit information.

In addition to applying the magnetic field from the outside of the body, the magnetic field may also be generated by a coil implanted within the body. Such coil needs a driver unit strong enough to drive the current needed to create the magnetic field that provides a perception of force, vibration or sound to the bone to which the cured electrode is mechanically anchored.

Such a magnetically conductive cured electrode may be placed flat on the bone as “disk” or “smear-on” shaped transducer (magnetic field to force or vibration or sound), or the cured electrode is placed into a groove, a dimple or hole (naturally occurring or man-made during the surgical intervention (which too may be done laparoscopically) or a foramen that allows the proper mechanical anchoring of the cured electrode.

In yet another implementation, a strong ferromagnetic material of e.g. a cylindrical or spherical form such as a neodymium magnet is anchored with electrically non-conductive material (i.e. hydrogel, cyanoacrylate based glues, fibrin-glue, silicone, PMMA based glues, i.e., bone cement, or others) after manufacturing a cavity (i.e. by drilling a cylindrical hole into the bone of a slightly larger inner diameter than is the outer diameter of the cylindrically or spherically formed magnet) specifically for that sole purpose of a stable mechanical anchoring of said magnet into the bony cavity using nonconductive material 9A such as hydrogel, a surgical glue such as cyanoacrylate based glue systems, silicone, hyaluronic acid, PMMA, fibrin glue based systems (fibrinogen+thrombin based systems). This nonconductive material 9A is placed before or after the permanent magnet has been deployed into the bony cavity, or the magnet is pressed into the bony cavity to be anchored by a mechanical press fit or tight fit, only to then optionally be covered with nonconductive material to improve mechanical stability long term, bio-integration and the best possible reception by the subject.

In one embodiment, a system utilizing a helmet or band is worn to indicate direction and distance. The cured electrode is used as a signaling device when implanted into the body near proprioceptive cells. A coil controlled with an external signal excites an implanted/injected cured electrode placed e.g. near tendons (or around or into tendons), near muscles (or around or into muscles), near the outer skin of a person or other locations that are prone to sense proprioceptive, or vibrational or movement information.

In another embodiment an implanted magnetically conductive cured electrode 1 is configured with a helmet or head band or arm band system components. In one embodiment, one or more cured electrodes is injected or otherwise placed just below the skin (FIG. 105B). These placements are in any shape such as blobs 26 around the circumference of the head at a location where a head band or a helmet is worn, and likewise for the arm and the armband. The arm or head band contains a plurality of coils 86 providing a magnetic field independently from each other and allowing the means to activate one or more implanted cured electrodes that are placed into the subcutaneous tissue. Such a system conveys a direction to the subject by vibrating one or more of the cured electrode at the same time. The direction may point north. The direction may indicate the location of a friend or a foe. The direction may indicate an impending danger, such as a car approaching from the right behind a bicycle rider who would be able to perceive the car thanks to a rear-facing camera system sensing the car, intelligent electronics processing and reducing the information and controlling the coils on the back of the subject's head band to produce an alternating magnetic field that in turn slightly vibrates the implanted cured electrode in the subcutaneous tissue, thereby stimulating cutaneous afferents and signaling the user that a car is approaching under conditions programmed in a controller outside the body. In FIG. 105B, the head band with coils, placed around the circumference of the head of a person with magnetically conductive cured electrodes placed as small beads, in one embodiment, about 1 inch apart from each other. The magnetically conductive cured electrodes themselves have a tendency to swing in an oscillating magnetic field and can indicate to a person which coil is active at a specific (i.e. resonant) frequency, optimized for the cured electrodes. In a similar fashion, cured electrodes are molded onto an ear, teeth, fingers, toes and other body locations to magnetically vibrate the body part, providing a mechanical as well as an acoustic interface for the subject. The vibration is used to communicate a yes/no signal, a speed (slower vs. faster vibration) or a force or otherwise non-directional information; or may be used to communicate a direction if e.g. a specific finger were to be vibrated to communicate left, or right, or up or down etc.

The thermally conductive cured electrode also has many uses and embodiments. In contrast to the primarily electrically conductive cured electrode described elsewhere herein, this cured electrode transfers heat from one end to another and therefore guides thermal energy, dissipating and equalizing thermal energy. In the most simple form, highly thermally conductive materials such as diamond, graphene, or metal elements may be mixed in with the liquid nonconductor prior to curing or another thermally more resistive carrier medium that provides the mechanical stabilization but not necessarily the thermal high conductivity needed to optimally transfer, conduct, direct, dissipate or concentrate heat from one location to another. This cured electrode, in several embodiments, is used in conjunction with a Peltier element either implanted or external to the body. The cured electrode is placed between a location deep inside the body and a location close to the surface of the body and used in conjunction with an externally placed cooler or heater that transfers heat through the skin to the cured electrode, and thereby to the deeper tissue surrounding the cured electrode.

In physics, thermal conductivity (often denoted k, λ, or κ) is the property of a material to conduct heat. It is evaluated primarily in terms of Fourier's Law for heat conduction. Heat transfer occurs at a lower rate across materials of low thermal conductivity than across materials of high thermal conductivity. Correspondingly, materials of high thermal conductivity are widely used in heat sink applications and materials of low thermal conductivity are used as thermal insulation. The thermal conductivity of a material may depend on temperature. The reciprocal of thermal conductivity is called thermal resistivity. Examples of a suitable thermal carrier include without limitation graphene and diamond. FIG. 106 is a graph showing thermal conductivity of various materials. Graphene, diamond gold, and other metals are an excellent thermal conductors, compared to rubber (similar to silicone) or water.

In FIG. 106, three examples of materials stand out: Diamond and the two metals, aluminum and copper. While these metals may not be better than stainless steel or gold, for example, they suffice to illustrate the superiority of metallic thermal conductivity in comparison to polymers. Hence, by adding a sufficient amount (i.e. 60 to 80% as measured by either weight or by volume) of the thermally more conductive material in i.e. powder form to a biocompatible polymer (where the monomers too are biocompatible), a fibrin glue, a hydrogel or other carriers of significantly lower thermal conductivity, then the total thermal conductivity may be much closer to the conductivity of the thermal conductor (i.e. metal powder) added to the carrier.

TABLE NINE
Examples For Thermal Conductivity Under Standard
Conditions (Similar To Body Temperature)

| | Thermal conductivity |
|---|---|
| Material | [W · m−1 · K−1] |
| Acrylic Glass (Plexiglas V045i) | 0.170-0.200 |
| Alcohols OR Oils | 0.1 |
| Aluminum | 237 |
| Copper, pure | 401 |
| Diamond | 1000 |
| Fiberglass or Foam-glass | 0.045 |
| Polyurethane foam | 0.020-0.021 |
| Expanded polystyrene | 0.033-0.046 |
| Manganese | 7.81 |
| Water | 0.591 |
| Marble | 2.070-2.940 |
| Silica Aero gel | 0.02 |
| Snow (dry) | 0.050-0.250 |
| Teflon | 0.25 |

In one embodiment, a Peltier element is used to transfer heat from one location to another. In that case, the thermally conductive cured electrode becomes the interconnect between one side of the Peltier element and the biological tissue that is intended to be either cooled or heated. A Peltier element which has the ability to transport heat from one side to the other side when electrical current is flowing through the Peltier element. This type of cured electrode is placed in such a way around the nerve that it encompasses an entire nerve, thereby distributing any thermal energy to and from the nerve in a more or less homogeneous temperature field, minimizing hot- or cold spots that may be damaging to the nerve, while simultaneously averaging out the thermal energy that is being applied to a nerve or carried away from a nerve by cooling one part of the nerve too far (i.e. creation of ice crystals) while other parts remain conductive as may be the case in nerves of several millimeters of thickness, such as the human sciatic nerve. A thermally conductive cured electrode that is placed as a cuff around an entire nerve ensures that the temperature throughout the nerve is more or less homogenous, thus providing more repeatable and reproducible on-target effects with less unwanted side- and off-target effects (cooling other tissues). Furthermore, thermally insulating cured electrodes may be used to encase a formerly thermally connected nerve with its nearby Peltier element, providing a thermal insulation vs. the surrounding tissues and thereby increasing thermal efficacy.

The cured electrode thereby can cool or heat an entire nerve in contrast to only heating or cooling one side of a nerve (such as by a Peltier element on its own), while the other side may remain more or less at the same temperature if the heating or cooling were to be supplied by a Peltier element located only on one side of the nerve. By placing the thermally conductive cured electrode first around the nerve before more thermally conductive material is used to contact the Peltier element, homogenous cooling or heating may be ensured as thermal energy is first transferred (conducted) preferentially inside the cured electrode before the energy travels outside the cured electrode to the nerve or other target. This approach avoids an unintended heating or cooling of parts of the nerve at locations touching the Peltier element, which could unintentionally cause damage to the nerve.

The thermally conductive cured electrode may also be placed on part of a nerve, thereby allowing the intentional cooling or heating of only that part of the nerve. This embodiment of the cured electrode may similarly comprise a two-part version, one thermally highly conductive cured electrode is contacting the nerve in part (or all around) and a thermally non-conducting material 9A is placed around the combination of previously placed thermally conductive cured electrode and the nerve, the overall combined target+thermally conductive cured electrode+non-conductive material representing a mechanically very sound structure.

In one embodiment, a thermally conductive cured electrode 1 is furthermore placed around neural plexi, ganglia, nerves along organs, nerves along blood vessels, or between a nerve and a blood vessel. In one embodiment, a Peltier element 90 is placed between a nerve and a blood vessel, both of which were first freed from each other carefully, to be able to transfer thermal energy from the nerve to the blood vessel or in the opposite direction. Thermally conductive material placed around the nerve and/or the blood vessel is used here to ensure both, an optimal thermal as well as a stable mechanical interface. The thermally conductive cured electrode further provides an element of mechanical cushioning between the nerve (or blood vessel) and the Peltier element which by itself is very mechanically hard and stiff. By being more mechanically flexible, the body is less likely to grow connective tissue (which is somewhat thermally insulating) between the thermally conductive cured electrode than it would be when in direct contact with the hard and stiff Peltier element. Furthermore, as the thermally conductive cured electrode has formed like a glove around the anatomical shape of the neutral structure of interest, there are less mechanical pressure points than would be on a Peltier element.

The thermally conductive cured electrode 1 may be placed on the outside of organs (i.e. the stomach) and organ systems, or placed around air filled vessels (such as bronchi) or placed around glands (i.e. adrenal gland) to modulate the metabolic activity within the tissue of the organ or organ system, and thereby regulate the organ's (system's) output, be it force, hormones, glandular secretions, or other bodily fluids or results said organs/organ systems provide.

The cured electrode can further comprise thermally non- or low-conducting carrier mixtures to provide an insulation wherever needed. Examples for such thermal insulation may be placed as a thin layer on the outside of a nerve-cured electrode connection, thereby limiting dissipation of heat from the nerve to the surrounding tissue or heat traveling from the outside into the nerve.

It is essential for the thermally conductive cured electrode to have a sufficient content of temperature conducting material components in comparison to the not-primarily-heat-conducting conductive elements.

The conductive elements, in a preferred range, represent approximately 60 to 80% of the weight and in various cases 60 to 80% of the volume, and in one preferred embodiment 70%, of the liquid conductor or cured electrode.

This cured electrode may or may not utilize a surfactant similar (as described elsewhere herein) to achieve a good electrical conductivity; heat transfer is especially optimal when the thermally (more) conductive elements are able to move somewhat freely before any curing happens due to i.e. polymerization effects of the carrier or due to the body's natural encapsulation.

Thermal energy (heat) is conducted pre-curing, in one embodiment. This effect is utilized to verify optimal placement, effects of applying cooling or heating during curing as well as an optimal mechanical adaptation to the surrounding tissue.

The thermally conductive electrode modulates the metabolic activity (rate) in biological tissue the metabolic activity of tissue and metabolic rate in tissue is dependent on the temperature of the tissue. Most biological activity in the body is optimized for a very specific temperature range (36 to 37 deg. Celsius or approximately 97 to 98.5 deg. Fahrenheit). Tissue that is cooled or heated to temperatures outside that range will experience a change in enzyme activity inside the tissue, will show an increased or decreased metabolic rate of processing calories, changes in energy throughput as well as changes in output of mechanical forces, chemical reaction products or the production of hormones and other bodily substances to name a few. The cured electrode may be used to intentionally increase or reduce the temperature of tissue through local contact with the tissue whose temperature is to be modulated, or by modulating the temperature of media entering a specific tissue, such as blood, CSF or interstitial fluid. This embodiment can uses blood vessels as heat sink. The body's circulatory system, both on the arterial and on the venous side, provides an excellent way of draining excess heat from the hot side of a Peltier element as blood is continuously flowing by the location of temperature injection to be distributed throughout the body. The piping, so to say, is already present and asking to be utilized as a medium to transport heat (or cold) from or to a location of interest. In comparison to other vessels in the body, the rate of flow in blood vessels is the highest, thus offering an optimal clearance option for heat that is injected (transported) into (or from) the blood vessel. The ability of a blood vessel to drain heat injected into the blood vessel wall from the outside of the wall without raising the temperature of the blood vessel wall is a direct function of the amount of heat injected into the wall, the heat transported through the wall from the outside to the inside and the amount of heat transported away from the inside of the wall by the blood flowing by. By encasing a blood vessel with thermally conductive material, the entire vessel's outer wall may be used in the transfer of heat from e.g. a Peltier element to the blood flowing inside the blood vessel. Using as much of the available surface area of the outside wall ensures the minimization of local concentration of heat points and allows for a more even distribution of heat from the Peltier element to the outside wall of a blood vessel. Furthermore, using the cured electrode is capable of interfacing two or more blood vessels as well as other fluid transporting or fluid containing vessels, vesicles or organs with either a partial or full encasing of the outer wall of said vessels, vesicles or organs. An example of a combination of such vessels, vesicles or organs is blood vessels entering the bladder, parts of the outer wall of the bladder itself, as well as one or two of the ureters transporting urine into the bladder and/or the urethra as a channel of urine out of the bladder.

The cured electrode, in one embodiment, is used to provide a temperature bridge to the urine stored inside the bladder, or the urine entering the bladder through one or both of the ureters, or the urine on its way out of the bladder via the urethra. Cooling urine prior to entry into the bladder or urine present inside the bladder induces a urinary contraction, but warming urine in the bladder prolongs the time between reflexive bladder contractions.

The thermally conductive cured electrode is used to sink heat from a Peltier element into more than one blood vessel simultaneously utilizing more volumetric flow than the volume passing through only one blood vessel, especially if these blood vessels are passing along each other (even if one is an artery and the other is a vein).

Various enzymes operating inside specific tissue or specific organs require a set temperature within a very narrow band (i.e. 36 to 37 degrees Celsius) to perform their intended function. For cases where enzyme activity is too low, raising the temperature inside the tissue and/or organ may increase the enzyme activity. This may be achieved by heating blood vessels that supply blood to the tissue or organ.

The thermally conductive cured electrode, in one embodiment, sinks heat into the trachea or bronchi and/or further smaller sub-bronchi of the breathing apparatus, thereby heating air entering or leaving the lungs.

In another embodiment, the thermally conductive cured electrode is used to cool bronchial tissue to induce a temporary neural paralysis to reduce the amount of mucous production in the bronchi, trachea and connected air vessels lined with smooth muscle and glandular cells. A temporary, non-damaging and controlled reduction of neural activity, glandular activity and muscular activity in the trachea, bronchi and sub-bronchial structures (smaller air vessels leading to the lung) may be used to aid patients with COPD, asthma and other lung disorders by modulating the mucous production in the lining of the trachea, bronchi and sub-bronchial structures as well as modulating (preferentially: reducing) contracting frequency and contracting forces of the smooth musculature wherever present in these structures. By reducing the contraction of smooth muscles and either producing no or only thin mucous (low viscosity) inside the trachea, bronchi and sub-bronchial structures, this allows for a reduced air flow resistance for the inhalation and exhalation in COPD and asthma patients.

The achieved neural effects may range from activation (heightened probability to self-fire neural action potentials after temporary heating), deactivation or block (by cooling or heating outside the temperature window most optimal for neural conduction to be provided based on the neural metabolic activity) in both, full or partial nerve block.

The thermally conductive cured electrode is capable of using arterial blood vessels as a heat source, thereby cooling the blood vessels and any organs and/or bodily tissue which is supplied by the blood passing through the arterial vessels as cooling is being applied (meaning heat is being sourced from the blood in the arterial vessels). Arterial blood that enters an organ (i.e. stomach, heart, kidney, adrenal gland) or muscle tissue (i.e. striated muscles such as in the arm or smooth muscles such as in the lower intestines) indirectly controls the temperature of the organ or tissue it is entering. This method provides the ability to use convection cooling of an organ inside a living organism.

By cooling blood prior to entering a specific organ or tissue to a temperature below the preferential operating temperature of the organ and/or tissue, metabolic rate (metabolic activity), organ and/or tissue activity as well as organ/tissue output (force, heat and other energy, hormones, etc.) may be modulated as needed.

Various enzymes operating inside specific tissue or specific organs require a set temperature within a very narrow band (i.e. 36 to 37 degrees Celsius) to perform their intended function. For cases where enzyme activity is too high, lowering the temperature inside the tissue and/or organ may decrease the enzyme activity. This may be achieved by cooling blood vessels that supply blood to the tissue or organ.

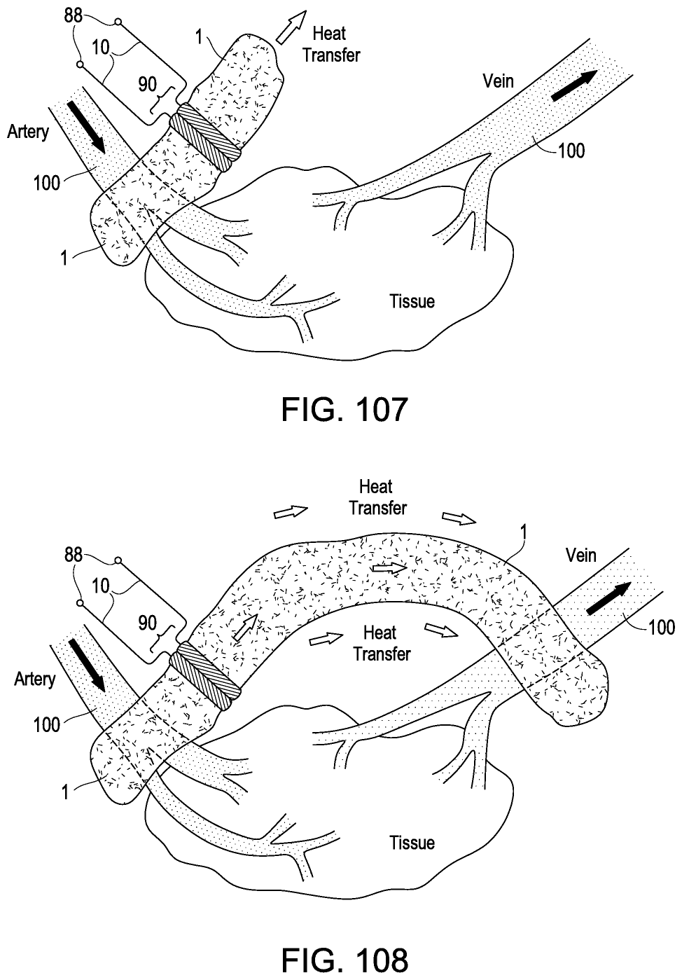

FIG. 107 is a depiction of an organ or a tissue whose metabolic rate is modulated with temperature adjustments. Generally, blood vessels 100 supply arterial blood to the organ and transfer venous blood out of the organ (in addition to potentially other blood streams entering and leaving such as is the case with the heart, the intestines, the lungs, and other organs that add or subtract chemicals into or from the blood stream in addition to taking energy from the blood stream to perform their work). FIG. 107 shows how thermally conductive cured electrodes 1 establish an optimal temperature path from a Peltier element 90 to an artery with the goal to provide a heat drain from arterial blood before this blood enters a target tissue or organ. The first steps of heat transfer is accomplished by the cured electrode (1) to the Peltier element, which in turn transports the heat to the second cured electrode (2) which may either distribute, carry away to another tissue, or simply otherwise function as a drain of heat. The blood passing by cured electrode (1) is cooled and then passes into the target organ or tissue of interest where it reduces metabolic rate. Similarly, heat may be transferred in the opposite direction if an immediate (rapid) warming of the organ or target tissue of interest is desired or if the thermally conductive cured electrode and the Peltier element function as part of a control system, where the temperature of the blood into the target organ/target tissue of interested is regulated to achieve a specific modulation of i.e. metabolic activity over time.

The thermally conductive cured electrode also can be used to heat or cool an organ by differential application of heat between inbound and outbound blood vessels of the same target tissue or target organ Every organ possesses an inbound (arterial) and an outbound (venous) blood vessel. If a thermally conductive cured electrode is used to connect thermally one side of a Peltier element 90 to the inbound (arterial) blood flow, while the other side of the same Peltier element is connected thermally to the outbound (venous) blood flow coming from the same organ, then the excess heat (or cold) that needs to be drained, may be drained into the venous blood flow from said organ, as in FIG. 108. Alternatively, the heat transfer may be draining to blood vessels that connect to other organs on either the arterial or the venous side. Furthermore, the thermally conductive cured electrode material itself as well as additionally implanted thermal masses (be it made from metal for quick thermal energy dissipation within the thermal mass; or be it made from a substance of large specific thermal capacitance such as water at 4.184 J*g−1*K−1; or be it made from thermally conductive cured electrode-like material that may combines a large heat storage capacitance by being in part water-based with metallic flakes that allow the more rapid transmission of thermal energy along the thermally conductive cured electrode material, thereby facilitating quick dissipation).

FIG. 108 shows one (preferentially two) or more thermally conductive cured electrodes connecting a temperature bridge from an inbound blood vessel to a Peltier element to an outbound blood vessel. The “inbound” is to be understood as delivering blood to or into an organ or tissue that may be temperature modulated. The “outbound” is to be understood as delivering blood from an organ or tissue that may be temperature modulated. The concept of inbound and outbound in this context is that they apply to the same organ or tissue. FIG. 108 shows only the heat transfer out of the arterial blood vessel and into the venous blood vessel, but the process may be reversed, transporting heat from the vein to the artery delivering blood to the tissue or organ the vein is fed from.

In one embodiment, the thermally conductive cured electrode can cool and heat blood that is entering cancerous tissue. The target tissue may be, but is not limited to, healthy organs such as the stomach to reduce digestive speed (providing longer satiety), other components of the digestive system, glands producing various hormones, as well as unhealthy collection of cells such as collections of cancerous cells whose activity may be intentionally down- or unregulated to aid with cancer treatment or minimization of damage to other organs during application of anti-cancer drugs (regulating other organ activities down and cancerous activity up to increase cancer drug uptake in the cancer) or down-regulate temperature into a cancer to affect inoperable cancers and their spread, metabolic activity and aggressiveness. Cooling may be applied preferentially to reduce cancer activity. This is especially of interest for inoperable cancers in order to reduce their growth and spread over time as well as providing the potential to apply analgesia locally in the vicinity of the cancer. Heating may be utilized to preferentially increase cancer treatment drug uptake in the cancerous tissue during heating.

The tissue being supplied in FIG. 107 and FIG. 108 can be cancerous tissue that may be part of a non-operable cancer may be cooled down to a level acutely or chronically with the goal to reduce the cancer's ability to grow, metastasize or spread in other ways. Shown here is the cooling of the cancer indirectly by cooling blood vessels feeding the cancer. Alternatively, the cancer may be artificially heated with or without the additional application of anti-cancer-drugs (“chemo therapy” or “immunotherapy” drugs) that may be preferentially docking to more metabolically active cancerous tissue, which may be activated to become more active as heated blood is provided to the cancer locally. In this case, by cooling the blood down as it leaves the cancerous tissue, a preferential binding of a cancerous drug would affect the cancerous tissue but not other bodily tissue as the blood leaving the cancer would be cooled back to normal or intentionally below to avoid unintended treatment of healthy tissue with the anti-cancerous drugs (“chemo therapy” or “immuno-therapy”).

The thermally conductive cured electrode regulates the temperature of neural tissue down by transporting heat away or towards the tissue. By cooling the tissue or arterial blood vessels 100 supplying it, the activity of neural tissue may be reduced to a level that a partial or full block of neural action potential activity is achieved. This is possible by placing a thermally conductive cured electrode near, around or into the to-be-cooled neural target tissue, placing a Peltier element 90 in proximity to the thermally conductive cured electrode and completing the temperature bridge with more thermally conductive cured electrode material as needed to bring the neural target tissue in direct thermal contact with the cooling side of the Peltier element. On the heating side of the Peltier element, more thermally conductive cured electrode material may be used to increase the surface area of the heat drain provided to the Peltier element, as well as using the thermally conductive cured electrode to make a temperature bridge to other surrounding tissue between the hot side of the Peltier element and fatty tissue (that is full of fluids and blood vessels, representing a good location to drain excess heat to) or the outside skin (by placing the hot side of the Peltier (with or without further added thermally conductive cured electrode material) towards the outside of the skin, potentially embedding it in the subcutaneous tissue, or by facing the hot side of the Peltier element towards a blood vessel or a multitude of blood vessels, which may be encased with thermally conductive cured electrode material to provide an even better heat sink between the hot side of the Peltier element and the blood vessels.

The source of heat may in this case be considered the neural tissue, while the drain of the transported heat (thermal energy) may be a nearby or distant blood vessel, fatty tissue, or other kind of organ or tissue with a reasonably large heat capacitance.

In one implementation, a Peltier element 90 may be placed between a target 5 and one or more blood vessel(s) and thermally conductive cured electrode material may or may not be used to mechanically stabilize the Peltier element between the blood vessel(s) and the nerve, while increasing the thermal conductivity for an optimal transfer of thermal energy from the neural tissue.

In yet another implementation, the heat may be transported to the fatty tissue of the body, where it is stored temporarily (seconds to minutes) and from where it dissipates via blood vessels (and other vessels such as those for interstitial and lymphatic fluid) passing through the fatty tissue as well as convection of the heat energy via conduction from fatty to the surrounding tissues.

In yet another implementation, the heat may be transported from the deep tissue location to a location just underneath the skin (subcutaneous tissue), from where it passes through the skin of a mammal, user, person or subject. With additional cooling devices placed on the outside of the skin, this excess heat is lead away i.e. via fans into the surrounding air.

Applications of thermally conductive cured electrodes include nerve block and others include: (1) short term block to get rid of onset response; (2) long term block; (3) cool down blood going through blood vessel and from there to the nerve to produce a cold block via blood cooling the tissue; (4) partial nerve block to reduce the perception of pain; and (5) triggering a stimulus change in another implant made from thermally responsive material (i.e. a device made from shape memory alloys)

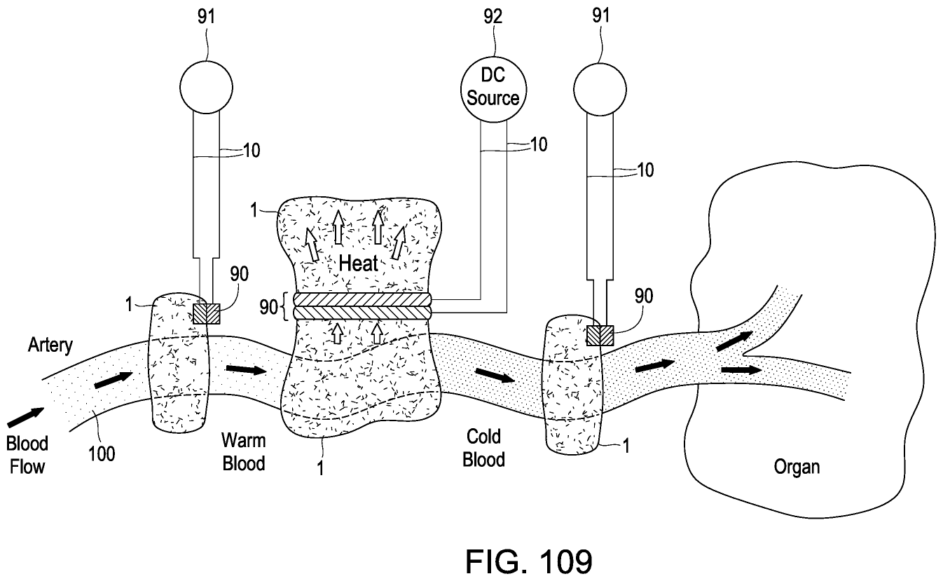

While a primary Peltier element may provide the heating or the cooling to achieve a specific effect to the thermal target interface tissue, a secondary Peltier element or a Thermocouple or a thermally resistive element (i.e. thermally resistive diode, thermally resistive impedance) may be used to provide the achieved temperature change to i.e. a blood vessel. FIG. 109 depicts placement of a temperature sensitive sensor onto a blood vessel or into thermally conductive cured electrode material surrounding a blood vessel, information regarding the true modulation of a blood vessel's temperature may be acquired. These information may be used in a feedback circuit to increase or decrease the current amplitude delivered to a Peltier element (left in the figure), thereby providing a weaker or stronger drive to transport thermal energy to or from a blood vessel. A secondary temperature measurement similar to the one described downstream (right) to the temperature controlling thermally conductive cured electrode may be placed proximal to the temperature-controlling thermally conductive cured electrode in order to acquire the temperature of the blood prior to applying a cooling or heating.

A feedback circuit may determine the temperature that blood has prior to temperature modulation using the thermally conductive cured electrode, apply sufficient current to a Peltier element to transfer heat from or to a blood vessel and then measure the resulting temperature change with a temperature sensor downstream (distally to the thermally conductive cured electrode temp intervention). The feedback circuit may be programmed to apply a specific temperature profile over time (of day or week), or it may be programmed to apply a specific temperature change/temperature profile based on additional sensors placed inside the body or be directed and adjusted by external user input.

Increasing the thermal conductance of a subject's skin can be increased by doping the skin with thermally conductive material, with or without additional carrier media. One way to achieve and increase in thermal conductance is to tattoo the skin with graphene or diamond based thermally conductive cured electrode mixtures that provide a preferential path for heat energy to travel through the skin. This skin may be the outside skin of a person or subject, or it may be the skin of an organ, thereby allowing for a thermal bridge across the organ's skin from the outside of an organ to the inside of an organ without (substantially) impacting or even damaging the organ.

The cured electrode 1 utilizes materials that may be both, electrically and thermally conductive. As such, the cured electrode may be utilized to transmit temperature from one location to another inside the body or apply the same temperature around an organ. The cured electrode can (1) be around a nerve, ganglia, plexus, gland, organ part, entire organ, vessels of or inside an organ, (2) transport temperature energy from one location to another, (3) affect e.g. neural tissue by modifying metabolic rate up or down or stimulating or blocking neural activity. Combinations of thermally conductive cured electrode with other forms of energy conduction (electric, light, etc.). The neural block, in different embodiments, is accomplished either by cooling the nerve directly or by cooling the arterial blood that flows to the nerve, thereby indirectly cooling the nerve. A partial neural block is achieved by lowering the temperature only by a differential of only 1 to 5 degrees, and full nerve block by lowering the temperature by 5 to 10 degrees Celsius. The thermally conductive cured electrode may provide a heat conducting glue for in-body applications.

The thermally conductive cured electrode 1 can be applied to stabilize CNS structures with cooling temporarily. The thermally conductive cured electrode may be injected into the vicinity of the spinal cord or the brain for patients who suffered a traumatic injury (spinal cord injury SCI, traumatic brain injury TBI) or stroke where neural tissue is preserved if cooled down substantially locally. While flushing with cold saline may not be applicable, the local application of a thermally conductive cured electrode would not introduce dilution to CSF and allow for a controlled cooling and potentially electrical stimulation and block in addition to thermal modulation of metabolic activity.

The cured electrode 1, in other embodiments, enables methods comprising combinations of energies or steps. One is conducted sequentially, such as full nerve block without onset. The steps comprise cooling the nerve the down through means of a temperature block, then initiating electrical nerve block (charge-balanced direct current nerve block, 300 Hz stim depletion block/KHFAC nerve block), then heating the nerve back up, and retaining block via electric means only. Another sequential combination comprises partial nerve block and stimulation without onset: cooling the nerve down through a temperature block, then Initiating electrical stimulation to affect only the nerves that are not cooled enough to provide an inside vs. outside fascicle selective stimulation. An alternation of energies is a full nerve block without onset comprising switching between thermal and electric nerve block, cooling nerve down by means of a temperature block, then initiating electrical nerve block (charge balanced direct current 300 Hz stim/KHFAC nerve block), then heating the nerve back up, and retaining block via electric means only, then, at the appropriate time (e.g., when the electric block may not be advised to avoid unwanted side effects) cooling the nerve down by means of a temperature block, then initiating electrical nerve block (300 Hz stim/KHFAC nerve block), then heating the nerve back up, retaining block via electric means only, then, at the appropriate time.

Thermally conductive cured electrodes 1 can vary in their material combinations. The nonconductive material 9 can comprise a hydrogel, silicone, cyanoacrylate, fibrin (fibrinogen+thrombin), PMMA, Hyaluronic acid, hydrogels that may or may not polymerize, non-heat conducting, biocompatible polymers made from biocompatible monomers and others described herein. This liquid nonconductor may be curing or non-curing inside the body. Thermally conductive cured electrode heat transmitting media may be the same as for those conducting electricity, such as elements composed of Graphene (carbon nanotubes, graphene powder, etc.), Diamond, various metals such as Gold, Silver, stainless Steel, Platinum and others

In another embodiment the thermally conductive cured electrode reduces the temperature of glands to reduce glandular activity. Glandular activity may be reduced in the digestive tract to slow down digestion and provide a user with a longer perception of satiety. By reducing the metabolic rate in the stomach or the various intestines, food intake will stay longer within these food processing locations within the body, thereby reducing the drive to eat again. This approach may be utilized for a weight loss approach. Similarly, by cooling the stomach and/or lower intestines during the digestion process, the digestion process itself may be slowed down due to reduced enzymatic activity as a result of sub-optimal temperatures for the digestion process inside the organs. The resulting prolongation of the digestion process may lead to a longer feeling of satiety as well as weight loss in the process.

A thermally conductive cured electrode 1 can conduct warm blood to a patient's arms and legs. The thermally conductive cured electrode may be injected around blood vessels in the periphery of the body of patients who suffer suboptimal activity in their autonomic control of blood flow to their limbs and digits. An example are patients with Raynauds syndrome (https://www.niams.nih.gov/Health_Info/Raynauds_Phenomenon/raynauds_ff.asp). The thermally conductive cured electrode may help to either induce thermal input to the blood vessels in the periphery, triggering a reflexive response to normalize the action, possibly in combination with electrical stimulation or block of neural activity along the blood vessels, thereby opening the contracted blood vessels back up (the smooth muscles of a blood vessel may be blocked by temperature (cold) block as well as electrical block). Here the thermally conductive cured electrode increases blood flow to the periphery. The thermally conductive cured electrode also helps to heat up blood flowing towards the periphery. The warm side of a Peltier element is used to connect with thermally conductive cured electrode material to a blood vessel, thereby allowing for the transfer of heat into the pathologically smaller flow of blood which in turn would still be able to provide more of a heating effect into the periphery than no intervention at all.

The thermally conductive cured electrode enables a temporary cold block anesthesia to an organ. The thermally conductive cured electrode may be injected around blood vessels in the limbs/periphery of the body of patients or around blood vessels providing the blood supply to an organ that is to be receiving surgery to allow for a reduced metabolic rate in the limbs or organ of interest, especially if such reduction of metabolic rate is advantageous for post-surgical recovery. The thermally conductive cured electrode may here function to provide a localized temporary “coma”-like state to the limb or organ, allowing less anesthetic drugs during and aiding post-surgery.

The light conductive cured electrode 1 is another embodiment of the invention herein. In general terms, muscle tissue and many other tissues in the body filter a large spectrum of wavelengths and permit only a longer wavelengths to pass through the body (red light and infrared light spectrum). To manufacture a light conductive cured electrode, light conducting elements are mixed with a less-light conducting carrier, the overall mixture being significantly more conductive for the same red to infrared light spectrum, but in addition to that offering the ability to conduct wavelengths even up into the green, blue, and violet spectrum, in extreme cases even ultraviolet as long as the light conducting elements are packed dense enough and themselves highly transmissive (conductive) for the wavelength(s) of interest. Examples are clear diamond, polished glass in form of beads, round or oval, or cubic in shape, or metallic elements such as germanium based alloys that can conduct specific wavelengths of the visible and non-visible spectrum of light. Such a light conductive cured electrode may be placed as a seal of a skull bur hole to allow an external light generator to be placed on the skull when needed and thereby transfer energy to a deeper structure inside the skull, such as a sulcus next to gyri of the cortex, or even deeper brain structures.

The light conductive cured electrode is applied in optogenetic modification of PNS nerves, ganglia or CNS nuclei. This cured electrode functions as light-waveguide and allows the same intensity of light to penetrate one or more structures as either directed light or as a diffused light to be provided from one source to one or more neural target structures.

To achieve an optimal conduction of light, a minimum number of phase boundaries need to be present throughout the volume of the light conductive cured electrode. In order for elements with high optical conductance (clearance) to be able to touch each other and conduct light without excessive scatter, a surfactant (described elsewhere herein) is used to minimize the encasing of the highly optical conducting (clear) elements with the less conducting mechanical carrier and stabilization medium (i.e. fibrin).

As used throughout herein, the term “vibratory cured electrode” means a cured electrode comprising elements whose conductive capacity includes that of vibration energy including without limitation acoustic and ultrasound.

The electrically conductive cured electrode utilizes materials that may be both, electrically and conductive to light in specific wavelengths. As such, the cured electrode may be utilized to transmit light, photonic energy, of one or more wavelengths from one location to another inside the body or apply the same photonic energy around a nerve, an organ or any other specific anatomical structure of interest inside the body. The application of sounds or mechanical forces such as mechanical vibrations has been shown to evoke action potentials when applied to axons in the periphery. Ultrasound (US) is one form of mechanical vibration that may be conducted by various media. Some media function as good, others as bad conductors to US energy. The combination of good-conducting and bad-conducting materials allows the formation of US-lenses that may focus US energy.

By combining materials (i.e. metals, graphene and others) that are characterized with a high conduction velocity and slow damping factor for sound waves with a surfactant it is possible to make these conducting materials (i.e. as a powder) still have contact throughout an cured electrode when a sound-damping material is used as a carrier (i.e. silicone). While silicone alone would function as a damper of US waves, it is possible to change the conducting properties of the silicone by adding the elements and either form a reflective or conductive mixture. If the elements have only small amount of direct mechanical interactions with each other but large amount of mechanical interaction surface with the silicone (achieved at smaller concentrations of elements, such as 5% to 40% of the volume is formed from elements, the remainder from silicone (and optionally surfactant), then the overall mixture appears more as a reflector of US energy. In contrast, if concentrations of approximately 60 to 80% elements, 0 to 10% surfactant and the remainder silicone (or other carrier) are used then the elements have a large number of mechanical interface points that are not damped by a thin silicone layer between the elements, allowing the mix to function as a conductor of US from one location to another.

Guiding and focusing US energy is used, in one embodiment, to transmit US energy from the outside of the body and to increase energy conversion efficacy at the implant. Some recent developments utilize ultrasound (US) to transmit power to implantable electronics such as signal generators to stimulate neural tissue or biopotential or bio-signal recording sensors that may acquire and then transmit biosignals back out from the body. These devices when powered by US require a certain ultrasound energy density to be applied at the device in order to convert US to electrical energy which then in turn powers the implant. The vibratory cured electrode captures, concentrates and/or guides US energy from a larger cross-sectional area (or volume inside the body) to the relatively small volume of the implant. This may be accomplished by utilizing materials that conduct US preferentially to the surrounding biological tissue, offer a path with less reflections of US energy along the path and potentially utilize reflection of US energy from the edges of the path back into the path, thereby functioning as a US waveguide. In certain cases, the US conducting cured electrode may be used to cover a bur hole in the skull, allowing a better transmission of US energy to implanted electronic devices that utilize US energy as their primary power source. The US conducting cured electrode reduces the losses of US energy between the skin and the deeply implanted US powered devices. This may be further aided by utilizing small needles (diameter <0.5 mm) to transmit the US energy from the outside device through the outer layers of the skin into the US conducting cured electrode, from where the US energy is transmitted towards the US powered electronics implanted more deeply inside the body.

One implementation of such a vibratory cured electrode comprises the physical form of a cylinder made from a liquid mixture comprising elements with the capability to conduct, for example, US sent into the body to be captured into the vibratory cured electrode and transmitted to the implant which touches the vibratory cured electrode on one end. The other end of the vibratory cured electrode, in one embodiment, comprises a coating comprising a US reflective material, thereby offering a closed end to a standing US wave while the open end of the vibratory cured electrode represents an output location of captured US energy to the implant to be powered.

In one embodiment, the vibratory cured electrode conducts US energy from the skin and transports it preferentially to a nerve. This cured electrode is able to conduct US energy around corners and to focus the US around a whole nerve; having reflective properties for the US to depolarize the whole nerve instead of just a part of it. In another embodiment the vibratory cured electrode can also transduce and conduct non-US vibrations from inside or outside the body.

It is possible to focus US energy with a US lens made from vibratory conductive material. In one embodiment, the vibratory cured electrode has a shape (e.g., a cone) from which sound energy is reflected off the edges and focuses towards a specific target located for example at its tip.

The US conducting (vibratory conducting) cured electrode may further have ferromagnetic elements included and the magnetically conductive cured electrode are combined. That is, a cured electrode in one embodiment comprises elements which conduct vibratory and magnetic energy. A mechanical signal or waveform can be conveyed by an implanted ferromagnetic element mix with a liquid nonconductor (e.g., hydrogel, cyanoacrylate or silicone or PMMA (Polymethyl methacrylate), or pellet (of 0.1 mm<diameter<10 mm), or marble (of 0.1 mm<diameter<10 mm), or cylinder (of 0.1 mm<diameter<10 mm), star-shaped or i.e. T20 torx bit shaped implant (may have different T-values) or otherwise logical implantable structure easily deployed into a cylindrical hole drilled prior to implanting the described device. In another embodiment, the magnetized ferromagnetic cured electrode is placed in a muscle to measure muscle activity. In one embodiment a magnetically conductive cured electrode is placed in a body (i.e., subcutis) to convey information via vibration for more information and a better understanding. The materials which are suitable for vibratory conductive elements are hard and dense, such as biocompatible metals including without limitation gold, silver, platinum, platinum-iridium, titanium, titanium oxide and iron and also graphene and diamond.

The present invention also comprises optimized wires, leads and connectors to interface with the cured electrode. For example, with the electrically conductive cured electrode, there is a need to avoid a galvanic cell between the materials present in a cured electrode (especially one for conducting electricity) and the connecting lead wire conducting electrical (or other) energy into the cured electrode with the goal to have the cured electrode transfer that energy to i.e. a neural target tissue.

The present invention also comprises capabilities of avoiding the formation of a galvanic cell with the cured electrode inside the body. When a metals is submerged in an aqueous solution then a chemical half-cell is formed between the ions in the aqueous solution and the atoms in the metal. This half-cell features a specific half-cell potential between the metal and the solution. When two different metals are submerged in an aqueous solution then two half-cells are formed between the ions in the aqueous solution and the specific atoms in the two bulk metals. The two half cells in turn form a whole cell, or galvanic cell, between each other, the basis for any battery, with a metal-metal specific chemical cell potential between the two metals. If an electrical connection is made between the two metals that allows for electrons to flow from one metal to the other, then reversible and irreversible chemical processes are initiated at the surface boundaries between each metal and the ionic aqueous solution. These chemical reactions may dissolve one metal and deposit it on the other one with metal atoms from what is called the less noble metal going into solution, traveling as ions towards the more noble metal, and depositing themselves as metal atoms again on the other metal. This process is used industrially to electroplate one metal onto another (such as covering stainless steel with zinc to further add to the minimally corrosive nature of a stainless steel rod for applications in contact with nature). In the body, the formation of a Galvanic cell as a result of combining two different metals that are connected through an electronic circuit on the one hand and the body's interstitial fluid on the other hand is to be avoided. This minimizes the corrosion of electrodes, lead wires, contacts and implant housings in general, and, equally importantly, minimizes or prevents the formation of metal ions and corrosive reaction products from going into solution and causing a prolonged inflammatory response around a neural implant in the long run.

The electrically conductive cured electrode provides the electronic bridge as the direct interface between a signal generator's signal traveling on metallic conductors (e.g., lead wires) and the neural or other target tissue. Such an electrically conductive cured electrode can be an injected cuff surrounding a nerve, ganglion or plexus on the one end and a lead wire on the other hand. If the lead wire were to be made simply from stainless steel and the electrically conductive cured electrode mixture were gold flakes as conductive elements in a fibrin, or a silicone, or a cyanoacrylate carrier matrix, then a galvanic cell may develop between the lead wire's stainless steel (an Iron Fe alloy) and the more chemically stable, more noble, gold of the cured electrode mix. To avoid the development of a galvanic cell, the lead wire is optimized to achieve a gold-to-gold interface between the cured electrode mix and the lead wire, both of which may be in contact to some degree with the aqueous solution of the interstitial fluid and/or other fluids in the body.

Optimized wires for the cured electrode, in one embodiment, comprise metal plating (electro-plating, heat-plating, etc.). In one embodiment, steel (or other metal) wire is electroplated or hot-dip galvanized on the outside with gold to a sufficient thickness (e.g., >1 um) to allow a mechanically stable metallurgical bond between the gold atoms on the steel wire matrix even when subjected to forces between the cured electrode and the wire. For cured electrode embodiments combining, for example, metal flakes and a somewhat flexible carrier, relative motion between the lead wire and the cured electrode may cause abrasive effects as the metal flakes apply shear forces against the lead wire. In order for the metal plating to retain an excellent interface and not be seared off, a sufficient thickness and metal-to-metal bond such as metal plating (hot plating, electro plating) is utilized. Wire with electroplated/heat-plated gold include those as described herein. Gold (or other metals) is heat-plated or electroplated onto another wire (e.g., steel) for the last few mm to cm of the wire, the length of the overall wire that will be in direct contact with the cured electrode plus a reasonable distance (i.e. 5 mm) to allow the encasing of the wire with a pacifying agent that increases the impedance to the high kOhm or even MOhm range and is adhering to both of the metals of the wire in such a way that water does not easily penetrate along the metal-to-pacifier boundary. In one embodiment, the pacifier comprises layers of parylene C or other substances that provide a close to hermetic covering of the two metals of the wire interfacing prior to encasing the overall wire in i.e. silicone to finalize the lead. CVD/PVD covering with gold and/or other metals or materials are deposited on the outside of a carrier-wire (e.g., steel based) to achieve a strong metal-to-metal bond.

In another embodiment, a wire “core” is placed inside another wire “tube” to allow for the interface to the cured electrode to be of the same metal as the metal elements used in the cured electrode. “Hiding” the metal to metal transition beneath insulating structures. The transition point from one metal to another metal is covered with insulating and potentially hermetic structures (i.e. polyimide with parylene C deposition around the metal to metal interface) to expose only the same-metal-interface metal (e.g., gold) to the cured electrode. Such transitions are manufactured with thin film, lithographical or even thick film technology to some degree, allowing mass manufacture of small, flexible lead wires of high reproducibility.

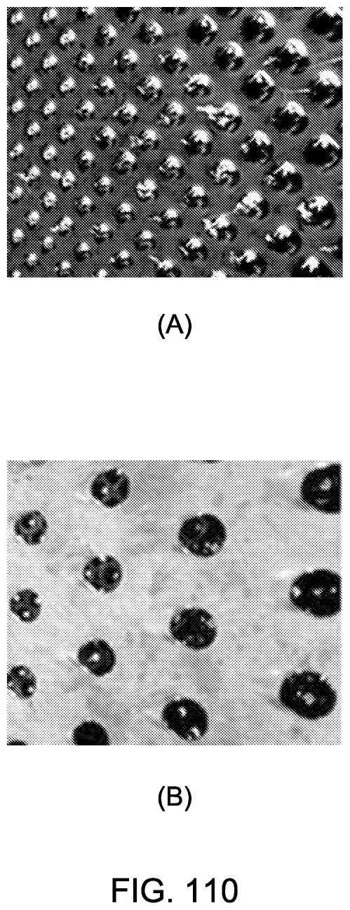

A thin film lead wire with grated surface structure for optimal mechanical interface is used with the cured electrode herein in one embodiment, To achieve an optimal mechanical integration/interface between the lead wire and the cured electrode, the lead wire is flattened or manufactured with thin film technology approaches using i.e. polyimide or silicone carrier (cured around the wire) that encases a metal conductor and then is pressed into a shape that has openings similar to a grated structure, examples of which are shown in FIG. 110. This figure contains photographs of two examples for grated structures that allow a strong mechanical bond. Conceptual representation of how a thin-film lead wire may have high and low structures (A) or holes (B) to allow the liquid mixture to bond more securely as it cures to a solid.

Another embodiment of the cured electrode herein combines hard and soft material in one or more cured electrodes. Combining hard, less flexible, with soft, more flexible, materials allows formation of a cured electrode (or combination of cured electrodes) with a graded transition from a hard, inflexible structure to integrate better with bone, to a softer structure that may better integrate mechanically with soft tissue (such as various neuromodulation target tissues). In one embodiment, a soft, more pliable cured electrode in a first step is formed around a neural target structure with a more flexible material such as hydrogel, silicone or fibrin. In a second step, the lead wire is placed onto the softer material and encased with softer material. Then, in a third step, some less-pliable material including, without limitation, cyanoacrylate is added to form either a shell on the outside or, at least in part, seep into the softer material, or a mixture or softer material with less-pliable material to encase the formerly placed softer material. The resulting cured electrode features qualities of both, the more pliable and softer material towards the neural interface tissue, while offering a harder shell and mechanically stronger interface to e.g. a lea wire or a boney structure to which the now harder-shelled cured electrode may be bonded to reasonably well.

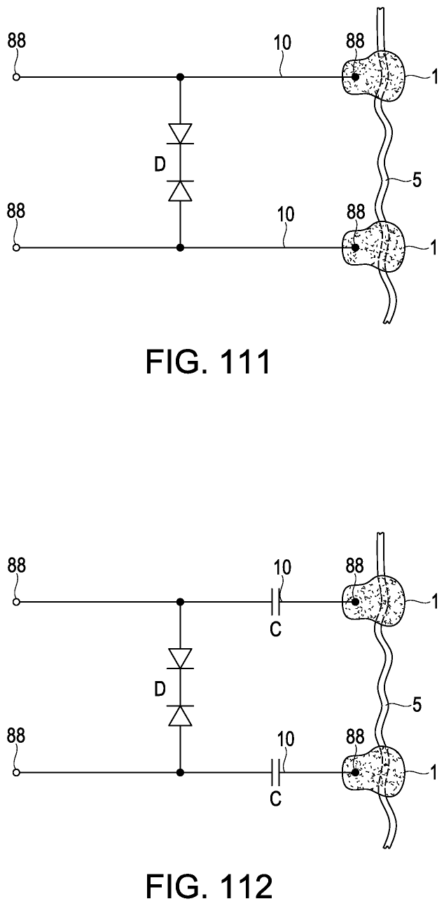

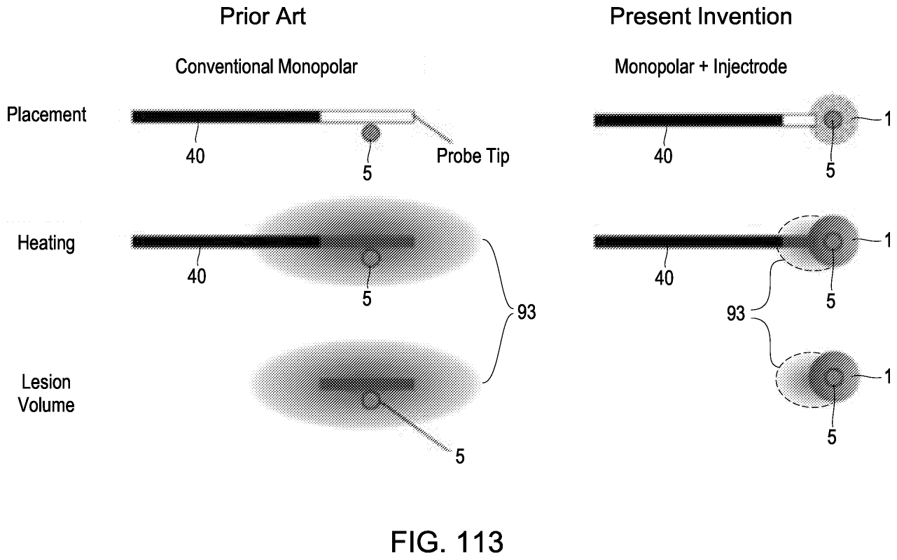

In a further embodiment a current and/or voltage limiter and “predetermined mechanical breaking point” is provided. The wire/lead can comprise a mechanical weak point, or predetermined breaking point to ensure that the cured electrode around a neural or otherwise interface structure is not inflicting damage to said structure when the wire is being pulled or pushed on, i.e. from the outside of the skin such as i.e. on an arm. In another embodiment, the lead/wire comprises a current limiting circuit and/or a voltage limiting circuit. Diodes may be placed to function as a short for high voltages applied from the body as shown in FIG. 111 depicting an over voltage limiter using two diodes. FIG. 112 depicts an over voltage limiter and DC current limiter using diodes (D) and capacitors (C).

Other embodiments of optimizations of the mechanical integration between the lead wire intended to connect with a liquid electrode prior to curing and thereby forming a mechanically strong bond with the cured electrode is to utilize grates, holes or mesh-like structures at the end of the wire right at the interface location between the lead wire and the liquid electrode prior to becoming the cured electrode. FIG. 110 shows two examples, (A) for a grated structure that allow a strong mechanical bond and (B) a flattened metal with holes to allow the particles and carrier material of the liquid electrode to mechanically interface better prior to curing and achieve a better mechanical integration of the cured electrode with the lead wire.

Non-curing carrier materials enable a cured electrode for special circumstances, and are a sub-class of electrode variants which do not require a curing of the electrode inside the body. This is similar to the electrically-conductive powder-only based cured electrode disclosed herein in which the powder itself is a carrier as well as other sections where only the conductive elements is placed into/around/into-the-vicinity-of the neural target tissue using for example pellets filled with the powder that are deployed (implanted as resorbable pellet leaving only the powder that integrate over time or the pellet being opened during the implantation procedure to release the powder or an auger system to deploy the powder or powder mix). In addition to these implementations described herein and before, the carrier material itself can be non-curing inside the body. In that sense, the carrier material only functions as a delivery vehicle of the energy-conducting elements or mixtures through the channel of the delivery device (i.e. syringe with needle) that would not be able to dispense/deliver a powder by itself, but is able to deliver a powder that is suspended in a viscous solution such as a hydrogel, a gel in general forms, a sugar solution (including complex less convertible sugars such as trehalose), or any other form of pharmacologically inactive ingredient to temporarily suspend the powder. Post-delivery to the target tissue, the target tissue itself provides the mechanical stabilization that the non-curing carrier does not provide. Such a cured electrode has the advantage that a variety of FDA cleared inactive ingredients is used to deliver a non-curing material allowing the conduction of specific energies to or from neural target tissue. (See FDA Database of Approved Pharmaceutical Excipients).

Two aspects allowing a cured electrode to be formed inside the body with a non-curing (i.e. non-polymerizing) material, be it for electrical, thermal, magnetic or other means of energy transfer include: (1) the body provides the temporary mechanical stabilization during the material placement and for the first few days until a strong encapsulation has been provided by the growing fibrous tissue. Such a “temporary mechanical stabilization” is provided by injecting the liquid mixture into the nerve sheath surrounding a nerve, nerves, or ganglia within the body. This temporary anchoring is aided by the bleeding at the cured electrode placement site, forming a conglomerate of blood and electrically conductive liquid mixture; and (2) The body encapsulates the injected material with fibrous tissue within days to weeks to mechanically stabilize the cured electrode in its temporary location and make the temporary placement location a permanent one.

Additional aspects for embodiments with noncuring carrier materials: (1) a hydrogel as a carrier helps with delivery of the metal elements, (2) the gel can be electrically conductive by adding ions, electrically conductive elements such as e.g. gold flakes, (3) injection can be into the nerve sheath so the body absorbs the gel while metal elements stay in place, (4) FDA cleared hydrogels are suitable including without limitation PLGA, PEG, NIPAM and do not swell much or are already swollen, (5) they can be mixed with gold elements, (6) they augment the process of pushing elements through the syringe & (thin) needle, (7) the mechanical stabilization is primarily provided by nerve sheath, (8) it is polarized, may be any viscous solution or sugar such as trahalose, a complex sugar not immediately taken up by cells and which is used in pharmaceuticals as a stabilizer, (9) this may be a viscous liquid suspending the gold elements, and which is enzymatically broken down over time into glucose, fructose, sucrose, and (10) and applications include without limitation, around or into DRG, around nerve stump for neuronal axons, etc.

The noncuring elements include some of those on the FDA's approved list of inactive ingredients at

https://www.fda.gov/drugs/informationondrugs/ucm113978.htm. A ZIP file of all inactive ingredients is at:
https://www.fda.gov/downloads/Drugs/InformationOnDrugs/UCM080154.zip Of these ingredients, only a few may be called out as practical, depending on their viscosity (carriers may preferentially be more viscous than i.e. water), inert, thermal, electrical, optical or magnetic conductance, as well as other properties. Inactive ingredients are included to serve as competitive molecules for impeding the speed of crosslinking reaction, serve as surfactants for the organic-to-inorganic interfaces, plasticizers, coloring agents, imaging contrast, or imparting other useful properties to the precursor or cured materials. Inactive ingredients must be biocompatible (bio-inert), and be either stable and cleared by excretion or degradable/metabolized into non-toxic byproducts.

Electric Epi-Pen is one of the embodiments of the present invention. Allergies are chronic conditions, and often a standard intervention to an allergic reaction is the increase of sympathetic activity by injecting epinephrine (adrenaline). By stimulating either elements of the sympathetic chain or by electrically stimulating the adrenal gland, the concentration of epinephrine in the blood stream is increased. In contrast to the drug-based version, the electric Epi-pen is turned off (turn off stim, quick stop of the induced epinephrine release). A feedback circuit measures heat-rate and blood pressure, indicating heightened stress levels and allows the user to operate a push-button system to electrically increase adrenaline in the blood stream and thereby allow the user to avoid perceiving heightened stress for too long automatically. In one embodiment, differing levels of the electric epi pen are achieved by stimulating the sympathetic chain at various locations along the spine.

As described elsewhere herein, the cured electrode interfaces with the PNS at locations formerly not feasible with prior art electrodes, such as behind structures which the needled-based or laparoscopic approaches can go through and/or around structures, and through a laparoscopic approach with air opening up a cavity and visualization via camera.

The cured electrode interfaces with the Adrenal Glands, which sit on top of or next to the kidneys, through the celiac and renal plexus. Delivery approaches include without limitation contacting plexi and innervation points with the cured electrode and wires/BION like signal generator, and encasing the whole Adrenal Gland with liquid nonconductor to provide mechanical stability and integration, all done with laparoscopic approach. The Adrenal Gland secretes Adrenalin (Epinephrine) and Ghrelin (growth hormone; works with Dopamine and others on happiness; works on metabolism). Injection of a cured electrode in one embodiment can be into the Adrenal Gland to the middle of the gland where the Ganglion sits. Or, the cured electrode can be injected to surround the ganglion by injecting into Adrenal Gland and using the gland as the mold for the liquid mixture that surrounds the Ganglion. The adrenal gland is innervated by celiac plexus and renal plexus which stimulate the plexi and release epinephrine, dopamine and nor-epinephrine. One may choose to stimulate directly via efferent innervation, or indirectly via reflex drive, especially with the utilization of sympathetic and parasympathetic fibers. One may choose to increase the effect by combining sympathetic stimulation of nerves heading to the gland, running nearby the gland or even sympathetic neural tissue distant from the gland, while modulating (stimulating and/or blocking partially or fully) the parasympathetic innervation to the gland or globally at one or more locations in the body. The effect will be increase in epinephrine (adrenaline) in the blood stream as well as generally heightened sympathetic activity and side effects such as increased blood pressure, heart rate, alertness, energy and metabolism throughout the body.

Interfacing the cured electrode with the Renal Plexus of the autonomic nervous system allows a number of applications to modulate both, parasympathetic and sympathetic activity locally as well as throughout the entire body. Renal activity may be modulated directly by stimulating efferent nerves and via reflex pathways stimulating afferent nerves. Nearby plexi may be activated as needed to control gut and intestinal activity. In contrast to traditional electrodes, the cured electrode is flexible and the specific neural interface may be crafted/designed by the surgeon during the procedure. Interfacing with the renal plexus allows for means to interface with the ovarian plexus in females, providing a direct neural interface that may use electrical stimulation and/or block as well as heat or cold block to modulate ovarian activity over time. A direct access to the ovarian plexus may be chosen, though reflexive activity modulating other SNS and ANS responses may be avoided by additional stimulation and/or block of neural activity at the location of the renal plexus.

The renal plexus is formed by filaments from the celiac ganglia and plexus, aorticorenal ganglia, lower thoracic splanchnic nerves and first lumbar splanchnic nerve and aortic plexus. The nerves from these sources, fifteen or twenty in number, have a few ganglia developed upon them. It enters the kidneys on arterial branches to supply the vessels, renal glomerulus, and tubules with branches to the ureteric plexus. Some filaments are distributed to the spermatic plexus and, on the right side, to the inferior vena cava. The ovarian plexus arises from the renal plexus, and is one of two sympathetic supplies distributed to the ovary and fundus of the uterus.

Another application for the cured electrode is stimulation of the Ovarian Plexus with Stim and Block waveforms, to limit fertility in women, reduce abdominal pain during monthly period. This application can use a thermally conductive cured electrode to apply temperature block, alternatively or in conjunction with an electrically conductive cured electrode.

The large Surface Area leads to large charge injection capacity and low impedance, such as disclosed elsewhere herein. The large charge is due to large metal-to-electrolyte interface inside the electrically conductive cured electrode, and the low resistance is due to metal to metal direct contact throughout the electrically conductive cured electrode and to the location right next to the nerve without the several hundreds of microns of space the electrodes are separated from a nerve in traditional (i.e. cuff) electrodes. This provides the optimal electrode interface for both sensing and stimulation applications, and is suitable for charge-balanced direct current (CBDC) block. Carbon based conductive elements may be used alone or in conjunction with metal elements. also: Silicone/carbon based liquid mixtures curing inside the body

Delivering a line of liquid mixture epi-durally through a small skull bur-hole allows applications for seizures. In this instance, there is a need for a line of stim or a blob of stim at a cortical location. There are options to go sub-durally or epi-durally: sub-durally transmits to the sulci and fill around gyri, and epi-durally: more safety and use of flexible material. The advantages include (1) delivery can be via flexible tube, (2) during delivery, the liquid mixture is very malleable and thus optimized for the anatomy below. Post-cure the cured electrode can be flexible or somewhat stiff as needed (3) one bur-hole allows addressing a large neocortical area, (3) a signal generator may be implanted into the bur-hole as a cap, (4) the liquid mixture may be used to form the cap with PMMA and a gold wire (roughened) going through, preventing bacteria to travel along the gold wire but allowing the conduction of electricity from the outside via a TENS like device and (5) the liquid mixture may be used to form the cap and may include a current-limiter, voltage-limiter or an embedded signal generator.

The cured electrode enables control of the ANS by simultaneously stimulating and blocking the sympathetic and parasympathetic systems. At a local level:
Sympathetic nerves innervating an organ are accessed with cured electrode 1 (with or without implanted stimulator or Peltier element or an externally placed signal generator)
Parasympathetic nerves innervating an organ are accessed with cured electrode 2 (with or without implanted stimulator or Peltier element or an externally placed signal generator)
Stimulation may be provided by a magnetically conductive cured electrode an external coil as well
A control unit decides
   if the sympathetic innervation to the organ should be stimulated, while the parasympathetic innervation is stimulated in parallel (but maybe both at either same or different frequencies for different levels of activation) and what the time course of the stimulation and block with respect to each other are (at same time or one then the other)
   if the sympathetic innervation to the organ should be stimulated, while the parasympathetic innervation is blocked in parallel (maybe both at either same or different frequencies for different levels of activation) and what the time course of the stimulation and block with respect to each other are (at same time or one then the other)
   if the sympathetic innervation to the organ should be blocked, while the parasympathetic innervation is blocked in parallel (maybe both at either same or different frequencies for different levels of activation) and what the time course of the stimulation and block with respect to each other are (at same time or one then the other)
   if the sympathetic innervation to the organ should be blocked, while the parasympathetic innervation is stimulated in parallel (maybe both at either same or different frequencies for different levels of activation) and what the time course of the stimulation and block with respect to each other are (at same time or one then the other)
   cured electrode 1 may then stimulate and/or block as needed; similarly cured electrode 2
An external or implanted sensor may be used to close the loop for a closed-loop-control system
The user or physician may fine tune the levels of stimulation and block (within limits set by the physician in conjunction with a technician)

At the global level:
Sympathetic nerves in the body (i.e. on the sympathetic chain) are accessed with an cured electrode One (with or without implanted stimulator or Peltier element or an externally placed signal generator)
Parasympathetic nerves in the body (i.e. the vagal nerve and/or the sacral plexus providing parasympathetic access) are accessed with an cured electrode Two (with or without implanted stimulator or Peltier element or an externally placed signal generator)
Stim may be provided by a magnetically conductive cured electrode with an external coil as well
A control unit determines:
   if the sympathetic innervation to the organ should be stimulated, while the parasympathetic innervation is stimulated in parallel (but maybe both at either same or different frequencies for different levels of activation) and what the time course of the stimulation and block with respect to each other are (at same time or one then the other)
   if the sympathetic innervation to the organ should be stimulated, while the parasympathetic innervation is blocked in parallel (maybe both at either same or different frequencies for different levels of activation) and what the time course of the stimulation and block with respect to each other are (at same time or one then the other)
   if the sympathetic innervation to the organ should be blocked, while the parasympathetic innervation is blocked in parallel (maybe both at either same or different frequencies for different levels of activation) and what the time course of the stimulation and block with respect to each other are (at same time or one then the other)
   if the sympathetic innervation to the organ should be blocked, while the parasympathetic innervation is stimulated in parallel (maybe both at either same or different frequencies for different levels of activation) and what the time course of the stimulation and block with respect to each other are (at same time or one then the other)
   cured electrode two may then stimulate and/or block as needed; similarly cured electrode two
An external or implanted sensor may be used to close the loop for a closed-loop-control system
The user or physician may fine tune the levels of stimulation and block (within limits set by the physician in conjunction with a technician)

The present invention provides custom-designed energy interfaces for implantable electrical devices. Implantable signal generators, implantable data acquisition systems and other forms of implantable electrical devices that may interface with the body by transferring energy into the body or recording electrical, electro-chemical, biological or mechanical data from the body come in many different form factors, sizes and appearances. Currently, all of them have a pre-configured form or shape. Many do not allow a modification of the interface in vivo during the implantation procedure or beyond that. Most electrically interfacing devices, such as cardiac pacemakers, neuromodulation signal generators or implanted drug supplying pumps do not make use of technologies that would allow a better electrical, mechanical, magnetic, thermal or otherwise energy-specific coupling with the body.

Described herein is the modification of a device that is finished (completely manufactured) outside the body and that interfaces in a more optimal form when an cured electrode, either as electrically conductive cured electrode, magnetically conductive cured electrode, thermally conductive cured electrode, vibratory cured electrode or other energy based cured electrode is placed in direct mechanical contact with the implant and cures inside the body to form an energy waveguide that better transfers the specific energy of the implant to the body.

One implementation of such a combination product is to use an electrically conductive cured electrode to optimize the electrical interface of an implant, such as a small signal generator or a small signal recorder that itself has only a small electrode (or electrodes) but would benefit from a larger electrode interface with the body that shows the typical electrically conductive cured electrode features such as a high charge capacitance and low added resistance of the complex impedance between the signal generator's small electrode and the body. An electrically conductive cured electrode may be used to optimize the electrical characteristics of the electrode to body interface, thereby allowing for a more controlled direction of the current by controlling the bio-response and resulting inflammation around the electrode.

The present invention allows the cooling down of blood flow into a neural structure such as a ganglion

The cured electrode has the capability for sensory applications. In one embodiment, the electrically conductive cured electrode improves contact impedance and the large volume instead of surface area interface allows the whole volume of the cured electrode to pick up and transduce the voltage information near the nerve, thereby reducing the interface impedance.

Compression of the electrically conductive cured electrode may lead to a reduction of electrical or impedance magnetic permeability that can be used as a measure for mechanical pressure applied to an electrically conductive cured electrode.

The cured electrode can be used for intermittent stimulation (and/or block) of the sympathetic nervous system. The cured electrode may be used, as described herein, to stimulate or block as well as selectively change firing and metabolic activity in the sympathetic nervous system by interfacing with the nerves and ganglia of the sympathetic chain. One application resulting from such an interaction is the reduction of pain, the reduction of the perception of chronic pain, as well as the reduction of the perception of short-term acute pain. As such, the cured electrode may be used for palliative care in the treatment of terminal cancer with the goal to give the patient a better control over their pain in general as well as a reduction in drug dosage and choice of drugs used for their treatment of pain, offering the option to use drugs with a lesser habit-forming potential.

In very general terms, pain and the response of the Sympathetic Nervous System (SNS) are inter-connected. The perception of pain raises SNS activity and SNS activity may influence how a painful stimulus may be perceived by a patient. Fundamentally, there are two forms of SNS activity: Acute, short term activity, a temporarily higher firing rate and duration of a production of short bursts followed by extended periods of low-level SNS neural firing activity on the one hand; and chronically elevated neural firing rates with fewer pauses between neural spikes and fewer bursts or just continuous, repetitive bursting of neural activity, meaning the average action potential count per unit of time is either not as low as during low-level SNS activity periods following the acutely raised activity or it is generally higher. In simplified terms, acutely modulated SNS activity from very low firing rates during extended periods of time interrupted by moments of high firing rates are acute responses of the SNS and an indication of a healthy SNS. Chronic, repetitive, somewhat tonic SNS activity may manifest itself similar to neural firing patterns observed in deep brain structures during Alzheimer's disease or in auditory structures following the development of tinnitus.

Similar to heart rate in a healthy patient, acute high variability of SNS activity may be interpreted as a sign of a healthy individual, whereas chronically raised activity (without the low lows and high highs in activity) represents a reduction in SNS variability and is a sign of a less healthy individual. Acutely raised SNS activity may be the result of an acute stimulus such as a painful stimulus, hearing a beautiful song (and feeling the urge to dance), seeing a beautiful individual (causing a fight or flight response) or being challenged to perform a physically demanding activity such as in a sports competition. Chronically high sympathetic activity is correlated with the perception of chronic stress, chronic pain, potentially fear, continuous tiredness, lower heart rate variability but generally increased heart rate compared to the healthy norm as well as the inability to respond as well to acute stimuli to the SNS.

Chronically elevated SNS activity changes the overall perception of pain: small stimuli may be over-interpreted to require a major response by the body, somewhat similar to an allergic reaction where a small stimulus may result in a major response and as a result may lead to an anaphylactic shock.

The results of this observation is in stark contrast to the acute suppression of pain by temporary activation of sympathetic activity, which is a natural response of the central and autonomic nervous system to acute, short painful stimuli.

Similarly how a state of oncoming or established anaphylactic shock may be treated with the administration of epinephrine, electrical or other stimulation of the sympathetic nervous system may result in a change of chronic over activity in the SNS and rebalance the system. Short term, acute, high intensity stimulation of the SNS by activating neural fibers or ganglia of the sympathetic chain, followed either by non-stimulation of said neural fibers or ganglia or followed by intentional block of neural activity in the neural fibers or ganglia may be enough to push the autonomic nervous system out of the chronic over activity and into a more response-driven acute activity production, thereby normalizing the state of the SNS from a less to a more healthy level.

Similarly, acute stimulation of the adrenal gland, releasing epinephrine and having other glandular and neural feedback effects back to the CNS (here composed of spinal cord, brainstem and brain with added sympathetic chain along the spine), causes an acute increase in SNS activity, which later results in a parasympathetic counter activation, thereby reducing the overall SNS activity during the time of absent neural- or neuro-glandular stimulation. The acute, short term, high intensity stimulation of the SNS (with or without followed block or reduction of neural activity and/or glandular activity) thereby normalizes the SNS activity from an elevated neural firing tone of low variability to a more variable neural firing activity with lower lows (more relaxation) and higher highs (more SNS bursts when needed). The modulation of the SNS activity may be achieved by
electrical means, electrically stimulating the SNS neurons, ganglia and plexi of i.e. the sympathetic chain and connected peripheral nerve fibers directly,
electrical means, electrically blocking the SNS neurons, ganglia and plexi of i.e. the sympathetic chain and connected peripheral nerve fibers directly,
thermal means by reducing or increasing metabolic activity and thereby increasing the firing probability of SNS neurons, ganglia and plexi of i.e. the sympathetic chain and connected peripheral nerve fibers directly,
electro-magnetic means by increasing the firing probability of SNS neurons with the application of changing magnetic fields in the direct proximity of said SNS neurons, ganglia and plexi of i.e. the sympathetic chain and connected peripheral nerve fibers directly,
electro-magnetic means by stimulating SNS neurons with the application of changing magnetic fields in the direct proximity of said SNS neurons, ganglia and plexi of i.e. the sympathetic chain and connected peripheral nerve fibers directly,
optical means by increasing the firing probability of SNS neurons with the application of optical energy used to flood the neuronal tissue of said SNS neurons, ganglia and plexi of i.e. the sympathetic chain and connected peripheral nerve fibers directly,
optical means by increasing the firing probability of SNS neurons with the application of optical energy used to flood the neuronal tissue that may have been modified with optogenetic means to react to light with the production of action potentials in said SNS neurons, ganglia and plexi of i.e. the sympathetic chain and connected peripheral nerve fibers directly,
magnetic means by increasing the firing probability of SNS neurons with the application of magnetic energy used to flood the neuronal tissue that may have been modified with magneto-genetic means to react to changing magnetic fields with the production of action potentials in said SNS neurons, ganglia and plexi of i.e. the sympathetic chain and connected peripheral nerve fibers directly, and/or
acoustic means by increasing the firing probability of SNS neurons with the application of acoustic energy used to flood the neuronal tissue pressure changes with the production of action potentials in said SNS neurons, ganglia and plexi of i.e. the sympathetic chain and connected peripheral nerve fibers directly.
vibration affecting proprioceptive cells in the body; the vibration transduced by electro-magnetic means of moving an implanted/injected thermally conductive cured electrode with an oscillating magnetic field and thereby activating proprioceptive receptors in muscles, tendons or the skin of inner organs or the outer skin of the body.

It is also possible, in one embodiment to place a cured electrode on the external surface of the ear to achieve therapeutic results. The auricular nerve allows for an interface with the autonomic nervous system. Current technologies rely on gluing metal electrodes to the ear with sticky tape or, to some degree, using TENS electrodes that are further fixed with tape to specific locations on the ear. There is a need for an electrode that conforms to the ear's anatomy in a way that itself may support the mechanical stability of an electrode assembly on the ear. The cured electrode, especially the electrically conductive cured electrode, may provide such an interface when cured outside, not inside of the body, and in this case as a prime example location the ear. The ear offers various crevices, bumps and dents that allow for a unique mechanical fixation as long as the cured electrode is conforming well to the anatomy and is able to cure, partially or fully, to provide a structure that may hold itself up (i.e., support itself against the forces of gravity and or other forces of acceleration) while allowing to transfer electrical energy to the skin of the organ (here the ear) with the specific goal of providing enough of a current density outside the skin that the nerves just below the skin are activated as required. A mold may be used during the curing process, where liquid mixture is placed onto the organ's skin outside of the body (i.e. ear) and the mold is (and or additional tools may be or the physician's fingers may be) used to push liquid mixture into the various crevices, to ensure both, an excellent mechanical as well as optimal electrical interface to the body. A surgeon may choose to massage the liquid mixture into place during the placement, while only partially using a mold; another surgeon may only want to rely on a mold. A mold may be used to shape where (electrically) conductive and where non-conductive (non-permeable) liquid mixture is placed to form the final shape of the cured electrode on the outside skin of said organ.

It is also possible, in one embodiment, to spray-on a cured electrode comprised of fibrin either by (1) paste on/paint on/sprinkle conductive elements (metal grains, graphene, etc.), or: (2) wire conductive matrix/mesh around the nerve and then spray on the Fibrin. Delivery speed of metal (or other elements) vs fibrin needs to be controlled and optimized for each specific application location. By providing cooled fibrinogen and thrombin as a cooled mixture, the reaction rate can be reduced so that conductive particles or wires may be embedded to create a continuous electro-conducting path in the fibrin/thrombin mixture before it fully reacts to fibrinogen. Alternatively, by dispensing fibrin snow and thrombin snow with conductive elements/matrices/meshes/wires interwoven, the fibrin forming reaction is only initiated after melting, an approach that is only feasible as long as the underlying tissue does not get frozen and damaged in the process. It may be surgeon controlled or it may be automatic as speed of delivery of one being a function of the delivery speed of the other. In one embodiment, pulsed air is used to push elements out from a reservoir with an auger moving conductive elements into position to be sprayed. Spraying on the fibrin is advisable before wiring the mesh around the nerve/pasting on the metal/using air to spray on the metal powder.

In a second approach, fibrin is sprayed on electrically or magnetically or optically or acoustically conductive powder is sprayed/thrown on, and is repeated until desired thickness is achieved.

A cured electrode guide reaches underneath nerve, holding it up while delivering cured electrode mix. A delivery device is described with elements of a “hand” or “guide” that can be used to reaching underneath” a nerve, hold the nerve away from other structures and inject liquid mixture around the nerve without injecting it onto other surrounding structures, similar to a mold. This mold or “hand” or “guide” bends back when it is retracted away from the neural interface target, either with liquid mixture coming out beneath the nerve while the retraction happens or after the liquid mixture has been injected around (beneath) the nerve and has at least partially cured. Such a flexible mold is temporarily flexible by threading a wire as a guide into a flexible sheath, whereas the wire may be retracted from the sheath, thereby making the sheath more flexible (up to the point where it is floppy) which aids with the subsequent retraction of the sheath.

Delivery methods of the pre-mixed liquid mixture include, without limitation:

1) Two separate components in two syringes that are to be combined. Similar to the way Fibrin is sold as fibrin and thrombin, just that we add silver/gold/graphene or other conductive elements as well as a surfactant. That means that, in the embodiment with silicone, the conductive elements are first mixed with the surfactant. The surfactant ensures the conductive elements will be able to touch in the final mix, improving conductivity. This conductive element-surfactant-mix is then split in half, with one half being mixed, as with fibrinogen and the other half with thrombin. For a silicone based system that uses a two component silicone, then the mixing follows a similar approach: ½ of conduct elements with one part of silicone premixed, similarly the other ½ of the conductive elements mixed with the other part of the silicone; in the OR both mixtures are combined allowing about 2 minutes until the complete silicone/Ag/surfactant mix hardened).
Example for two-component systems that are non-conductive but feasible as carriers:
http://www.ethicon.com/healthcare-professionals/products/biosurgery/evicel-fibrin-sealant-human and https://www.wpiinc.com/products/top-products/kwik-sil-kwik-sil-adhesive/
2) Three components are mixed together: The conductive elements come pre-mixed with the surfactant as “part 1”, then the other two parts (e.g. silicone part 1, silicone part 2) are added all together.
3) Similar to how amalgam is mixed in a vibrating mixer, components may come separated in a two-part or three-part capsule. The parts of the capsule are separated by a thin layer that is damaged when the capsule is shaken rapidly. Hence the vibrating mixer: it breaks the barrier(s) inside the capsule and then mixed the components. The capsule may then be loaded in a dispensing device that empties the contents as needed by the surgeon or the capsule may be opened and the mixed contents may be extracted by hand to be dispensed/deposited onto the target (neural etc.) tissue.
4) If light cured polymers are used as a carrier then the final mix of carrier and conductive elements (+potentially surfactant) may come completely pre-mixed or in parts as well. The complete mix is dispensed onto the target tissue prior to illumination with visible, IR or UV light.

Those are a few versions of how the cured electrode may be delivered that are practical in the OR. The basic principle is to save the surgeon time and stress as much as possible, thus anything we may do to partially or fully automate the mixing/preparation of the cured electrode without impacting shelf life is advantageous.

Vibration is a method to compact the conductive elements in a liquid mixture. This allows the use of lower viscosity liquid mixture (lower mix, such as 50% Au or Ag or similar instead of 70%) as the compacting happens around the nerves only but not in the syringe. This facilitates thinner syringe/needle diameters and allows a delivery of lower viscosity (“more liquid”) mixtures. This too allows the use of carriers that are rather liquid as long as there is a stirring happening inside the syringe to keep the elements more or less equally in suspension. The user can place low viscosity liquid mixture around the nerve and helical wire end near nerve. Next the user must make sure the nerves and wire are at the lowest point of the setup and that vibration will not make the liquid mixture “flow away” off to the side. If need be, rotate the subject/animal. Then vibrate the mix by touching nearby tissue, the liquid mixture itself or other mechanically connected structures (table the animal is on etc.). Care should be taken to ensure the more dense mixture fully encapsulates the nerves and the wire. If target nerves or wire appear to be left “out in the open” without proper contact to the conductive elements, then the user must dispense more liquid mixture on top and resume vibration. The positive side effect is that the conductive liquid mixture that encapsulates the nerves and wire, electrically (thermally, mechanically, etc.) connecting both is encased by a layer of non-conductive material that floats on-top. This ensures a stronger impedance (likely in the MΩ range) against the sensory fibers in the skin or other tissues above the cured electrode.

Vibration may be used during curing to just condense conductive elements but also to speed up curing itself

Electrically conductive Silk can be combined with a liquid nonconductor material to produce another embodiment of the cured electrode. There are reasonably simple ways to manufacture electrically conductive silk. The electrically conductive silk may further be used to conduct thermal energy along the path of the cured electrode. Electrically conductive silk may be combined with a nonconductive carrier in two ways to form a cured electrode in at least the following ways:
1. The electrically conductive silk is spun into the carrier before it is deployed at the target tissue, and the silk represents a substantial part of the volume of the cured electrode as it is being delivered (injected/extruded) into or onto the patient or subject.
2. The electrically conductive silk is “shot” (e.g. rolled off a spool) into the carrier with a sufficient enough velocity that a curling or meandering of the silk happens inside the carrier during the injection of the liquid mixture or encapsulation of target tissue with the liquid mixture.

Gelatin-methacrylamide/hyaluronic acid-methacrylate (GelMa/HAMa) hydrogel provides a liquid nonconductor suitable for a cured electrode. A hydrogel formed from two components, one being gelatin-methacrylamide, the other one being hyaluronic acid-methacrylate, allows for the combined GelMa/HAMa hydrogel to be cross-linked with 365 nm UV light. As all of the components of this hydrogel lack a high magnetic permeability, a high electrical conductivity, and a high thermal conductivity, an excellent sound wave conductivity, and an optimal optical transmittance, but specific elements may be added to optimize the various capabilities of interest. Magnetic permeability may be increased by adding ferromagnetic, ferrimagnetic or otherwise magnetically permeable material containing elements in the shape of fine dust (i.e. stainless steel dust), micrometer-size grains or flakes, microspheres (1 to 500 um in diameter), oval and round structures with similar dimensions or high aspect ratio volumes shaped similar to a rod or a wire. Electrical conductivity may be increased by adding electrically highly conductive material containing elements in the shape of fine dust (i.e. gold dust), micrometer-size grains or flakes, microspheres (1 to 500 um in diameter), oval and round structures with similar dimensions or high aspect ratio volumes shaped similar to a rod or a wire. Thermal conductivity may be increased by adding thermally conductive material containing elements in the shape of fine dust (i.e. gold dust), micrometer-size grains or flakes, microspheres (1 to 500 um in diameter), oval and round structures with similar dimensions or high aspect ratio volumes shaped similar to a rod or a wire. Mechanical or sound wave conductivity may be increased by adding mechanically conductive material containing elements in the shape of fine dust (i.e. gold dust), micrometer-size grains or flakes, microspheres (1 to 500 um in diameter), oval and round structures with similar dimensions or high aspect ratio volumes shaped similar to a rod or a wire. Optical transmittance may be increased by adding optically conductive or reflective material containing elements in the shape of self-aligning fine dust (i.e. platinum dust), micrometer-size grains or flakes, microspheres (1 to 500 um in diameter), oval and round structures with similar dimensions or high aspect ratio volumes shaped similar to a rod or a wire.

Graphene microfibers (5-7 um in diameter, 100 um long), single or wound together as a cord, may be used to form thermally conductive cured electrode and electrically conductive cured electrode material mixtures.

Electrically conductive elements are, in one embodiment, optimized with PVD, CVD or electroplating. Electroplating is used to put a coating of a highly conductive metal on a less conductive metal or non-conductive non-metal substrate. Physical Vapor Deposition (PVD) may be used to put a coating of a highly conductive metal on a less conductive metal or non-conductive non-metal substrate. Chemical Vapor Deposition (PVD) may be used to put a coating of a highly conductive metal on a less conductive metal or non-conductive non-metal substrate. The highly conductive metal may be gold or another noble metal. These elements may further be coated in a surfactant, then washed to rinse off any excess surfactant before being added to a liquid nonconductor to form the electrically conductive cured electrode mix.

Although described with a focus on the applications for the electrically conductive cured electrode, other means of transferring energy with the cured electrode but the electrical pathway optimization may be achieved with this method, while providing a more noble, chemically more inert (fractal) surface on the inside of an cured electrode.

In another embodiment a metal-blood based electrically conductive cured electrode and thermally conductive cured electrode are used in combination. Whole blood as well as simply plasma may be used as a carrier and mixed with the specific elements to achieve the desired properties. A silver-blood based electrically conductive cured electrode was constructed and evaluated for electrical conductivity. Impedance values of less than 10 ohms were measured. Generally, putting silver powder alone into a wound may make the wound electrically and thermally conductive. Similar results may be obtained by adding gold powder, or carbon nanotubes. Such a metal based electrically conductive cured electrode would likely equally be a thermally conductive thermally conductive cured electrode and could be used to provide the combined temperature and electric nerve block. As the blood in the cured electrode gets resorbed over time, the electrically conductive cured electrode may feature an increasingly porous structure that is filled in part with fibrous tissue, making the whole cured electrode more mechanically flexible as time (in weeks) passes by.

The present invention enables modulation of the Inflammatory Response to add impedance at the cured electrode. Encapsulation is the body's response to an implanted object, and Encapsulation happens in stages over time. Encapsulation creates within minutes of placing a foreign object into a living organism a network of cells (i.e. platelets) and biological and chemical bonds, connections and elements (i.e. fibrin bonds) that are able to exert mechanical forces onto the foreign object as a whole as well as parts/elements/components of the foreign object as singular entities as well. If a foreign object is made up of subunits (such as i.e. micrometer and sub-micrometer (0.1 um to 0.99 um) size components of an cured electrode) then these subunits experience mechanical forces holding them in place of their implantation location post implantation if they were to having become dislodged from the bulk of the cured electrode placement location, even if the distance between the main bulk cured electrode location and the dislodged component (such as an Au element for example) is only a few micrometers or even if the distance is several millimeters or centimeters. The inflammatory response effects any size implanted object within the body with an exposed surface area to the biological tissue that initiates an inflammatory reaction leading to an encapsulation of the foreign object.

Encapsulation creates within 1 week a network of mechanically somewhat robust fibers of sufficient tensile strength to hold micrometer size elements in place and prevent them from diffusing away from the implantation site. Encapsulation may be enhanced by adding mechanical input in the form of small vibrations to a cured electrode. Encapsulation may be enhanced by adding biological or chemical input to a cured electrode (such as cells and cell fragments enhancing the inflammatory response).

The encapsulation ensures a reduced bio-availability of the foreign object within the body. The modulation (i.e. increase or decrease of the encapsulation) may change the bio-availability and chemical availability of the foreign object (i.e. cured electrode or components of the cured electrode) within the body. A thicker and/or denser encapsulation may lead to a reduced bio-availability, further limiting the body's cells being subject to unwanted interactions with the liquid mixture, cured electrode components or the cured electrode as a whole over time.

Modulation of the cured electrode may be accomplished by:
Thickness of encapsulation affects the impedance for electrical effects on tissue
Modifying inflammation changes the thickness of the body's encapsulation, affecting Impedance
Corticosteroids to minimize inflammation; dexamethasone or others
Collagen and other cells to increase inflammation
Modulating inflammation to affect electrical impedance, magnetic impedance, light impedance as well as temperature impedance

The thickness of encapsulation affects the impedance for electrical effects on tissue. The impedance of the encapsulating tissue is significantly higher than the impedance of the surrounding, moister and less dense tissue (i.e. muscle tissue). As such, the body's reaction to the implanted foreign object (i.e. the cured electrode) may be used to provide an impedance modulation by achieving a thinner or thicker encapsulation. By adding autologous cells (e.g. cartilage collected earlier and purified) to the liquid mixture, a thick encapsulation may be achieved around the cured electrode. This thicker encapsulation provides a higher electrical impedance and stronger mechanical strength than a thin encapsulation would. As such, the thick encapsulation is to be used on the outside facing part of the cured electrode if an cured electrode is employed in a two-step approach, where a thin-encapsulation liquid mixture may be placed towards the neural target tissue and the thicker, stronger encapsulating liquid mixture (all other components the same but added i.e. collagen or cartilaginous cells) on the outside.

This multi-step cured electrode may be provided in one applicator such as syringe with the frontal part (injected first) of the chamber being filled with liquid mixture that is intended to provide a thin encapsulation and the following (back) past of the chamber (of i.e. a syringe) being filled with the liquid mixture that incorporates an additional component that increases the inflammatory response.

Reducing the bio-availability of cured electrode components by modified encapsulation is a capability of the cured electrode. In a similar approach to the cured electrode facilitating a thicker encapsulation for a stiffer mechanical integration or thicker (and thereby higher) electrical impedance, the encapsulation is used to control the timed release of drugs or other components from the liquid mixture. In that case, the combined cured electrode uses a component that causes a quick growth of encapsulating tissue around the cured electrode which can later be resorbed to allow a long-term thin encapsulation. Modulating the inflammatory response with volatile components in the cured electrode can facilitate such inflammation profile over time.

Additives to the cured electrode can reduce post-injection pain and other effects To further aid with the integration of the cured electrode post-injection and combat pain, various agents may be added to the liquid mixture that are released slowly, allowing a control of post-injection pain, inflammation, and other effects.

Pain controlling additives include Lidocaine, Marcaine, Carbamazepine, Topiramate, Lamotrigine, Oxcarbazepine, Gabapentin, Morphine derivatives, Hydrocodone/acetaminophen, Ibuprophene and Propofol. These and other pain medicines may be added to the liquid mixture in combined or further encapsulated form to allow a slow release profile over time. Steroids including steroids, corticosteroids, glucocorticoids (dexamethasone) may be added to the liquid mixture to minimize post injection pain, reduce local inflammation, tissue and neural swelling in the vicinity of the injection/placement location and have other positive side effects on localized wound healing and patient satisfaction. Antibiotics may be added to the liquid mixture to minimize post injection infections, pain, reduce local inflammation, tissue and neural swelling in the vicinity of the injection/placement location and have other positive side effects on localized wound healing and patient satisfaction.

The present invention also comprises leads, wires and connections to other energy guiding materials in addition to the cured electrode itself. Avoiding corrosion between the cured electrode and the electrical lead wire is very important to a chronically successful connection between them. Silver or gold plating of a e.g. stainless steel or copper core wire are one solution. Thin film leads also have the capability to change metal within an isolated part of the lead:
Interface to the cured electrode conductive elements comprises the same material (i.e. silver, gold, etc.) as the conductive elements within the cured electrode.
Polyimide (with or without parylene C coating) on the outside, at the transition point between the metals (silver, or gold connected to stainless steel, copper, or others) on the inside of a sandwiched micromachined structure
The structure may be manufactured using photo-lithographical techniques
Method of manufacturing the sandwich comprises the following steps:
   Use a Polyimide substrate.
   Place photoresist, expose and etch to leave the area for the later wire open.
   Deposit metal 1 (i.e. steel or copper) via physical vapor depositioning PVD or chemical vapor depositioning (CVD) for a distance of just over the first half of the open “later wire opening”.
   Deposit metal 2 (i.e. gold or silver depending on the flake material in the cured electrode) via PVD or CVD for a distance of just over the second half of the open “later wire opening”.
   Etch remaining Photoresist.
   Cover with more Polyimide.
   Seal everything in at least 4, or as many as 8 to 10 layers of Parylene C to achieve a status close to hermeticity.

### RF Ablation

Radiofrequency (RF) Ablation utilizes the application of alternating current in the high kilohertz range (350 to 400 kHz) to heat and destroy tissue and, in the case of nerves, prevent the tissue from conducting signals such as pain. Although damaged (ablated) nerves grow back, there is a permanent effect on the nerve that may last for months until a conduction, often reduced, may be reestablished by the body as nerves in the periphery have the ability to grow back. Nerves in the periphery often grow back in their original tracks or along other neural tissue which is why nerve grafts (from the same person or other people) are often used to provide a path that a person's own injured/damaged/severed nerve can grow back on or in.

The cured electrode described elsewhere herein, with and without modifications, functions as an Ablation interface: RF energy applied to the cured electrode is dispersed throughout the cured electrode and generates heat at the surface boundary between the low impedance electron-conductor cured electrode and the higher impedance ionic conductor bodily tissue. By placing the cured electrode as an injection near and/or around a nerve, or other bodily tissues, uniform heating of the nerve or other tissue can be achieved during ablation application.

Applications range from a neuromodulation interface using pulsed RF to temporarily heat a nerve and reduce or increase neural conduction (i.e. as pain or spasticity treatment); to a neuromodulation interface using RF ablation aimed to permanently destroy neural tissue (with the understanding that Peripheral nervous system nerves will grow back, at least in part) that allows for an easy re-ablation thanks to the improved visibility of the cured electrode near and/or around a nerve; to a tissue ablation interface that can be used to affect non-neural tissue such as cartilage, muscle, blood vessels, glia; and the ability to use this interface at, near, inside and/or around collections of cancerous cells to ablate tumors in varying stages, shrinking them, minimizing their growth or preventing them from growing beyond a certain barrier. Applications are also found in the realm of treating blood vessels that supply other unwanted tissues inside the body such as fatty tissue and others. Applications are also found in using the cured electrode to deliver either direct current with the intent of destroying tissue or RF energy with the intent of heating and destroying tissue, such as neural tissue, that innervates muscles, thereby providing the means for a muscle relaxation similar to a phenol or botulinum-toxin nerve block. Applications may thus range from the medically necessary to the aesthetically desired.

While a prior electric lead 40 placed next to a nerve will not be able to fully depolarize the nerve but generally only cause a full depolarization of fibers near the lead and an incomplete depolarization of the nerve more distant from the location of the lead, the cured electrode in its way to encase the nerve at low impedance values (<100 Ω or even <10Ω) provides a simple surgical approach to connect to neural tissue in various locations, different patients and both within a short application/implantation/injection time. For some ablation applications, the optimal impedance of such an cured electrode may be in the range of 50 to 250Ω, for some other ablation applications, the optimal impedance of such a cured electrode 1 may be in the range of 5 to 10 kΩ, all impedance measured either at 1 kHz for compatibility to earlier patent applications or at 100 kHz to be in the similar range as RF ablation frequencies.

Radiofrequency Ablation in general utilizes the application of alternating current in the high kilohertz range (350 to 400 kHz). Radiofrequency ablation of the neural tissues is a method using electrical energy transmitted through an electrode inside a RF needle which is broad into contact with the tissue inside the body that is to be ablated or broad into contact with tissue that is close (2 to 3 mm) to the tissue inside the body that is intended to be ablated. During the ablation procedure, the energy transmitted from the signal generator causes heat around the tip of the needle. The increase in temperature close to the needle tip to above 80 degrees Celsius usually is sufficient to induce protein denaturation and tissue necrosis. In many cases, temperatures above 65 degrees are enough as long as they are applied for long enough. Generally, the higher the applied temperature, the shorter the necessary ablation duration and vice versa. The volume of tissue necrosis is proportional to the size of the active needle tip. Close proximity to a nerve can cause the equivalent of a neurotomy. Neurotomy of the sensory nerve fibers innervating the capsule of a joint is thought to inhibit pain signals traveling to brain and result in pain relief

Traditional RF probes are insulated needles with a 5 or 10 mm active tip. An electrode is placed inside the RF needles to transmit the electrical current. The RF needle is positioned close to the targeted nerve and it produces an oval lesion along the active tip. The RF ablation of the medial branches that innervate the facet joint produces a significant improvement in pain intensity for 6 months on average since it is hypothesized that the nerves regrow to bridge the gap. Repetitive lesions have shown to be equally effective.

Needle gauges 16 g-20 g are typically used along with lessoning durations of 90 s to 150 s and at temperatures of 60-80 degC, depending on the specifications of the electrode used and the lesion volume desired. Prior art RF ablation systems include (1) cooled RF (CRF) (17 g), “V” shaped active cannula and protruding electrode (PE) (18 g and 20 g), and monopolar RF (16 g, 18 g, and 20 g). These systems when tested in chicken breast tissue were found to be capable of producing lesion volumes ranging from approximately 143 mm³ (monopolar 20 g, 90 s @ 80 degC) to 595 mm³ (CRF 17 g, 150 s @ 60 degC). Information on prior art ablation devices is found in “Pain Physician: September/October 2017: 20:E915-E922” which is incorporated in its entirety by reference herein.

The cured electrode 1 has great advantages for ablation over the prior art. Prior art ablation devices require minimally invasive repetitive procedures, especially for the case where insufficient nerve ablation is achieved during a procedure or when the nerve has recovered after e.g. months post a traditional procedure. Each time a patient must undergo an ablation procedure, the physician must map out the anatomy, verify the proper ablation needle placement and can only then apply the RF energy. The cured electrode 1 on the other hand is placed under US or fluoroscopy visualization for the first time and repeat procedures are applied wirelessly, transcutaneously, or with a simplified needle-based procedure. Prior art ablation devices confront anatomical challenges for accessing the nerves, but the cured electrode has the ability to be placed near or around nerve structures and conform to their geometry. For the prior art, the needle geometry and consequently the lesion geometry does not allow for appropriate and easy targeting making the procedure less effective, i.e., the oval shape lesions 93 originating from the side of the nerve 5 are not optimized for the specific lesion volume desired, but the cured electrode lesion 93 (right side) is confined to a smaller area. FIG. 113 depicts the ability of the cured electrode (right side) to confine the lesion volume to a smaller region than prior art monopolar electrodes 40 (left side). The cured electrode, being highly thermally conductive allows greater energy transfer from the probe to the cured electrode and from there diffusion into the surrounding tissues. This enables a shorter duration of lesioning, which injects an overall smaller amount of energy into the tissue. A smaller RF ablation tip is shown for the cured electrode combination since the primary mode of action is transfer from the electrode to the cured electrode to the tissues, concentration of heat in center of the cured electrode in case of donut/cuff shape.

Additional advantages of the cured electrode for ablation include (1) implanted mini-electrodes delivered in selected patient by diagnostic procedure as per standard of care to avoid any false positive, (2) the possibility for self-administered pulses on demand, and (3) no need for repeated minimally invasive procedures, no discomfort and no need for hospital or outpatient surgery center resources. The cured electrode's lesion is smaller and closer to the targeted tissue and exact location is quite significant. This is especially important in treatment of chronic pain due to osteoarthritis of the lumbar facet joints, sacroiliac joints or event hip and knee joints. Delivery of the “cured electrode” is performed under fluoroscopic guidance. The “cured electrode” is opaque which makes it easy for specific delivery. The cured electrode can be placed at many locations including without limitation (1) Facet joint location of medical branches in the lumbar spine, (2) sacroiliac joint either along the sacral articular surface or at the 6, 9 and 12 o'clock position of the foramina S1, S2 or S3, (3) hip articular branches of obturator nerve, (4) knee genicular nerve medial and lateral as well as recurrent, (5) genitofemoral nerve, (6) Ilioinguinal nerve, (7) suprascapular nerve, (8) greater occipital nerve, (9) vagal nerve and trigeminal nerve. The cured Electrode is able to conform/molded according to the anatomical location it is delivered. Once implanted the cured electrode is available for multiple uses: (A) RF ablation to deliver heat multiple times, 2 pulses every 20 seconds, (B) Pulsed Radiofrequency, to deliver the heat not just around the electrode but multiple times, 2 pulses every minute—thereby less temperature increase, and (c) Neuromodulation, stimulation or block,

The energy to the cured electrode 1 can be transmitted wirelessly and transcutaneously with a patch electrode with a transmitter with a needle (small gauge) insulated that touches the cured implanted electrode and connects with an energy source via a “hook” connector” or via magnetic or microwave energy transfer by using a filler that has a ferromagnetic core (e.g., Fe3O4, iron (II, III) oxide). The shape of the cured electrode and the targeted ablation volume further determines the delivery tool, be it a needle with an insulated tip, a V-shaped one or other. Control of the procedure can be performed (1) via a mobile Phone “app” application platform what controls a small IPG that can be charged regularly in conventional manner of pulsed-RF potentially used this way to heat the nerve to provide neuromodulation and not ablation or RF-ablation (non-pulsed) with such a system to be used by a physician, (2) by a patient therapy device comprising the battery and the patch electrode placed on the skin above the cured electrode, and/or (3) software and electrical pulse design. The present invention is introducing a novel approach to ablation and neuromodulation via the cured electrode. It includes a new device, delivery instruments, application, method of treatment, software and hardware.

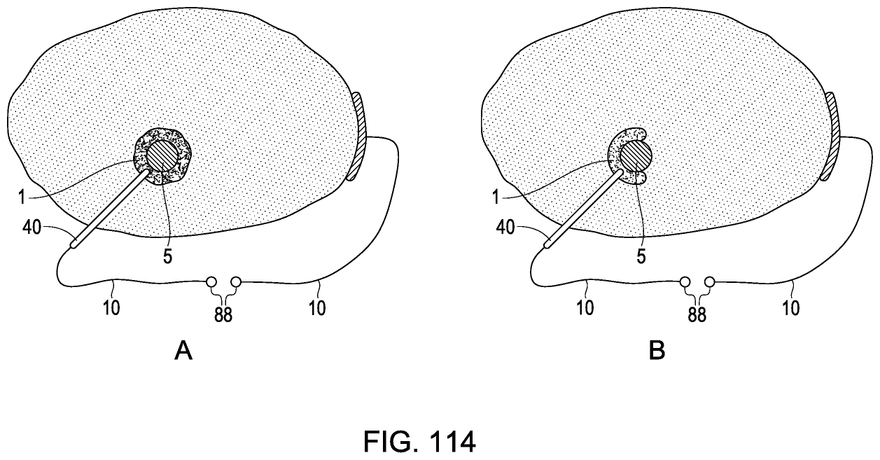

FIG. 114A-B depicts an embodiment of the contact-based Ablation approach described herein. The cured electrode 1 in (A) fully surrounds the nerve 5 whereas the cured electrode in (B) only passes by the nerve or partially surrounds the nerve. By touching the cured electrode during RF application heat is generated either inside the cured electrode 1 or at the interface between the cured electrode 1 and the surrounding bodily fluid and bodily tissue (the effect can be steered by choosing a lower or higher impedance cured electrode 1, as described further herein).

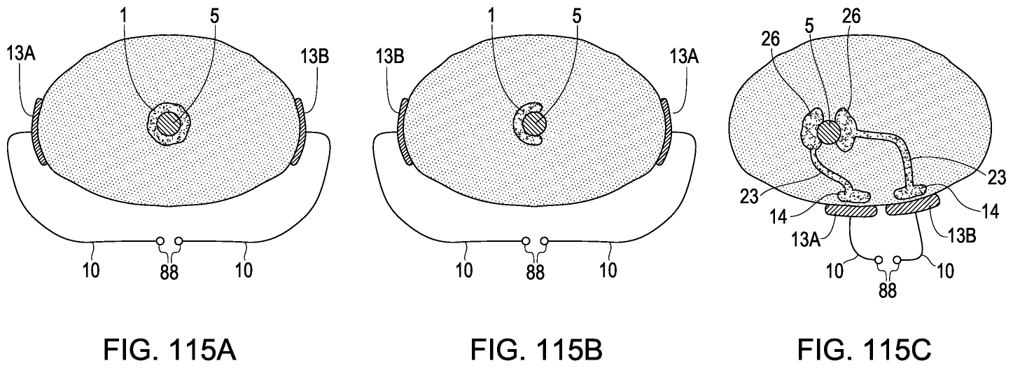

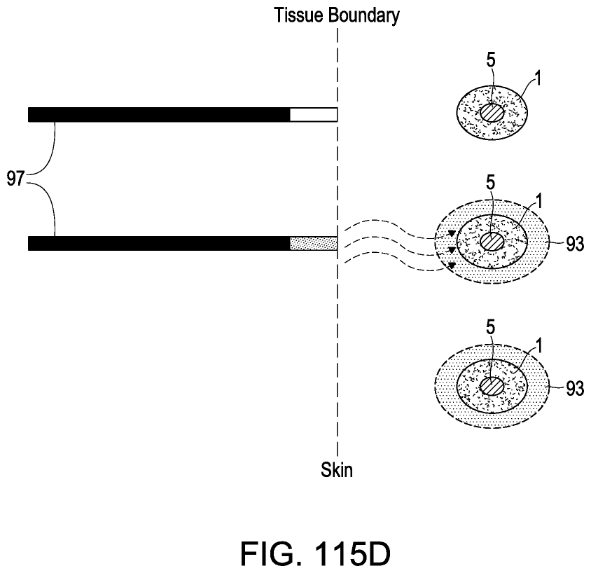

The cured electrode enables Ablation with patch electrodes from the outside of the skin. By overlaying energy signals applied from the outside of the skin aiming at a common crossing point inside the body at the cured electrode, ablation can be applied without breaking the skin with a needle, or the needle can be only very superficially breaking the skin to induce optimal electromagnetic coupling to the body. FIG. 115A-C depicts patch electrodes 13A/13B with this contact-based ablation approach: The cured electrode in (A) fully surrounds the nerve whereas the cured electrode in (B) only passes by the nerve or partially surrounds the nerve, and whereas the energy transmission can be further aided as depicted in (C) by drawing the liquid mixture out closer to a contact pad 14 in the subcutaneous tissue (shown to its full possible extent, or exaggerated). By transferring the energy to the cured electrode as electromagnetic waves during RF application heat is generated either inside the cured electrode material or at the interface between the cured electrode material and the surrounding bodily fluid and bodily tissue (the effect can be steered by choosing a lower or higher impedance cured electrode material mix, described herein). The energy applied to the cured electrode which concentrates the energy on the target tissue (be it neural or non-neural, healthy or cancerous) to achieve an ablation effect of the tissue or at the minimum a heating effect of the tissue (such as with pulsed RF).

FIG. 115D depicts wireless or transcutaneous coupling with a cured electrode. Energy may be transferred transcutaneously, whether electrically, electromagnetically, RF, or US coupled.

An electrically conductive cured electrode may be used for DC ablation of superficial peripheral efferent and/or afferent nerves. For efferent nerves providing neural input to muscles (i.e. facial muscles) and for afferent nerves that provide sensory input from i.e. a painful region such as in the face via trigeminal afferents. One method for doing so comprises the steps of (1) placing the electrically conductive liquid mixture by needle injection around or near the target nerve, (2) optionally while extracting the needle extending the liquid mixture towards the skin but not leave the skin as the needle exits the puncture wound, (3) waiting for the liquid mixture to cure, (4) optionally waiting for the cured electrode to be encapsulated by the body for the period of 2 to 8 weeks, (5) placing a hydrogel or metal electrode on top of the skin overlaying the injected cured electrode and a distant return electrode, (6) applying stimulation current through these electrodes to verify the stimulation threshold for the nerve by increasing the current or voltage amplitude of current or voltage controlled, 200 μs pulse width wide charge balanced stimulation pulses at e.g. 10 Hz stimulation frequency while observing for muscle contractions (to ablate an efferent nerve) of the target muscle or while observing for reports of pain perception by the patient (to ablate an afferent nerve) and measuring said stimulation threshold for a later comparative measurement, the (7) applying a DC signal through the same electrode with an amplitude sufficient enough to ablate the tissue of approximately 5 to 50 milliampere depending on nerve diameter for a period of tens of seconds, and (8) measuring the current/voltage stimulation threshold for the nerve in the same way as done before (in “6” above) to verify that the stimulation threshold has increased considerably. A considerable increase may be understood as a doubling (2×) or quadrupling (4×) of the stimulation threshold, but in certain cases and incorporating the clinician's experience, a targeted post-ablation threshold of 10× or more may be desired. Note that the application of DC (in step 7 above) may comprise a ramping of the DC in such a way that the patient does not experience a painful perception during the DC ablation procedure, enabling the application of said method without the need to apply any analgesia or anesthesia and further enabling a graded or stepped ablation procedure protocol. If the physician or patient chooses to only target a partial ablation of the nerve and intents to verify the success by targeting a 2× increase of the stimulation threshold post DC ablation, but then notices that the clinical (medical/aesthetic) outcome of the ablation was insufficient (muscle still contracts partially and enough to be of concern, or the pain from an afferent nerve is still too strong), then an immediate re-application of the DC ablation is possible with the intent to subject the nerve with a longer cumulative DC ablation and target a post-second ablation threshold to be a 4× of the initial threshold measured. Figuratively, the 4× stimulation threshold may be understood as more tissue having been ablated than for the case where a 2× threshold has been measured, as more current is needed to activate nerve bundles on the edge of the ablated tissue volume.

The materials for the thermally conductive elements and the liquid nonconductor of the thermally conductive cured electrode as described elsewhere herein include without limitation, PEG, Silicone, Cyanoacrylate, PMMA, other Hydrogels and the conductive elements include without limitation Silver/Gold/Alloys of one or both, Platinum and platinum alloys such as Platinum-Iridium, Titanium and titanium alloys, Stainless steel (less electrically conductive but good) which dissipates heat well and has higher impedance than silver and is cheaper than gold or silver, and other metals that are bioinert or biocompatible and sufficiently electrically and/or thermally conductive

Different experiments were performed with different mixtures. In one embodiment, mixtures of resulting impedances between 4.1Ω (at 100 kHz) and 400Ω (at 100 kHz) were optimal, producing at least a millimeter of denatured (cooked) tissue as identified by whitening when compared against the pink original tissue. Mixtures of 1.2 kΩ at 100 kHz were effective but not as optimal as the lower impedances mentioned before. Mixtures of up to 34.5 kΩ (at 100 kHz) were useable in that they were able to cause whitening of the tissue during application. Mixtures less than 10Ω (at 100 kHz) were found to be effective but a heating of the current feeding wires from the signal generator to the applicator was observed. Conductive element size and shape of finished cured electrode were significant variables—¼ HF wavelength etc. to selectively heat and selectively disperse, form an antenna optimized for specific frequencies, and/or a shield or a reflector of specific frequencies and thereby had the ability to concentrate heat generation more on one side if this side is concave.

A Peltier element may be present either in close proximity or in direct contact with the liquid mixture, allowing a readout of the temperature during the RF ablation procedure. It is important that the Peltier element is electrically insulated against the RF energy, but thermally connected well enough to the mixture. This may be achieved by placing the Peltier element in a ceramic case that is in mechanical contact with the conductive finished mixture on one side (the “hot” side) of the Peltier element and the other side being exposed to the surrounding tissue. A real time feedback control of the ablation process is enabled by providing such a temperature readout from the conductive finished mixture during the application of RF energy, providing the operator data that may be used to direct how much and/or long ablation energy is applied. The regulation of the ablation energy may be done by an operator directly or by electronic circuitry and processing unit utilizing the temperature data over time.

Experimental data has been obtained via Coagulation Ablation and Microwave Ablation. A surgical coag unit (Stryker Serfas Energy RF Generator #279-000-000) with a corresponding hand piece (Stryker 279-351-250 Endoscopy Serfas Energy 50-S Hand Piece 3.5 mm) was used to apply 200 kHz RF energy to tissue and initiate an ablation reaction with a cured electrode present in tissue.

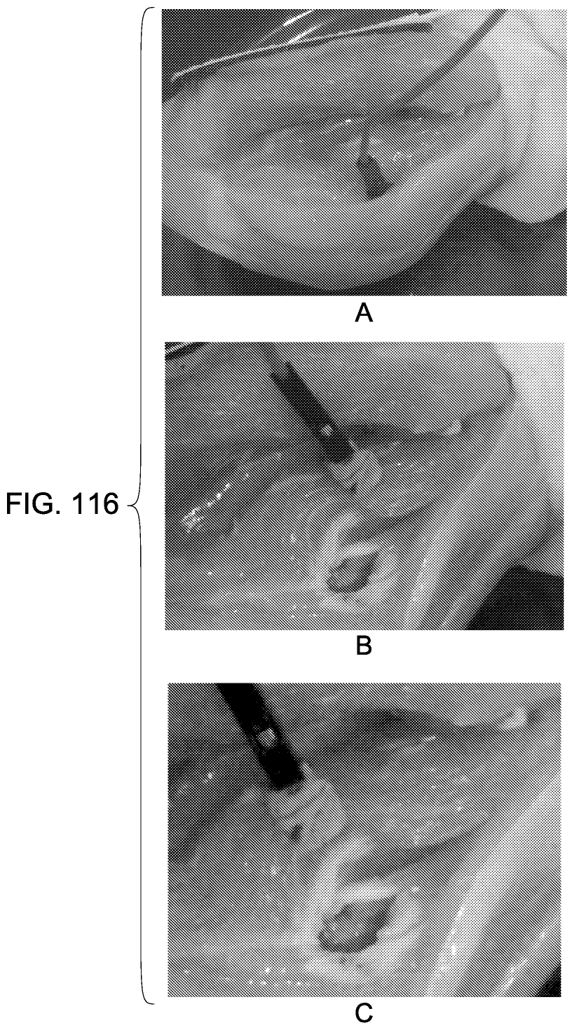

FIGS. 116-127 show RF Ablation Experiment data including images for ablation results using the cured electrode as an Ablation interface, either in chicken, or pork muscle tissue as well as in saline to show the dispersion of heat. The cured electrode was either applied as a pre-cured block of material that was placed into contact with the cadaver tissue (Experiment 1), or as pre-cured block of material placed into contact with saline (Experiment 2), or as uncured cured electrode material that was placed as a slow-meandering structure on-top of cadaver tissue (Experiment 3), or as uncured cured electrode material that was placed as a meandering mesh on-top or into of cadaver tissue (Experiment 4).

FIG. 116A-C are visible light images of experiment 1, ablation of chicken tissue with pre-cured cured electrode attached to a wire. Note the return electrode on the opposite side of the leg. A before ablation and B (C is a zoom view of B) is after, with re-extracted cured electrode on a wire, in which the light colored (meaning ablated) tissue all around the interface area with the cured electrode and note that there is no such whitening near the return electrode on the opposite side of the leg. Note the light colored (meaning ablated) tissue all around the interface area, both on the sides as well as deep inside the hole.

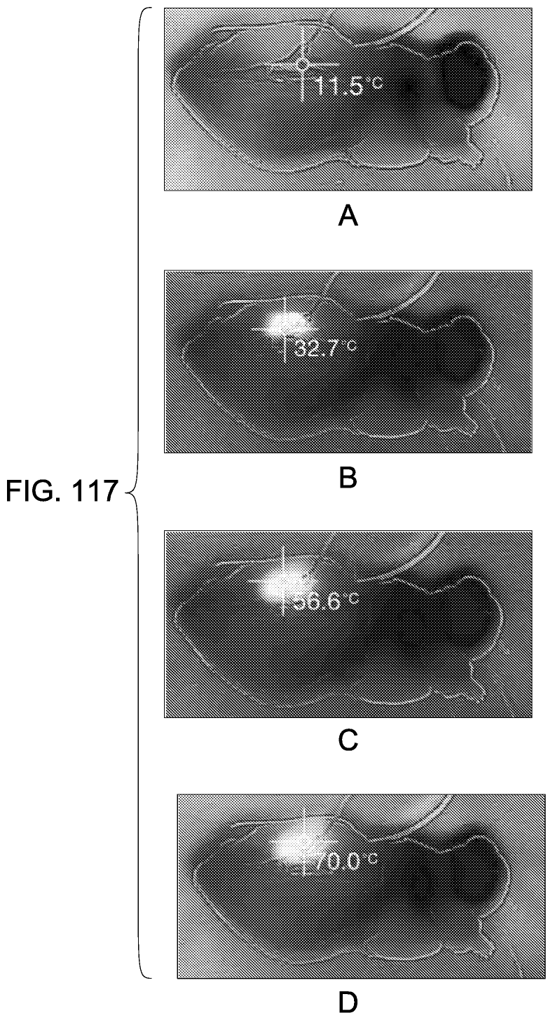

FIG. 117A-D are infrared (IR) images from RF Ablation Experiment 1 also shown in FIGS. 116A-C: Ablation of chicken tissue with pre-cured cured electrode. Images were taken with a Flir Infra-Red temperature device, the images acquired from an IR video. Temperature in FIG. 117A before ablation was 11.5 degrees C. 117B shows the temperature of the chicken tissue during the application of the ablation procedure after a few seconds is 32.7 degrees C., a difference of about 20 degrees C. This temperature is not capable of ablating tissue, but it provides an appreciation of the surrounding volume of tissue that is heated during the ablation procedure. FIG. 117C shows the temperature of the chicken tissue during the application of the ablation procedure after a few more seconds is 56.6 degrees C., a difference of about 45 degrees C. vs. the initial temperature. The ablation process has begun and could be kept at this temperature if low-temperature ablation is targeted. FIG. 117D shows the temperature of the chicken tissue during the application of the ablation procedure after more time is 70 degrees C., a difference of almost 60 degrees C. vs. the initial temperature. The ablation process is happening inside the tissue surrounding the cured electrode material.

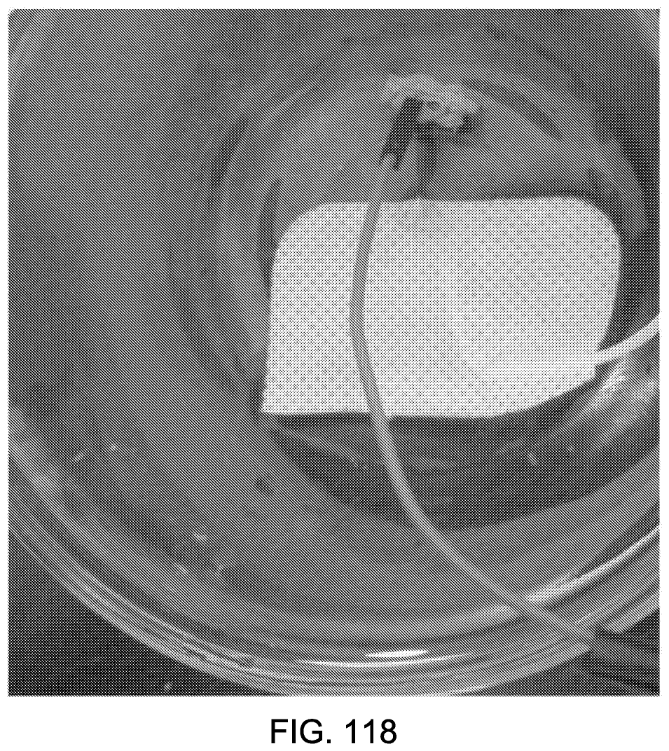

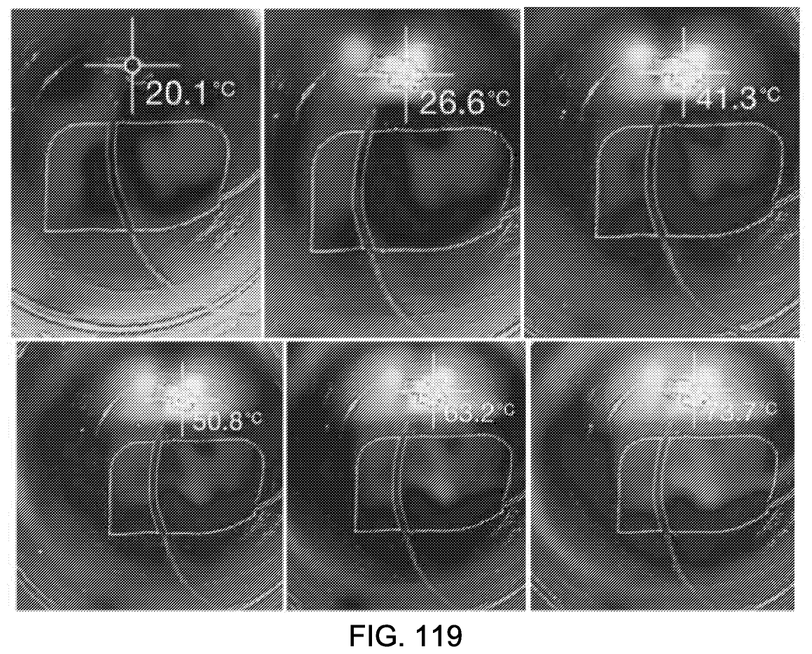

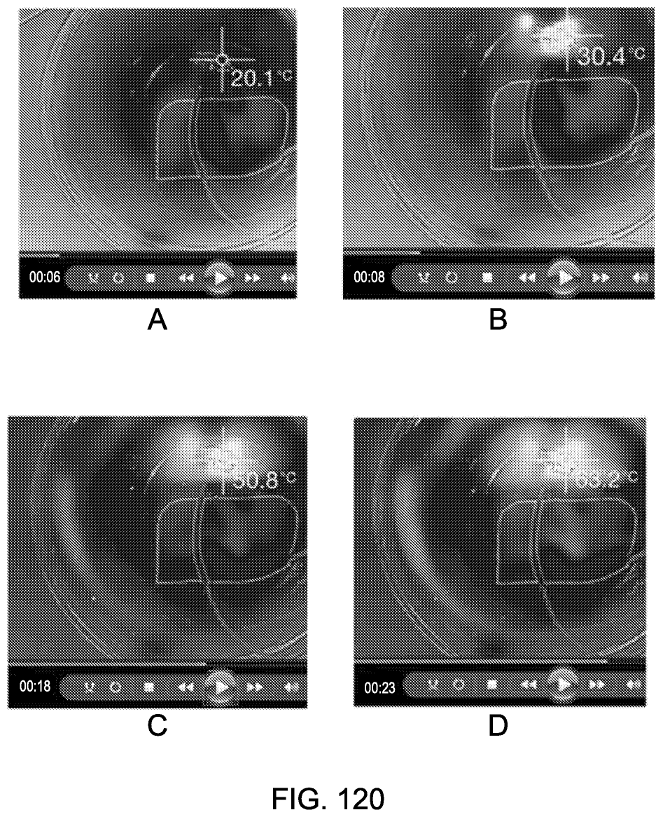

RF Ablation Experiment 2 included temperature (IR) recording of active cured electrode and counter electrode, both immersed in saline. This experiment assessed the generation and dissipation of thermal energy at the surface boundary between the cured electrode and the surrounding higher-impedance material, in which the active pre-cured electrode was placed into 0.9% saline, as shown in FIG. 118. A counter electrode (approximately ½ TENS electrode) was immersed in the same saline. A Flir IR video camera was used to record the temperature change over time. Saline cover applied was approximately 5 mm, thus fully immersing both the electrodes. FIG. 119 contains six photographs from before (upper left) application of the ablation RF energy to during the application of RF energy (consecutive images). The temperature readings went from 20.1 deg C. (upper left) to 26.6 deg. C (upper mid) to 41.3 deg C. (upper right) to 50.8 degrees C. (lower left) to 63.2 deg. C (lower middle) to 73.7 degrees Centigrade (lower right) for a maximum delta temperature of 53.6 degrees centigrade. To assess the time course, the images in FIG. 120 provide time stamps from the same video that showed the progression of the heat application shown in FIG. 119. FIG. 120A: Time stamp of the IR video is 6 seconds. No RF ablation energy has been applied at this point. The temperature is room temperature approximately 20.1 degrees C. for the entire experiment. FIG. 120B: Time stamp of the IR video is 8 seconds. The RF ablation energy has been applied for two seconds at this point. FIG. 120C: Time stamp of the IR video is 18 seconds. The RF ablation energy has been applied for twelve seconds at this point. Ablation is achieved at this temperature if the saline is substituted with tissue. FIG. 120D: Time stamp of the IR video is 23 seconds. The RF ablation energy has been applied for 17 seconds at this point. At this temperature, ablation is expected to occur rapidly.

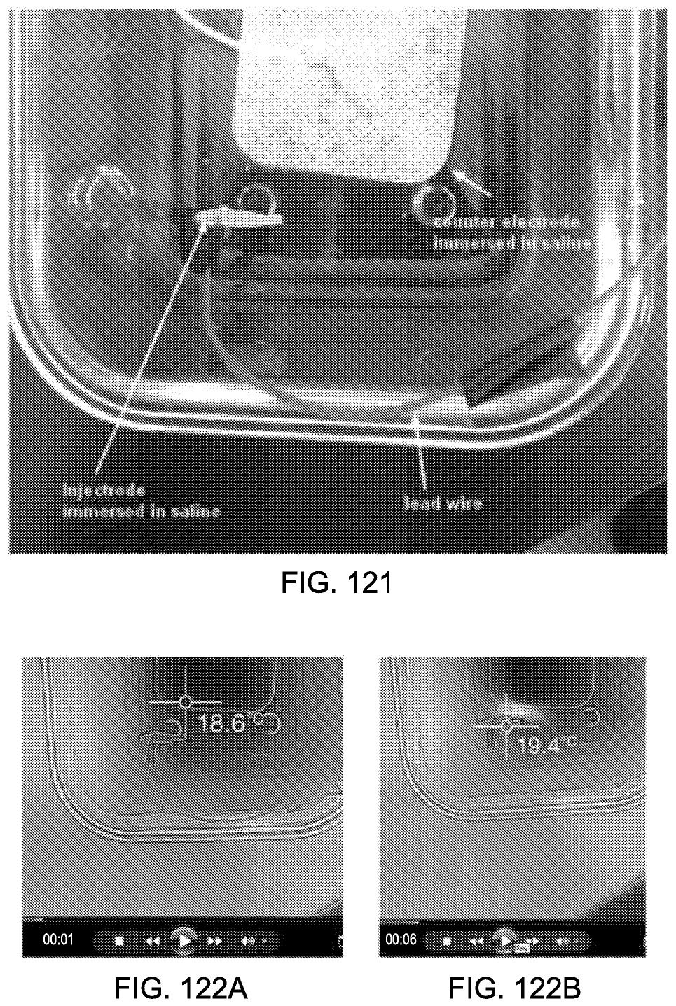

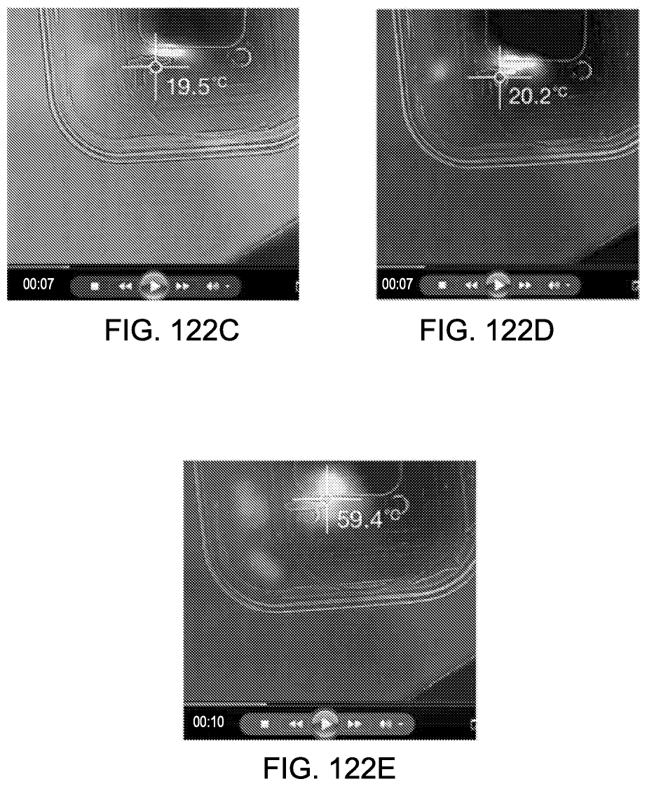

Experiment 3 was to investigate temperature generation and tissue ablation along the cured electrode. The ablation effect follows the shape of the cured electrode placed into tissue. A meandering snake could thus be placed just as well as a line of cured electrode material. As the setup is depicted in FIG. 121, a simple line of cured electrode material was placed below saline in a beaker with a standard TENS electrode as a counter. FIGS. 122A-E are taken from a video that has been recorded with an IR sensitive camera (Flir One) which unfortunately does not perfectly align the heat signature it records with one IR camera and the black and white edge-detected image it overlays the IR signal on. There is thus a delta in space of about 1 cm due to the close proximity of the IR camera to the beaker. The images from the video still bring the data across that heat is released all along the line of cured electrode material into saline and then the heat dissipates through the beaker as the warm saline moves. An observation of experiment 3 is that the generated heat is along the entity of the cured electrode line. FIG. 122A: IR recording of the cured electrode Line. Image taken before RF ablation is applied. Note the similar temperature of 18.6 degrees C. all around the cured electrode line (placed just to the left and below the temperature crosshairs. FIG. 122B: image taken 2 seconds post initiation of RF ablation application. Note the temperature generation just to the right and above the temperature reader crosshairs and that the temperature generation of 19.4 degrees C. is linear in nature. Due to the misalignment of the IR camera and the black-and-white edge detection camera there is a delta between the cured electrode location and the heat signature (a common problem of the Flir One when recording IR from targets closer than one meter; here the distance was about 20 cm and thus there is misalignment). FIG. 122C: Image taken 3 seconds post initiation of RF ablation application. Note the temperature generation of 19.5 degrees C. just to the right and above the temperature reader crosshairs and that the temperature generation is linear in nature (again, note the misalignment). FIG. 122D: Image taken about 3.4 seconds post initiation of RF ablation application. Note the temperature generation of 20.2 degrees C. just to the right and above the temperature reader crosshairs (misaligned) and that the temperature generation is linear in nature. FIG. 122E: Image taken 5 seconds post initiation of RF ablation application. Note the temperature generation of 59.4 degrees C. just to the right and above the temperature reader crosshairs and that, although the temperature generation was previously linear, it is now beginning to float around with the saline in the beaker.

RF Ablation Experiment 4 was to investigate whether a cured electrode generates heat sufficient for ablation from microwave energy application. (2.4 GHz) energy without ablating tissue from outside of the section of interest.

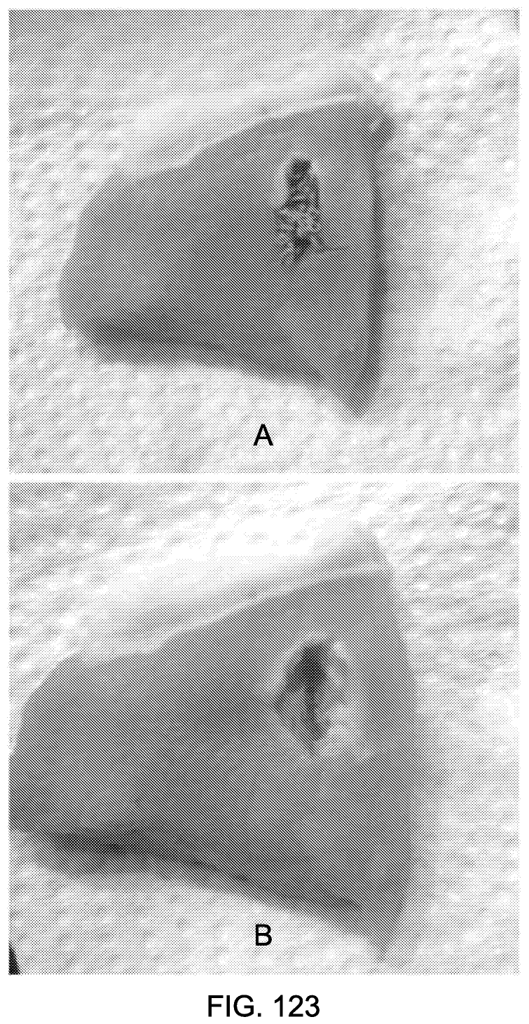

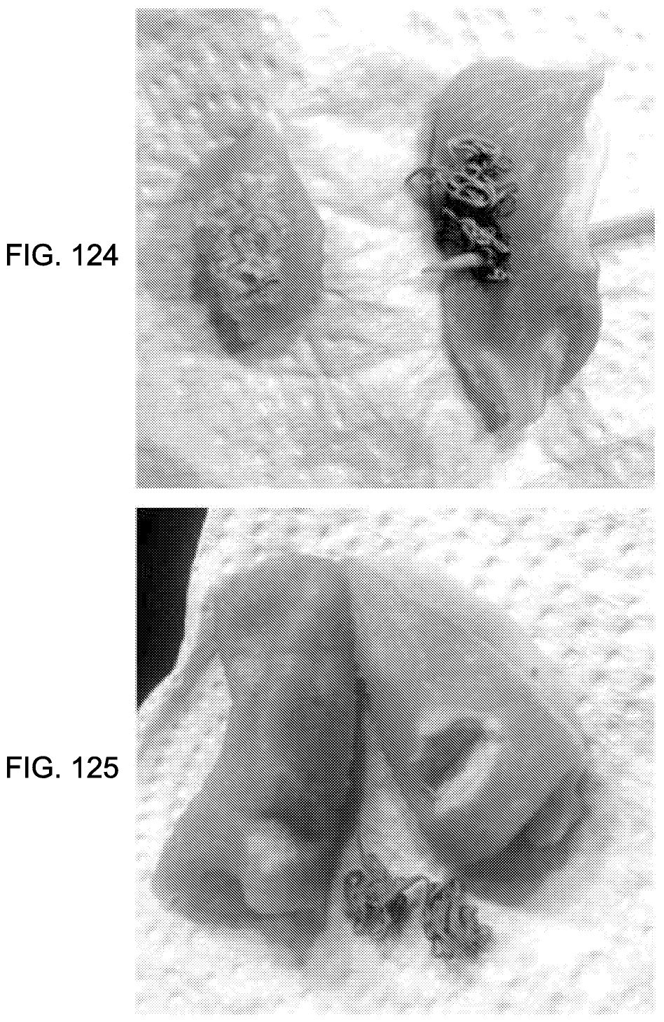

Pork muscle tissue was sectioned in a horizontal plane, then incised in a sagittal plane to form a pocket. This pocket was filled with cured electrode material via syringe (inner diameter 0.8 mm), hence the intentional filling here with a “worm-like” structure. The cured electrode material had a linear impedance of approximately 50Ω per cm while the pork muscle tissue had a linear impedance of about 500Ω per cm. To prove that there is an optimal range for the cured electrode impedance, experiments were repeated with crumbled aluminum foil which has a much lower (<1Ω) impedance than the cured electrodes that showed the strongest ablation effect. FIG. 123 shows the experimental set up: Pork muscle tissue with cured electrode material placed (injected) into a cavity (top) and removed from the cavity (bottom). Both images were taken post microwaving for approximately 10 seconds at 10% of maximum energy (1200 W max output, 10% being 120 W). Note how only the tissue in close proximity has denatured from pink (here: grey) to white while the surrounding tissue remained pink (here: grey), indicating that the energy applied to ablate tissue was limited to a few millimeter near the placed cured electrode. FIG. 124 shows a cured electrode mix stuck between two pieced of pork muscle tissue. Note how the tissue in contact turned white (meaning: was denatured—or—ablated) while the surrounding tissue was not. The two pieced of pork tissue were held in place by a wooden tooth pick. FIG. 125: another cured electrode mix stuck between two pieces of pork muscle tissue. Note how the tissue in contact turned white (meaning: was denatured—or—ablated) while the surrounding tissue was not. The pork chop was sliced in half post ablation right through the ablated area with care to not cut the cured electrode material too much.

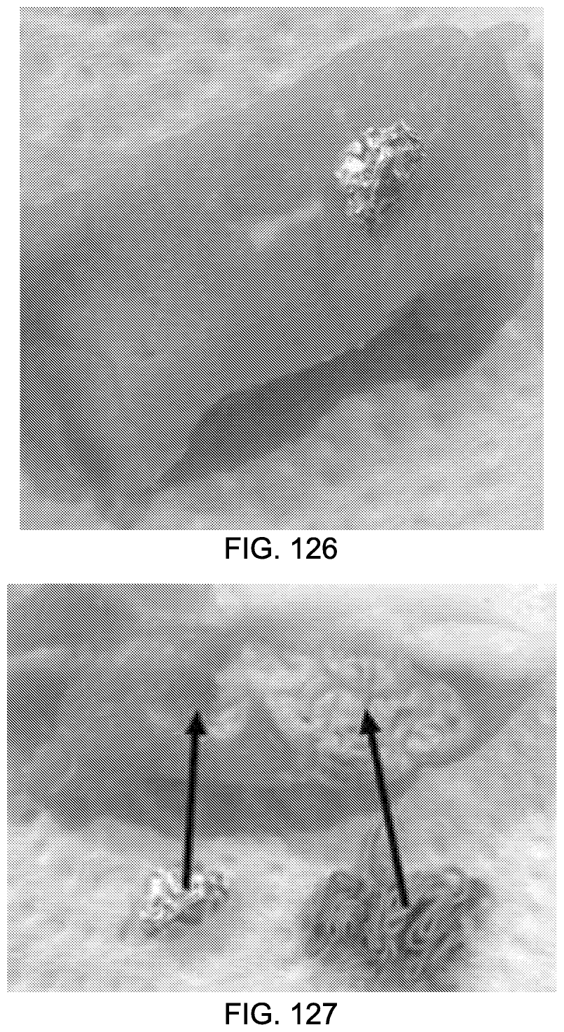

FIG. 126 and FIG. 127: For comparison, aluminum foil was crumbled and placed into sliced pieces of pork muscle tissue or in-between pieces of pork chop prior to subjecting them to the same microwave energy. Only very minimal ablation was observed (the thin white s tripe in the middle) even at microwaving durations that led to much more visible denaturation/ablation effects when cured electrodes were used instead of the crumbled aluminum foil. FIG. 126: Pieces of pork muscle tissue were microwaved with crumbled aluminum foil and a cured electrode present, the latter showing far more ablation as shown by the much greater whitened area visible clearly in FIG. 127. The much greater ablation effects from the cured electrode in FIG. 127 comparing crumbled aluminum foil (left) vs. the cured electrode (right) with both of them present during the microwaving process at the same time. It was concluded that the aluminum foil which had an impedance between any two points 1 cm apart of <0.2Ω (measured with an impedance meter at sinusoidal frequency of 100 kHz), most of which may have been the contact impedance between the probes and the aluminum foil, was too good of a conductor to heat up as quickly and efficiently as the electrically conductive cured electrode subjected to the same microwave energy at the same time, likely due to an impedance that was in the range of 1<X<100 Ohms. The impedance of the cured electrode at any given location of the meandering structure depended, among other factors, primarily on the length of each meandering loop that was measured, the thickness of next connecting points, and the diameter of the meander itself. Generally, the cured electrode was at least 5 times larger in impedance than the comparative measurement in aluminum foil, and at least 5 times smaller than the impedance of the animal tissue. This lead to a concentration of the current in the cured electrode (when compared to the animal tissue) and the current inside the cured electrode generated enough heat to warm the surrounding tissue beyond the point of whitening in a large area indicating a significant heating of the cured electrode (when compared to the aluminum foil).

Impedance values for RF and DC ablation cured electrodes have some common principles but some differences. The optimal impedance range and thermal conductance range for cured electrodes used for ablation depends on the mode of ablation that will be used. In general terms, low impedance cured electrodes (<10 Ohm) are more favorable to conduct electrical energy to the ablation location without having significant off-target ablation effects. This is especially true when the cured electrode for ablation utilizes the approach of forming a cuff around the target (nerve/blood vessel/etc.) and then extending a contacting wire-like structure towards the skin that terminates in the sub-cutaneous tissue in a contact pad.

For RF ablation, both thermal and electrical parameters matter and depend on the type of cured electrode interface that is placed. (Additional discussion of these same parameters for DC ablation are discussed elsewhere herein). On the one hand there is the cuff placed around a target with a wire-like extension from the cuff to a subcutaneously placed contact pad (same setup as for the DC ablation described below). This specific implementation is contacted by an RF ablation needle to drive the RF current from an outside-the-body device through the skin-penetrating needle into the cured electrode, from where the RF energy is transmitted into the target surrounded by the cuff. For this approach, the high frequency electrical impedance of the cured electrode is to be as low as possible, meaning the optimum for electrical conductivity is again located at the higher end of the spectrum of potential parameters (<10 Ohm). The thermal conductivity shall be on the high end as well, though not as critical as the electrical conductivity as large currents and voltages are transmitted at kilohertz frequency into the tissue. Larger off-target effects than with DC ablation may be expected with this approach if the same geometry cured electrode is utilized. On the other hand, there is the approach of placing a cuff around a target structure without any extensions and apply RF from two or more sources as radiofrequency injection into the tissue (read: radio transmission into the body), which may be further added by overlapping nearby frequencies similar to interferential current. Without relying on the wire-like extension to guide the electrical or electromagnetic energy into the cuff and thereby into the target, the cured electrode cuff may be, but does not need to be, formed from higher electrical impedance material (feasible: <10 Ohms as well as 10 Ohms <Z<100 Ohms). The thermal conductivity shall be on the high end to allow for an even distribution of the heat generated inside the cured electrode cuff as well as the heat generated on the interface between the cured electrode material and the interstitial tissue to be distributed all along the interface by the cured electrode material for an even ablation effect to happen.

Following a variety of experiments and mixture optimizations, the optimal parameters for ablation are that the cured electrode's impedance values must be within a specific range for optimal effects, aiming for an impedance match (all values seen for a line impedance, Ω cm or Ωm; all measured at 100 kHz):
optimally to be greater than >1Ω, the impedance of a Copper Wire (connecting the signal generator to the waveform applicator; if the cured electrode impedance is similar to that of the connecting wire (which is often made out of copper) between the signal generator and the actual applicator electrode to be inserted into the body to make contact with the cured electrode by touching it then the wire too heats up in the process, an unwanted side effect) and larger than the impedance of very low conductors that may be implanted inside the body in form of pacemaker leads or similar. This was confirmed by the absence of significant heating of the aluminum foil (electrically in parallel to the cured electrode) in the microwave experiments and the copper wire connecting the return electrode (electrically in series with the cured electrode) in the RF ablation 200 kHz experiments.
1Ω<X<100Ω cured electrode=first optimal range as it is larger than the copper wire (see above) but still reasonably low impedance to achieve good conduction throughout the cured electrode without significant voltage drop across the cured electrode material before the voltage drops across the cured electrode—bodily fluid interface, ensuring that the heat generation is at that interface and thus heating up the surrounding bodily fluids with a rather homogeneous heat distribution around the entire cured electrode. This is especially important for cured electrodes of large dimension but potentially smaller cross sections, meaning that the impedance across the cured electrode is still significantly smaller than the impedance from cured electrode to bodily fluids and impedance across bodily tissues, primary goal being to generate and radiate heat homogeneously with the entire cured electrode being a heat point source. This ensures that touching the cured electrode even at a remote spot with the energy applicator (inserted needle) allows a very small temperature gradient across the entire cured electrode and a large temperature gradient from anywhere on the cured electrode to and into the bodily tissue surrounding the cured electrode.
100Ω<X<500Ω cured electrode=second optimal range this impedance being somewhat larger than the first optimal range but still smaller than the typical impedance of bodily tissue (usually about or >500Ω for bodily tissue) as well as smaller than the impedance drop when the current takes the path from the electron conducting cured electrode to the ionic conductor bodily fluid. This impedance range has the advantage of larger heating in the direct close proximity of the cured electrode, achieving a faster heating and more intense heating right at the interface. This impedance range for cured electrode material can be more optimal than the ones above for the application with patch electrodes that send electrical fields into the body without a direct mechanical metal (needle) to metal (cured electrode) connection.
500Ω<X<10,000Ω cured electrode=third optimal range this impedance being larger than the impedance across bodily tissues but still allowing a heating inside the cured electrode as well as in the directly adjacent bodily fluids and adjacent tissues. This impedance range too is suitable for both, direct connecting to the cured electrode with a needle applicator as well as contactless heating of the implanted cured electrode from the outside via patch electrode that do not break the skin (or breaks the skin to a depth of <5 mm with very fine needles that allow a better coupling and have an outer diameter or less than 500 μm).

The impedance across the cured electrode is optimally in a window characterized on the low end of being significantly larger (2×-10×) than the wire impedance of the RF applicator (wire from RF generator to applicator), yet on the high end of the window being significantly smaller (2×-10×) than the impedance to and/or across bodily tissue. The impedance is optimally measured at frequencies in the same range as the RF that is applied (i.e. 100 kHz measurement frequency as a representation of 200 Hz RF to 400 Hz RF). Other aspects of impedance values have been covered elsewhere herein.

The cured electrode embodiments for neuromodulation comprise lower impedances than for RF ablation. Optimal impedance for neuromodulation with the cured electrode is <1Ω. The preferred impedance is as low as possible to not lose electrical energy to bridge the path to the nerve. The goal is to transfer current without major voltage loss across the cured electrode, the cured electrode being either a contact point to the nerve or a cuff around the nerve or even including a wire that is drawn out from cured electrode material inside the body to electrically connect the contact point or the cuff to a more distant location without having to place a wire in the traditional sense but instead drawing it out (with or without wire core inside or adjacent to the drawn out cured electrode). The cured electrode used for neuromodulation is traditionally used to apply energy to the neural tissue that is not necessarily heat but instead electrical or magnetic fields). Optimal impedance for Ablation with the cured electrode, however, is ≥10Ω, and ≤100Ω or even ≤1 kΩ. Impedance can be larger to allow heating of the cured electrode material itself during the RF application because, up to a point, the higher the impedance the more heat is generated. Impedance can be larger as long as there is large enough impedance jump between the cured electrode and the ionic conductor of the biological tissue. The goal is to achieve a heating at the location of the cured electrode either by heating the tissue or bodily fluid in direct proximity to the cured electrode or by heating the cured electrode itself and have heat generated inside the cured electrode then radiate to the tissue or bodily fluid in direct proximity to the cured electrode, thereby again providing an ablation effect. The cured electrode used as an ablation tool (or applicator) is intended to heat the surrounding tissue to either change their metabolic rate (see elsewhere herein) of cells inside the body or affect neural activity inside the body via heat, cooling, pulsed RF heating or RF ablation.

### Tumor and Cancer Treatment

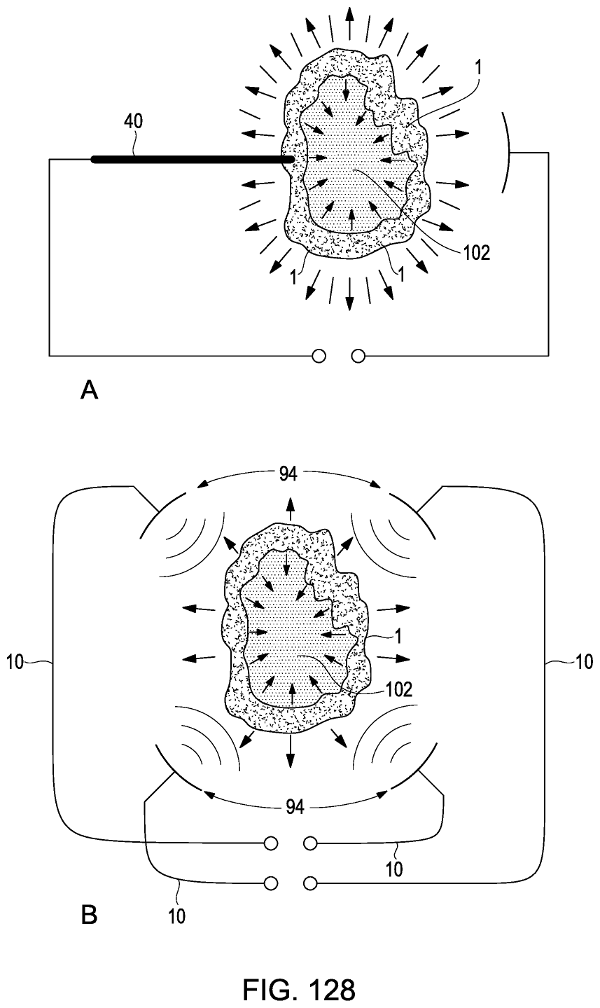

While the cured electrode can be applied to temporarily or permanently block neural communication by heating neural tissue, the RF ablation used of the cured electrode go beyond neural applications. Cancer and Tumor treatment is one of the primary goals of this ablation technology. The cured electrode enables the shrinking of tumors from the Outside-In or Inside-Out. Ablation can be applied to cancerous cells by using the cured electrode to heat a tumor from multiple points (voxels) to achieve a joint heating of the tumor. The heating can be applied to blood vessels supplying nutrients to the tumor. The ablation of these blood vessels and subsequent starving of the tumor may enhance the physician's control over cancerous tissue inside the body that has already grown very large. While traditional surgery and/or traditional ablation may not be an option, ablating tumors from the outside inward (or inside outward) and at its supply lines can be a very efficient and very minimally invasive path to destroying a tumor. FIG. 128A-B depicts use of the cured electrode to treat cancer from the Outside-In or from the Inside-Out. Approach A is a representation of the direct touching of an cured electrode volume that has been placed in direct proximity to the cancerous cells, whereas Approach B uses wireless means such as RF transmitters 94 to heat the cured electrode mass and thereby ablate cancerous and/or surrounding non-cancerous tissue before the cancer is able to infiltrate those tissues. The effect is to seal off the cancer from tissues it may want to invade, to cut of nutrient and energy supply and to start ablating the cancer inward.

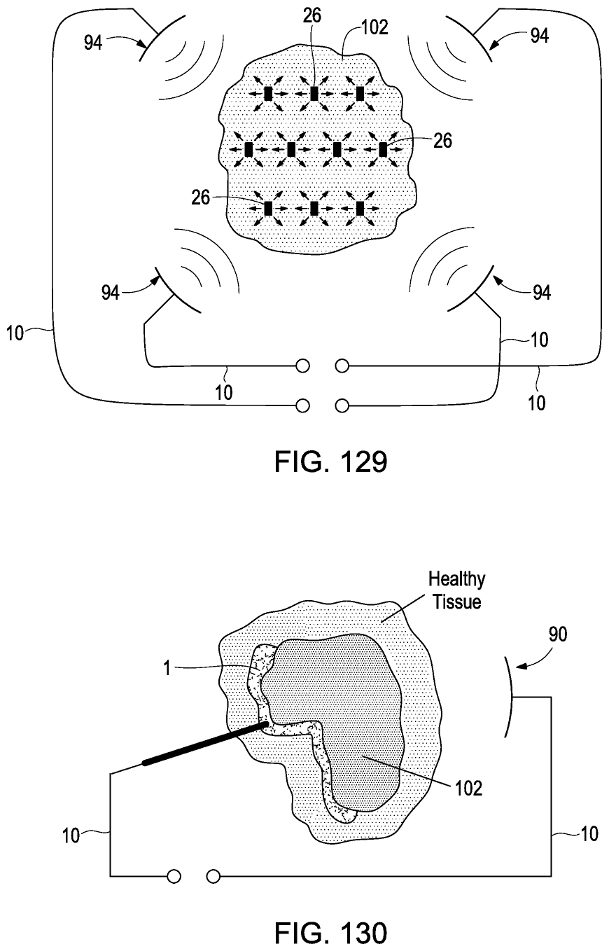

If a tumor is reasonably small then applying sufficient heat can be achieved by seeding the tumor with cured electrode blobs 26 prior to applying thermal energy to the tumor for a larger volume heating of the tumor than is possible with traditional RF alone (inside-out approach). FIG. 129 shows use of the cured electrode to treat cancer from the Inside-Out. The heating is here shown via the contact-less approach like RF transmitters 94 and applied without breaching the skin. The RF is induced from the outside and heats the cured electrode blobs 26 which are injected inside the tumor, with the option to place it near blood vessels adjacent to the tumor and further increase effectiveness.

The cured electrode can also generate mechanical barriers for tumor cells. Tumor growth can be slowed down by placing new cells or ablated cells in the pathway of an existing tumor, thereby reducing its spread beyond a certain border. Such a border can be generated by placing a line or a sheet of cured electrode material at the edge of a tumor, then applying an RF ablation of the tissue and thereby generating a wall in the tumor's path. FIG. 130 depicts the cured electrode as a therapy to prevent cancer from growing beyond a certain “line in the sand.”

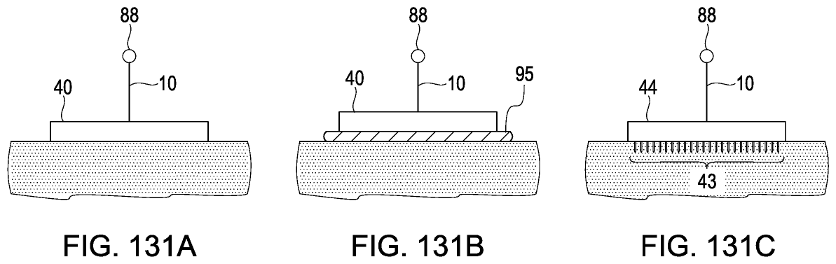

Delivery devices, systems, methods and associated devices for use in ablation using the cured electrode have been covered elsewhere herein. External electrodes used in the four experiments with ablation reported herein were standard TENS electrodes. Other (e.g. full-metal electrodes instead of hydrogel electrodes) can also be used. The electrodes here were either simple wires touching the cured electrode material, or was cured electrode material that was drawn out to be a wire (conducting either thermal or electrical energy to the target location). FIG. 131A-C shows the various types of electrodes which can be used for the approach that injects energy from the outside of the skin inward. The electrode can be a metal contact 40 placed on the skin (A) or with an external hydrogel layer 95 (B) or with additional micro needles 43 that may aid in communicating the electromagnetic field for the RF energy through the first hundred microns of skin and aid with injecting a larger energy density into the body without heating the outer layer of the skin (C).

### DC Ablation

Direct current (DC) has been shown to temporarily block nerves by depolarization or hyperpolarization of neurons. Using a (very) large charge injection capacitance, several second long DC nerve block has been shown to achieve a temporary interruption of neural conduction without permanently affecting neural conduction, thus being a fully reversible nerve block. On the other hand, DC applied with prior art electrodes has also been shown to permanently destroy nerve cells when the applying electrode did not feature a large enough charge injection capacity or the applied charge exceeded the capacitance that can be stored in the Helmholtz double layer of the water in direct contact with the electrode.

In one embodiment, the present invention enables a temporary nerve conduction block without any neural onset response, followed by the intentional continuation of DC injection with the goal of destroying neural tissue conduction. Such a waveform is presented herein. Furthermore, a gradual and slow, then potentially accelerating and larger charge injection with the intent to chemically burn and thus ablate tissue in any location and of any type of tissue has not been described to our knowledge. The cured electrode disclosed herein allows for the mechanical and fundamental electrical means to achieve such a DC nerve block, as well as DC based tissue ablation, and this application describes electrical waveform specifications to achieve the intended effects.

The invention herein enables magnetically induced heating to (1) first place an interface in a non-solid and very flexible form in contact with a ablation target, then (2) have that non-solid cure to a solidified structure that may or not may be absorbed by the body over time and can be heated by a changing magnetic field. This field can be applied from the outside or the inside of the body.

Furthermore, as disclosed elsewhere herein, the cured electrode can be used to convert US energy into heat at the location of the implanted (injected) cured electrode within tissue. The heat that is generated can be used to ablate tissue.

In another embodiment for a system comprising one or more cured electrodes, and the system further comprises “cage” for a cured electrode that allows deploying the biocompatible, electrically conductive elements with the use of a non-conductive but biocompatible carrier into a volume that is pre-formed in the expected measurements of the target tissue, thereby allowing the cured electrode to assume a pre-determined shape and allowing the carrier to not undergo phase transition to hold the conductive carrier elements in place as this function is to be performed by the cage.

There is a need for an injectable neural interface that can be casted, or formed, at runtime during a medical procedure, by a minimally, yet sufficient skilled physician and within a short period of time to improve the ease of tissue ablation, especially repeated applications of ablation.

By forming a cuff shaped or donut shaped interface with the liquid mixture which cures in place around a tubular or circular target structure, the physician only needs to find the ablation target structure once, whether peripheral nerves, arteries supplying healthy tissue, arteries supplying cancerous tissue, tissue margins of a cancer or other tubular or circular ablation targets. The procedure to optimally place the liquid mixture may be done with the aid of US or fluoroscopy/angiography as the liquid mixture may have added or inherent elements that function as contrast agent in the specific visualization modality. The physician may further use commonplace contrast injections into the arterial supply of the target tissue of interest to further increase visibility vs. the background while placing or verifying proper placement of the liquid and curing electrode. Once the electrode has cured in a shape that fully surrounds/encases a circular or tubular target structure in the shape of a cuff or a donut (the circular or tubular target structure in the center), an ablation procedure as described elsewhere herein allows for the concentration of energy in the center of the cuff/donut, shortening the time the ablation energy may need to be applied to provide an effect. With the cured electrode or at a minimum (following the body's inflammatory response to the cured electrode) the majority of the conductive elements of the cured still in place, the cured electrode may still be very easily visualized on US or angiography and thereby ease a repeated, needle based RF ablation months later, or may ease a non-contact ablation (RF/DC/others as described).

Offering a characteristically different impedance from the surrounding tissue, a cured electrode may be found and its location verified with a needle that features two contacts: once inserted into the body, the impedance between the two contacts drops characteristically once both contacts are touching the electrically conductive cured electrode.

The injectable direct current and magnetism tissue heating and ablation interface with optional cage—or—simply cured electrode provides such an interface: While a conventional electric lead placed next to a nerve will not be able to fully depolarize the nerve but generally only cause a full depolarization of fibers near the lead and an incomplete depolarization of the nerve more distant from the location of the lead, the cured electrode in its way to encase the nerve at low impedance values (<100 Ω or even <10Ω) provides a simple surgical approach to connect to neural tissue in various locations, different patients and both within a short application/implantation/injection time. For some DC and/or magnetic ablation applications, the optimal impedance of such a cured electrode can be in the range of 50 to 250Ω, for some other ablation applications, the optimal impedance of such an cured electrode can be in the range of 5 to 10 kΩ, all impedance measured either at 1 kHz for compatibility to earlier patent applications or at 100 kHz to be in the similar range as RF ablation frequencies.

A significant advantage of the cured electrode is that it conforms to the anatomical structures present at a given location of interest. While traditional electrodes impose forces on the neural tissue to achieve a mechanical “holding” or “anchoring” effect (e.g. a cuff electrode rolled around a nerve in the periphery), it's the unique ability of the cured electrode to envelope a nerve with or without additional blunt dissection of said nerve prior to the cured electrode placement. The cured electrode has the ability to form a “negative” from the “positive” forms of the biological tissues of interest. With some of the tissues being excellent candidates to become mechanically interface, such as muscles, fibrous tissue, bone near the nerves intended to be electrically stimulated or blocked, it is the cured electrode that features specific abilities such as adhering to muscles or connective tissue which enable the cured electrode to connect both electrically where needed and mechanically where advantageous to have a stable interface at one location while not putting strain on another such as a nerve.

Disclosed herein is the use of the cured electrode as an interface around neural tissue that may or may not be simple or uniform in nature, that may or may not be easily accessible and that may or may not need repeat applications of RF ablation to achieve the most optimal outcome for a patient.

Short term and long term permanent pain-free DC tissue ablation can be achieved with the cured electrode

This embodiment applies earlier technologies and, where not yet described, defines specific optimizations to achieve ablation where ablation was not achieved before, or neuromodulation where that was not achieved in that form before as well as metabolic modulation if not stated otherwise.

Direct current has been reported to permanently destroy nerve conduction Direct current has also been reported to temporarily interrupt nerve conduction, yet presented herein are efforts first to block nerve conduction temporarily and then achieve a permanent DC induced chemical ablation of the tissue of interest, be it neural or otherwise tissue inside the body. Also presented are the steps to first place a liquid electrode near the innervated target tissue of interest, then temporarily block nerve conduction in the innervation to the target tissue while potentially reducing metabolic activity as a side effect in the target tissue (which too may be neural) and then achieve a permanent DC induced chemical ablation of the tissue of interest, be it neural or otherwise tissue inside the body.

Temporary DC nerve block functions by either slowly depolarizing or hyperpolarizing a nerve. This can be achieved without an onset response, meaning a nerve block is possible without an initial activation period. Such an initial activation period in the prior art is inducing or initiating neural action potentials when e.g. kilohertz frequency alternating current (KHFAC) nerve block is applied to reduce neural firing following the initial onset response. Prior art direct current nerve block uses a slow ramp to initiate a short term nerve block, but requires the current injection in the opposite direction to achieve a charge balancing and avoid chemical ablation following the application of the DC nerve block. By using the cured electrode with its high charge injection interface, however, a partial and eventually full temporary nerve block can be achieved without an onset response. This method can use either charge balance for the injected charge or continue the charge injection to switch from a temporary nerve block to a permanent effect. In contrast to prior art methods, the DC nerve ablation method of the present invention achieves a chemical ablation of the nerve without the need for the application of a chemical nerve block agent prior to initiating the DC nerve block. Moreover, this DC nerve block can be applied near or around any target tissue (especially any neural target tissue) and can be applied without the need for prior analgesics (pain blocks such as lidocaine or marcaine).

In one embodiment needle gauges of 16 g-20 g are used to contact the previously-placed cured electrode in order to inject the DC charge first to temporarily block nerve conduction without the initiation of a neural onset response, next followed by continuing DC injection to eventually destroy the neural tissue. In other embodiments, the DC current can be applied directly from the skin when the placed cured electrode uses the “cured electrode+sub-cutaneous contact pad” approach as described elsewhere herein.

While the waveform and method DC block is described in the context of the cured electrode, it can be used without the cured electrode to first temporarily and then permanently block nerve conduction with conventionally placed neural electrodes which have been placed by other means than a needle injection, where applicable.

A method of short term and permanent pain-free Ultrasound tissue ablation can be administered using the cured electrode. As described elsewhere herein, Ultrasound can be guided by an ultrasound-transparent cured electrode to a neural or a different bodily tissue target. While elsewhere herein there is disclosure of stimulation of neural and or other tissue or on temporary heating of said tissue to temporarily change their metabolic activity or to induce a short term nerve block (such as controlled short term heating of neural tissue to approximately 42+degrees C.), we are also disclosing a method of using the Ultrasound energy to generate heat to ablate tissue. As long as the generated heat is causing a tissue heating in the range of 50 to 90 degrees centigrade, more optimally 60 to 80 degrees centigrade, then tissue ablation is achievable.

The cured electrode may function both, as a guide as well as a reflector of Ultrasound energy and thereby provide the means to concentrate ultrasound energy at a location of interest that is to be ablated. A guide type transduces (transfers, guides) ultrasound energy from a location near the outside of the skin (but the cured electrode still below the skin, e.g. in the sub-cutaneous tissue) to an ablation target. A reflector type either blocks ultrasound energy from reaching tissues, or it concentrates the ultrasound energy by placing the cured electrode reflector in a concave shape, thereby functioning like a lens. In addition, the reflector type may help to generate heat right next to the cured electrode volume on one side while the other side remains more or less temperature neutral.

Short term and long term or permanent pain-free magnetic tissue ablation can be obtained by using the cured electrode. As described elsewhere herein, magnetic energy can be passed by a magnetically conductive cured electrode to a tissue target. While elsewhere herein there is disclosure of stimulation of target tissue or on temporary heating of said tissue to temporarily change metabolic activity or to even induce a short term nerve block (such as controlled short term heating of neural tissue to approximately 42+ degrees centigrade), the following method describes using the magnetic energy to generate heat to ablate tissue. As long as the generated heat is causing a tissue heating in the range of 50 to 90 degrees centigrade, more optimally 60 to 80 degrees C., then tissue ablation is achievable.

The cured electrode may function both, as a guide as well as a reflector of magnetic energy and thereby provide the means to concentrate magnetic energy at a target to be ablated. A guide type transduces (transfers, guides) magnetic energy from a location near the outside of the skin (but the cured electrode still below the skin, e.g. in the sub-cutaneous tissue) to an ablation target. A reflector type helps either to block magnetic energy from reaching tissues, or to concentrate the magnetic energy by placing the cured electrode reflector in a concave shape, thereby functioning like a lens. In addition, the reflector type generates heat right next to the cured electrode volume on one side while the other side remains more or less temperature neutral. Furthermore, the cured electrode, in one embodiment, contains iron oxide to heat in changing magnetic fields and allow a temperature increase up to the 60 to 80 degrees C. range.

No matter which energy is use to ablate tissue with the cured electrode, the application of the energy can be graded to achieve an onset-free nerve block preventing the patient from feeling any effects from the ablated or neighboring tissue even without a chemical nerve block (e.g., lidocaine, marcaine).

At least some methods of ablation with the cured electrode comprise the steps of (1) the energy applied is chosen such that first a patient stimulation threshold (with whichever energy is used) is recorded by (2) applying the energy at a level that is expected to induce a neural response of the tissue to be ablated or the nerves innervating the tissue to be ablated, then (3) a slow ramp of the same energy is applied to drive the tissue and/or innervating nerve fibers into a onset-free nerve block and then (4) the ramp is continued to a plateau of applied energy of sufficient time to achieve a permanent tissue ablation.

Other embodiments of ablation methods comprise steps of (1) selecting a first blocking form of energy (different than the energy for the actual nerve ablation) to first find a patient sensory stimulation threshold by applying said energy in a pulsed fashion, then (2) inducing a nerve block by ramping up said first blocking energy until a full nerve block from the target tissue is expected or verified (e.g. by overlaying additional electrical pulses), after which (3) the ablation energy is applied (either as a slow ramp, or pulsatile application, or other) of sufficient time to achieve a permanent tissue ablation.

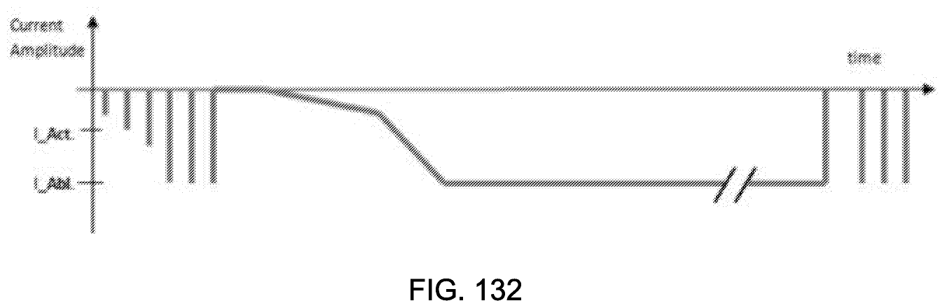

The methods for ablation and the waveforms comprise the steps of
First, a cured electrode is placed on, nearby or around an ablation target.
Secondly, stimulation pulses are applied to find an activation threshold “I_Act.” that causes some form of biological response, be it sensation of the stimulation, a muscle twitch, or other reflex or response by the body. See FIG. 132.
Thirdly, the current amplitude of the stimulation pulses is increased further beyond at least 150% of the current needed to elicit any changes in that biological response, indicating a saturation threshold beyond which no more neural fibers are being activated. The ablation current amplitude shall be defined as 150% of this saturation threshold, thereby ensuring that all fibers will be activated during the DC nerve block. FIG. 132 is a depiction of one representative shape of a nerve block waveform in one embodiment of the ablation method first to lame and then to ablate tissue.

FIG. 132 depicts the application of unidirectional direct current, meaning there is no application of charge balancing with the intent of ablating the nerve with DC. At first, the current amplitude is raised to the level of the nerve activation threshold which allows the recording of said threshold. Then the current amplitude is raised higher to record the current value at which no more change in i.e. muscle activity results from increasing the unidirectional DC stimulation amplitude. At this point, all nerve fibers are being recruited by the stimulation pulses (of i.e. 200 us pulse width). Once the clinician/researcher/experimenter has found the activation and saturation thresholds, ablation current will be applied at or above the saturation threshold equaling the ablation threshold. The current for the ablation is ramped slowly, either in a linear or in an exponential or in a quasi-stepped form by using a linear increase but increasing the rate of linear increase over time (FIG. 132 shows two linear rates, first a smaller and then a larger rate of current amplitude change over time). Once the ablation current reaches the ablation threshold that was determined earlier, the ablation current may be held constant for a desired period of time, i.e. 10 to 100 seconds, to provide the intended amount of nerve ablation. After the application of the desired amount of unidirectional current has been delivered, the nerve's ability to conduct neural signals is tested by stimulating the nerve, i.e. at the initial activation threshold, or as shown in FIG. 132 at the ablation threshold. If there is no response from neural stimulation at the specified amplitude, the procedure may be considered a success and finished. If a stimulation at a specific amplitude (i.e. ablation threshold) shows an organ response (connected muscle, or pain sensed by the patient) then the ablation procedure may be repeated with the same or higher targeted current amplitudes for stimulation and ablation.

There are additional embodiments of cured electrode placements and applicator electrode placements. Cured electrodes 1 can be placed (e.g., injected) around an ablation target, near an ablation target or into an ablation target. DC energy applicator electrodes are then placed in direct contact with these previously placed cured electrodes to achieve DC ablation. In contrast to RF ablation, the cured electrode is not subjected to significant changes in temperature during the DC application and thus allows for different mixture formulations that do not require a thermal stability beyond 42 degrees centigrade (normal body temperatures being 35 to 42 deg. C). FIG. 114A-B illustrates embodiments of the contact-based DC Ablation approach. The cured electrode in 114A fully surrounds the nerve whereas the cured electrode in 114B only passes by the nerve or partially surrounds the nerve. By touching the cured electrode during DC application chemical species (acids, bases) are generated either inside the cured electrode material or at the interface between the cured electrode material and the surrounding bodily fluid and bodily tissue.

In another embodiment the DC Ablation method with the cured electrode further comprises the steps of applying patch electrodes from the outside of the skin. By overlaying DC current signals applied from the outside of the skin aiming at a common crossing point inside the body at which the cured electrode material shall be located, ablation can be applied without breaking the skin with a needle, or the needle can be only very superficially breaking the skin to induce optimal electromagnetic coupling to the body. FIG. 115A-C illustrates embodiments of non-contact-based DC Ablation methods. The cured electrode in 115A fully surrounds the nerve whereas the cured electrode in 115B only passes by the nerve or partially surrounds the nerve, and whereas the energy transmission can be further aided as depicted in 115C by drawing the cured electrode material out closer to the subcutaneous tissue (shown to its full possible extent, or exaggerated). By transferring the energy to the cured electrode as electromagnetic currents during DC application chemical species (acids, bases) that damage bodily cells are generated either inside the cured electrode material or at the interface between the cured electrode material and the surrounding bodily fluid and bodily tissue.

The methods and materials described elsewhere herein for formulating, injecting and curing electrodes for neurostimulation and block and for energy wave guides are similar to those for Ablation cured electrodes.

For the DC based ablation, the cured electrode comprises materials providing optimal impedances in the range of <10Ω, preferably <1Ω measured across the material. The charge injection capacitance does not need to be very large and can be in the range of a single wire being placed near, next to or around a nerve, though the placement of the cured electrode for ablation purposes is understood to be made of either many small elements held in place by a nonconductive carrier, a mechanical holder (“cup” or “mesh” or “sock”), or a very thin wire such as a gold bonding wire that is injected or placed onto, into, or around a nerve to form a meandering continuous string in the direct vicinity of the cells intended to be ablated. Materials used can be more iron or steel based as very little charge injection capacitance is needed for this technology to function properly.

For DC ablation, the current must travel through the skin from the outside of the body into the contact pad 14, though the wire-like extension 23 from the contact-pad to the cuff around the target structure, where it affects the target structure. The electrical return path may be a second cuff around the same target structure or another wire-like extension from a second subcutaneously placed contact pad a few centimeters separated from the first contact pad. For an effective DC ablation to occur within a time that is feasible in an in-office or out-of-office but not at-home procedure, currents of about 10 mA may be required to achieve a fast and long lasting (weeks to 3 months) DC ablation of i.e. a nerve or other metabolically very active tissues. For at-home applications, where the DC current may be supply by a patch on the outside of the body that carries i.e. a 3.7 to 20V battery supply on the inside, smaller currents such as 100 uA over extended periods of time may be supplied to the target to achieve a sufficient effect. Additionally, if a nerve is to be blocked or ablated, then low current amplitudes (i.e. 50 to 200 uA) that may be below the nerve's activation threshold (i.e. 100 to 500 uA) may be used to block temporarily (minutes to days) or ablate more permanently (weeks to months) a nerve without the patient experiencing the sensation of nerve activity during the blocking and ablation application. Patients may be given patches that have different “strengths” by applying a variety of driving voltages (electrically: a voltage limiter) and having pre-set maximum current values (electrically: a current limiter) to allow for a continuous and reproducible nerve block perception or ablation effect. The specific currents driven by the specific voltages needed depend on each patient's anatomical specifics (i.e., a large nerve that needs to be blocked requiring larger currents etc.) as well as how deep the nerve is located and how far the contact pad is located below the skin, both factors defined in the implantation/injection procedure that vary from one physician to the next as well as from one procedure to the next. The outside-the-skin worn patch electrodes, an active device with battery supply, optional current and voltage regulating elements as well as optional microelectronics to allow for non-constant current delivery such as pulsed or ramped DC, account for these variabilities. In either case, the electrical conductivity of the cured electrode shall be on the higher end of the spectrum to achieve a minimum of voltage drop from the subcutaneous contact pad through the wire-like extension to the cuff around the target. The thermal conductivity in this configuration does not matter for the efficacy of the DC ablation effect.

An injectable electrode consists of a liquid nonconductor and conductive elements. Here the conductive elements are themselves “Super-elements” or larger elements formed of a mixture of smaller conductive elements. The super-elements provide a temporary scaffold to hold together the percolated network of conductive material that forms the injectable electrode. The superelements are of a size that is too large to allow for element migration (e.g. <100 microns). However, the individual conductive elements that comprise the super-elements are individually each smaller than 5 microns allowing them to be phagocytosed and cleared or isolated by macrophages.

These super-elements have the following advantages compared to solid metal elements of the same size: less precious metal material per given volume, better element mechanics/flexibility, lower percolation threshold for bulk injected electrode.

An example system consists of superelements made by conventional alginate bead encapsulation techniques. In brief, the metallic elements (less than 5 microns diameter) are added above their percolation threshold to a sterile 1% sodium alginate solution. The alginate-metal mixture is then dropped (or blown or otherwise processed) into a cross-linking solution such as calcium chloride. The super-elements are then thereby formed and cured for 1-2 hours until fully gelled. These super-elements are then used in combination with any of the previously described liquid nonconductors to create an injectable electrode.

Magnetized elements can form an interconnected network, particularly in the embodiment of Gold Coated Magnetic Iron Oxide as Conductive elements.

Since element migration may cause tissue reactions in some people as shown in metal-on-metal hip implants, it is beneficial to comprise the conductive elements of the injectable electrode out of magnetic (or magnetically induced) elements such that each conductive element is attracted to one another, thereby limiting diffusion of individual elements away from the bulk device. The magnetic elements will be sized such that they may still flow easily through a standard minimally invasive needle or cannula. The magnetic elements will then naturally self-assemble into the most energetically stable formation upon injection into the body cavity. If placed in areas of moderate to low motion or externally applied forces, the magnetic force between elements is sufficient to keep the device bulk integrity intact.

In the case of need for surgical removal of the device (or its shed elements), a strong magnet can be used to retrieve the device or its shed elements.

Advantages of the magnetically conductive cured electrode include that they are a low Cost and Effective Conductive Filler, and 2) Magnetic Elements are Attracted to One Another Prevent Individual Element Migration Away from the main bulk of the cured Electrode. Magnetic iron oxide (Fe3O4), or Iron (II, III) oxide, nano elements have been used as imaging contrast agents for decades, specifically for enhancing T2-weighted MRI imaging applications. Others have shown that it is possible to coat magnetic iron oxide elements with tens of nanometer thick gold to increase their stability and inertness. Multiple approaches have been outlined by Silva et al Chem. Commun. 2016, 52, 7528. At the same time, Bastus et al Langmuir 2011, has shown that gold Nano elements can be grown in a controlled fashion from seed templates. The seeds they showed were themselves gold Nano elements. Combining (1) and (2), and making modifications to the protocols accordingly, we propose to coat magnetic iron oxide flakes with a gold layer ˜50 nm thick. Example Materials: Black iron oxide, Alpha Chemicals, Natural=40 um avg size; Synthetic=300 nm avg. size; 7 m2/g surface area compared to 1-5 m2/g micron-sized silver flakes used for conductive paste applications). 25.99 and 11.99 respectively for 10 lbs and 1 lb respectively

Magnetic elements such as gold-coated magnetic nanoelements (AuMNPs) have been used for multiple biomedical applications, including as imaging contrast agents. These contrast agents are generally well tolerated with intravenous administration and are cleared from the blood stream rapidly. Ultimately clearance occurs primarily in the liver and spleen and may take extended periods of time (>1 year). However, the process appears to be well-tolerated given the clincial experience with these agents. The elements remained confined in lysosomal compartments in the cells of these organs. Coating of the MNPs with a thin layer of gold further increases the biocompatibility and circulation time of the MNPs.

Gold coating can be accomplished in several ways including a direct coating method and an indirect method. The direct method uses the iron core as a “seed” substrate and attaching gold atoms to the seed with reducing agents such as sodium citrate and sodium borohydride. The indirect method utilizes a third material as a “glue” to generate a coating around the MNP core, which can then form interactions with a gold shell. In both cases expansion of the shell thickness can be accomplished by a layer-by-layer growth procedure that uses a heated solution of sodium citrate and a gold salt (e.g. HAuCl4) to deposit sequential layers of gold. Size of the elements can be initially measured by UV-vis spectroscopy and confirmed by TEM for verification.

Gold-coated elements have the additional advantage of providing an easily modifiable surface via thiol-chemistry. Gold surfaces form a complex bond with thiol and di-thiol ligands. Typical modifications include pegylation to enhance the hydrophilic properties and anti-fouling properties of the surface or attaching proteins or enzymes for the purpose of biosensor applications.

### Additional Applications

The injectability of the liquid mixture allows interfacing with the PNS at locations formerly not feasible with prior art devices. Many nerves of the PNS originate on the ventral side of the spine, running along the bones of the ribcage as intercostal nerves or diverge into the abdominal cavity. Most of these nerves may not be interfaced with current technologies, such as common cuff electrodes, unless a major surgery first grants access to such a deep tissue nerve, generally only possible from the ventral (abdominal) side, and then places the cuff electrode around the nerve with the need to then find a place to anchor the cuff electrode via suturing it to tissue that does not move much in relation to the nerve the cuff was placed on. Prior art devices do not allow a dorsal access as the surgical field required to gain access to a nerve in order to place a cuff electrode is simply not available between the bones of the rib cage 78 or the muscles of the back without causing major damage to the movement and stabilization apparatus of the patient or subject in the process. Yet, many nerves of the autonomic nervous system (“ANS”), especially the majority of sympathetic nerves are located on the ventral side of the spine 77 and run along as the ganglia of the sympathetic chain (FIG. 133). The sympathetic chain 76 and its ganglia are depicted as positioned near to both sides laterally of the vertebrae at the rear of the thoracic cavity. To reach the sympathetic chain ganglia ventrally in this area would be massive surgery going around the heart and the lungs and the largest and most critical blood vessels, so the sympathetic chain is not reachable ventrally. Dorsally, though, through a thin needle may be placed through the muscles of the back through the ribs and so deliver injectable liquid mixture/cured electrodes to the sympathetic chain ganglia, instead of requiring an thoracic/abdominal surgical procedure. The present invention thus allows access to many locations formerly believed impossible to access such as, for example: (1) pre-ganglionic fibers exiting the spinal cord before synapsing in the ganglia of the sympathetic chain. (2) ganglia of the sympathetic chain. The liquid mixture/cured electrode may be injected to encase a specific ganglion allowing for the complete and or selective/partial depolarization using uniform electrical fields achieved by fully encapsulating the ganglion with the cured electrode and stimulation versus a distal return electrode. (3) connecting fibers between adjacent ganglia of the sympathetic chain. (4) post-ganglionic fibers that exit the ganglion of the sympathetic chain and travel to the inner organs, organ systems and neural ganglia or plexi inside the abdomen and other locations inside the body. (5) Foramen that Ganglia are located in. These foramen may function as mold pre-cure as well as added mechanical protection of the cured electrode. (6) Foramen that function as passage ways for nerves and nerve bundles. These foramen may equally function as mold pre-cure as well as added mechanical protection of the cured electrode. (7) tissue plains between muscle bundles that have nerves and nerve bundles pass in-between may become a mold to form a cured electrode in and attach a cured electrode to. The placement of the cured electrode does not require the same number of surgical steps needed to achieve a blunt separation of the nerve tissue from surrounding other tissue as well as may not require separate steps to anchor an electrode as the liquid electrode may provide this as an innate property, interfacing with the surrounding as well as the encapsulated neural tissue.

The present invention enables interface for ganglia in the PNS as never before. Ganglia are intersections of nerves in specific locations of the body. These intersections may be formed of afferent only, efferent only or combined afferent and efferent nerves. Ganglia contain axons and cell bodies of neurons and represent small processing units, somewhat similar to neural plexi, in the periphery of the body, meaning computational units that may perform signal analysis, combination, reduction and processing outside the central nervous system. Ganglia thus represent a highly desirable target for neural interfacing to stimulate or block activity within them fully or partially.

The prior art method of interfacing with a ganglion is to stab it, meaning injecting a sharp electrode, such as a microelectrode, into the ganglion. This unfortunately only allows for the interfacing with a few of the neurons passing through or connecting inside the ganglion. If a comparably large electrode were to be stabbed into a comparatively small ganglion then irreparable compression damage is to be expected for the ganglion, and injury from irritation during body movement.

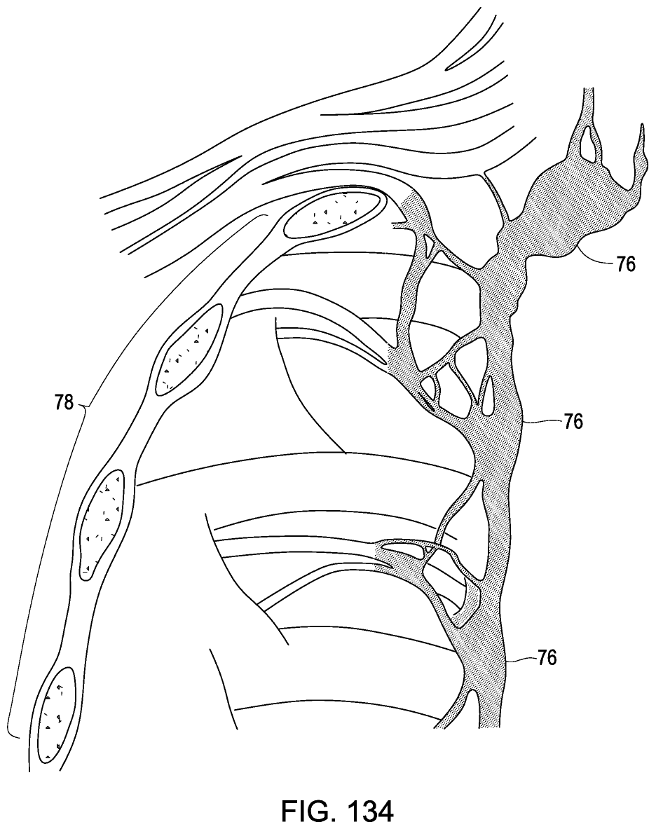

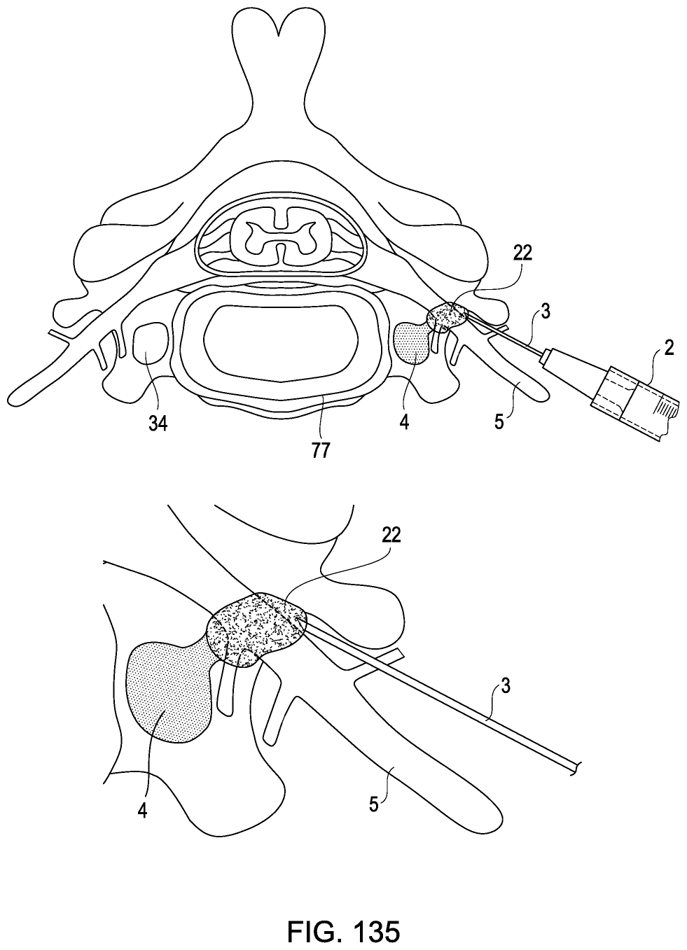

FIG. 134 shows greater detail of the highly irregular shapes of the sympathetic chain ganglia 76 at the back of the rib cage 78. Cuff electrodes for ganglia in contrast do not exist as such, for cuffs form a cylindrical volume when deployed. Cuff electrodes are not very desirable, but they can form a circular structure around a cylindrical nerve. They do not interface at all, however, with a ganglion that is irregularly shaped and has more than two neural entry points. If a cuff electrode were to exist for a ganglion, it would require substantial surgical access around the ganglion to encompass all entering and exiting nerve endings. Such a procedure would require more than a simple blunt dissection lasting less than 5 minutes. Prior art cuffs often require a suturing of the cuff around the nerve or to neighboring tissues which require the supporting mechanical biological environment as well as additional surgical time.

The advantages of the present invention in comparison include, without limitation, the ability to (1) provide a blunt dissection by placing the liquid mixture next to, behind and around a biological structure which reduces surgical time needed to free up a target from its surrounding tissue, (2) encase a irregularly shaped target inside the body with a liquid mixture that flows initially and, after undergoing a phase change, flows less (e.g. malleable) or even ceases to flow (fully cures) and allows to place a neural interface all or partially around a ganglion with the expectation of having a reliable mechanical interface in place, (3) place a connecting wire into the liquid mixture at any location within the liquid mixture to fit the anatomy of the patient, (4) place liquid mixture via needle, with added instruments using a laparoscopic approach and under ultrasound or angiographic/x-ray visualization, allowing minimally invasive placement of liquid mixture on/around ganglia not surgically accessible before, e.g., deep inside the abdomen such as the ganglia of the sympathetic chain adjacent to the spine and on the ventral (abdominal) side of the spine.

Dorsal root ganglia (DRG) at the spinal cord may be interfaced in a similar fashion, (a) requiring a blunt dissection of the DRG first, (b) or using the blunt separation abilities of the liquid mixture placement to save time on the OR table. The DRG may be encased with liquid mixture, then also fully or partially encased with liquid nonconductor anchored elsewhere to raise mechanical stability or improve selective neural stimulation of the DRG.

The present invention has the capability to provide electrical stimulation of ganglia and connecting nerves of the sympathetic chain. The sympathetic chain innervates virtually every organ in the body and, additionally, is connected to blood vessels throughout the body, allowing a coordinated, body-wide effect with one or more neural interfaces that stimulate or block either ganglia or the connecting nerve fibers between ganglia of the sympathetic chain or the nerves connecting ganglia with the organs in the body. The sympathetic chain is commonly understood to regulate the “fight-or-flight” response. Some of the applications resulting from stimulating the sympathetic chain with the present invention include, without limitation: (1) an “electronic caffeine,” i.e., waking a person within seconds, (2) a “boost of energy” to the subject, including a raised heart rate, respiratory rate, sweat production, modulation of blood pressure and modulations of the iris diameter, (3) an anti-depressant and mood regulator, (4) combating the sensation of hunger, (5) raising the body's base metabolism and metabolic rate when a subject exercises, (6) an electric analgesic, (there is a directly correlation between parasympathetic over activity and perception of pain and modulating sympathetic activity can reduce the duration and intensity of pain) (7) modulation of sympathetic activity indirectly leads to a modulation of parasympathetic activity in the body. By temporarily blocking sympathetic activity, parasympathetic activity will decrease as a response due to the body's own regulatory pathways, allowing a reduction of parasympathetic activity by both, fully stimulating, partially stimulation, partially blocking, fully blocking as well as partially blocking and partially activating specific ganglia and connecting nerve branches of the sympathetic chain. The present invention provides all three, a medical diagnostic, a medical treatment and an academic research tool to directly interface with the sympathetic chain of the human body, as well as animal preparations, while relying on a minimally invasive surgery to place the cured electrode.

The present invention enables neuromodulation of all the organs connected to the sympathetic chain, by attaching one or more embodiments of the present invention to the sympathetic ganglia. The ability of the present invention to encase a ganglion completely with all entry and exit nerve branches allows for the application of nerve block waveforms uniformly to depolarize or hyperpolarize fibers and cell bodies within the ganglion similar to the uniform depolarization that may be achieved with a 360-degree encased cuff on a PNS nerve trunk: while a partially encased nerve trunk may be partially depolarized and hyperpolarized with fibers close to the electrodes perceiving the effects of the electrical field first, it is the 360-degree encapsulation that allows for a uniform field (of rotational symmetry) within the nerve trunk inside the 360-degree coverage of the liquid mixture/cured electrode to provide the uniform depolarization and hyperpolarization. It is this ability to produce the uniform depolarization and hyperpolarization that is key to a controlled and reproducible nerve block, especially with chronically placed electrodes.

The ability of the present invention to adhere very closely to a neural stimulation or block target of interest in combination with the ability to fully encase said target is vital to achieving a controlled and reproducible (partial or full) nerve block using waveforms such as kHz-frequency for KHFAC nerve conduction block, 200 Hz (range: about 150 to 900 Hz) for neurotransmitter depletion block to anodic block without damaging the ganglion by penetrating it with an electrode. The present invention thus provides the only interface needed for a controlled, partial or full and repeatable nerve block without the need to breach the membrane of the ganglion. The present invention achieves this for every ganglion with a proper surgical placement around the ganglion, with a partial or a full covering/encasing of the ganglion to achieve the ability to block as described.

In one embodiment, the present invention is a much more feasible alternative to prior art methods of sympathetic ablation that uses an endoscopic approach with a dorsal access. Some patients receive a surgical cauterization/dissection of the sympathetic chain to treat autonomic disorders or intractable pain. While this may treat the initial underlying condition, sympathetic ablation will generally yield a non-reversible result for the patient: it may not be undone and any resulting side effects caused by ablating the wrong or too much neural tissue of either the chain links between the ganglia or the ganglia itself may not be reversed. In contrast, the cured electrode provides a means to deliver a therapy (with a dorsal or ventral or combined dorsally-ventral approach) to reversibly stimulate or block neural tissue of the sympathetic chain or ganglia in the periphery or neural plexi in the abdomen as well as other peripheral locations. Use of the cured electrode is fully reversible by switching the waveforms off and, further, may be adapted very specifically to the patient since waveforms may be changed by the physician by noninvasive means. Additionally, the present invention's cured electrodes may be removed more easily through a minimally-invasive procedure if the patient or physician desires so, an advantage over prior cuff-like electrodes or electrodes that would penetrate the membrane of a ganglion, thereby leaving an indentation and scar tissue inside the ganglion when removed.

The present invention enables stimulation of spinal nerves formerly not accessible. The ability of the cured electrode to be delivered via needles placed through keyhole incisions is vital to a new treatment paradigm presented to the neural engineering community. Surgery to place a prior art electrode on a neural target in the PNS required a comparatively large incision as well as a significant amount of spreading of tissue inside the body to gain access to the nerve and have enough space left to place the electrode into or around the nerve, the liquid mixture/cured electrode is capable of providing a minimally invasive procedure to access and interface neural tissue of interest for stimulation and block.

The present invention provides the first reliable interface for deep tissue nerves in the PNS at hard to reach locations such as the spinal nerves exiting the spinal foramen and running along the intercostal space between the ribs or along other bony structures of the body.

The present invention enables a neural interface for a foramen 34 (plural, foramina) which are naturally occurring openings, holes, or passage ways for arteries, veins, nerves and alike in or through bony tissue. Foramen come in many forms, shapes and sizes and are different from one human to another. As such, it is very complicated to provide a one-size fits-all” pre-made electrode that may be placed into a foramen to interface with a nerve. On the other hand, a foramen may represent an ideal mechanical anchor point for a neurostimulation electrode to nerve interface, as electrodes that integrate with the foramen do not experience any movement in relationship to the nerve passing through the foramen. The presence of bony tissue around an electrode and nerve further adds protection for the fragile neural interface. One method of placing and securing a liquid mixture/cured electrode is the injection of liquid mixture around a nerve and also into a foramen. The procedure of placing a connecting wire into a foramen, injecting liquid mixture around that wire and the nerve, encasing both, in some embodiments, alleviates removal of bone near the cured electrode location (e.g., a laminectomy). FIG. 135 is a drawing showing foramina 34 as exit points for spinal nerves with placement of liquid nonconductor 9 as an anchor 4 in a foramen. The anchor 4 is connected to a ring-like cured electrode 22 surrounding a target 5. The needle 3 of a dispenser is able to reach these locations in the lower spine.

The present invention enables interfacing sacral nerves and branches for parasympathetic and bladder control. The human body has two primary interface points to the parasympathetic nervous system: (a) several cranial nerves (vagal, trigeminal, occipital, auricular and others), and (b) nerves of the sacral level spinal cord. For lack of easy surgical access to the nerves to the sacral spinal nerves, most current neuromodulation technologies aiming to utilize a modulation of parasympathetic activity focus on interfacing primarily with the vagal nerve and to some degree with the trigeminal, occipital and auricular nerve. Current interfaces, such as the Medtronic Interstim device for bladder modulation or the Brindley Vocare System for bladder control in spinal cord injured individuals, require a major surgery to gain access to the sacral nerves (and/or nerve roots), in part requiring a laminectomy and removal of spinal bone structure to place prior art electrodes. In contrast, the present invention provides a minimally invasive approach that allows the injection of the primary interface as a liquid mixture around the target. This liquid mixture may then either connect to an injected signal generator, or it may encase the de-insulated tip of a lead wire that in turn may connect to an implantable signal generator, or it may be connected to a subcutaneous connection pad as described herein.

Additional embodiments for the present invention include, without limitation, (1) Stimulating parasympathetic fibers from the sacral level and modulate HR, BP etc. (2) producing calm under stress by being able to stimulate parasympathetic fibers (sacral and cranial level), (3) creating alertness when needed, (4) reducing hunger sensation even with food volume deprivation by stimulating the sympathetic chain, and (5) stimulation of the duodenum to provide the sensation of satiety for low-volume eating.

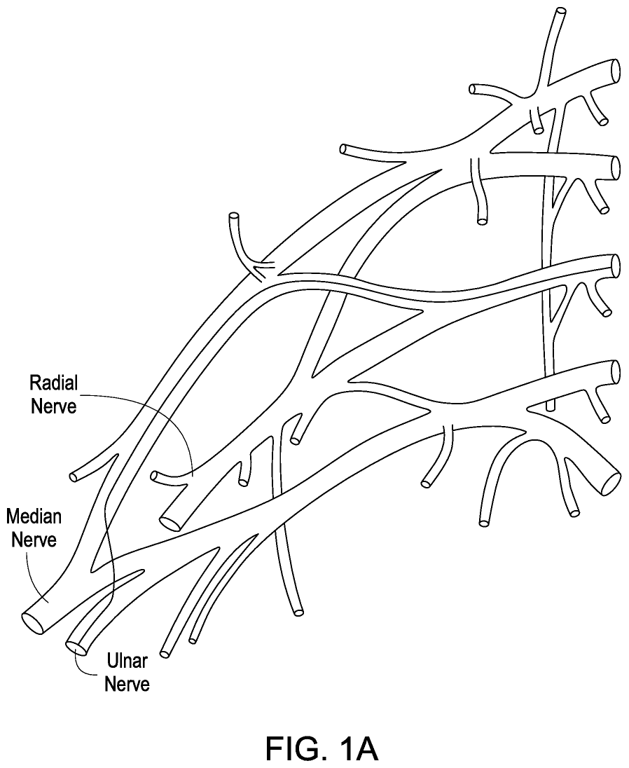

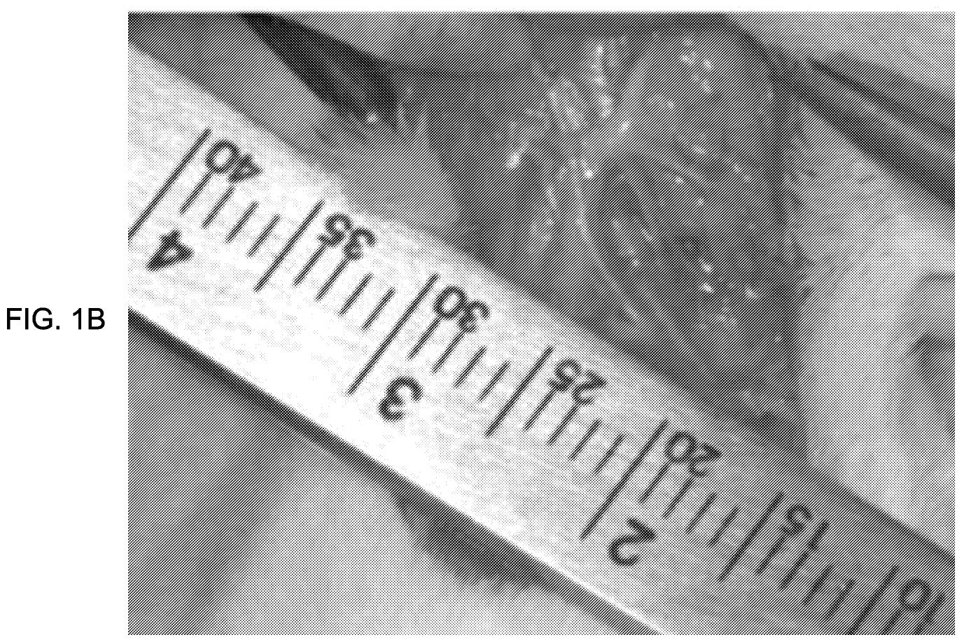

The present invention enables a novel neural interface for neural plexi in the PNS and CNS. A neural plexus is a collection of nerves in the same location, often with crossovers and interfacing between said nerves. Ganglia may be part of a neural plexus. All of these structures are shaped differently, have different nerve thicknesses and often slightly different contours and locations for nerve entrances and exits. It is virtually impossible with conventional technologies to interface in a simple and straight forward way with the nerves of a neural plexus and utilizing the exact same technology across multiple patients, each having unique anatomy. An example of the above application is for the brachial plexus, a collection of nerves near the clavicle bone towards the arm pit and from there innervate the entire arm, as shown in FIG. 1A. The majority of nerves that innervate the arm are very easily accessible at the brachial plexus. The advantage of using the liquid mixture/cured electrode, as contrasted with prior art electrodes, is the capability of encasing one or more or nerves here. A patient suffering from chronic arm pain due a traumatic nerve injury, or from phantom limb pain, will benefit from the placement of a cured electrode on the nerves of the brachial plexus (image below). Especially for cases where the entire arm was lost due to traumatic injury it may be advantageous to utilize a neural interface that may stimulate all the nerves involved at one central interface location. The cured electrode, placed as injection at a perpendicular angle to the longitudinal axis of the involved nerves, aiming to inject the liquid mixture below and behind the nerves, offers an interface that may be adapted by the operating physician at runtime in the ER or and under localized anesthesia at the injection site alone. Wherever needed, nerve or fascicle specific selectivity may be increased by combining liquid mixture and liquid nonconductor.

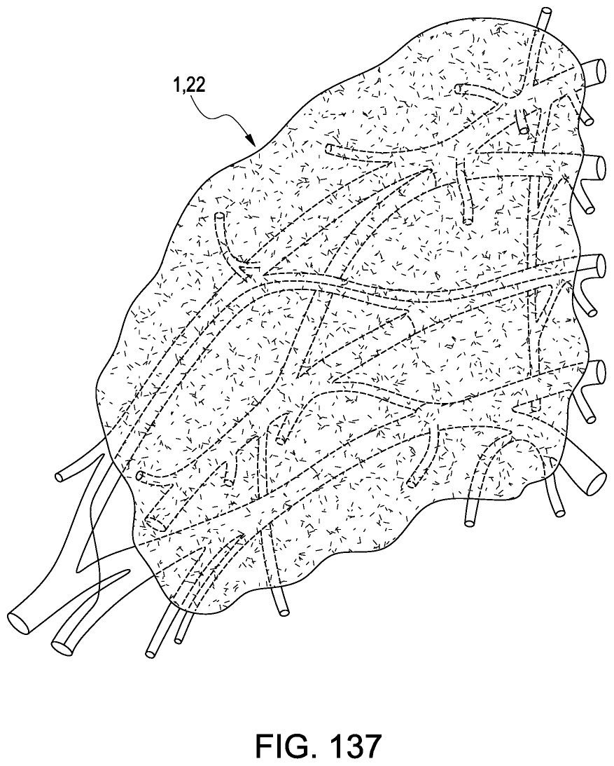

FIG. 137 is a drawing of placement location for a liquid mixture/cured electrode on the brachial plexus in a human (shown without a cured electrode in FIG. 1A) with an IPG implanted to electrically connect to the cured electrode and thereby fully depolarize all fibers of the brachial plexus on demand.

Other targets for the present invention include the cervical, thoracic, abdominal, and pelvic plexi. Prior art electrodes are designed to interface, for example, with a cylindrical nerve fiber (via a cuff) or a nerve nucleus (via a needle). The distribution of many fine nerves connected together and forming a neural network in the physical structure of a mesh requires a different type of interface. The cured electrode provides such an interface by allowing the physician to spray, paint, inject below, through and on-top of the body's irregular structures. For example, the sacral plexus and other plexi in the abdominal cavity, especially within the pelvic region, represent neural stimulation targets for specific organs (e.g., bladder, bowel, sexual organs) or the dualism between the sympathetic and parasympathetic arm of the autonomic nervous system.

Another example of applications for the present invention is stimulating baroreceptors in the abdominal and thoracic cavity. The body utilizes baroreceptors in two locations to drive sympathetic inhibition and parasympathetic activation with the goal of causing a combination of bradycardia and vasodilation to lower blood pressure when needed: one group of baroreceptors sits on the carotid sinus, the second one on the aortic arch. The aortic arch catches system high blood pressure (body wide) whereas the carotid sinus catches a stronger component of blood pressure differences heading cranially.

A selective stimulation (and/or potential temporary partial or full nerve block) of the baroreceptors or innervating nerve fibers connecting to said baroreceptors of the Aortic arch may provide a simple and more effective way to lower systemic blood pressure as compared to stimulation (and/or potential temporary partial or full nerve block) of the baroreceptors or innervating nerve fibers connecting to said baroreceptors of the carotid sinus.

The aorta may be surrounded with a cured electrode, focusing the liquid at the aortic arch and liquid nonconductor as mechanical stabilizer without impeding the ability of the aorta to stretch and contract as cardiac contractions push pressure waves through the aorta. Such a cured electrode is one again achieved under ultrasound or angiographic visualization in a laparoscopic approach using sterile, minimal-invasive technique.

Additionally, stimulation of the baroreceptors is possible at tissues connected to the sternum. Sternal thrusts are commonly performed on patients for whom breathing has stopped and conventional means of resuscitation are unavailable, impractical, or exhausted. The inferior vena cava which extends from the heart and connects to the liver through the diaphragm, may stimulate the baroreceptor reflex when stretched.

### Anchoring

The present invention prevents or minimizes migration of miniaturized prior art neural implants. Similar to the approach of securing a signal generator to a bone or other biological structures passing through or nearby a foramen, small neural signal generators may be secured very easily with liquid mixture or liquid nonconductor at their specific neural interface location. Liquid nonconductor may be used to provide an optimal mechanical anchoring of the small neural signal generator, whereas the active electrode area to the neural target of interest or the distant return electrode (which may just be 5 to 25 mm away from the neural target of interest on the other end of the small signal generator) may be formed using the liquid mixture which may use the same carrier medium as used to form the liquid nonconductor to provide an optimal mechanical integration of the small neural signal generator. A neural signal generator, anchored with liquid nonconductor, and/or liquid mixture, retains its mechanical position better than when held in place by only one suture or by conforming to anatomical structures of the implantation target. This is true for placement locations near single small nerves, single large nerves, and many collinear running nerves, near or inside a foramen, near or inside a neural plexus.

### Cardiac Applications

Many cardiac applications range from arrhythmias (heart not at correct rhythm, incorrect timing of contractions or of partial contractions of the heart), to bradycardia (slow heart), to tachycardia (fast heart), to bundle branch block before heart failure develops. In general, hearts age with each person/patient and as the muscle (and neuro-muscle) tissue in the heart undergoes small damages, fibrosis sets in and healthy heart tissue slowly becomes non-conductive and needs to be electrically bridged. Cardiac resynchronization pacemakers and other pacemakers as well as cardiac defibrillators are implanted to combat effects caused by this loss of conductivity inside the heart.

The present invention further comprises a dispenser and methods for improving electrical connectivity for the above cardiac pathologies:

(1) A dispenser is provided which comprises a catheter comprising a needle to sense electrical signals and stimulate cardiac muscles, with the dispenser being connected to a controller outside the body.
(2) A physician advances the catheter needle into the septum to a point where it senses the natural progression of the neural stimulation signal still reaching, and the physician location is marked as P1 on the controller.
(3) The catheter is advanced into the septum at a point a few millimeters (2.5 mm) distally to a point where stimulation, coordinated in time with the sensed signal earlier at P1, provides normal cardiac rhythm, this location being marked as P2
(4) The physician then actuates a flow of liquid mixture between P1 and P2 through the catheter needle to establish the electrical connection again
(5) The physician uses ECG data to verify electrical connectivity between P1 and P2

In addition to injecting liquid mixture through the catheter into the heart, the physician may also dispense liquid mixture from the outside of the heart through another dispenser adapted from a syringe, to establish electrical connectivity in a manner analogous to that described above in the ER/OR at runtime. Alternatively, the liquid mixture may be dispensed to temporarily block nerve conduction for open heart surgery comparable to current techniques where a fork is used to bypass electrical conduction for a given region of the heart, rendering it still and allow a surgeon to complete a procedure.

A further optional goal is to combat arrhythmias by utilizing a similar approach of injecting liquid mixture into the heart at locations of broken conductivity to prevent or combat arrhythmias, essentially providing parallels to the Purkinje fibers or hiss bundles and the like, using a fine needle to inject the liquid mixture, providing a conductive bypass around areas which should conduct but do not.

### Post-Surgical Pain

Many surgeries are associated with longer-lasting deep tissue pain, especially when bony structures, tendons and muscle pathways were re-aligned as part of the procedure. Examples are hip replacements, fixing and stabilizing a broken femur bone, knee and/or ankle surgeries, or procedures on the lower back such as fusing vertebrae or fixing a herniated disk. This pain may last several weeks as a result of a successful surgery and months to years as a result of a suboptimal surgical outcome. While post-op care generally involves the supply of opioids to the patient for follow-up pain treatment, the present invention provides to the localized pain block instead of systemic opioid use.

Some liquid mixture embodiments described herein provide a temporary cured electrode resorbed by the body over time. The temporary cured electrode, when placed as part of the surgery (likely towards the end of the procedure) and connected to nerves, tendons, or larger tissue groups within the surgical wound itself, does not require an excessive amount of surgeon time while providing the option for a local pain block relying on e.g. a transcutaneous stimulation paradigm later on with the added benefit that the waveforms needed for the specific patient's needs may more easily be designed and adjusted with a device outside the body.

In yet another implementation, liquid mixture not temporary and described herein is placed into the wound to connect to the same tissues when the physician determines that a long term electrical neural interface is required.

There is a need for a stable mechanical interface to organs and/or organ systems which may flexibly or rigidly move with the organ within the body without putting excessive strain on the organ's walls.

Pain treatment via the cured electrode connected to tendons and muscles for proprioception. Tendons are innervated with Golgi Tendon Organs, reporting information on the tendon strain and thereby the strain (and in part the stretch) of the muscle connected to the tendon. Similarly, muscles are innervated by nerves connecting to muscle spindles that measure the amount of stretch of the muscle. Together, Golgi tendon organs and muscle spindles are the primary sensory input organs as part of the body's peripheral afferent innervation providing the proprioceptive input to the spinal cord and brain that allow the calculation of one's body's position. When phantom limb pain is present in an individual having suffered muscle loss or damage to the limb's neural, muscular, body or other tissue structures, then the amount of information provided by Golgi tendon organs and muscle spindles may be reduced or otherwise changed in comparison to the amount and type of signals that were presented to the CNS from the periphery before.

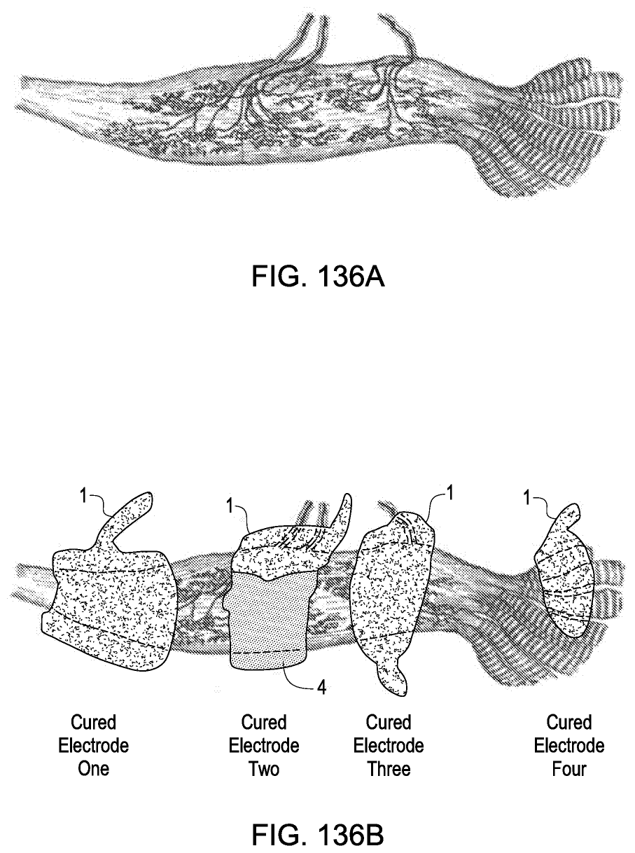

The present invention allows the placement of the liquid mixture around entire tendons as well as the surrounding or crisscrossing of muscles with liquid mixture to interface with the afferent nerves within the tendons and muscles of interest. This is especially of interest for cases where the afferent fibers form a mesh around and inside the tendon and/or muscle and do not allow a simple interface with prior art electrodes. FIG. 136A is a drawing of the basic anatomy of tendons and the Golgi tendon organs at the interface to the muscle fibers. These nerves are often small and a cuff electrode would only insufficiently cover the nerves as needed while micro-needle based electrode would need to be poked into the actual Golgi tendon organs or around the nerves to have any chance of effect. The present invention in contrast may be formed with conductive and non-conductive mixtures where needed to surround the tendon, the Golgi tendon organs as well as any muscle fibers of interest to provide a mechanically stable, flexible as needed, neural interface that lasts. FIG. 136A is a drawing of Golgi Tendon Organs (GTO) inside tendons which are locations for placement of cured electrodes. GTO are part of the Body's proprioceptive neural sensory input system. They acquire information about the tension of a tendon and the connected muscle, thereby allowing the body to assess muscle forces and indirectly through computation in the CNS the location and orientation of the body's arms, legs and other body parts. Image source: https://upload.wikimedia.org/wikipedia/commons/a/a1/Gray938.pn The liquid mixture may be placed as part of a surgery that takes place on the muscle, tendon or other tissues in an open wound or needle-based delivery approach; or liquid mixture may be placed separately with the needle-based or similar approaches. FIG. 136B is a diagram of Golgi Tendon Organs (GTO) with four cured electrode locations. Cured electrode #1 interfaces with the tendon and some GTO inside the tendon. Cured electrode #2 interfaces electrically with the nerves connecting to the GTO while mechanical stability is provided by liquid nonconductor surrounding the tendon with GTO inside. Cured electrode #3 interfaces with both, the GTO inside the tendon and the nerves connecting to the same GTO on the outside of the tendon. Cured electrode #4 interfaces with muscle fibers to stimulate sensory fibers such as muscle stretch receptors primarily and efferent fibers to drive muscle contraction second.

FIG. 137 is a diagram of a cured electrode surrounding the brachia plexus shown in FIG. 1A.

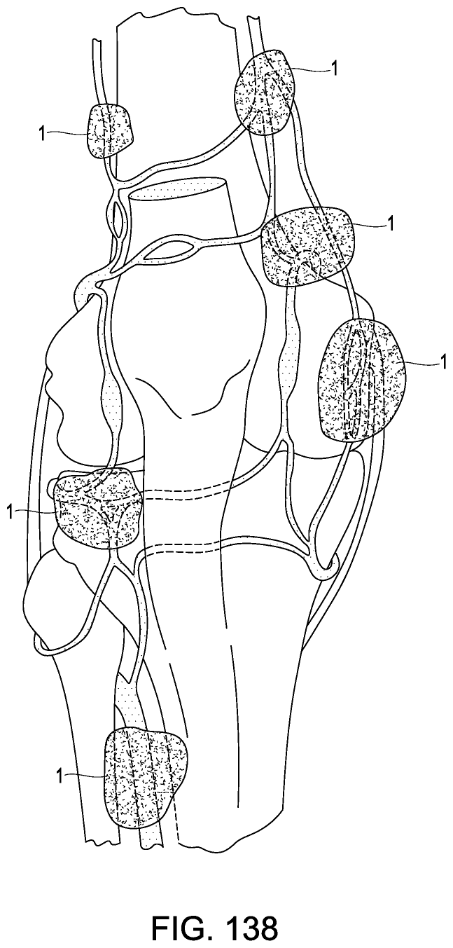

The thermally conductive cured electrode is also capable of cooling and heating blood that is entering a joint. The target tissue may be, but is not limited to, tissue that is part of a joint or multiple joints, potentially with adjacent tissue. By modulating the temperature of blood entering a joint, adjacent tissue or the nerves themselves, the perception of pain may be reduced, the overall inflammatory activity in a joint may be reduced, and a healing process of joint tissue may be facilitated, especially by cooling blood supplying joints and their adjacent tissue. One example is the knee joint. The femoral artery and the popliteal artery help form the arterial network or plexus, surrounding the knee joint. There are six main branches: two superior genicular arteries, two inferior genicular arteries, the descending genicular artery and the recurrent branch of anterior tibial artery. The medial genicular arteries penetrate the knee joint. FIG. 138 depicts the arteries of the knee joint and placement locations for thermally conductive cured electrodes in order to provide cooling to the knee joint for sufferers of an excessive localized inflammatory response such as in knee arthritis. Pain resulting from the localized hyper-inflammation may be reduced, angular range of motion as well as exerted forces across the knee joint may be increased with the aid of cooling the joint on demand following patient input activating i.e. current to a Peltier element, or following a repetitive cycle, over-night or other application paradigms to apply cooling to the knee joint at various locations. Examples of thermally conductive cured electrode placement locations indicated with six cured electrodes 1.

### Bladder and Bowel

Bladder and bowel control utilizing the cured electrode and TENS stimulation is generally achieved by stimulating the efferent (or afferent) sacral S1, S2, or S3 roots with the aim to either modulate activity in the sacral spinal cord and thereby initiate bladder contraction or relaxation on this indirect route, or by stimulating at least one of these sacral roots for an efferent contraction of the bladder. Bladder activity may further be modulated by stimulating the hypogastric plexuses and nerves or the inferior hypogastric plexus, for these plexi are in direct and indirect connection with both, the bladder as well as the sacral nerve roots. Specifically, the bladder receives motor innervation from both sympathetic fibers, most of which arise from the hypogastric plexuses and nerves, and parasympathetic fibers, which come from the pelvic splanchnic nerves and the inferior hypogastric plexus. There are no prior art treatments available that place an electrode into, near or around one of these plexi. There are also no treatment approaches that just connect to the bladder wall for stimulation, as there are no general electrode systems available that are flexible enough or versatile enough to be placed on or into an organ that stretches and flexes continuously throughout the day. For similar reasons, gastric and lower gut applications are limited. The injectable and cured electrode provides a new way of connecting to the bladder (as well as other organs) on the outside as well as the option for an injection of some liquid mixture partially into the organ wall for better integration. The liquid mixture may be placed on or around nerves that innervate the bladder, but likewise a placement around and into the before-mentioned hypogastric and inferior hypogastric plexi becomes possible as the liquid mixture requires the surgeon to simply distribute the electrically liquid mixture where she perceives e.g. a bladder contraction following a low-level stimulation from the liquid mixture dispenser. The ability to immediately verify that the just injected liquid mixture equally causes e.g. a bladder contraction provides the ability to verify interoperatively that the connection target was indeed hit and the liquid mixture may be assumed to having been properly placed.

The liquid mixture may then be dispensed from the target to a location just below the skin to essentially provide a connection point for an externally applied TENS stimulator. Utilizing a variety of electrical waveforms from 0.5 to 10 Hz for bladder relaxation to i.e. 30 Hz for bladder contraction may be applied with a TENS unit and thereby provide a simple pathway for bladder control. Furthermore, this approach may be used to achieve a connection to the pudendal nerve innervating the external urethral sphincter to provide bladder control, prevent unintended bladder leakage, and aid with bladder voiding when intended by utilizing KHFAC, depletion or non-destructive DC nerve block bilaterally applied to the pudendal nerve.

A similar approach may be utilized for bowel control with connection targets in the lower pelvic floor, the lower intestine walls (the cured electrode may be glued as a meander around or along the intestinal tube), the pudendal nerve, plexi in the vicinity of the bowel, as well as PNS nerves entering and exiting from the sacral spinal cord.

### Externally Cured and Manufactured Electrode

The cured electrode can also be applied to the outside of the body. This embodiment is “external” but uses the same principles and is the same invention as the same material which is injected or placed inside the body surgically. This embodiment can conform to structures on the outside of the body or structures on the inside of the body that form a somewhat stiff mechanical shape on the outside of the body, such as through elements of bony tissue or cartilage on the inside of the body that may be felt by palpation from the outside of the body. It can attach to micro-structures as a glue flows into small crevices and attaches itself, or it may form around more macroscopic structures, encasing these and conforming to them. One or more components of the cured electrode may cure a less viscous (mechanically less rigid) to a more viscous (mechanically more rigid) structure on the outside of the body while certain volume elements of the cured electrode become or remain electrically (thermally, optically, sound-conducting) highly conductive and other volume elements may be electrically (thermally, optically, sound-conducting) less conductive.

In one embodiment the external Cured electrode may be used to conform to the mechanically to-some-degree rigid structures of the ear, which possess a specific shape defined primarily by the cartilaginous tissue below the skin of the ear.

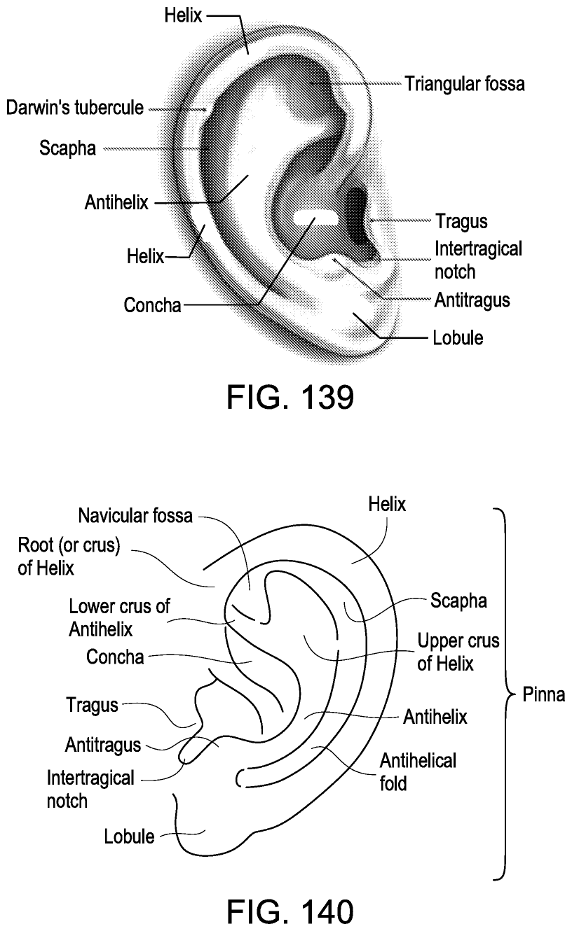

The external Cured electrode may conform to the grooves, wedges, notches and hollows near the canal of the ear. It also can conform to, surround or engulf one or more of all the structures of the ear including without limitation pinna, helix, antihelix, scapha, triangular, tragus, intertragical notch, antitragus, lobule, Darvin's Tubercle, ear notch, concha, root (crux) of the helix, nacivular fossa, and other grooves, wedges, notches and hollows. FIG. 139 and FIG. 140 show some anatomical structures of the outer ear.

In one embodiment the external cured electrode conforms to, surrounds or engulfs the Helix of the ear to reach from the lateral side (away from the head) of the ear (essentially the inside of the “sound funnel” the pinna of the ear forms) to the medial side (towards the head) of the ear (essentially the outside of the “sound funnel” the pinna of the ear forms).

While parts of the volume of the external cured electrode can be electrically conductive to provide a preferential current path from the output side of a neuro-stimulation signal generator to specific target nerves inside the ear (below the skin of the ear) or nerves of the near in the vicinity of the ear, other parts of the volume of the external cured electrode can be electrically non-conductive to limit the preferential current path from the output side of a neuro-stimulation signal generator to specific target nerves inside the ear (below the skin of the ear) or nerves of the near in the vicinity of the ear.

While parts of the volume of the external cured electrode can be electrically conductive to provide a preferential current path from the output side of a neuro-stimulation signal generator to specific target nerves inside the ear (below the skin of the ear) or nerves of the near in the vicinity of the ear, other parts of the volume of the external cured electrode can be electrically non-conductive and primarily mechanically adhering (akin to a glue) or mechanically fitting (akin to a wedge or anchor) that holds the external cured electrode (with or without active electronics, or simply connection wires being part of it).

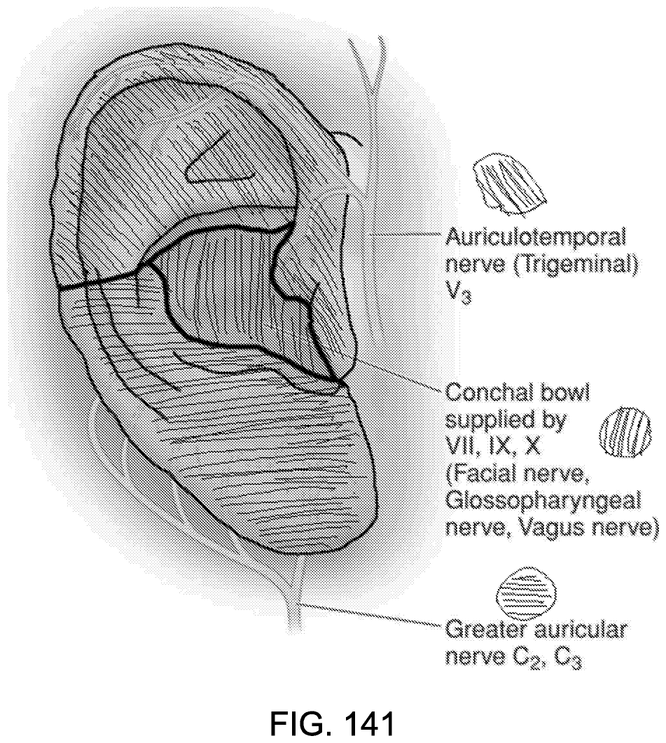

The external cured electrode can be applied for interfacing with Cranial Nerves. The ear is innervated by the Auriculotemporal nerve (a branch of the trigeminal nerve CN-V3), the Greater Auricular Nerve (C2, C3), the facial nerve (especially in the conchal bowl), glossopharyngeal nerve (especially in the conchal bowl) and the vagal nerve (especially in the conchal bowl). Some innervations of the ear are shown in FIG. 141. Nerves follow blood supply closely. Furthermore, influencing the amount of blood supply or the temperature of the blood supply may impact the neural activity of nerve fibers the blood supply is fed to.

Interfacing with the PNS at locations formerly not feasible is available with the external cured electrode. The external cured electrode allows for means of combining mechanical and electrical interfacing in one within a package that offers an optimum of patient comfort and electrical contact to the tissue.

The external cured electrode can be used to stimulate the vagal pathway to affect the clotting behavior in e.g. patients with hemophilia or other clotting disorders. The external cured electrode can be used to stimulate the vagal pathway to speed up blood clotting at an injury site. The external cured electrode can be placed prior to a potential injury and allow stimulation prior to a potential injury

Described herein are processes of molding the external cured electrode onto, into, on, in, near the anatomical structures of the ear with and without the use of molds.

The method of molding the external cured electrode directly onto a part of the ear or other part of the body's exterior comprises the steps of:
The physician places the electrically conductive mix at the location of preferential neural interfacing and may embed a wire/contact lead/electronics to provide the stimulation signal to the cured electrode which then connects this signal to the nerve below the ear's skin's surface.
The physician may place the electrically non-conductive mix directly in contact with the electrically conductive mix and provide added mechanical structure/interface/hold to and with the patient's ear or surrounding tissue of the head. Or the Physician may choose to only deploy the electrically conductive mix.
The external cured electrode is allowed to cure on the outside of the patient's skin.
Electrical stimulation may be used the same day or on another day to assess effects.
The external cured electrode may be left with the patient for a shorter time (seconds to minutes) or an extended time (hours to days). The patient may chose to leave the external cured electrode attached to the ear, or temporarily remove it before placing it back on/in/into contact with the ear, or permanently remove and/or replace with a new external cured electrode whenever needed.

An example of in-ear molded external cured electrode is depicted in FIG. 142. The cured electrode is placed on the ear to facilitate an interfacing with various neural target structures while providing specificity, selectivity and adhesive stability. Here the targeted nerve is the Auricular Temporal Nerve (CN-V3).

A method of creating molding an external cured electrode to the ear indirectly through a negative mold formed from the ear can comprise these steps:
A “negative mold” is formed off the ear of a patient. The negative mold is removed from the patient and retained by the physician. The negative mold may include just the ear, parts of the anatomy of the ear, and/or even parts of the anatomy of the patient's head near the ear.
A “negative mold” is processed to form a “positive” representation of the patient's ear. The positive representation of the patient's ear is retained by the physician or lab it is made by.
The positive mold may be painted/marked to allow a technician understand where the conductive and where the non-conductive elements of the cured electrode shall be located.
An external cured electrode is formed into/onto/at/near the positive mold of the ear by e.g. a technician. The technician may rely on markings on the positive mold to determine where the non-conductive elements of the cured electrode may be located. More details in 4.1.
The external cured electrode is allowed to cure in/on/at the positive mold.
The external cured electrode is removed from the positive mold with care to prevent damage.
The finished external cured electrode is given to the patient.
The finished external cured electrode may be further fitted and tested by the physician, and/or it may be further optimized in fit and electrically tested for stimulation effects such as sensation and/r and/or mechanically tested for optimal mechanical hold/attachment/support of the external cured electrode in/on/at the ear.

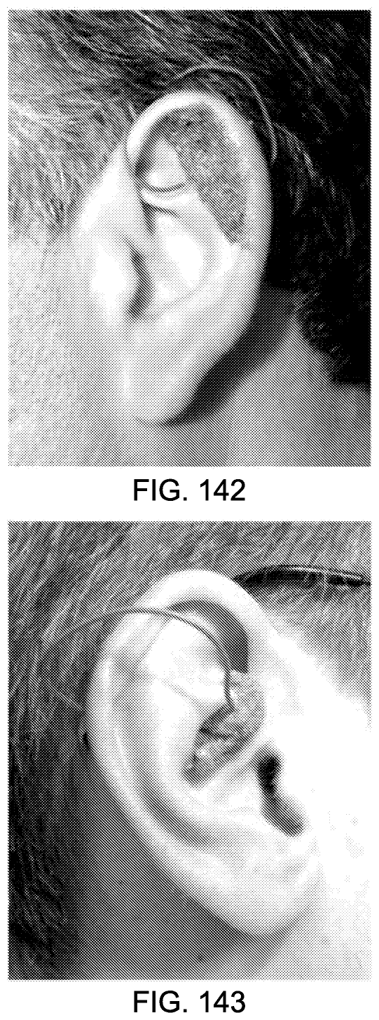

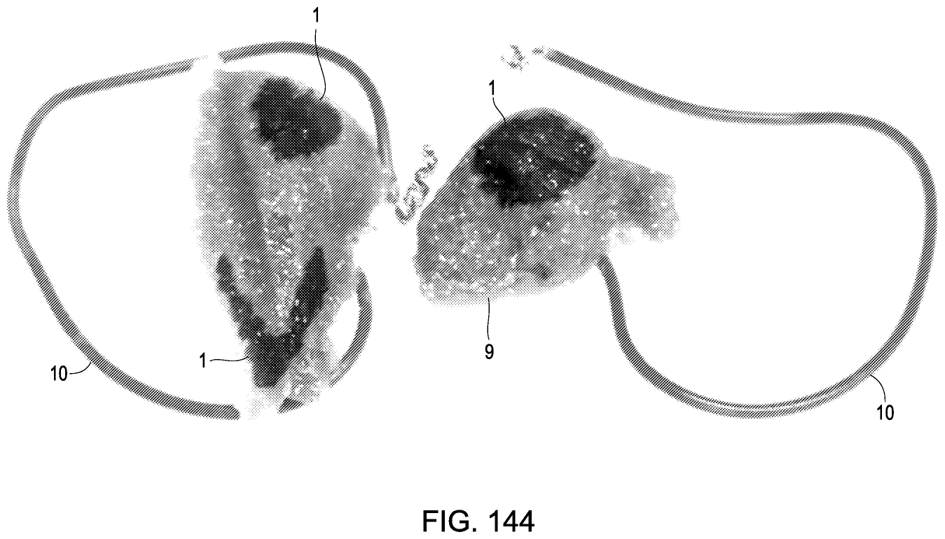

An example of in-positive-mold formed (molded) external cured electrode is presented in FIG. 143. The cured electrode placed on the ear to facilitate an interfacing with various neural target structures while providing specificity, selectivity and adhesive stability. Here the targeted nerve is the Vagal nerve (concha). FIG. 144 contains images of two external cured electrodes. Note the conductive wire to a signal generator and the central (darker) part of the cured electrodes with higher and the surrounding (lighter) part with lower electrical conductivity. Instead of electrical conductivity, one may utilize thermal conductivity and provide a cooling device to reduce the temperature of neural tissue or blood vessels in the ear. The cured electrode on the left is a two-contact version, the one on the right is a one-contact version (utilizing a distal return to close the electrical circuit).

The present invention includes novel combinations, mixtures, electrodes, kits, and delivery devices, and methods relating thereto, for injecting or otherwise placing into or on a living body where they are molded, cured and manufactured in or on the body itself, so that when they have been cured and manufactured they have conformed to the bodily structures, as follows:
1. a cured electrode for a target with contours in bodily tissue, said cured electrode comprising a mixture comprising fractional weights of conductive elements and a liquid nonconductor comprising a liquid phase and a solid phase, said liquid phase curable to said solid phase, said mixture during said liquid and solid phases of the mixture capable of conducting energy through the conductive elements, and said liquid mixture capable of being molded in the bodily tissue against and retaining at least a portion of the contours of the target;
2. a cured electrode for a target with contours in bodily tissue, said cured electrode comprising a mixture of fractional weights of conductive elements and a cured nonconductor, said cured electrode capable of conducting energy through the conductive elements, and said cured electrode being molded against and retaining at least a portion of the contours of the target;
3. a cured electrode for a target with contours in bodily tissue, said cured electrode comprising a mixture of fractional weights of conductive elements and a cured nonconductor, said cured electrode capable of conducting energy through the conductive elements, and said cured electrode being molded against and retaining at least a portion of the contours of the target; the volume impedance of the cured electrode being less than 10 Ohms per 1 mm of length and 1 mm\hat{ }2 in length times width.
4. a liquid mixture comprising fractional weights of conductive elements and a nonconductor in a liquid phase, said mixture in the liquid phase being moldable to a target with contours in bodily tissue and curable to a solid phase, said mixture capable of conducting energy in the liquid and solid phases;
5. a mixture comprising conductive elements, a hydrogel and water, said hydrogel comprising a liquid phase responsive to a cross-linker for curing to a solid phase, said mixture capable of conducting energy in the liquid and solid phases;
6. an electrode for a target with contours in bodily tissue comprising conductive elements, said conductive elements at least partially molded against the contours of the target, and said conductive elements capable of being encapsulated by the bodily tissue and receiving ingrowth of the body's cells, and capable of conducting energy; and
7. a kit for forming a moldable electrode that conforms at least a portion of its shape to a contour of a target in a bodily tissue, the kit comprising, a nonconductor in a liquid phase or capable of being dispersed in water, conductive elements, and optionally a set of instructions for mixing the nonconductor and conductive elements forming a moldable electrode from the hydrogel composition and the conductive elements in a body;
8. a dispenser for injecting a liquid or a liquid mixture into a bodily tissue against a target with contours, said dispenser comprising a first and a second chamber, a needle comprising a tip, a first and a second exit point near the tip, and a first plunger seated slideably with the first chamber and communicating fluidly with the first exit point, a second plunger seated slideably with the second chamber and communicating fluidly with the second exit point, so that when a user pushes the first plunger the said liquid or liquid mixture exits the first exit point and when the user pushes the second plunger the said liquid or liquid mixture exits the second exit point; further wherein the first and second chambers are coaxial; further wherein the first and second chambers each comprise a conical frustum connected to the needle; further wherein the dispenser comprises means for vibrating the dispenser or the liquid or liquid mixture, said means is selected from a group consisting of a sound transducer, an ultrasound transducer or a mass out of midline; further comprising an electrical stimulator near the tip; further comprising a light near the tip for curing the liquid or liquid mixture in the bodily tissue; further comprising an auger in the first or second chamber; further comprising a sensor for detecting speed of dispensing of the liquid or liquid mixture; and further comprising a third chamber for dispensing wire connected to a third exit point near the tip, said third chamber receiving a feed of the dispensing wire.
9. a method of forming an electrode in bodily tissue for a target with contours, comprising the steps of (a) introducing into the bodily tissue a liquid mixture comprising conductive elements and a nonconductor in a liquid phase, said carrier material capable of curing to a solid phase, said conductive elements being capable of conducting energy, (b) placing the mixture in the bodily tissue against at least a portion of the contours of the target, (c) molding the mixture against at least a portion of the contours of the target, and (d) allowing the carrier material to cure to the solid phase, such that the mixture during the solid phase of the carrier material is capable of retaining at least a portion of the contours of the target, and the conductive elements are capable of conducting current to the target; the method further comprises step z, prior to step a, of cooling the carrier material in the liquid phase to retard curing; the method further comprises step x, between steps b and c, of supplying symmetrical charge balanced pulse trains at 10 Hz, 10V to 50V amplitude voltage, controlled cathodic first. The method as in claim 1 further comprising step v, between steps b and c, of vibrating the liquid mixture by means selected from a group consisting of a sound transducer, an ultrasound transducer or a mass out of midline; the method further comprises step e, after step d, of placing a line of the mixture in the liquid phase of the carrier material from the target to a subcutaneous region; the method further comprises step t, after step f, of forming a contact pad in the subcutaneous region; and
10. a method of restoring or supplementing function for an electronic device in bodily tissue comprising: a. locating the electronic device in the bodily tissue, b. placing on the electronic device a mixture comprising conductive elements and a carrier material in a liquid phase, said liquid carrier material capable of curing to a solid phase, said conductive elements being capable of conducting current, c. positioning the mixture so as to restore or supplement a current flow between the electronic device and a target in the bodily tissue, and d. allowing the carrier material to cure to the solid phase, such that conductive elements are capable or restoring or supplementing the current flow between the electronic device and the target; and
11. a method of finding a target for stimulation in bodily tissue comprising a. introducing into the bodily tissue a liquid mixture comprising conductive elements and a carrier material in a liquid phase, said carrier material capable of curing to a solid phase, and said conductive elements being capable of conducting current, b. placing the liquid mixture in the bodily tissue against at least a portion of the contours of the target, c. providing the current through the liquid mixture, and d. verifying that the target has been stimulated by the current through the liquid mixture;
12. a method of providing current while manufacturing an electrode in bodily tissue for a target with contours comprising the steps of a. providing a dispenser comprising an exit point, a chamber containing a liquid mixture comprising conductive elements and a carrier material in a liquid phase, and an electrical stimulator near the exit point, said chamber communicating fluidly with the exit point, said electrical stimulator connected to a current source, said conductive elements being capable of conducting current, b. placing the exit point of the dispenser in the bodily tissue against the contours of the target, c. dispensing the liquid mixture from the exit point of the dispenser, d. molding the liquid mixture against the contours of the target, and e. providing current to the electrical stimulator in contact with the liquid mixture against the contours of the target, such that the electrical stimulator is capable of stimulating the conductive elements and the conductive elements are capable of conducting current to the target. The method as in above wherein the current comprises anodic or cathodic current, or comprises a repetitive or intermittent pulse of approximately 200 us width, 0.1 to 10 mA current amplitude, cathodic first vs. distal return, symmetrical charge.
13. a method of implanting an electrode with an anchor in a bodily tissue comprising a. placing an electrode near a target in the bodily tissue, b. introducing into the bodily tissue a liquid mixture comprising conductive elements and a carrier material in a liquid phase, said carrier material capable of curing to a solid phase, and said conductive elements being capable of conducting current, c. placing the liquid mixture in the bodily tissue against at least a portion of the contours of the target, d. placing a liquid nonconductor in a first area in the bodily tissue which is secure, e. connecting the liquid nonconductor to the liquid mixture, and f allowing the liquid mixture and the liquid nonconductor to cure and bond together;
14. a method of delivering a liquid mixture with vibration to a target in bodily tissue comprising a. providing a dispenser containing a liquid mixture comprising conductive elements and a carrier material in a liquid phase, said carrier material capable of curing to a solid phase, and said conductive elements being capable of conducting current, and b. vibrating the dispenser or the liquid mixture, such that the liquid mixture is capable of flowing to the target in bodily tissue;
15. a method of installing a suture for removal of a cured electrode comprising a. introducing into the bodily tissue a liquid mixture comprising conductive elements and a carrier material in a liquid phase, said carrier material capable of curing to a solid phase, said conductive elements being capable of conducting current, b. placing the liquid mixture in the bodily tissue against at least a portion of the contours of the target, c. molding the liquid mixture against at least a portion of the contours of the target, d. tying a suture on the liquid mixture, and e. allowing the carrier material to cure to the solid phase, such that by pulling the suture during the solid phase of the carrier material is capable of severing at least a portion of the cured electrode;
16. a method of placing a liquid mixture into or near or around a tissue target in a living body, the liquid electrode undergoing a phase transition at said location inside the body and forming a cured electrode, enabling at a later point the contacting of the cured electrode with a probe to supply current to ablate the tissue in contact with the cured electrode, a) the current being radio frequency ablation current (200 to 400 kHz) that is able to heat tissue in the direct vicinity of the cured electrode; the probe being a needle that passes through the skin and mechanically connects to the cured electrode placed inside the body, b) the current being radio frequency transmission ablation current (800 to 5000 kHz) that is able to heat tissue in the direct vicinity of the cured electrode; the probe placed as an electrode on the outside of the skin from more than one point with the intent to concentrate the current through crossover or interference below the skin at the location of the cured electrode placed inside the body, c) the current being direct current that is able to either temporarily block nerve tissue in the direct vicinity of the cured electrode or able to permanently ablate tissue in direct vicinity of the cured electrode by chemical means through changes in pH that result from prolonged exposure of tissue and surrounding fluids to a DC field; the probe being an electrode on the outside of the skin form where the current travels through the skin to a implanted cured electrode that may have an extension towards the skin for a reduction in impedance from the skin to the cured electrode around the target tissue to be ablated.
17. A method of manufacturing and dispensing the mixture by first freezing the components of the liquid mixture before mixing them into a homogeneous blend which may be kept frozen for transport to the injection location, were the frozen liquid electrode is thawed, may be stirred during thawing and dispensing and being dispensed as need be.
18. A device able to thaw a frozen liquid mixture that measures the temperature of the liquid electrode during thawing, records time and reports on both, readiness for injection of the liquid electrode and end of work time for the liquid electrode as the work time comes to an end, ensuring physician comfort and patient safety.
19. A liquid mixture that may be placed adjacent to cancerous tissue inside a living body where it cures in place to form a cured electrode, to electrically, magnetically or thermally excite or ablate the tissue nearby the cured electrode to reduce tumor growth or destroy cancerous tissue.
20. A liquid mixture that may be placed near, around or into cancerous tissue inside a living body where it cures in place to form a cured electrode nearby blood vessels that may provide oxygenated and nutrient rich blood to specific tissue, organs or cancerous tissue, with the intent to either cool or heat or ablate the tissue nearby the cured electrode to reduce tumor growth, tumor spread (i.e. by cooling) or destroy cancerous tissue.
   and, for these combinations, materials, electrodes, kits, delivery devices and methods, the following further conditions apply:
a. The liquid nonconductor or carrier material can be a hydrogel, a silicone, fibrin glue, a tissue gluea protein glue, a conducting polymer, a cyanoacrylate, a bone cement, a dental resin, a dental cement, or a bone cement.
b. In the case of a hydrogel it comprises a polyethylene glycol comprising the general structure X—(O—CH2-CH2)n-Y where n is a variable number of repeat units and X and Y are functional groups at terminal ends.
c. The energy conducted can be electrical current, electromagnetic energy, heat, vibratory including sound, or light.
d. When electrical current is the energy conducted, said current constitutes charge transfer by direct exchange of electrons between said conductive elements.
e. The cured electrode further comprises pores, and the cured electrode comprises outermost dimensions defining an overall volume, and the pores define spaces which together total a subset volume of the overall volume, and the subset volume is within a range of about 0.1% to 20% or 0.1% to 40% of the overall volume.
f. Where the energy conducted is electrical current and heat, said conductive elements comprise a material selected from a group comprising gold, gold bonding wire bits, silver, platinum, graphene, graphite, carbon tubes, stainless steel, 316 stainless steel, copper, aluminum oxides, bronze, vanadium, niobium, iron, rhodium, tungsten, titanium, tantalum, gallium, arsenic, antimony, bismuth, nitinol, diamond-coated sand, gold-coated steel or iron, iridium, iridium oxide, ionized proteins, and poly(3,4-ethylenedioxythiophene) polystyrene sulfonate. Where the energy is heat but not electrical current, said conductive elements comprise a material selected from a group comprising diamond and graphene.
g. Where the energy is electromagnetic, said conductive elements comprise a material selected from a group comprising a sintered Nd2Fe14B compound of high saturation magnetization, a rare-earth magnet, an alloy of neodymium, iron and boron to form the Nd2Fe14B tetragonal crystalline structure, stainless steel with ferromagnetic iron components such as 440 or 420 stainless steel, and ferrite elements in stainless steel, cobalt or nickel, a transition metal-metalloid component (Fe, Co or Ni) and (B, C, S, P or Al) and alloys of the foregoing, ferromagnetic materials, ferromagnetic metallic alloys and ferromagnetic materials.
h. Where the energy conducted is light, said conductive elements comprise a material selected from a group consisting of clear diamond, polished glass beads, or metallic elements such as germanium based alloys.
i. Where the energy conducted is vibratory including sound, said conductive elements comprise a material selected from a group consisting of metals and other non-compressible materials that do not function as a damper but instead as conductor at the chosen vibratory frequencies.
j. In some embodiments, the fractional weight of said conductive elements is within a range of about 65-85% or, in another embodiments, within a range of about 50-64%.
k. The said conductive elements comprise a longest and a shortest dimension, and the conductive elements comprise an aspect ratio of the longest dimension divided by the shortest dimension, and the shorter or longer dimensions of at least a portion of the conductive elements is at least one micron, and the aspect ratio of at least a portion of the conductive elements is less than 3:1 in some embodiments, and in other embodiments is within a range between 3:1 and less than 10:1, and in other embodiments is at least 10:1.
l. In some embodiments, said conductive elements comprise interlocking features selected from a group comprising hooks, loops and coils.
m. The cured electrode may further comprise an immunoreactive agent selected from a group comprising cells, whole blood, blood serum, biodegradable polymers sugars, amino acids, proteins, iron, lipopolysaccharides, collagen, hyaluronic acid, fibrin and pharmacological agents.
n. The cured electrode may further comprise an anti-inflammatory agent selected from a group comprising steroids, anti-oxidants, superoxide dismutase mimetics and non-steroidal anti-inflammatory drugs.
o. The cured electrode may further comprise a hemostatic agent selected from a group comprising microfibrillar collagen hemostat, chitosan, kaolin, zeolite, anhydrous aluminum sulfate and fibrin glue.
p. The cured electrode may further comprise a pharmacological agent selected from a group comprising an antibiotic, an analgesic and an anesthetic.
q. The cured electrode may further comprising a radio-opaque agent selected from a group comprising fluroscein and platinum.
r. When the energy conducted is electrical current, the electrical resistance of the conductive elements when formed as an electrode does not exceed 10 ohms, and the base conductivity of the cured elements before being mixed into the electrode does not exceed 1 ohm.
s. The cured electrode may further comprise a nonconductive layer selected from the group comprising a hydrogel, a silicone, fibrin glue, a protein glue, a conducting polymer, a cyanoacrylate, a bone cement, a dental resin, a dental cement, or a bone cement, all of which are capable of bonding to the mixture and the target and the bodily tissue, and the nonconductive layer may further comprise nonconductive elements selected from a group consisting of ceramic and glass.
t. When the liquid nonconductor or carrier material is a hydrogel, the hydrogel in the solid phase is capable of resorption by the bodily tissue, such that fibrous tissue is capable of attaching to the conductive elements.
u. Further, for the cured electrode whose liquid nonconductor or carrier is a hydrogel, the liquid nonconductor further comprises a cross-linker for the hydrogel, wherein the cross-linker may comprise trilysine, and the liquid nonconductor may further comprise glycerine.

1. A method of modifying a metabolic activity of a tissue within a body comprising
a. providing a mixture comprising thermally conductive elements and a carrier, said carrier comprising a liquid phase at a first time and a biocompatible solid phase at a second time, said liquid phase of the carrier capable of curing by a phase transition to the solid phase in said body,
b. introducing the mixture in the liquid phase of the carrier into said body so that the carrier and conductive elements can be molded against contours of a target within said body distinct from and communicating with said bodily tissue,
c. allowing the carrier in the liquid phase to cure to the solid phase,
d. connecting the conductive elements to a source or a drain of thermal energy, and
e. allowing thermal energy to transmit through the thermally conductive elements and to change a temperature of the target, thereby modifying the metabolic activity of said bodily tissue.

2. The method of claim 1 wherein introducing the mixture into the body is performed through an injection.

3. The method of claim 1 wherein the thermal energy transmits toward the target.

4. The method of claim 1 wherein the thermal energy transmits away from the target.

5. The method of claim 1 wherein the target is a blood vessel.

6. The method of claim 5 wherein a change in the temperature of blood flow in the blood vessel to said bodily tissue modifies the metabolic activity of said bodily tissue.

7. The method of claim 5 wherein a change in the temperature of blood flow is a decrease resulting in a reduction of the metabolic activity of said bodily tissue.

8. The method of claim 5 wherein a change in the temperature of blood flow is an increase resulting in a raising of the metabolic activity of said bodily tissue.

9. The method of claim 1 wherein said bodily tissue is an organ.

10. The method of claim 1 wherein said bodily tissue is a nerve.

11. The method of claim 1 wherein said bodily tissue is a gland.

12. The method of claim 1 wherein said bodily tissue is cerebrospinal fluid.

13. The method of claim 1 wherein said bodily tissue is skeletal muscle.

14. The method of claim 5, wherein step d further comprises locating a temperature sensor on said blood vessel downstream from a point on the blood vessel connected to the source or the drain, said temperature sensor being in communication with a controller for the source or the drain.

15. The method of claim 1 wherein the target is a nerve, and the change in the temperature of the nerve modifies the probability of the nerve to generate action potentials.

16. The method of claim 15 wherein the change in the temperature of the nerve results in an at least partial pain blockade.

17. The method of claim 16 wherein the pain blockade is temporary.

18. The method of claim 16 wherein the pain blockade is permanent.

19. The method of claim 15 wherein the change in the temperature of the nerve results in a reduction in spasticity in muscle connected directly or indirectly to said nerve.

20. The method of claim 19 wherein the reduction in spasticity is temporary.

21. The method of claim 19 wherein the reduction in spasticity is permanent.

22. The method of claim 7 wherein said bodily tissue is a tumor and the decrease in the metabolic activity of the tumor results in slowing or stopping growth of the tumor.

23. The method of claim 7 wherein said bodily tissue is a tumor and the decrease in the metabolic activity of the tumor results in slowing or stopping of metastatic activity of the tumor.

24. The method of claim 8 wherein said bodily tissue is a tumor and the increase in the metabolic activity of the tumor increases the uptake of an anti-cancer compound by the tumor.

25. The method of claim 1 wherein the target is a lymphatic vessel and the change in the temperature of the lymphatic vessel results in modulation of regulation of an immune system.

26. The method of claim 15 wherein the change in the temperature of the nerve results in modulation of regulation of an organ.

27. The method of claim 1 wherein the source or the drain is located internal to the body communicating thermally with a source or a drain located external to the body.

28. The method of claim 1 wherein the source or the drain is located external to the body, and the conductive elements communicate thermally with the source or the drain located external to the body.

29. The method of claim 1 wherein step 1a further comprises locating at least a portion of the mixture in a region which is subcutaneous or underneath the surface of an organ.

30. The method of claim 15 further comprising step f, initiating an electrical block of the nerve.

31. The method of claim 30 further comprising step g, heating the nerve.
